(19) United States
(12) Patent Application Publication
(10) Pub. No.: US 2014/0073017 A1
(43) Pub. Date: Mar. 13, 2014

(54) CELLULASE COMPOSITIONS AND METHODS OF USING THE SAME FOR IMPROVED CONVERSION OF LIGNOCELLULOSIC BIOMASS INTO FERMENTABLE SUGARS

(71) Applicants: Thijs Kaper, Half Moon Bay, CA (US); Igor Nikolaev, Noordwijk (NL); Suzanne E. Lantz, San Carlos, CA (US); Meredith K. Fujdala, San Jose, CA (US); Megan Y. Hsi, San Jose, CA (US)

(72) Inventors: Thijs Kaper, Half Moon Bay, CA (US); Igor Nikolaev, Noordwijk (NL); Suzanne E. Lantz, San Carlos, CA (US); Meredith K. Fujdala, San Jose, CA (US); Megan Y. Hsi, San Jose, CA (US)

(73) Assignee: DANISCO US INC., Palo Alto, CA (US)

(21) Appl. No.: 14/004,872

(22) PCT Filed: Mar. 16, 2012
(86) PCT No.: PCT/US12/29498

§ 371 Date: Nov. 19, 2013

Related U.S. Application Data
(60) Provisional application No. 61/453,918, filed on Mar. 17, 2011.

(51) Int. Cl. C12N 9/42

(52) U.S. Cl. CPC C12N 9/2437
USPC 435/99; 435/209; 536/23.2; 435/320.1; 435/252.3; 435/254.11; 435/252.31; 435/252.33; 435/254.6; 435/254.3

(57) ABSTRACT

The present invention relates to compositions that can be used in hydrolyzing biomass such as compositions comprising a polypeptide having β-glucosidase activity, methods for hydrolyzing biomass material, and methods for improving the stability and saccharification efficacy of a composition comprising such β-glucosidase polypeptides and/or activity.

### CROSS REFERENCE TO RELATED APPLICATIONS

This application claims the benefit of U.S. Provisional Application No. 61/453,918, filed Mar. 17, 2011, which is hereby incorporated by reference in its entirety.

### FIELD OF THE INVENTION

The present disclosure generally pertains to certain β-glucosidase enzymes, and engineered β-glucosidase enzyme compositions, β-glucosidase fermentation broth compositions, and other compositions comprising such β-glucosidases, and methods of making or using the same in a research, industrial or commercial setting, e.g., for saccharification or conversion of biomass materials comprising hemicelluloses, and optionally cellulose, into fermentable sugars.

### BACKGROUND OF THE INVENTION

Bioconversion of renewable lignocellulosic biomass to a fermentable sugar that is subsequently fermented to produce alcohol (e.g., ethanol) as an alternative to liquid fuels has attracted the intensive attention of researchers since the 1970s, when the oil crisis occurred (Bungay, H. R., “Energy: the biomass options”. NY: Wiley; 1981; Olsson L, Hahn-Hagerdal B. Enzyme Microb Technol 1996, 18:312-31; Zaldivar, J et al., Appl Microbiol Biotechnol 2001, 56: 17-34; Galbe, M et al., Appl Microbiol Biotechnol 2002, 59:618-28). Ethanol has been used as a 10% blend to gasoline in the U.S. or as a neat fuel for vehicles in Brazil in the past decades. The importance of fuel bioethanol will increase in parallel with increasing oil prices and gradual depletion of its sources. Additionally, fermentable sugars are increasingly used to produce plastics, polymers and other bio-based products. Thus, the demand for abundant low cost fermentable sugars, which can be used in lieu of petroleum-based fuel feedstock, grows rapidly.

Chiefly among the useful renewable biomass materials are cellulose and hemicellulose (xylans), which can be converted into fermentable sugars. The enzymatic conversion of these polysaccharides to soluble sugars, e.g., glucose, xylose, arabinose, galactose, mannose, and/or other hexoses and pentoses, occurs due to combined actions of various enzymes. For example, endo-1,4-β-glucanases (EG) and exo-cellobiohydrolases (CBH) catalyze the hydrolysis of insoluble cellulose to cellooligosaccharides (e.g., with cellobiose being a main product), while β-glucosidases (BGL) convert the oligosaccharides to glucose. Xylanases together with other accessory proteins (hemicellulases; non-limiting examples of which include L-α-arabinofuranosidases, feruloyl and acetylxylan esterases, glucuronidases, and β-xylosidases) catalyze the hydrolysis of hemicelluloses.

The cell walls of plants are composed of a heterogenous mixture of complex polysaccharides that interact through covalent and noncovalent means. Complex polysaccharides of higher plant cell walls include, e.g., cellulose (β-1,4 glucan) which generally makes up 35-50% of carbon found in cell wall components. Cellulose polymers self associate through hydrogen bonding, van der Waals interactions and hydrophobic interactions to form semi-crystalline cellulose microfibrils. These microfibrils also include noncrystalline regions, generally known as amorphous cellulose. The cellulose microfibrils are embedded in a matrix formed of hemicelluloses (including, e.g., xylans, arabinans, and mannans), pectins (e.g., galacturonans and galactans), and various other β-1,3 and β-1,4 glucans. These matrix polymers are often substituted with, e.g., arabinose, galactose and/or xylose residues to yield highly complex arabinoxylans, arabinogalactans, galactomannans, and xyloglucans. The hemicellulose matrix is, in turn, surrounded by polyphenolic lignin.

In order to obtain useful fermentable sugars from biomass materials, the lignin is typically permeabilized and the hemicellulose disrupted to allow access by the cellulose-hydrolyzing enzymes. A consortium of enzymatic activities may be necessary to break down the complex matrix of a biomass material before fermentable sugars can be obtained.

Regardless of the type of cellulosic feedstock, the cost and hydrolytic efficiency of enzymes are major factors that restrict the commercialization of biomass bioconversion processes. The production costs of microbially produced enzymes are tightly connected with the productivity of the enzyme-producing strain and the final activity yield in the fermentation broth. The hydrolytic efficiency of a multienzyme complex can depend on a multitude of factors, e.g., properties of individual enzymes, the synergies among them, and their ratio in the multienzyme blend.

There exists a need in the art to identify enzyme and/or enzymatic compositions that are capable of converting plant and/or other cellulosic or hemicellulosic materials into fermentable sugars with sufficient or improved efficacy, improved fermentable sugar yields, and/or improved capacity to act on a greater variety of cellulosic or hemicellulosic materials. The improved methods and compositions described herein provide such enzymatic compositions, capable of yielding fermentable sugars at low cost and from renewable sources.

Patents, patent applications, documents, nucleotide/protein sequence database accession numbers and articles cited herein are incorporated herein by reference in their entirety.

### BRIEF SUMMARY OF THE INVENTION

Provided herein are a number of β-glucosidase polypeptides, including variants, mutants, hybrid/chimeric/fusion enzymes, nucleic acids encoding these polypeptides, compositions comprising such polypeptides and methods of using these compositions. The compositions herein are, in some aspects, non-naturally occurring cellulase compositions. The compositions can further comprise one or more hemicellulases, and as such are hemicellulase compositions. In some aspects, the compositions can be used in a saccharification process, converting various biomass materials into fermentable sugars. In some aspects, the compositions herein provide improved saccharification efficacy or efficiency and other advantages. Also provided herein are cells, e.g., recombinantly engineered host cells, fermentation broths derived from these cells, and methods or processes of using these cells or fermentation broths. Furthermore business methods of using such polypeptides, nucleic acids encoding these polypeptides, and compositions comprising such polypeptides are described and contemplated in the present invention.

In certain aspects, the disclosure provides for a non-naturally occurring cellulase composition comprising a β-glucosidase polypeptide, which is a chimera (or hybrid, or fusion, which terms are used interchangeably herein to refer to the same concept) of at least two β-glucosidase sequences. In some aspects, the non-naturally occurring cellulase composition comprises β-glucosidase activity. The composition may further comprise one or more of xylanase, β-xylosidase, and/or L-α-arabinofuranosidase activities. Thus the composition may be a hemicellulase composition. The non-naturally occurring cellulase/hemicellulase composition comprises components derived from at least two different sources. In some aspects, the non-naturally occurring cellulase/hemicellulase composition comprises one or more naturally occurring hemicellulases. The β-glucosidase polypeptides in the composition may further comprise one or more glycosylation sites. In some aspects, the β-glucosidase polypeptide comprises an N-terminal sequence and a C-terminal sequence, wherein each of the N-terminal sequence or the C-terminal sequence comprises one or more sub-sequences derived from different β-glucosidases. In certain aspects, the N-terminal and C-terminal sequences are derived from different sources. In some embodiments, at least two of the one or more sub-sequences of the N-terminal and the C-terminal sequences are derived from different sources. In some aspects, either the N-terminal sequence or the C-terminal sequence further comprises a loop region sequence of about 3, 4, 5, 6, 7, 8, 9, 10, or 11 amino acid residues in length. In certain embodiments, the N-terminal sequence and the C-terminal sequence are immediately adjacent or directly connected. In other embodiments, the N-terminal and C-terminal sequences are not immediately adjacent, but rather, they are functionally connected via a linker domain. In certain embodiments, the linker domain is centrally located (e.g., not located at either the N-terminal or the C-terminal) of the chimeric polypeptide. In certain embodiments, neither the N-terminal sequence nor the C-terminal sequence of the hybrid polypeptide comprises a loop sequence. Instead, the linker domain comprises the loop sequence. In some aspects, the N-terminal sequence comprises a first amino acid sequence of a β-glucosidase or a variant thereof that is at least about 200 (e.g., about 200, 250, 300, 350, 400, 450, 500, 550, or 600) residues in length. In some aspects, the N-terminal sequence comprises one or more or all of the polypeptide sequence motifs represented by SEQ ID NOs:136-148. In some aspects, the C-terminal sequence comprises a second amino acid sequence of a β-glucosidase or a variant thereof that is at least about 50 (e.g., about 50, 75, 100, 125, 150, 175, or 200) amino acid residues in length. In some aspects, the C-terminal sequence comprises one or more or all of the polypeptide sequence motifs represented by SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In some aspects, either the C-terminal or the N-terminal sequence comprises a loop sequence, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the C-terminal nor the N-terminal sequence comprises a loop sequence. In some embodiments, the C-terminal sequence and the N-terminal sequence are connected via a linker domain that comprises a loop sequence, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In certain embodiments, the β-glucosidase polypeptide comprises a sequence that has is at least about 65%, (e.g., at least about 65%, 70%, 75%, 80%, 85%, 90%, 91%, 92%, 93%, 94%, 95%, 96%, 97%, 98%, 99%, or 100%) identity to SEQ ID NO:135. In some embodiments, the polypeptide having β-glucosidase activity (i.e., the β-glucosidase polypeptide) is encoded by a nucleotide that has at least about 65% (e.g., at least about 65%, 70%, 75%, 80%, 85%, 90%, 91%, 92%, 93%, 94%, 95%, 96%, 97%, 98%, 99%, or 100%) identity to SEQ ID NO:83, or by a polynucleotide capable of hybridizing under high stringency conditions to SEQ ID NO:83 or a complement thereof. In some aspects, the β-glucosidase polypeptide(s) in the non-naturally occurring cellulase or hemicellulase composition has improved stability over any of the native enzymes from which each C-terminal and/or the N-terminal sequences of the chimeric polypeptide was derived. In some aspects, the improved stability comprises an improvement in proteolytic stability during storage, expression or production processes. In some aspects, the improved stability comprises a decrease in rate or extent of an associated enzymatic activity loss during storage or production conditions, wherein the enzymatic activity loss is preferably less than about 50%, less than about 40%, less than about 30%, or less than about 20%, more preferably less than 15%, or less than 10%.

The polypeptides of the disclosure can suitably be obtained and/or used in “substantially pure” form. For example, a polypeptide of the disclosure constitutes at least about 80 wt. % (e.g., at least about 85 wt. %, 90 wt. %, 91 wt. %, 92 wt. %, 93 wt. %, 94 wt. %, 95 wt. %, 96 wt. %, 97 wt. %, 98 wt. %, or 99 wt. %) of the total protein in a given composition, which also includes other ingredients such as a buffer or solution.

In some aspects, the disclosure provides nucleic acid encoding the β-glucosidase polypeptide, including the variants, mutants and hybrid/fusion/chimeric polypeptides. For example, the disclosure provides isolated nucleic acid encoding the β-glucosidase polypeptide, wherein the nucleic acid is one that has at least about 65% (e.g., at least about 65%, 70%, 75%, 80%, 85%, 90%, 91%, 92%, 93%, 94%, 95%, 96%, 97%, 98%, 99%, or 100%) identity to SEQ ID NO:83, or is one that is capable of hybridizing under high stringency conditions to SEQ ID NO:83 or to a complement thereof. The disclosure also provides host cells comprising such nucleic acid molecules. In some embodiments, the disclosure further provides promoters and vectors suitable for use with the nucleic acid molecules and the host cells. In certain aspects, the disclosure provides compositions prepared by fermenting the host cells, including cellulase compositions or hemicellulase compositions. As such the disclosure provides fermentation broth compositions.

In some aspects, the disclosure provides methods of using the compositions, polypeptides, cells, or nucleic acids encoding the polypeptides herein to achieve saccharification of biomass substrates/materials. In certain embodiments, the biomass substrates/materials are suitably pre-treated or subject to a suitable pretreatment methods. In some embodiments, the disclosure also provides certain commercial or business methods associated with the compositions, polypeptides, cells, or nucleic acids described herein.

### BRIEF DESCRIPTION OF THE DRAWINGS

The following figures and tables are meant to be illustrative without limiting the scope and content of the instant disclosure or the claims herein.

FIG. 1: provides a summary of the sequence identifiers used in the present disclosure of various enzymes and nucleotides encoding certain of these enzymes

FIG. 2 provides conserved residues among certain β-glucosidase (e.g., Fv3C) homologs, predicted based on the crystal structure of T. neapolitana Bgl3B complexed with glucose in the −1 subsite (crystal structure at Protein Data Bank Accession: pdb:2X41).

FIG. 3: provides the enzyme composition of a fermentation broth produced by the T. reesei integrated strain H3A.

FIGS. 4A-4E: FIG. 4A lists the enzymes (purified or unpurified) that were individually added to each of the samples in Example 2, and the stock protein concentrations of these enzymes. FIG. 4B depicts the amount of glucose release following saccharification of dilute ammonia pretreated corncob by adding enzyme compositions comprising various purified or non-purified enzymes of FIG. 4A, which were added to T. reesei integrated strain H3A, in accordance with Example 2. FIG. 4C depicts the amount of cellobiose release following saccharification of dilute ammonia pretreated corncob by adding enzyme compositions comprising various purified or non-purified enzymes of FIG. 4A, which were added to T. reesei integrated strain H3A, in accordance with Example 2. FIG. 4D depicts the amount of xylobiose release following saccharification of dilute ammonia pretreated corncob by adding enzyme compositions comprising various purified or non-purified enzymes of FIG. 4A, which were added to T. reesei integrated strain H3A, in accordance with Example 2. FIG. 4E depicts the amount of xylose release following saccharification of dilute ammonia pretreated corncob by adding enzyme compositions comprising various purified or non-purified enzymes of FIG. 4A, which were added to T. reesei integrated strain H3A, in accordance with Example 2.

FIGS. 5A-5B: FIG. 5A lists β-glucosidase activity of a number of β-glucosidase homologs, including T. reesei Bgl1 (Tr3A), A. niger Bglu (An3A), Fv3C, Fv3D, and Pa3C. Activity on cellobiose and CNPG substrates were measured, in accordance with Example 4; FIG. 5B compares the activity of another group of β-glucosidase homologs, relative to T. reesei Bgl1, on cellobiose and CNPG substrates, in accordance with Example 5A.

FIG. 6: lists the relative weights of the enzymes in an enzyme mixture/composition tested in Example 5B-D.

FIG. 7: provides a comparison of the effects of enzyme compositions on dilute ammonia pre-treated corncob.

FIGS. 8A-8B: FIG. 8A depicts Fv3A nucleotide sequence (SEQ ID NO:1). FIG. 8B depicts Fv3A amino acid sequence (SEQ ID NO:2). The predicted signal sequence is underlined. The predicted conserved domain is in bold.

FIGS. 9A-9B: FIG. 9A depicts Pf43A nucleotide sequence (SEQ ID NO:3). FIG. 9B depicts Pf43A amino acid sequence (SEQ ID NO:4). The predicted signal sequence is underlined, the predicted conserved domain is in bold, the predicted carbohydrate binding module (“CBM”) is in uppercase, and the predicted linker separating the CD and CBM is in italics.

FIGS. 10A-10B: FIG. 10A depicts Fv43E nucleotide sequence (SEQ ID NO:5). FIG. 10B depicts Fv43E amino acid sequence (SEQ ID NO:6). The predicted signal sequence is underlined. The predicted conserved domain is in bold.

FIGS. 11A-11B: FIG. 11A depicts Fv39A nucleotide sequence (SEQ ID NO:7). FIG. 11B depicts Fv39A amino acid sequence (SEQ ID NO:8). The predicted signal sequence is underlined. The predicted conserved domain is in boldface type.

FIGS. 12A-12B: FIG. 12A depicts Fv43A nucleotide sequence (SEQ ID NO:9). FIG. 12B depicts Fv43A amino acid sequence (SEQ ID NO:10). The predicted signal sequence is underlined. The predicted conserved domain is in bold type, the predicted CBM is in uppercase, and the predicted linker separating the conserved domain and CBM is in italics.

FIGS. 13A-13B: FIG. 13A depicts Fv43B nucleotide sequence (SEQ ID NO:11). FIG. 13B depicts Fv43B amino acid sequence (SEQ ID NO:12). The predicted signal sequence is underlined. The predicted conserved domain is in boldface type.

FIGS. 14A-14B: FIG. 14A depicts Pa51A nucleotide sequence (SEQ ID NO:13). FIG. 14B depicts Pa51A amino acid sequence (SEQ ID NO:14). The predicted signal sequence is underlined. The predicted L-α-arabinofuranosidase conserved domain is in bold. For expression in T. reesei, the genomic DNA was codon optimized (see FIG. 27C).

FIGS. 15A-15B: FIG. 15A depicts Gz43A nucleotide sequence (SEQ ID NO:15). FIG. 15B depicts Gz43A amino acid sequence (SEQ ID NO:16). The predicted signal sequence is underlined, and the predicted conserved domain is in bold. For expression in T. reesei the predicted signal sequence was replaced by the T. reesei CBH1 signal sequence (MYRKLAVISAFLATARA (SEQ ID NO: 159)) in T. reesei.

FIGS. 16A-16B: FIG. 16A depicts Fo43A nucleotide sequence (SEQ ID NO:17). FIG. 16B depicts Fo43A amino acid sequence (SEQ ID NO:18). The predicted signal sequence is underlined. The predicted conserved domain is in bold. For expression in T. reesei, the predicted signal sequence was replaced by the T. reesei CBH1 signal sequence (MYRKLAVISAFLATARA (SEQ ID NO:159)).

FIGS. 17A-17B: FIG. 17A depicts Af43A nucleotide sequence (SEQ ID NO:19). FIG. 17B depicts Af43A amino acid sequence (SEQ ID NO:20). The predicted conserved domain is in bold.

FIGS. 18A-18B: FIG. 18A depicts Pf51A nucleotide sequence (SEQ ID NO:21). FIG. 18B depicts Pf51A amino acid sequence (SEQ ID NO:22). The predicted signal sequence is underlined. The predicted L-α-arabinofuranosidase conserved domain is in bold. For expression in T. reesei, the predicted Pf51A signal sequence was replaced by the T. reesei CBH1 signal sequence (MYRKLAVISAFLATARA (SEQ ID NO:159)) and the Pf51A nucleotide sequence was codon optimized for expression in T. reesei

FIGS. 19A-19B: FIG. 19A depicts AfuXyn2 nucleotide sequence (SEQ ID NO:23). FIG. 19B depicts AfuXyn2 amino acid sequence (SEQ ID NO:24). The predicted signal sequence is underlined. The predicted GH11 conserved domain is in bold.

FIGS. 20A-20B: FIG. 20A depicts AfuXyn5 nucleotide sequence (SEQ ID NO:25). FIG. 20B depicts AfuXyn5 amino acid sequence (SEQ ID NO:26). The predicted signal sequence is underlined. The predicted GH11 conserved domain is in bold.

FIGS. 21A-21B: FIG. 21A depicts Fv43D nucleotide sequence (SEQ ID NO:27). FIG. 21B depicts Fv43D amino acid sequence (SEQ ID NO:28). The predicted signal sequence is underlined. The predicted conserved domain is in bold.

FIGS. 22A-22B: FIG. 22A depicts Pf43B nucleotide sequence (SEQ ID NO:29). FIG. 22B depicts Pf43B amino acid sequence (SEQ ID NO:30). The predicted signal sequence is underlined. The predicted conserved domain is in bold.

FIGS. 23A-23B: FIG. 23A depicts nucleotide sequence (SEQ ID NO:31). FIG. 23B depicts Fv51A amino acid sequence (SEQ ID NO:32). The predicted signal sequence is underlined. The predicted L-α-arabinofuranosidase conserved domain is in bold.

FIGS. 24A-24B: FIG. 24A depicts T. reesei Xyn3 nucleotide sequence (SEQ ID NO:41). FIG. 24B depicts T. reesei Xyn3 amino acid sequence (SEQ ID NO:42). The predicted signal sequence is underlined. The predicted conserved domain is in bold.

FIGS. 25A-25B: FIG. 25A depicts amino acid sequence of T. reesei Xyn2 (SEQ ID NO:43). The signal sequence is underlined. The predicted conserved domain is in bold face type. FIG. 25B depicts nucleotide sequence of T. reesei Xyn2 (SEQ ID NO:162). The coding sequence can be found in Törrönen et al. Biotechnology, 1992, 10:1461-65.

FIGS. 26A-26B: FIG. 26A depicts amino acid sequence of T. reesei Bxl1 (SEQ ID NO:44). The signal sequence is underlined. The predicted conserved domain is in bold. FIG. 26B depicts nucleotide sequence of T. reesei Bxl1 (SEQ ID NO:163). The coding sequence can be found in Margolles-Clark et al. Appl. Environ. Microbiol. 1996, 62(10):3840-46.

FIGS. 27A-27F: FIG. 27A depicts amino acid sequence of T. reesei Bgl1 (SEQ ID NO:45). The signal sequence is underlined. The coding sequence can be found in Barnett et al. Bio-Technology, 1991, 9(6):562-567. FIG. 27B depicts deduced cDNA for Pa51A (SEQ ID NO:46). FIG. 27C depicts codon optimized cDNA for Pa51A (SEQ ID NO:47). FIG. 27D: Coding sequence for a construct comprising a CBH1 signal sequence (underlined) upstream of genomic DNA encoding mature Gz43A (SEQ ID NO:48). FIG. 27E: Coding sequence for a construct comprising a CBH1 signal sequence (underlined) upstream of genomic DNA encoding mature Fo43A (SEQ ID NO:49). FIG. 27F: Coding sequence for a construct comprising a CBH1 signal sequence (underlined) upstream of codon optimized DNA encoding Pf51A (SEQ ID NO:50).

FIGS. 28A-28B: FIG. 28A depicts nucleotide sequence of T. reesei Eg4 (SEQ ID NO:51). FIG. 28B depicts amino acid sequence of T. reesei Eg4 (SEQ ID NO:52). The predicted signal sequence is underlined. The predicted conserved domains are in bold. The predicted linker is in italic type fonts.

FIGS. 29A-29B: FIG. 29A depicts nucleotide sequence of Pa3D (SEQ ID NO:53). FIG. 29B depicts amino acid sequence of Pa3D (SEQ ID NO:54). The predicted signal sequence is underlined. The predicted conserved domains are in bold.

FIGS. 30A-30B: FIG. 30A depicts nucleotide sequence of Fv3G (SEQ ID NO:55). FIG. 30B depicts amino acid sequence of Fv3G (SEQ ID NO:56). The predicted signal sequence is underlined. The predicted conserved domains are in bold.

FIGS. 31A-31B: FIG. 31A depicts nucleotide sequence of Fv3D (SEQ ID NO:57). FIG. 31B depicts amino acid sequence of Fv3D (SEQ ID NO:58). The predicted signal sequence is underlined. The predicted conserved domains are in bold.

FIGS. 32A-32B: FIG. 32A depicts nucleotide sequence of Fv3C (SEQ ID NO:59). FIG. 32B depicts amino acid sequence of Fv3C (SEQ ID NO:60). The predicted signal sequence is underlined. The predicted conserved domains are in bold.

FIGS. 33A-33B: FIG. 33A depicts nucleotide sequence of Tr3A (SEQ ID NO:61). FIG. 33B depicts amino acid sequence of Tr3A (SEQ ID NO:62). The predicted signal sequence is underlined. The predicted conserved domains are in bold.

FIGS. 34A-46B: FIG. 34A depicts nucleotide sequence of Tr3B (SEQ ID NO:63). FIG. 34B depicts amino acid sequence of Tr3B (SEQ ID NO:64). The predicted signal sequence is underlined. The predicted conserved domains are in bold.

FIGS. 35A-47B: FIG. 35A depicts the codon-optimized nucleotide sequence of Te3A (SEQ ID NO:65). FIG. 35B depicts amino acid sequence of Te3A (SEQ ID NO:66). The predicted signal sequence is underlined. The predicted conserved domains are in bold.

FIGS. 36A-36B: FIG. 36A depicts nucleotide sequence of An3A (SEQ ID NO:67). FIG. 36B depicts amino acid sequence of An3A (SEQ ID NO:68). The predicted signal sequence is underlined. The predicted conserved domains are in bold.

FIGS. 37A-37B: FIG. 37A depicts nucleotide sequence of Fo3A (SEQ ID NO:69). FIG. 37B depicts amino acid sequence of Fo3A (SEQ ID NO:70). The predicted signal sequence is underlined. The predicted conserved domains are in bold.

FIGS. 38A-38B: FIG. 38A depicts nucleotide sequence of Gz3A (SEQ ID NO:71). FIG. 38B depicts amino acid sequence of Gz3A (SEQ ID NO:72). The predicted signal sequence is underlined. The predicted conserved domains are in bold.

FIGS. 39A-39B: FIG. 39A depicts nucleotide sequence of Nh3A (SEQ ID NO:73). FIG. 39B depicts amino acid sequence of Nh3A (SEQ ID NO:74). The predicted signal sequence is underlined. The predicted conserved domains are in bold.

FIGS. 40A-40B: FIG. 40A depicts nucleotide sequence of Vd3A (SEQ ID NO:75). FIG. 40B depicts amino acid sequence of Vd3A (SEQ ID NO:76). The predicted signal sequence is underlined. The predicted conserved domains are in bold.

FIGS. 41A-41B: FIG. 41A depicts nucleotide sequence of Pa3G (SEQ ID NO:77). FIG. 41B depicts amino acid sequence of Pa3G (SEQ ID NO:78). The predicted signal sequence is underlined. The predicted conserved domains are in bold.

FIG. 42: depicts amino acid sequence of Tn3B (SEQ ID NO:79). The standard signal prediction program Signal P provided no predicted signal sequence.

FIGS. 43A-43B: FIG. 43A depicts an amino acid sequence alignment of certain β-glucosidase homologs. FIG. 43B depicts an alignment of β-glucosidase homologs, some of which are known to be susceptible to proteolytic clipping but others are not. The first underlined region contains residues that are approximately within a centrally-located loop sequence of this class of enzymes. The second underlined region downstream from the first underlined region contains residues that are frequently susceptible to initial proteolytic digestion or clipping.

FIG. 44: depicts a pENTR/D-TOPO vector with the Fv3C open reading frame.

FIGS. 45A-45B: FIG. 45A depicts the pTrex6g vector. FIG. 45B depicts a pExpression construct pTrex6g/Fv3C.

FIGS. 46A-46C: FIG. 46A depicts predicted coding region of Fv3C genomic DNA sequence. FIG. 46B depicts N-terminal amino acid sequence of Fv3C. The arrows show the putative signal peptide cleavage sites. The start of the mature protein is underlined. FIG. 46C depicts an SDS-PAGE gel of T. reesei transformants expressing Fv3C from the annotated (1) and alternative (2) start codons.

FIG. 47: compares the performance of a number of whole cellulase and β-glucosidase mixtures in saccharification of phosphoric acid swollen cellulose at 50° C. In this experiment, whole cellulase at 10 mg protein/g cellulose was blended with 5 mg/g β-glucosidase and the enzyme mixtures used to hydrolyze phosphoric acid swollen cellulose at 0.7% cellulose, pH 5.0. The sample labeled as background in the figure was the conversion obtained from 10 mg/g whole cellulase alone without added β-glucosidase. Reactions were carried out in microtiter plates at 50° C. for 2 h. The samples were tested in triplicates. This is according to Example 5A.

FIG. 48: compares the performance of a number of whole cellulase and β-glucosidase mixtures in saccharification of acid pre-treated cornstover (PCS) at 50° C. In this experiment, whole cellulase at 10 mg protein/g cellulose was blended with 5 mg/g β-glucosidase and the enzyme mixtures used to hydrolyze PCS at 13% solids, pH 5.0. The sample labeled as background in the figure was the conversion obtained from 10 mg/g whole cellulase alone without added β-glucosidase. Reactions were carried out in microtiter plates at 50° C. for 48 h. The samples were tested in triplicates. Experimental details are described in Example 5B.

FIG. 49: compares the performance of a number of whole cellulase and β-glucosidase mixtures in saccharification of dilute ammonia pretreated corncob at 50° C. In this experiment, whole cellulase at 10 mg protein/g cellulose was blended with 8 mg/g hemicellulases and 5 mg/g β-glucosidase and the enzyme mixtures used to hydrolyze the dilute ammonia pretreated corncob at 20% solids, pH 5.0. The sample labeled as background in the figure was the conversion obtained from 10 mg/g whole cellulase+8 mg/g hemicellulose mix alone without added β-glucosidase. Reactions were carried out in microtiter plates at 50° C. for 48 h. The samples were tested in triplicates. Experimental details are described in Example 5C.

FIG. 50: compares the performance of whole cellulase and β-glucosidase mixtures in saccharification of sodium hydroxide (NaOH) pretreated corncob at 50° C. In this experiment, whole cellulase at 10 mg protein/g cellulose was blended with 5 mg/g β-glucosidase and the enzyme mixtures used to hydrolyze the NaOH pretreated corncob at 17% solids, pH 5.0. The sample labeled as background in the figure was the conversion obtained from 10 mg/g whole cellulase mix alone without added β-glucosidase. Reactions were carried out in microtiter plates at 50° C. for 48 h. Each sample was run with 4 replicates. This is according to Example 5D.

FIG. 51: compares the performance of whole cellulase and β-glucosidase mixtures in saccharification of dilute ammonia pretreated switchgrass at 50° C. In this experiment, whole cellulase at 10 mg protein/g cellulose was blended with 5 mg/gβ-glucosidase and the enzyme mixtures used to hydrolyze switchgrass at 17% solids, pH 5.0. The sample labeled as background in the figure was the conversion obtained from 10 mg/g whole cellulase mix alone without added β-glucosidase. Reactions were carried out in microtiter plates at 50° C. for 48 h. Each sample was run with 4 replicates. Experimental details are described in Example 5E.

FIG. 52: compares the performance of whole cellulase and β-glucosidase mixtures in saccharification of AFEX cornstover at 50° C. In this experiment, whole cellulase at 10 mg protein/g cellulose was blended with 5 mg/gβ-glucosidase and the enzyme mixtures used to hydrolyze AFEX cornstover at 14% solids, pH 5.0. The sample labeled as background in the figure was the conversion obtained from 10 mg/g whole cellulase mix alone without added beta-glucosidase. Reactions were carried out in microtiter plates at 50° C. for 48 h. Each sample was run with 4 replicates. Experimental details are described in Example 5F.

FIGS. 53A-53C: depict percent glucan conversion from dilute ammonia pretreated corncob at 20% solids at varying ratios of β-glucosidase to whole cellulase, in an amount of between 0 and 50%. The enzyme dosage was kept constant for each of the experiments. FIG. 53A depicts the experiment conducted with T. reesei Bgl1. FIG. 53B depicts the experiment conducted with Fv3C. FIG. 53C depicts the experiment conducted with A. niger Bglu (An3A).

FIG. 54: depicts percent glucan conversion from dilute ammonia pretreated corncob at 20% solids by three different enzyme compositions dosed at levels of 2.5-40 mg/g glucan, in accordance with Example 7. Δ marks glucan conversion observed with Accellerase 1500+Multifect Xylanase, ⋄ marks glucan conversion observed with a whole cellulase from T. reesei integrated strain H3A, ♦ marks glucan conversion observed with an enzyme composition comprising 75 wt. % whole cellulase from T. reesei integrated strain H3A plus 25 wt. % Fv3C.

FIGS. 55A-55I: FIG. 55A depicts a map of the pRAX2-Fv3C expression plasmid used for expression in A. niger. FIG. 55B depicts pENTR-TOPO-Bgl1-943/942 plasmid. FIG. 55C depicts pTrex3g 943/942 expression vector. FIG. 55D depicts pENTR/T. reesei Xyn3 plasmid. FIG. 55E depicts pTrex3g/T. reesei Xyn3 expression vector. FIG. 55F depicts pENTR-Fv3A plasmid. FIG. 55G depicts pTrex6g/Fv3A expression vector. FIG. 55H depicts TOPO Blunt/Pegl1-Fv43D plasmid. FIG. 55I depicts TOPO Blunt/Pegl1-Fv51A plasmid.

FIG. 56: depicts an amino acid alignment between T. reesei β-xylosidase Bxl1 and Fv3A.

FIG. 57: depicts an amino acid sequence alignment of certain GH43 family hydrolases. Amino acid residues conserved among members of the family are underlined and in bold face.

FIG. 58: depicts an amino acid sequence alignment of certain GH51 family enzymes. Amino acid residues conserved among members of the family are underlined and in bold face.

FIG. 59A-59B: depict amino acid sequence alignments of a number of GH10 and GH11 family endoxylanases. FIG. 59A: Alignment of GH10 family xylanases. Underlined residues in bold face are the catalytic nucleophile residues (marked with “N” above the alignment). FIG. 59B: Alignment of GH11 family xylanases. Underlined residues in bold face are the catalytic nucleophile residues and general acid base residues (marked with “N” and “A”, respectively, above the alignment).

FIG. 60A-60C: FIG. 60A depicts a schematic representation of the gene encoding the Fv3C/T. reesei Bgl3 (“FB”) chimeric/fusion polypeptide. FIG. 60B depicts the nucleotide sequence encoding the fusion/chimeric polypeptide Fv3C/T. reesei Bgl3 (“FB”) (SEQ ID NO:82). FIG. 60C depicts the amino acid sequence encoding the fusion/chimeric polypeptide Fv3C/T. reesei Bgl3. (SEQ ID NO:159). The sequence in bold type is from T. reesei Bgl3.

FIG. 61: depicts a map of the pTTT-pyrG13-Fv3C/Bgl3 fusion plasmid.

FIG. 62: compares T. reesei Bgl1 (closed diamonds) and Fv3C produced in A. niger (open diamonds) in saccharification of dilute ammonia pre-treated corncob. In this experiment, T. reesei Bgl1 and Fv3C were loaded from 0-10 mg protein/g cellulose with a constant level of 10 mg/g H3A-5 and these mixtures used to hydrolyze dilute ammonia pre-treated corncob at 5% cellulose, pH 5.0. Reactions were carried out in microtiter plate at 50° C. for 2 days. Each sample was run with 5 assay replicates. Experimental details are shown in Example 13.

FIG. 63: DSC profiles of β-glucosidases T. reesei Bglu1 (Tr3A), Fv3C, and Fv3C/Te3A/Bgl3 (“FAB”) chimeric polypeptide collected with a 90° C./r scan rate (25° C.-110° C.) in 50 mM sodium acetate buffer, pH 5.

FIGS. 64A-64E: FIG. 64A: Performance of whole cellulase: T. reesei Bgl3 mixtures in saccharification of phosphoric acid swollen cellulose at 50° C. FIG. 64B: T. reesei Bgl3 mixtures in saccharification of phosphoric acid swollen cellulose at 37° C. FIG. 64C: T. reesei Bgl3 mixtures in saccharification of acid pre-treated corn stover at 50° C. FIG. 64D: T. reesei Bgl3 mixtures in saccharification of acid pre-treated corn stover at 37° C.

FIGS. 65A-65B. FIG. 65A: Comparison of T. reesei Bgl1 (closed diamonds) and T. reesei Bgl3 (open diamonds) in phosphoric acid swollen cellulose saccharification. FIG. 65B: Comparison of cellobiose (black bars) and glucose (white bars) produced by T. reesei Bgl1 (left panel) and T. reesei Bgl3 (right panel) in saccharification of phosphoric acid swollen cellulose.

FIG. 66: depicts the nucleotide sequences of a number of primers.

FIGS. 67A-67B: FIG. 67A depicts full length amino acid sequence of Fv3C/Te3A/T. reesei Bgl3 (“FAB”) (SEQ ID NO:135) (Te3A is in bold italic capital letters, T. reesei Bgl3 is in underlined capital letters). FIG. 67B depicts the nucleic acid sequence encoding the Fv3C/Te3A/T. reesei Bgl3 (“FAB”) chimera (SEQ ID NO:83).

FIGS. 68A-68C: FIG. 68A is a table listing structural motifs present in the N- and C-terminal domains of certain chimeric β-glucosidase polypeptides. FIG. 68B is a table listing certain amino acid sequence motifs used to design a suitable β-glucosidase polypeptide hybrid/chimera of the invention. FIG. 68C is a list of amino acid sequence motifs of GH61/endoglucanases.

FIG. 69: depicts nucleotide and protein sequences of Pa3C (SEQ ID NOs:80 and 81, respectively).

FIGS. 70A-G: FIG. 70A depicts 3-D superimposed structures of Fv3C and Te3A, and T. reesei Bgl1, viewed from a first angle, rendering visible the structure of “insertion 1.” FIG. 70B depicts the same superimposed structures viewed from a second angle, rendering visible the structure of “insertion 2.” FIG. 70C depicts the same superimposed structures viewed from a third angle, rendering visible the structure of “insertion 3.” FIG. 70D depicts the same superimposed structures, viewed from a fourth angle, rendering visible the structure of “insertion 4.” FIG. 70E is a sequence alignment of T. reesei Bgl1 (Q12715_TRI), Te3A (ABG2_T_eme), and Fv3C (FV3C), marked with insertions 1-4, which are all loop-like structures. FIG. 70F depicts superimposed parts of structures of Fv3C (light grey), Te3A (dark grey), and T. reesei Bgl1 (black), indicating conserved interactions of between residues W59/W33 and W355/W325 (Fv3C/Te3A). FIG. 70G depicts superimposed parts of structures of Fv3C (light grey), Te3A (dark grey), and T. reesei Bgl1 (black), indicating conserved interactions between the first pair of residues: S57/31 and N291/261 (Fv3C/Te3A); and among the second groups of residues: Y55/29, P775/729 and A778/732 (Fv3C/Te3A). FIG. 70H depicts superimposed parts of structures Fv3C (dark grey), and T. reesei Bgl1 (black), indicating hydrogen bonding Interactions of Fv3C at K162 with the backbone oxygen atom of V409 in “insertion 2,” an interaction that is conserved in Te3A, but not found in T. reesei Bgl1. FIG. 70I (a)-(b) depict conserved glycosylation sites within SEQ ID NO:168, shared amongst Fv3C, Te3A and a chimeric/hybrid β-glucosidase of SEQ ID NO:135, (a) depicts the same region superimposed with Te3A (dark grey) and T. reesei Bgl1(black); (b) depicts the same region superimposed with the chimeric/hybrid β-glucosidase of SEQ ID NO:135 (light grey), Te3A (dark grey) and T. reesei Bgl1 (black). The black arrow indicates the loop structure of “insertion 3” in Te3A (also present in the hybrid β-glucosidase of SEQ ID NO:135), which appeared to bury the glycosylation glycans. FIG. 70J depicts superimposed parts of structures of Fv3C (light grey), Te3A (dark grey), and T. reesei Bgl1 (black), indicating conserved interactions between residues W386/355 interacts with W95/68 (Fv3C/Te3A) of “insertion 2” of Fv3C and Te3A. The interaction is missing from T. reesei Bgl1.

FIGS. 71A-71C: FIG. 71A: depicts the amount of measured unbound proteins in soluble fraction (supernatant) following 50° C. incubation for 44 hrs, in accordance with Example 13. FIG. 71B: depicts the total protein (bound and unbound) in slurry following 50° C. incubation for 44 hrs, in accordance with Example 13. FIG. 71C: depicts the unbound protein in slurry after 30 min of additional incubation in buffer, in accordance with Example 13.

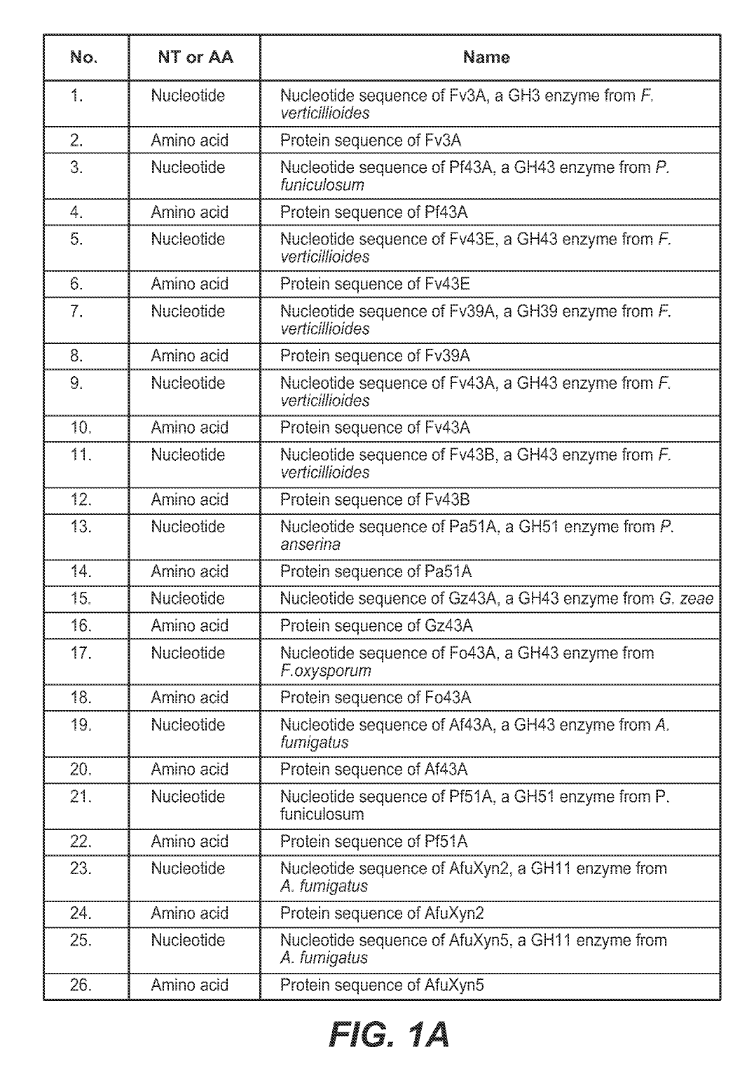

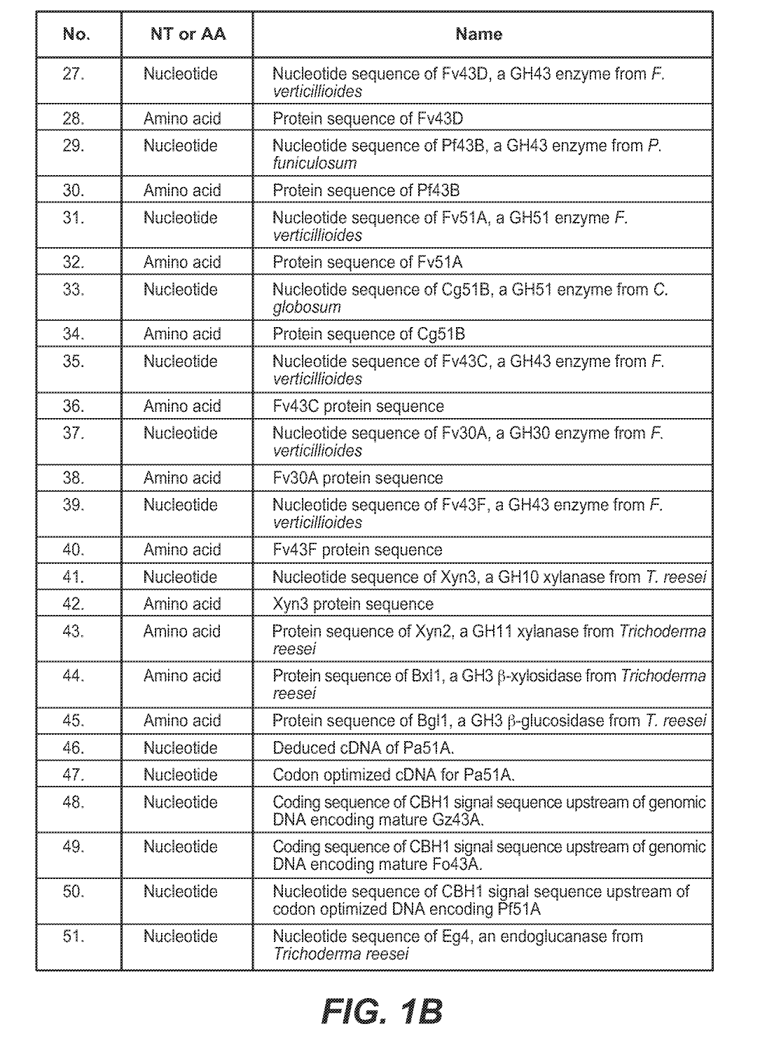

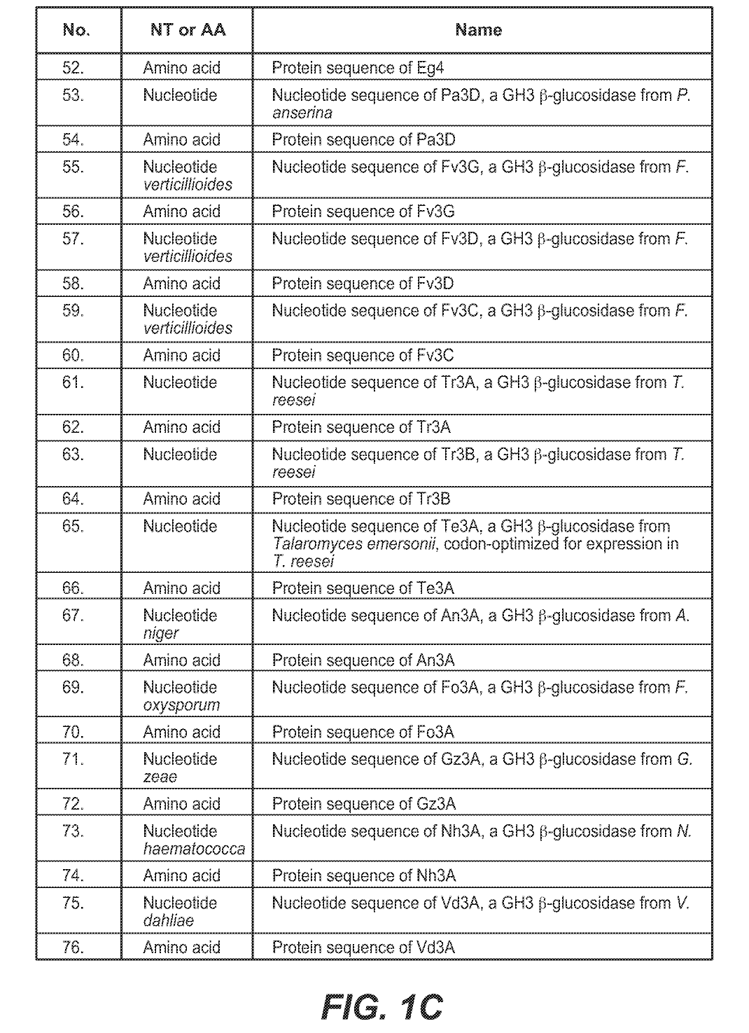

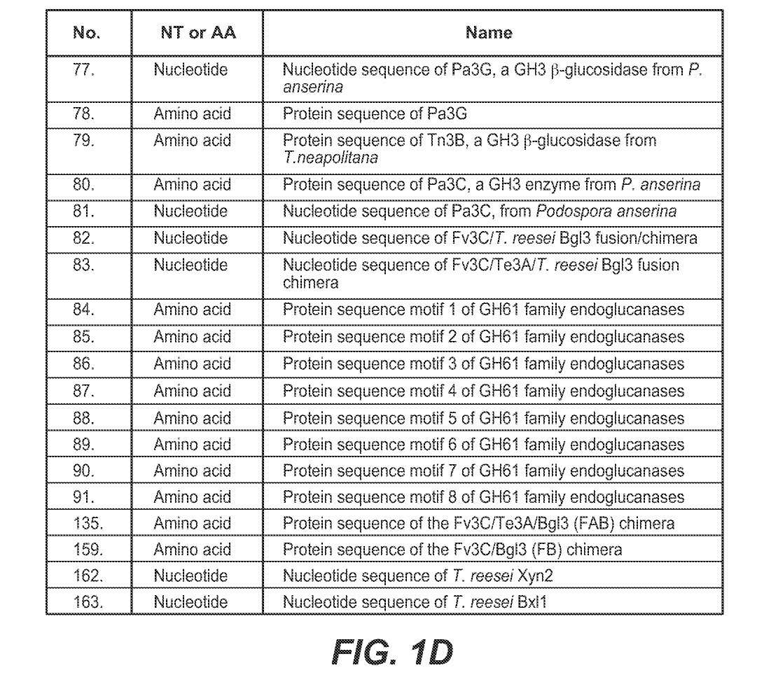

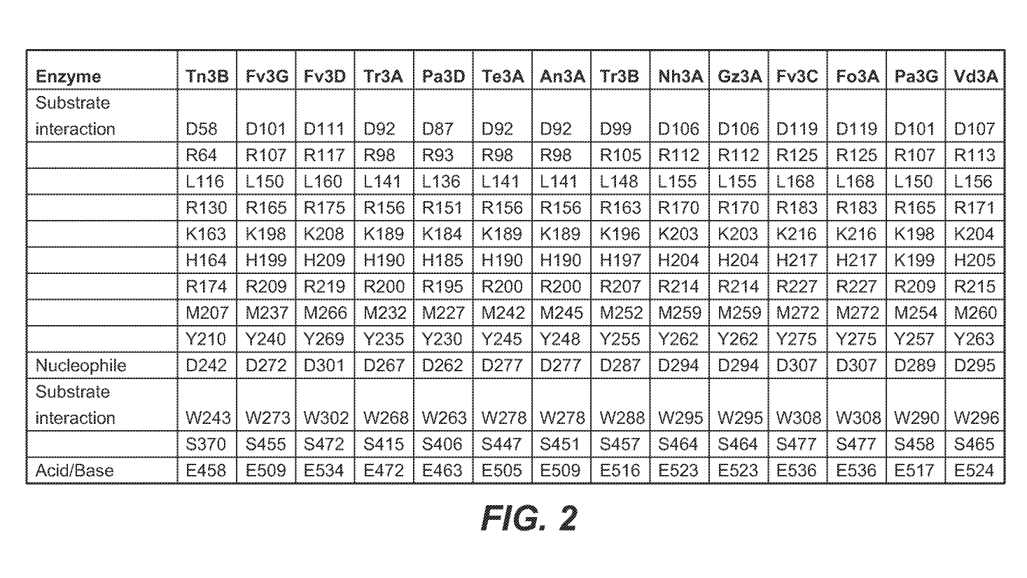

### DETAILED DESCRIPTION OF THE INVENTION

Enzymes have traditionally been classified by substrate specificity and reaction products. In the pre-genomic era, function was regarded as the most amenable (and perhaps most useful) basis for comparing enzymes and assays for various enzymatic activities have been well-developed for many years, resulting in the familiar EC classification scheme. Cellulases and other glycosyl hydrolases, which act upon glycosidic bonds between two carbohydrate moieties (or a carbohydrate and non-carbohydrate moiety-as occurs in nitrophenol-glycoside derivatives) are, under this classification scheme, designated as EC 3.2.1.-, with the final number indicating the exact type of bond cleaved. For example, according to this scheme an endo-acting cellulase (1,4-β-endoglucanase) is designated EC 3.2.1.4.

With the advent of widespread genome sequencing projects, sequencing data have facilitated analyses and comparison of related genes and proteins. Additionally, a growing number of enzymes capable of acting on carbohydrate moieties (i.e., carbohydrases) have been crystallized and their 3-D structures solved. Such analyses have identified discreet families of enzymes with related sequence, which contain conserved three-dimensional folds that can be predicted based on their amino acid sequence. Further, it has been shown that enzymes with the same or similar three-dimensional folds exhibit the same or similar stereospecificity of hydrolysis, even when catalyzing different reactions (Henrissat et al., FEBS Lett 1998, 425(2): 352-4; Coutinho and Henrissat, Genetics, biochemistry and ecology of cellulose degradation, 1999, T. Kimura. Tokyo, Uni Publishers Co: 15-23.).

These findings form the basis of a sequence-based classification of carbohydrase modules, which is available in the form of an internet database, the Carbohydrate-Active enZYme server (CAZy), at www.cazy.org (See Cantarel et al., 2009, The Carbohydrate-Active EnZymes database (CAZy): an expert resource for Glycogenomics. Nucleic Acids Res. 37 (Database issue):D233-38).

CAZy defines four major classes of carbohydrases distinguishable by the type of reaction catalyzed: Glycosyl Hydrolases (GH's), Glycosyltransferases (GT's), Polysaccharide Lyases (PL's), and Carbohydrate Esterases (CE's). The enzymes of the disclosure are glycosyl hydrolases. GH's are a group of enzymes that hydrolyze the glycosidic bond between two or more carbohydrates, or between a carbohydrate and a non-carbohydrate moiety. A classification system for glycosyl hydrolases, grouped by sequence similarity, has led to the definition of over 120 different families. This classification is available on the CAZy web site. The enzymes of the present invention belong to glycosyl hydrolase family 3 (GH3).

GH3 enzymes include, e.g., β-glucosidase (EC:3.2.1.21); β-xylosidase (EC:3.2.1.37); N-acetyl β-glucosaminidase (EC:3.2.1.52); glucan β-1,3-glucosidase (EC:3.2.1.58); cellodextrinase (EC:3.2.1.74); exo-1,3-1,4-glucanase (EC:3.2.1); and β-galactosidase (EC 3.2.1.23). For example, GH3 enzymes can be those that have β-glucosidase, β-xylosidase, N-acetyl β-glucosaminidase, glucan β-1,3-glucosidase, cellodextrinase, exo-1,3-1,4-glucanase, and/or β-galactosidase activity. Generally, GH3 enzymes are globular proteins and can consist of two or more subdomains. A catalytic residue has been identified as an aspartate residue that, in β-glucosidases, located in the N-terminal third of the peptide and sits within the amino acid fragment SDW (Li et al. 2001, Biochem. J. 355:835-840). The corresponding sequence in Bgl1 from T. reesei is T266D267W268 (counting from the methionine at the starting position), with the catalytic residue aspartate being the D267. The hydroxyl/aspartate sequence is also conserved in the GH3 β-xylosidases tested. For example, the corresponding sequence in T. reesei Bxl1 is S310D311 and the corresponding sequence in Fv3A is S290D291.

### Polypeptides of the Invention

Cellulases

The compositions of the disclosure can comprise one or more cellulases. Cellulases are enzymes that hydrolyze cellulose (β-1,4-glucan or β D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and β-glucosidases (β-D-glucoside glucohydrolase; EC 3.2.1.21) (“BG”) (Knowles et al., 1987, Trends in Biotechnology 5(9):255-261; Shulein, 1988, Methods in Enzymology, 160:234-242).

Cellulases for use in accordance with the methods and compositions of the disclosure can be obtained from, or produced recombinantly from, without limitation, one or more of the following organisms: Chrysosporium lucknowense, Crinipellis scapella, Macrophomina phaseolina, Myceliophthora thermophila, Sordaria fimicola, Volutella colletotrichoides, Thielavia terrestris, Acremonium sp., Exidia glandulosa, Fomes fomentarius, Spongipellis sp., Rhizophlyctis rosea, Rhizomucor pusillus, Phycomyces niteus, Chaetostylum fresenii, Diplodia gossypina, Ulospora bilgramii, Saccobolus dilutellus, Penicillium verruculosum, Penicillium chrysogenum, Thermomyces verrucosus, Diaporthe syngenesia, Colletotrichum lagenarium, Nigrospora sp., Xylaria hypoxylon, Nectria pinea, Sordaria macrospora, Thielavia thermophila, Chaetomium mororum, Chaetomium virscens, Chaetomium brasiliensis, Chaetomium cunicolorum, Syspastospora boninensis, Cladorrhinum foecundissimum, Scytalidium thermophila, Gliocladium catenulatum, Fusarium oxysporum ssp. lycopersici, Fusarium oxysporum ssp. passiflora, Fusarium solani, Fusarium anguioides, Fusarium poae, Humicola nigrescens, Humicola grisea, Panaeolus retirugis, Trametes sanguinea, Schizophyllum commune, Trichothecium roseum, Microsphaeropsis sp., Acsobolus stictoideus spej., Poronia punctata, Nodulisporum sp., Trichoderma sp. (e.g., T. reesei) and Cylindrocarpon sp. Cellulases may also be obtained from, or produced recombinantly from a bacterium, or may be produced recombinantly from a yeast.

For example, a cellulase for use in a method and/or composition of the disclosure is a whole cellulase and/or is capable of achieving at least 0.1 (e.g. 0.1 to 0.4) fraction product as determined by the calcofluor assay.

β-glucosidases

β-glucosidase(s) (or interchangeably herein “β-glucosidase polypeptide(s)”) catalyze the hydrolysis of terminal non-reducing residues in β-D-glucosides with release of glucose. Examples of β-glucosidase polypeptides include polypeptides, fragments of polypeptides, peptides, and fusion polypeptides that have at least one activity of a β-glucosidase polypeptide. Examples of β-glucosidase polypeptides and nucleic acids include naturally-occurring polypeptides (including, e.g., variants) and nucleic acids from any of the source organisms described herein, and mutant polypeptides and nucleic acids derived from any of the source organisms described herein that have at least one activity of a β-glucosidase polypeptide.

The compositions of the disclosure can comprise one or more β-glucosidase polypeptides. The term “β-glucosidase” as used herein refers to a β-D-glucoside glucohydrolase classified as EC 3.2.1.21, and/or members of GH family 3 which catalyze the hydrolysis of cellobiose to release β-D-glucose. The GH3 β-glucosidases of the present invention include, without limitation, Fv3C, Pa3D, Fv3G, Fv3D, Tr3A (also termed “T. reesei Bgl1” or “T. reesei Bglu1”), Tr3B (also termed “T. reesei Bgl3”), Te3A, An3A (also termed “A. niger Bglu”), Fo3A, Gz3A, Nh3A, Vd3A, Pa3G, or Tn3B polypeptide. In some embodiments, the GH3 β-glucosidase polypeptide herein has at least one activity of a β-glucosidase polypeptide.

Suitable β-glucosidase polypeptides can be obtained from a number of microorganisms, by recombinant means, or be purchased from commercial sources. Examples of β-glucosidases from microorganisms include, without limitation, ones from bacteria and fungi. For example, a β-glucosidase of the present disclosure is suitably obtained from a filamentous fungus.

The β-glucosidase polypeptides can be obtained, or produced recombinantly, from, inter alia, A. aculeatus (Kawaguchi et al. Gene 1996, 173: 287-288), A. kawachi (Iwashita et al. Appl. Environ. Microbiol. 1999, 65: 5546-5553), A. oryzae (WO 2002/095014), C. biazotea (Wong et al. Gene, 1998, 207:79-86), P. funiculosum (WO 2004/078919), S. fibuligera (Machida et al. Appl. Environ. Microbiol. 1988, 54: 3147-3155), S. pombe (Wood et al. Nature 2002, 415: 871-880), T. reesei (e.g., β-glucosidase 1 (U.S. Pat. No. 6,022,725), β-glucosidase 3 (U.S. Pat. No. 6,982,159), β-glucosidase 4 (U.S. Pat. No. 7,045,332), β-glucosidase 5 (U.S. Pat. No. 7,005,289), β-glucosidase 6 (U.S. Publication No. 20060258554), β-glucosidase 7 (U.S. Publication No. 20060258554)), P. anserina (e.g. Pa3D), F. verticillioides (e.g. Fv3G, Fv3D, or Fv3C), T. reesei (e.g. Tr3A, or Tr3B), T. emersonii (e.g. Te3A), A. niger (e.g. An3A), F. oxysporum (e.g. Fo3A), G. zeae (e.g. Gz3A), N. haematococca (e.g. Nh3A), V. dahliae (e.g. Vd3A), P. anserine (e.g. Pa3G), or T. neapolitana (e.g. Tn3B).

The β-glucosidase polypeptide can be produced by expressing an endogenous/exogenous gene encoding a β-glucosidase, a variant, a hybrid/chimera/fusion, or a mutant. For example, β-glucosidase polypeptides can be secreted into the extracellular space e.g., by Gram-positive organisms such as Bacillus or Actinomycetes, or by eukaryotic hosts such as fungi (e.g., Trichoderma, Chrysosporium, Aspergillus, Saccharomyces, Pichia). β-glucosidase polypeptides may be expressed in a yeast such as a Saccharomyces cerevisiae. The β-glucosidase polypeptide may be overexpressed or underexpressed.

The β-glucosidase polypeptide can also be obtained from commercial sources. Examples of commercial β-glucosidase preparation suitable for use in the present disclosure include, e.g., T. reesei β-glucosidase in Accellerase® BG (Danisco US Inc., Genencor); NOVOZYM™ 188 (a β-glucosidase from A. niger); Agrobacterium sp. β-glucosidase, and T. maritima β-glucosidase from Megazyme (Megazyme International Ireland Ltd., Ireland.).

Moreover, the β-glucosidase polypeptide can be a component of a cellulase composition, a whole cell cellulase composition, a cellulase fermentation broth, or a whole broth formulation cellulase composition.

β-glucosidase activity can be determined by a number of suitable means known in the art, including, in a non-limiting example, the assay described by Chen et al., in Biochimica et Biophysica Acta 1992, 121:54-60, wherein 1 pNPG denotes 1 μmoL of Nitrophenol liberated from 4-nitrophenyl-β-D-glucopyranoside in 10 min at 50° C. and pH 4.8.

β-glucosidase polypeptides suitably constitutes about 0 wt. % to about 75 wt. % of the total weight of enzymes in a cellulase composition of the invention. The ratio of any pair of enzymes relative to each other can be readily calculated based on the disclosure herein. Cellulase compositions comprising enzymes in any weight ratio derivable from the weight percentages disclosed herein are contemplated. The β-glucosidase content can be in a range wherein the lower limit is about 0 wt. %, 1 wt. %, 2 wt. %, 3 wt. %, 4 wt. %, 5 wt. %, 6 wt. % 7 wt. %, 8 wt. %, 9 wt. %, 10 wt. %, 12 wt. %, 15 wt. %, 17%, 20 wt. %, 25 wt. %, 30 wt. %, 40 wt. %, 45 wt. %, or 50 wt. % of the total weight of enzymes in the cellulase composition, and the upper limit is about 10 wt. %, 12 wt. %, 15 wt. %, 17 wt. %, 20 wt. %, 25 wt. %, 30 wt. %, 35 wt. %, 40 wt. %, 50 wt. %, 55 wt. %, 60 wt. %, 65 wt. %, or 70 wt. % of the total weight of enzymes in the cellulase composition. For example, the β-glucosidase(s) suitably represent about 0.1 wt. % to about 40 wt. %, about 1 wt. % to about 35 wt. %, about 2 wt. % to about 30 wt. %; about 5 wt. % to about 25 wt. %, about 7 wt. % to about 20 wt. %, about 9 wt. % to about 17 wt. %, about 10 wt. % to about 20 wt. %; or about 5 wt. % to about 10 wt. % of the total weight of enzymes in the cellulase composition.

Mutant β-Glucosidase Polypeptides:

The present disclosure provides for mutant β-glucosidase polypeptides. Mutant β-glucosidase polypeptides include those in which one or more amino acid residues have undergone an amino acid substitution while retaining β-glucosidase activity (i.e., the ability to catalyze the hydrolysis of terminal non-reducing residues in β-D-glucosides with release of glucose). As such, mutant β-glucosidase polypeptides constitute a particular type of “β-glucosidase polypeptides,” as that term is defined herein. Mutant β-glucosidase polypeptides can be made by substituting one or more amino acids into the native or wild type amino acid sequence of the polypeptide. In some aspects, the invention includes polypeptides comprising altered amino acid sequences in comparison with a precursor enzyme amino acid sequence, wherein the mutant enzyme retains the characteristic cellulolytic nature of the precursor enzyme but may have altered properties in some specific aspects, e.g., an increased or decreased pH optimum, an increased or decreased oxidative stability; an increased or decreased thermal stability, and increased or decreased level of specific activity towards one or more substrates, as compared to the precursor enzyme. Guidance in determining which amino acid residues may be substituted, inserted, or deleted without affecting biological activity can be found using computer programs known in the art, e.g., LASERGENE software (DNASTAR). The amino acid substitutions may be conservative or non-conservative and such substituted amino acid residues may or may not be one encoded by the genetic code. The amino acid substitutions may be located in the polypeptide carbohydrate-binding modules (CBMs), in the polypeptide catalytic domains (CD), and/or in both the CBMs and the CDs. The standard twenty amino acid “alphabet” has been divided into chemical families based on similarity of their side chains. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “conservative amino acid substitution” is one where the amino acid residue is replaced with an amino acid residue having a chemically similar side chain (i.e., replacing an amino acid having a basic side chain with another amino acid having a basic side chain). A “non-conservative amino acid substitution” is one where the amino acid residue is replaced with an amino acid residue having a chemically different side chain (i.e., replacing an amino acid having a basic side chain with another amino acid having an aromatic side chain).

Chimeric Polypeptides:

The present disclosure also provides hybrid/fusion/chimeric proteins that include a domain of a protein of the present disclosure attached to one or more fusion segments, which are typically heterologous to the protein (i.e., derived from a different source than the protein of the disclosure). Those hybrid/fusion/chemric enzymes may also be deemed a type of mutant β-glucosidase in that they very in sequence from the wild type reference β-glucosidase but retains β-glucosidase activity, albeit having other differing properties from the native or wild type reference β-glucosidase. Suitable chimeric segments include, without limitation, segments that can enhance a protein's stability, provide other desirable biological activity or enhanced levels of desirable biological activity, and/or facilitate purification of the protein (e.g., by affinity chromatography). A suitable chimeric segment can be a domain of any size that has the desired function (e.g., imparts increased stability, solubility, action or biological activity; and/or simplifies purification of a protein). A chimeric protein of the invention can be constructed from two or more chimeric segments, each of which or at least two of which are derived from a different source or microorganism. Chimeric segments can be joined to amino and/or carboxyl termini of the domain(s) of a protein of the present disclosure. The chimeric segments can be susceptible to cleavage. There may be advantage in having this susceptibility, e.g., it may enable straight-forward recovery of the protein of interest. Chimeric proteins are preferably produced by culturing a recombinant cell transfected with a chimeric nucleic acid that encodes a protein, which includes a chimeric segment attached to either the carboxyl or amino terminal end, or chimeric segments attached to both the carboxyl and amino terminal ends, of a protein, or a domain thereof.

Accordingly, the β-glucosidase polypeptides of the present disclosure also include expression products of gene fusions (e.g., an overexpressed, soluble, and active form of a recombinant protein), of mutagenized genes (e.g., genes having codon modifications to enhance gene transcription and translation), and of truncated genes (e.g., genes having signal sequences removed or substituted with a heterologous signal sequence).

Glycosyl hydrolases that utilize insoluble substrates are often modular enzymes. They usually comprise catalytic modules appended to one or more non-catalytic carbohydrate-binding modules (CBMs). In nature, CBMs are thought to promote the glycosyl hydrolase's interaction with its target substrate polysaccharide. Thus, the disclosure provides chimeric enzymes having altered substrate specificity; including, e.g., chimeric enzymes having multiple substrates as a result of “spliced-in” heterologous CBMs. The heterologous CBMs of the chimeric enzymes of the disclosure can also be designed to be modular, such that they are appended to a catalytic module or catalytic domain (a “CD”, e.g., at an active site), which can likewise be heterologous or homologous to the glycosyl hydrolase.

Thus, the disclosure provides peptides and polypeptides consisting of, or comprising, CBM/CD modules, which can be homologously paired or joined to form chimeric (heterologous) CBM/CD pairs. Thus, these chimeric polypeptides/peptides can be used to improve or alter the performance of an enzyme of interest. Accordingly, in some aspects, the disclosure provides chimeric enzymes comprising, e.g., at least one CBM of an enzyme, if available, of SEQ ID NO:2, 4, 6, 8, 10, 12, 14, 16, 18, 20, 22, 24, 26, 28, 30, 32, 34, 36, 38, 40, 42, 43, 44, 52, 54, 56, 58, 60, 62, 64, 66, 68, 70, 72, 74, 76, 78, or 79. A polypeptide of the disclosure, e.g., includes an amino acid sequence comprising the CD and/or CBM of the polypeptide sequence of SEQ ID NO:2, 4, 6, 8, 10, 12, 14, 16, 18, 20, 22, 24, 26, 28, 30, 32, 34, 36, 38, 40, 42, 43, 44, 52, 54, 56, 58, 60, 62, 64, 66, 68, 70, 72, 74, 76, 78, or 79. The polypeptide of the disclosure can thus suitably be a fusion protein comprising functional domains from two or more different proteins (e.g., a CBM from one protein linked to a CD from another protein).

The disclosure also provides a non-naturally occurring cellulase composition comprising a β-glucosidase polypeptide, which is a chimera of at least two β-glucosidase sequences. In some aspects, the non-naturally occurring cellulase composition comprises β-glucosidase activity. The composition may further comprise one or more of xylanase, β-xylosidase, and/or L-α-arabinofuranosidase activities. Thus the composition is a hemicellulase composition. In some aspects, the non-naturally occurring cellulase/hemicellulase composition comprises enzymatic components or polypetpides that are derived from at least two different sources. In some aspects, the non-naturally occurring cellulase/hemicellulase composition comprises one or more naturally occurring hemicellulases.

In some aspects, the β-glucosidase polypeptides in the composition further comprises one or more glycosylation sites. In some aspects, the β-glucosidase polypeptide comprises an N-terminal sequence and a C-terminal sequence, wherein each of the N-terminal sequence or the C-terminal sequence can comprise one or more sub-sequences derived from different β-glucosidases. In certain aspects, the N-terminal and C-terminal sequences are derived from different sources. In some embodiments, at least two of the one or more sub-sequences of the N-terminal and the C-terminal sequences are derived from different sources. In some aspects, either the N-terminal sequence or the C-terminal sequence further comprises a loop region sequence of about 3, 4, 5, 6, 7, 8, 9, 10, or 11 amino acid residues in length. In certain embodiments, the N-terminal sequence and the C-terminal sequence are immediately adjacent or directly connected. In other embodiments, the N-terminal and C-terminal sequences are not immediately adjacent, but rather, they are functionally connected via a linker domain. The linker domain may be centrally located (e.g., not located at either the N-terminal or the C-terminal) of the chimeric polypeptide. In certain embodiments, neither the N-terminal sequence nor the C-terminal sequence of the hybrid polypeptide comprises a loop sequence. Instead, the linker domain comprises the loop sequence. In some aspects, the N-terminal sequence comprises a first amino acid sequence of a β-glucosidase or a variant thereof that is at least about 200 (e.g., about 200, 250, 300, 350, 400, 450, 500, 550, or 600) residues in length. In some aspects, the N-terminal sequence comprises one or more or all of the polypeptide sequence motifs represented by SEQ ID NOs:136-148. In some aspects, the C-terminal sequence comprises a second amino acid sequence of a β-glucosidase or a variant thereof that is at least about 50 (e.g., about 50, 75, 100, 125, 150, 175, or 200) amino acid residues in length. In some aspects, the C-terminal sequence comprises one or more or all of the polypeptide sequence motifs represented by SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In some aspects, either the C-terminal or the N-terminal sequence comprises a loop sequence, which comprises about 3, 4, 5, 6, 7, 8, 9, 10, or 11 amino acid residues, and a sequence of FDRRSPG (SEQ ID NO:171), or of FD(R/K)YNIT (SEQ ID NO:172). In some aspects, neither the C-terminal nor the N-terminal sequence comprises a loop sequence. In some embodiments, the C-terminal sequence and the N-terminal sequence are connected via a linker domain that comprises a loop sequence, which comprises about 3, 4, 5, 6, 7, 8, 9, 10, or 11 amino acid residues, and a sequence of FDRRSPG (SEQ ID NO:171), or of FD(R/K)YNIT (SEQ ID NO:172). In some aspects, the β-glucosidase polypeptide(s) in the non-naturally occurring cellulase or hemicellulase composition has improved stability over any of the native enzymes from which each C-terminal and/or the N-terminal sequences of the chimeric polypeptide was derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 30%, or less than about 20%, more preferably less than 15%, or less than 10%.

The polypeptides of the disclosure can suitably be obtained and/or used in “substantially pure” form. For example, a polypeptide of the disclosure constitutes at least about 80 wt. % (e.g., at least about 85 wt. %, 90 wt. %, 91 wt. %, 92 wt. %, 93 wt. %, 94 wt. %, 95 wt. %, 96 wt. %, 97 wt. %, 98 wt. %, or 99 wt. %) of the total protein in a given composition, which also includes other ingredients such as a buffer or solution.

Fermentation Broths:

Also, the polypeptides of the disclosure can suitably be obtained and/or used in fermentation broths (e.g., a filamentous fungal culture broth). The fermentation broths can be an engineered enzyme composition, e.g., the fermentation broth can be produced by a recombinant host cell engineered to express a heterologous polypeptide of interest, or by a recombinant host cell that is engineered to express an endogenous polypeptide of the disclosure in greater or lesser amounts than the endogenous expression levels (e.g., in an amount that is about 1-, 2-, 3-, 4-, 5-, fold or more-greater or less than the endogenous expression levels). The fermentation broths of the invention may also be produced by certain “integrated” host cell strains that are engineered to express a plurality of the polypeptides of the disclosure in desired ratios. One or more or all of the genes encoding the polypeptides of interest may be intergrated into the genetic materials of the host cell strain, for example.

### Fv3C

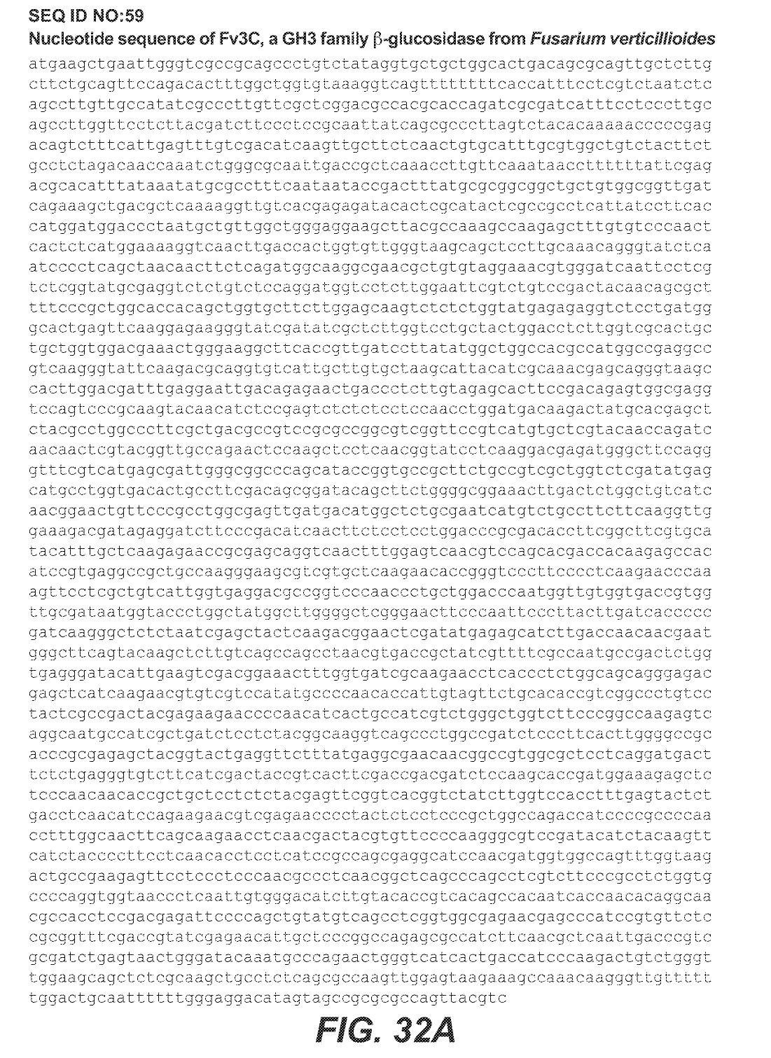

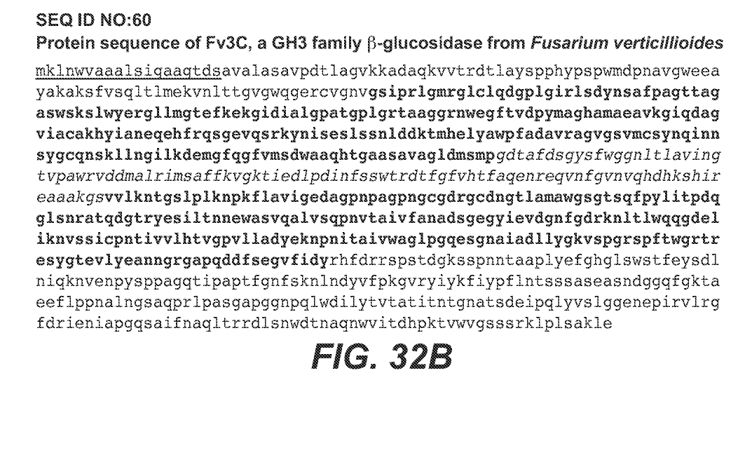

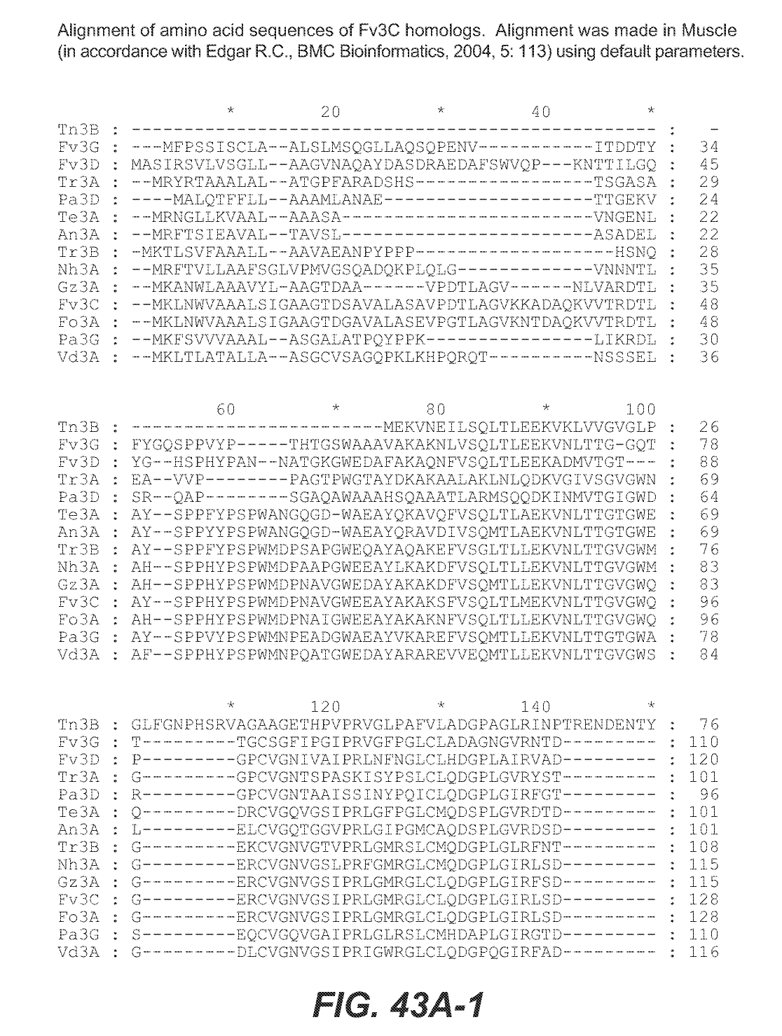

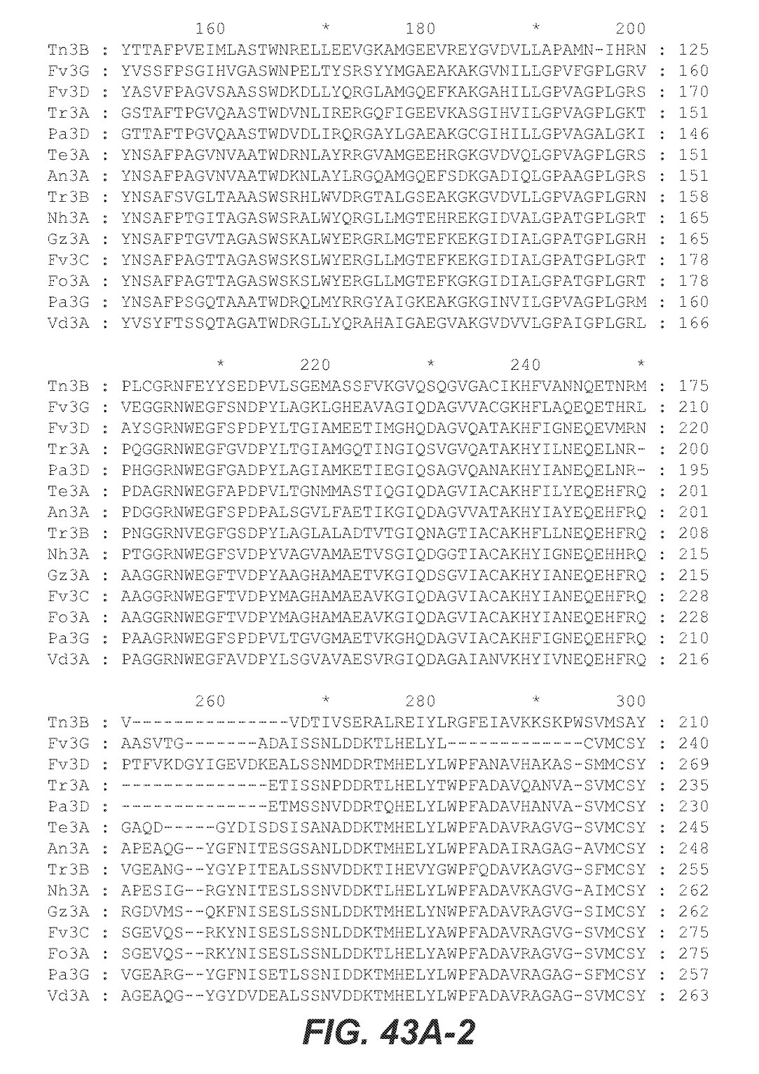

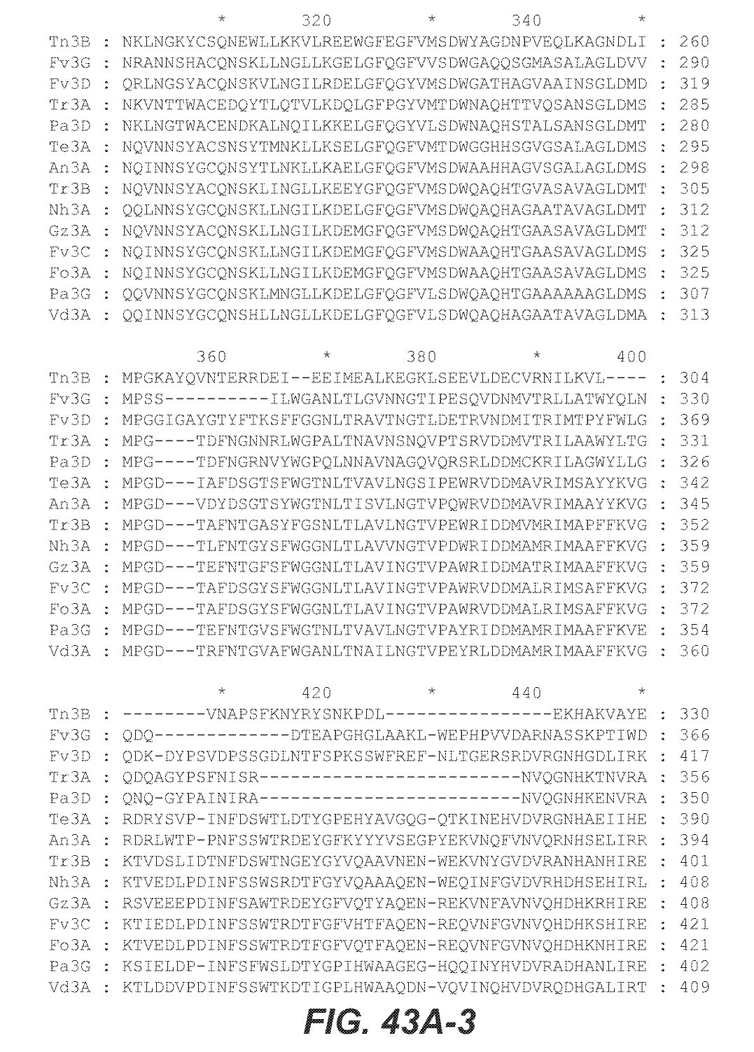

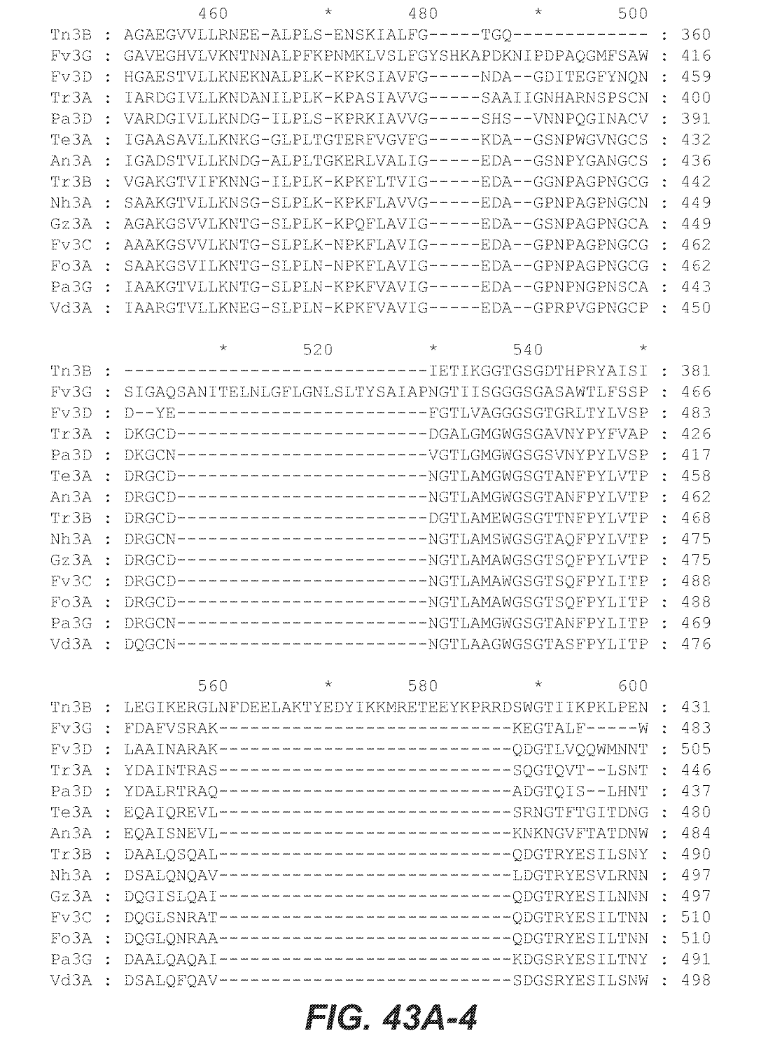

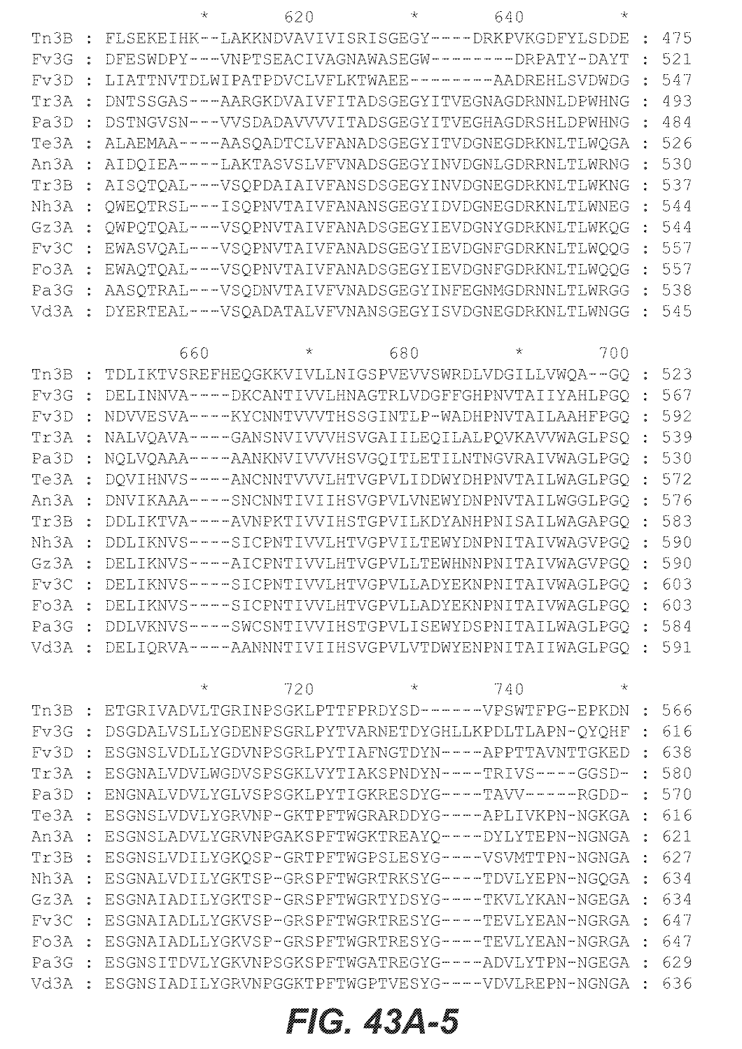

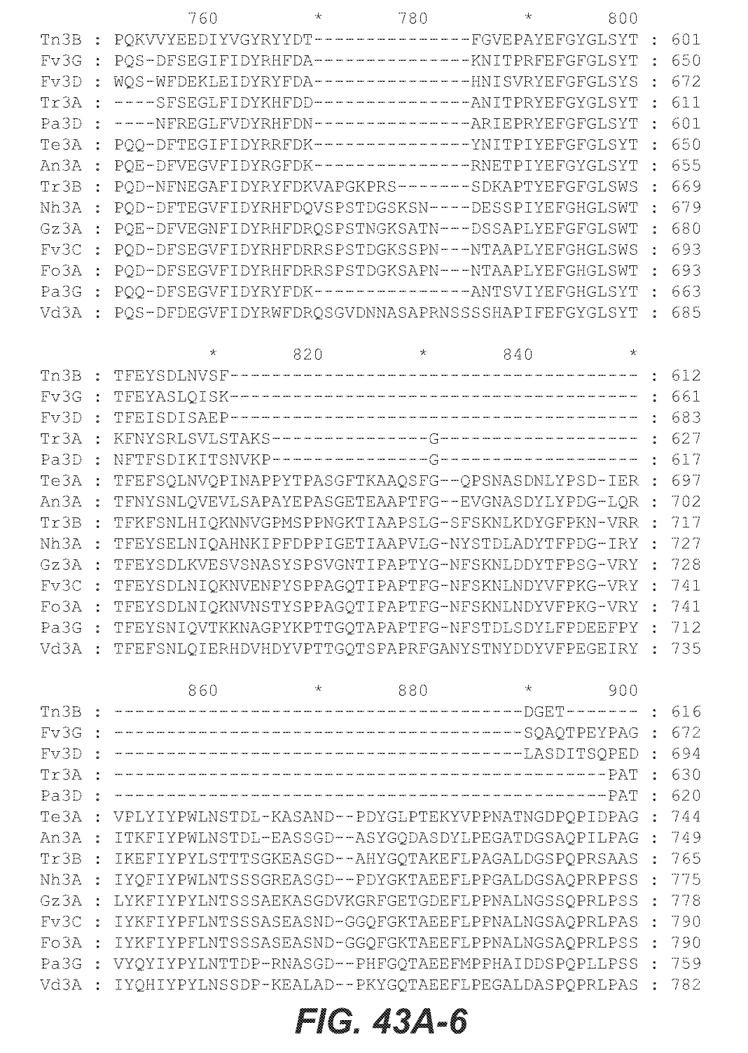

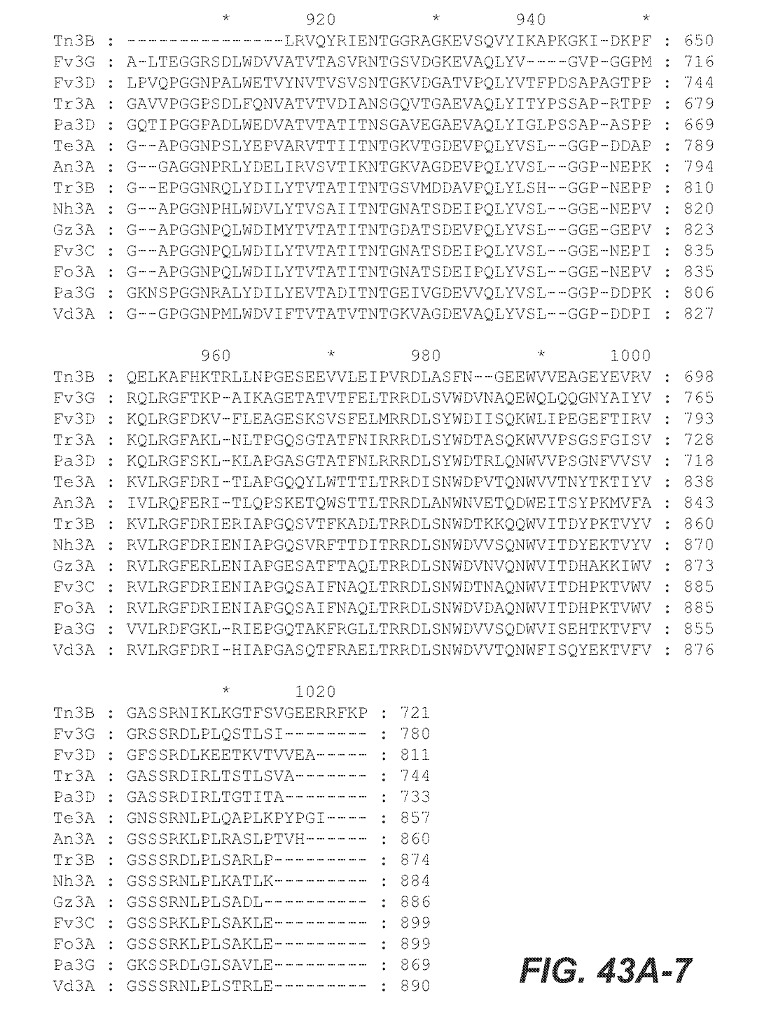

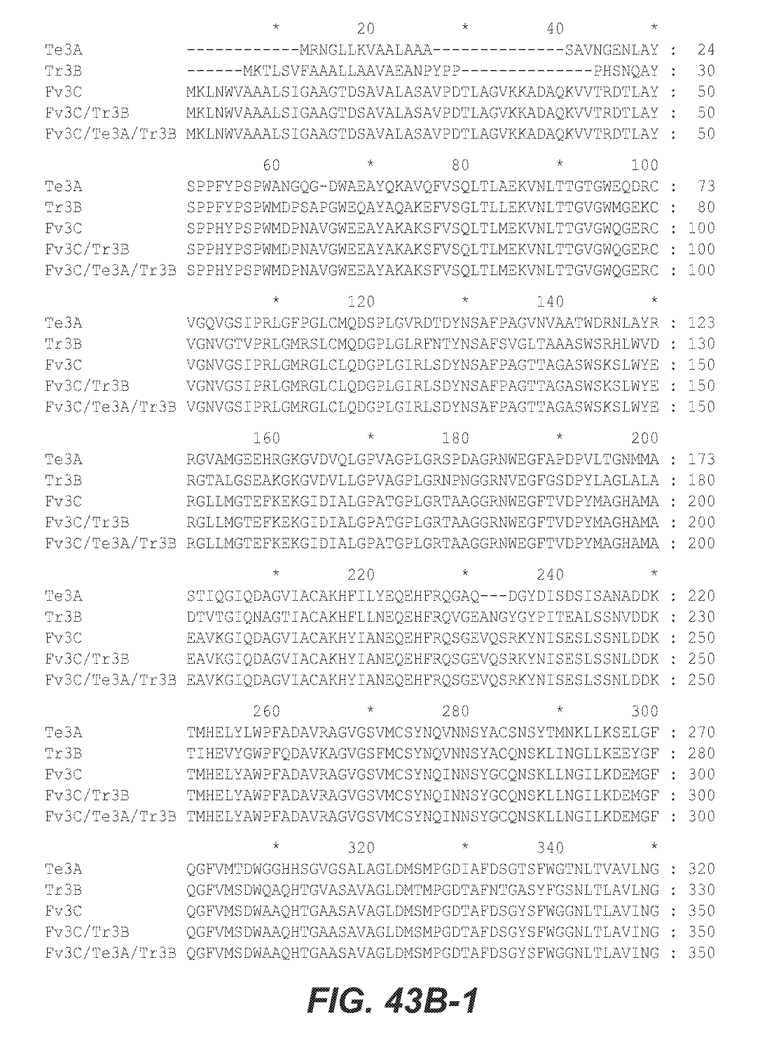

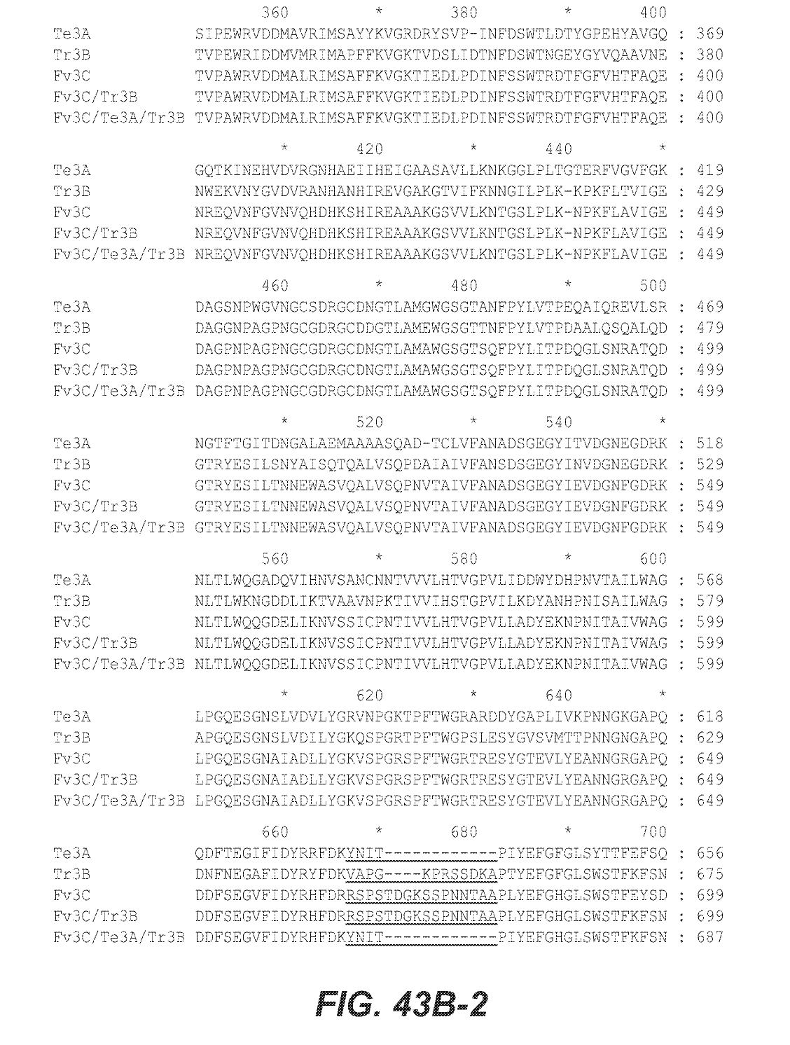

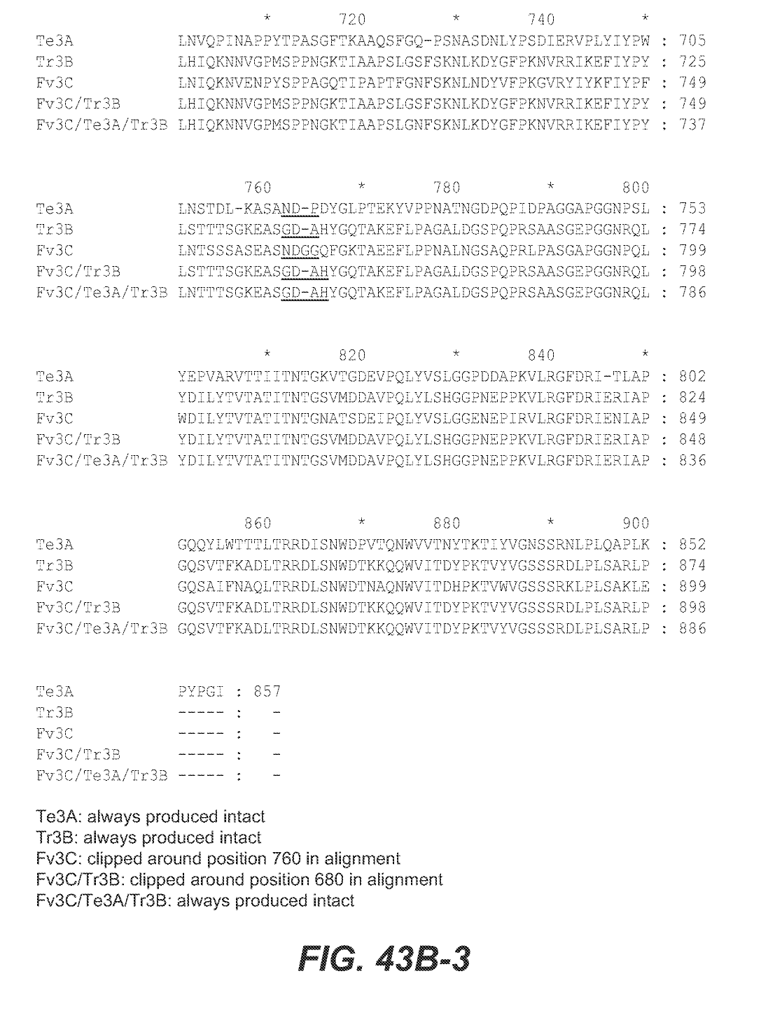

The amino acid sequence of Fv3C (SEQ ID NO:60) is shown in FIGS. 32B and 43. SEQ ID NO:60 is the sequence of the immature Fv3C. Fv3C has a predicted signal sequence corresponding to positions 1 to 19 of SEQ ID NO:60 (underlined); cleavage of the signal sequence is predicted to yield a mature protein having a sequence corresponding to positions 20 to 899 of SEQ ID NO:60. Signal sequence predictions were made with the SignalP-NN algorithm. The predicted conserved domain is in boldface type in FIG. 32B. Domain predictions were made based on the Pfam, SMART, or NCBI databases. Fv3C residues E536 and D307 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc (see, FIG. 43). As used herein, “an Fv3C polypeptide” refers, in some aspect, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, or 800 contiguous amino acid residues among residues 20 to 899 of SEQ ID NO:60. An Fv3C polypeptide preferably is unaltered, as compared to a native Fv3C, at residues E536 and D307. An Fv3C polypeptide is preferably unaltered in at least 70%, 80%, 90%, 95%, 98%, or 99% of the amino acid residues that are conserved among the herein described GH3 family β-glucosidases as shown in the alignment of FIG. 43. An Fv3C polypeptide suitably comprises the entire predicted conserved domains of native Fv3C shown in FIG. 32B. An exemplary Fv3C polypeptide comprises a sequence having at least 85%, 86%, 87%, 88%, 89%, 90%, 91%, 92%, 93%, 94%, 95%, 96%, 97%, 98%, 99%, or 100% identity to the mature Fv3C sequence shown in FIG. 32B. The Fv3C polypeptide of the invention preferably has β-glucosidase activity.

Accordingly an Fv3C polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:60, or to residues (i) 20-327, (ii) 22-600, (iii) 20-899, (iv) 428-899, or (v) 428-660 of SEQ ID NO:60. The polypeptide suitably has β-glucosidase activity.

In some aspects, an “Fv3C polypeptide” of the invention may refer to a mutant Fv3C polypeptide. Amino acid substitutions may be introduced into the Fv3C polypeptide to improve the β-glucosidase activity and/or stability of the molecule. For example, amino acid substitutions that increase the binding affinity of the Fv3C polypeptide for its substrate or that improve Fv3C's ability to catalyze the hydrolysis of terminal non-reducing residues in β-D-glucosides can be introduced into the polypeptide. In some aspects, the mutant Fv3C polypeptides comprise one or more conservative amino acid substitutions. In some aspects, the mutant Fv3C polypeptides comprise one or more non-conservative amino acid substitutions. In some aspects, the one or more amino acid substitutions are in the Fv3C polypeptide CD. Or the one or more amino acid substitutions are in the Fv3C polypeptide CBM. The one or more amino acid substitutions may be in both the CD and the CBM. In some aspects, the Fv3C polypeptide amino acid substitutions may take place at amino acids E536 and/or D307. In some aspects, the Fv3C polypeptide amino acid substitutions may take place at one or more or all of amino acids D119, R125, L168, R183, K216, H217, R227, M272, Y275, D307, W308, S477, and/or E536. The mutant Fv3C polypeptide(s) suitably have β-glucosidase activity.

In some aspects, the Fv3C polypeptide comprises a chimera/fusion/hybrid or a chimeric construct of two β-glucosidase sequences, wherein the first sequence is derived from a first β-glucosidase, is at least about 200 amino acid residues in length, and comprises about 60%, 65%, 70%, 75%, 80% or higher identity to a sequence of equal length of Fv3C (SEQ ID NO: 60), and wherein the second sequence is derived from a second β-glucosidase, is at least about 50 amino acid residues in length, and comprises about 60%, 65%, 70%, 75%, 80% or higher identity to a sequence of equal length of any one of SEQ ID NOs:54, 56, 58, 62, 64, 66, 68, 70, 72, 74, 76, 78, and 79, or comprises the amino acid sequence motif of SEQ ID:170. In some aspects, the first β-glucosidase sequence comprises an N-terminal sequence of at least about 200 contiguous amino acid residues of SEQ ID NO:60, and the second β-glucosidase sequence comprises a C-terminal sequence of at least about 50 contiguous amino acid residues of any one of SEQ ID NOs:54, 56, 58, 62, 64, 66, 68, 70, 72, 74, 76, 78, and 79, or comprises the amino acid sequence motif of SEQ ID NO:170.

In certain aspects, the Fv3C polypeptide may be a chimera/hybrid/fusion or a chimeric construct of two β-glucosidase sequences, wherein the first sequence is derived from a first β-glucosidase, is at least about 200 amino acid residues in length, and comprises about 60%, 65%, 70%, 75%, 80% or higher identity to a sequence of equal length of any one of SEQ ID NOs:54, 56, 58, 62, 64, 66, 68, 70, 72, 74, 76, 78, and 79, or comprises one or more or all of the amino acid sequence motifs of SEQ ID NOs: 164-169, wherein the second sequence is derived from a second β-glucosidase, is at least about 50 amino acid residues in length, and comprises about 60%, 65%, 70%, 75%, 80% or higher identity to a sequence of equal length of Fv3C (SEQ ID NO: 60). In some aspects, the first β-glucosidase sequence comprises an N-terminal sequence of at least 200 contiguous amino acid residues of SEQ ID NOs:54, 56, 58, 62, 64, 66, 68, 70, 72, 74, 76, 78, or 79, or comprises one or more or all of the amino acid sequence motifs of SEQ ID NOs: 164-169, and the second β-glucosidase sequence comprises a C-terminal sequence of at least about 50 contiguous amino acid residues of SEQ ID NO:60.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In some embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an Fv3C polypeptide or a variant thereof. In some aspects, the N-terminal sequence comprises one or more or all of the polypeptide sequence motifs represented by SEQ ID NOs:136-148. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In certain embodiments, the β-glucosidase polypeptide, the variant thereof, or the hybrid/chimera thereof further comprises one or more glycosylation sites. The one or more glycosylation sites can be located within the C-terminal sequence, within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including over Fv3C, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the rate or extent of enzymatic activity loss is preferably less than about 50%, less than about 40%, less than about 20%, more preferably less than about 15%, or even more preferably less than about 10%. In some aspects, the β-glucosidase polypeptide is a chimeric or fusion enzyme comprising a sequence of an Fv3C polypeptide operably linked to a sequence of a T. reesei Bgl3. In certain embodiments, the β-glucosidase polypeptide comprises an N-terminal sequence that is derived from an Fv3C polypeptide, and a C-terminal sequence that is derived from a T. reesei Bgl3 polypeptide. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. The non-naturally occurring cellulase composition may further comprise one or more of xylanase, β-xylosidase, and/or L-α-arabinofuranosidase activities.

### Pa3D:

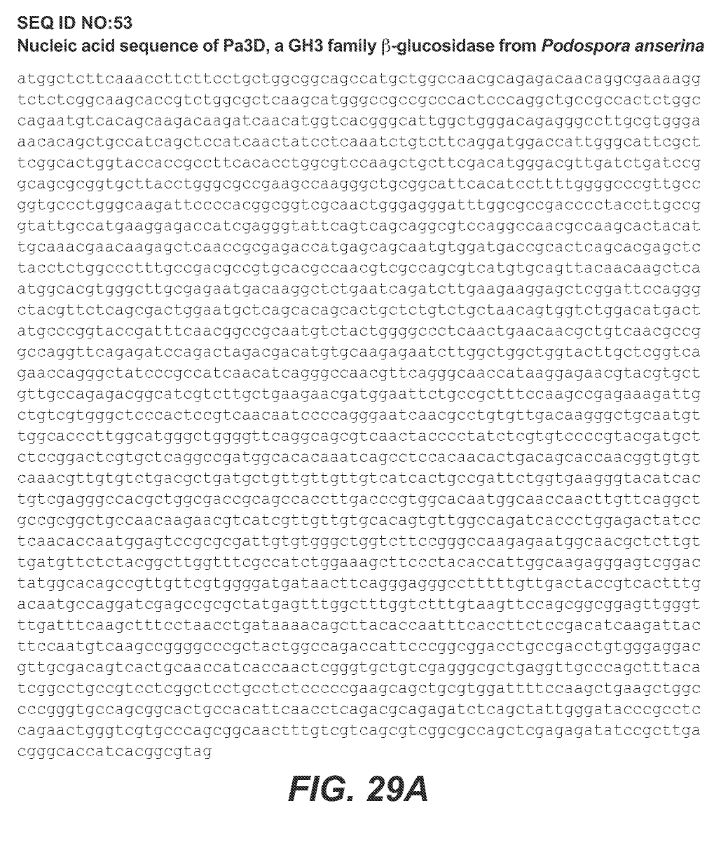

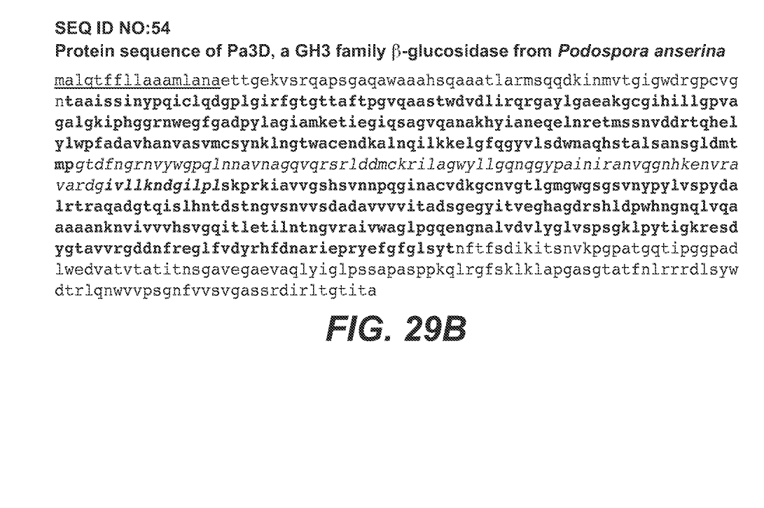

The amino acid sequence of Pa3D (SEQ ID NO:54) is shown in FIGS. 29B and 43. SEQ ID NO:54 is the sequence of the immature Pa3D. Pa3D has a predicted signal sequence corresponding to residues 1 to 17 of SEQ ID NO:2 (underlined); cleavage of the signal sequence is predicted to yield a mature protein having a sequence corresponding to residues 18 to 733 of SEQ ID NO:54. Signal sequence predictions for this and other polypeptides of the disclosure were made with the SignalP-NN algorithm (www.cbs.dtu.dk). The predicted conserved domain is in bold in FIG. 29B. Domain predictions for this and other polypeptides of the disclosure were made based on the Pfam, SMART, or NCBI databases. Pa3D residues E463 and D262 are predicted to function as catalytic acid-base and nucleophile, respectively, based on a sequence alignment of a number of GH3 family β-glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Pa3D polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650 or 700 contiguous amino acid residues among residues 18 to 733 of SEQ ID NO:54. A Pa3D polypeptide preferably is unaltered, as compared to a native Pa3D, at residues E463 and D262. A Pa3D polypeptide is preferably unaltered in at least 70%, 80%, 90%, 95%, 98%, or 99% of the amino acid residues that are conserved among the herein described GH3 family β-glucosidases as shown in the alignment of FIG. 43. A Pa3D polypeptide suitably comprises the entire predicted conserved domains of native Pa3D shown in FIG. 29B. An exemplary Pa3D polypeptide comprises a sequence having at least 85%, 86%, 87%, 88%, 89%, 90%, 91%, 92%, 93%, 94%, 95%, 96%, 97%, 98%, 99%, or 100% identity to the mature Pa3D sequence shown in FIG. 29B. The Pa3D polypeptide of the invention preferably has β-glucosidase activity.

Accordingly a Pa3D polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:54, or to residues (i) 18-282, (ii) 18-601, (iii) 18-733, (iv) 356-601, or (v) 356-733 of SEQ ID NO:54. The polypeptide suitably has β-glucosidase activity.

A “Pa3D polypeptide” of the invention may also refer to a mutant Pa3D polypeptide. Amino acid substitutions may be introduced into the Pa3D polypeptide to improve the β-glucosidase activity and/or other properties. For example, amino acid substitutions that increase binding affinity of the Pa3D polypeptide for its substrate or that improve Pa3D's ability to catalyze the hydrolysis of terminal non-reducing residues in β-D-glucosides may be introduced. In some aspects, the mutant Pa3D polypeptides comprise one or more conservative amino acid substitutions. Or the mutant Pa3D polypeptides may comprise one or more non-conservative amino acid substitutions. In some aspects, the one or more amino acid substitutions are in the Pa3D polypeptide CD. Or, the one or more amino acid substitutions are in the Pa3D polypeptide CBM. The one or more amino acid substitutions may be in both the CD and the CBM. In some aspects, the Pa3D polypeptide amino acid substitutions may take place at amino acids E463 and/or D262. The Pa3D polypeptide amino acid substitutions may take place at one or more or all of amino acids D87, R93, L136, R151, K184, H185, R195, M227, Y230, D262, W263, S406 and/or E463. The mutant Pa3D polypeptide(s) suitably have β-glucosidase activity.

In some aspects, the Pa3D polypeptide may be a chimera/hybrid/fusion of two β-glucosidase sequences, wherein the first sequence is derived from a first β-glucosidase, is at least about 200 amino acid residues in length, and comprises about 60% (e.g., about 60%, 65%, 70%, 75%, or 80%) or higher identity to a sequence of equal length of Pa3D (SEQ ID NO: 54), and wherein the second sequence is derived from a second β-glucosidase, is at least about 50 amino acid residues in length, and has about 60%, 70%, 75%, 80% or higher identity to a sequence of equal length of any one of SEQ ID NOs: 56, 58, 60, 62, 64, 66, 68, 70, 72, 74, 76, 78, and 79, or comprises an amino acid sequence motif of SEQ ID NO:170. In some aspects, the first β-glucosidase sequence comprises an N-terminal sequence of at least about 200 contiguous amino acid residues of SEQ ID NO:54, and the second β-glucosidase sequence comprises a C-termus sequence of at least about 50 contiguous amino acid residues of any one of SEQ ID NOs: 56, 58, 60, 62, 64, 66, 68, 70, 72, 74, 76, 78, and 79, or comprise an amino acid sequence motif of SEQ ID NO:170.

In some aspects, the Pa3D polypeptide of the invention comprises a chimera/hybrid/fusion or a chimeric construct of β-glucosidase sequences, wherein the first sequence is from a first β-glucosidase, is at least about 200 amino acid residues in length, and has about 60% (e.g., 60%, 65%, 70%, 75%, or 80%) or higher identity to a sequence of equal length of any one of SEQ ID NOs: 56, 58, 60, 62, 64, 66, 68, 70, 72, 74, 76, 78, and 79, or comprises one or more or all of amino acid sequence motifs SEQ ID NOs: 164-169, and the second sequence is from a second β-glucosidase, is at least about 50 amino acid residues in length, and has about 60%, 65%, 70%, 75%, 80% or higher identity to a sequence of equal length of Pa3D (SEQ ID NO:54). For example, the first β-glucosidase sequence comprises an N-terminal sequence of at least 200 contiguous amino acid residues of SEQ ID NOs: 56, 58, 60, 62, 64, 66, 68, 70, 72, 74, 76, 78, or 79, or comprises one or more or all of amino acid sequence motifs SEQ ID NOs: 164-169, and the second β-glucosidase sequence comprises a C-terminal sequence of at least 50 contiguous amino acid residues of SEQ ID NO:54.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Pa3D polypeptide or a variant thereof. In some aspects, the N-terminal sequence comprises one or more or all of the polypeptide sequence motifs represented by SEQ ID NOs:136-148, or preferably one or more or all sequence motifs SEQ ID NOs: 164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably a polypeptide sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including over Pa3D, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Fv3G

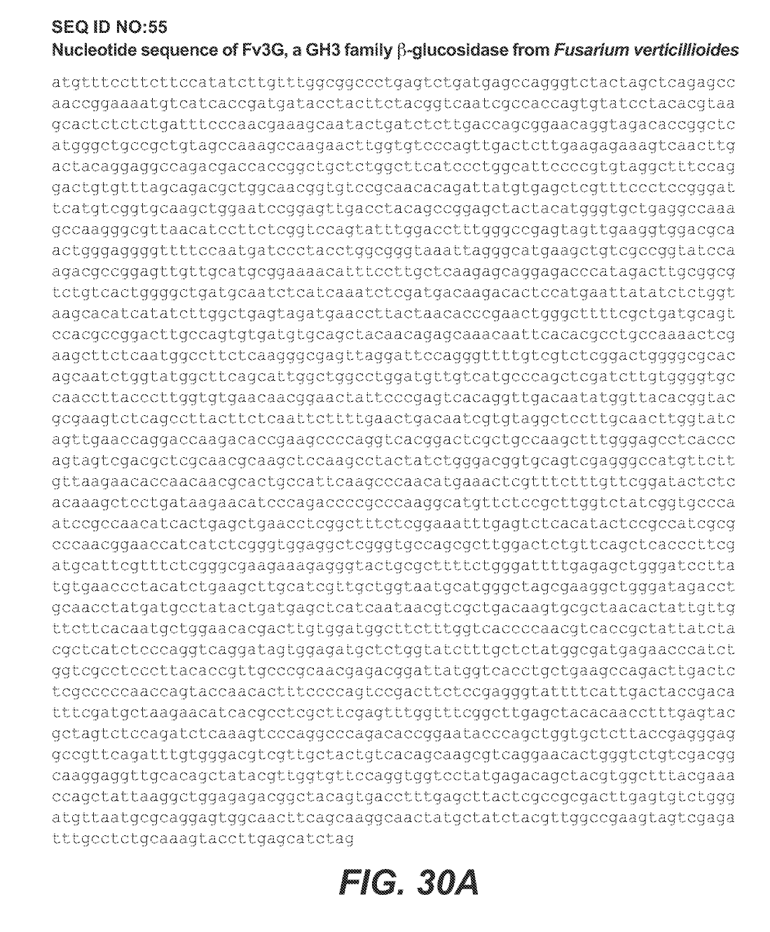

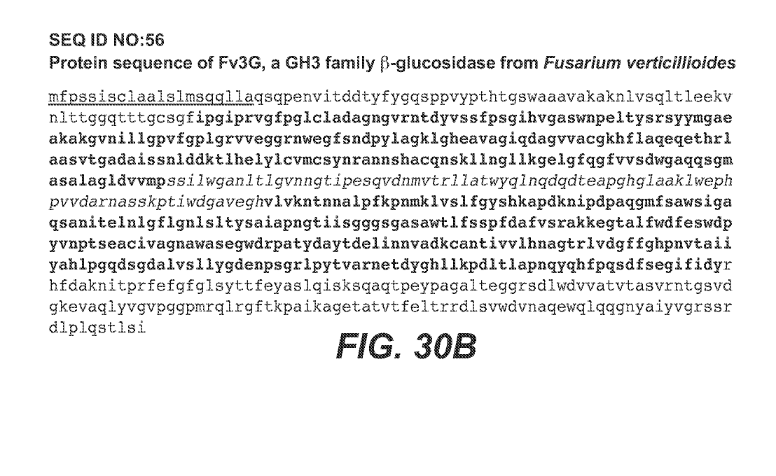

The amino acid sequence of Fv3G (SEQ ID NO:56) is shown in FIGS. 30B and 43. SEQ ID NO:56 is the sequence of the immature Fv3G. Fv3G has a predicted signal sequence corresponding to positions 1 to 21 of SEQ ID NO:56 (underlined); cleavage of the signal sequence is predicted to yield a mature protein having a sequence corresponding to positions 22 to 780 of SEQ ID NO:56. Signal sequence predictions were, as described above, made with the SignalP-NN algorithm (http://www.cbs.dtu.dk), as they were made for the other polypeptides of the disclosure herein. The predicted conserved domain is in boldface type in FIG. 30B. Domain predictions were made, as they were made with the other polypeptides of the invention herein, based on the Pfam, SMART, or NCBI databases. Fv3G residues E509 and D272 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “an Fv3 Gpolypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 20 to 780 of SEQ ID NO:56. An Fv3G polypeptide preferably is unaltered, as compared to a native Fv3G, at residues E509 and D272. An Fv3G polypeptide is preferably unaltered in at least 70%, 80%, 90%, 95%, 98%, or 99% of the amino acid residues that are conserved among the herein described GH3 family β-glucosidases as shown in the alignment of FIG. 43. An Fv3G polypeptide suitably comprises the entire predicted conserved domains of native Fv3G shown in FIG. 30B. An exemplary Fv3G polypeptide comprises a sequence having at least 85%, 86%, 87%, 88%, 89%, 90%, 91%, 92%, 93%, 94%, 95%, 96%, 97%, 98%, 99%, or 100% identity to the mature Fv3G sequence shown in FIG. 30B. The Fv3G polypeptide of the invention preferably has β-glucosidase activity.

Accordingly an Fv3G polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:56, or to residues (i) 22-292, (ii) 22-629, (iii) 22-780, (iv) 373-629, or (v) 373-780 of SEQ ID NO:56. The polypeptide suitably has β-glucosidase activity.

In some aspects, an “Fv3G polypeptide” of the invention can also refer to a mutant Fv3G polypeptide. Amino acid substitutions can be introduced into the Fv3G polypeptide to improve the β-glucosidase activity of the molecule. For example, amino acid substitutions that increase the binding affinity of the Fv3G polypeptide for its substrate or that improve Fv3G's ability to catalyze the hydrolysis of terminal non-reducing residues in β-D-glucosides can be introduced into the Fv3G polypeptide. In some aspects, the mutant Fv3G polypeptides comprise one or more conservative amino acid substitutions. In some aspects, the mutant Fv3G polypeptides comprise one or more non-conservative amino acid substitutions. In some aspects, the one or more amino acid substitutions are in the Fv3G polypeptide CD. In some aspects, the one or more amino acid substitutions are in the Fv3G polypeptide CBM. In some aspects, the one or more amino acid substitutions are in both the CD and the CBM. In some aspects, the Fv3G polypeptide amino acid substitutions can take place at amino acids E509 and/or D272. In some aspects, the Fv3G polypeptide amino acid substitutions can take place at one or more of amino acids D101, R107, L150, R165, K198, H199, R209, M237, Y240, D272, W273, S455, and/or E509. The mutant Fv3G polypeptide(s) suitably have β-glucosidase activity.

In some aspects, the Fv3G polypeptide comprises a chimera of two β-glucosidase sequences, wherein the first β-glucosidase sequence is at least about 200 amino acid residues in length, and comprises about 60%, 65%, 70%, 75%, or 80% or more sequence identity to a sequence of equal length of Fv3G (SEQ ID NO:56) and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 58, 60, 62, 64, 66, 68, 70, 72, 74, 76, 78, and 79, or comprises a polypeptide sequence motif SEQ ID NO:170. In some aspects, the first β-glucosidase sequence comprising an N-terminal sequence of at least 200 amino acid residues of SEQ ID NO:56, and the second β-glucosidase sequence comprising a C-terminal sequence of at least about 50 contiguous amino acid residues of any one of SEQ ID NOs:54, 58, 60, 62, 64, 66, 68, 70, 72, 74, 76, 78, and 79, or comprises the motif SEQ ID NO:170.

In certain aspects, the Fv3G polypeptide of the invention comprises a chimera or a chimeric construct of two β-glucosidase sequences, wherein the first β-glucosidase sequence is at least about 200 amino acid residues in length, and comprises about 60%, 65%, 70%, 75%, 80% or more sequence identity to a sequence of equal length of any one of SEQ ID NOs: 54, 58, 60, 62, 64, 66, 68, 70, 72, 74, 76, 78, and 79, or comprises one or more or all of the motifs SEQ ID NOs:164-169, whereas the second β-glucosidase sequence is at least about 50 amino acid residues in length comprises about 60%, 65%, 70%, 75%, 80% or more sequence identity to a sequence of equal length of Fv3G (SEQ ID NO:56). In some aspects, the first β-glucosidase sequence comprises an N-terminal sequence of at least 200 amino acid residues of any one of SEQ ID NOs: 54, 58, 60, 62, 64, 66, 68, 70, 72, 74, 76, 78, and 79, or comprises one or more or all of the sequence motifs SEQ ID NOs: 164-169, and the second β-glucosidase sequence comprises a C-terminal sequence of at least 50 contiguous amino acid residues of SEQ ID NO:56.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an Fv3G polypeptide or a variant thereof. In some aspects, the N-terminal sequence comprises one or more or all of the polypeptide sequence motifs represented by SEQ ID NOs:136-148, or preferably one or more or all of SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably SEQ ID NO:170. The β-glucosidase polypeptide, the variant thereof, or the hybrid or chimera thereof may further comprise one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Fv3G, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Fv3D

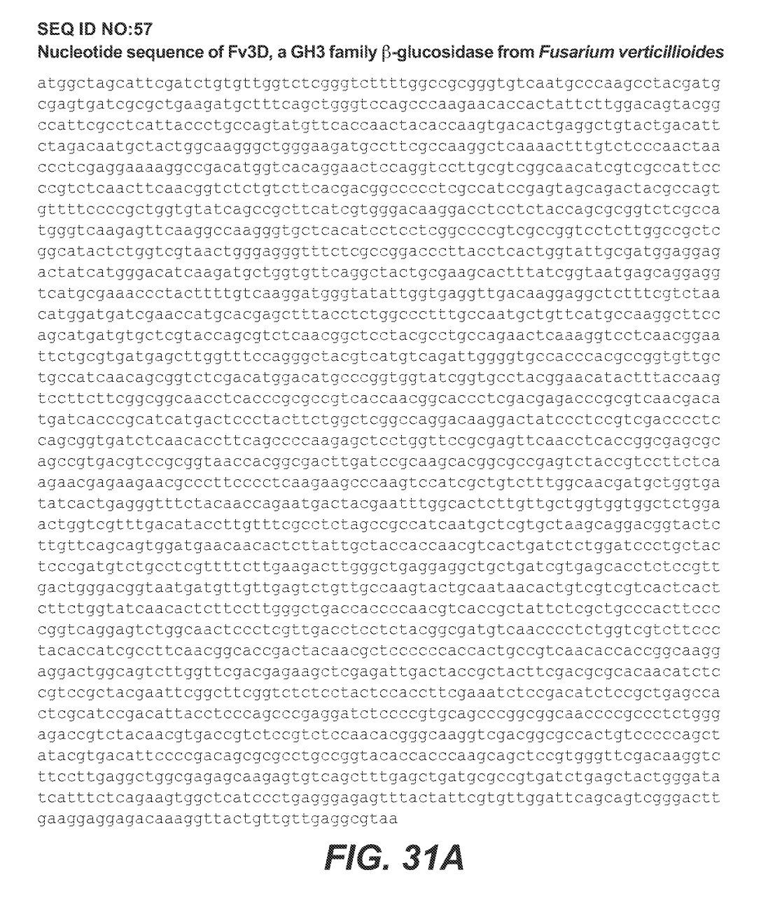

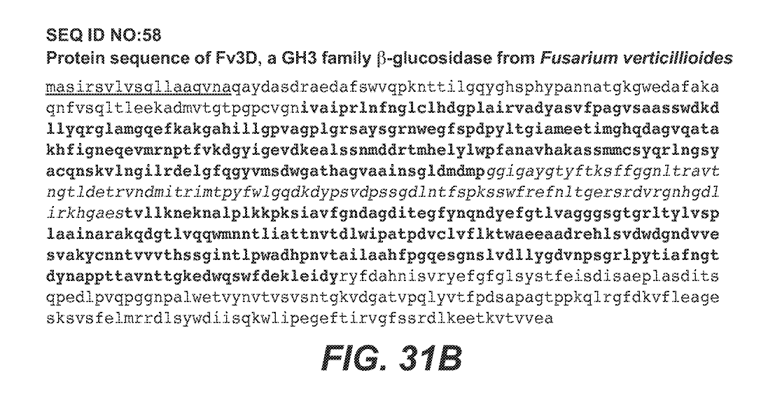

The amino acid sequence of Fv3D (SEQ ID NO:58) is shown in FIGS. 31B and 43. SEQ ID NO:58 is the sequence of the immature Fv3D. Fv3D has a predicted signal sequence corresponding to positions 1 to 19 of SEQ ID NO:58 (underlined); cleavage of the signal sequence is predicted to yield a mature protein having a sequence corresponding to positions 20 to 811 of SEQ ID NO:58. Signal sequence predictions were made with the SignalP-NN algorithm. The predicted conserved domain is in boldface type in FIG. 31B. Domain predictions were made based on the Pfam, SMART, or NCBI databases. Fv3D residues E534 and D301 are predicted to function as catalytic acid-base and nucleophile, respectively, based on a sequence alignment of the above-mentioned GH3 glucosidases from, e.g., P. (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “an Fv3D polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 20 to 811 of SEQ ID NO:58. An Fv3D polypeptide preferably is unaltered, as compared to a native Fv3D, at residues E534 and D301. An Fv3D polypeptide is preferably unaltered in at least 70%, 80%, 90%, 95%, 98%, or 99% of the amino acid residues that are conserved among the herein described GH3 family β-glucosidases as shown in the alignment of FIG. 43. An Fv3D polypeptide suitably comprises the entire predicted conserved domains of native Fv3D shown in FIG. 31B. An exemplary Fv3D polypeptide comprises a sequence having at least 85%, 86%, 87%, 88%, 89%, 90%, 91%, 92%, 93%, 94%, 95%, 96%, 97%, 98%, 99%, or 100% identity to the mature Fv3D sequence shown in FIG. 31B. The Fv3D polypeptide of the invention preferably has β-glucosidase activity.

Accordingly an Fv3D polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:58, or to residues (i) 20-321, (ii) 20-651, (iii) 20-811, (iv) 423-651, or (v) 423-811 of SEQ ID NO:58. The polypeptide suitably has β-glucosidase activity.

In some aspects, an “Fv3D polypeptide” of the invention can also refer to a mutant Fv3D polypeptide. Amino acid substitutions can be introduced into the Fv3D polypeptide to improve the β-glucosidase activity of the molecule. For example, amino acid substitutions that increase the binding affinity of the Fv3D polypeptide for its substrate or that improve Fv3D's ability to catalyze the hydrolysis of terminal non-reducing residues in β-D-glucosides can be introduced into the Fv3D polypeptide. In some aspects, the mutant Fv3D polypeptides comprise one or more conservative amino acid substitutions. In some aspects, the mutant Fv3D polypeptides comprise one or more non-conservative amino acid substitutions. In some aspects, the one or more amino acid substitutions are in the Fv3G polypeptide CD. In some aspects, the one or more amino acid substitutions are in the Fv3D polypeptide CBM. In some aspects, the one or more amino acid substitutions are in both the CD and the CBM. In some aspects, the Fv3D polypeptide amino acid substitutions can take place at amino acids E534 and/or D301. In some aspects, the Fv3D polypeptide amino acid substitutions can take place at one or more of amino acids D111, R117, L160, R175, K208, H209, R219, M266, Y269, D301, W302, S472, and/or E534 The mutant Fv3D polypeptide(s) suitably have β-glucosidase activity.

In some aspects, the Fv3D polypeptide comprises a chimera of two β-glucosidase sequences, wherein the first β-glucosidase sequence is at least about 200 amino acid residues in length and comprises about 60%, 65%, 70%, 75%, or 80% or more sequence identity to a sequence of equal length of Fv3D (SEQ ID NO: 58) and wherein the second β-glucosidase sequence is at least about 50 amino acid residues in length, and comprises at least about 60%, 65%, 70%, 75%, 80% or more sequence identity to a sequence of equal length of any one of SEQ ID NOs:54, 56, 60, 62, 64, 66, 68, 70, 72, 74, 76, 78, and 79. In some aspects, the first β-glucosidase sequence comprising an N-terminal sequence of at least 200 amino acid residues of SEQ ID NO:58, and the second β-glucosidase sequence comprising a C-terminal sequence of at least about 50 contiguous amino acid residues of any one of SEQ ID NOs:54, 56, 60, 62, 64, 66, 68, 70, 72, 74, 76, 78, and 79.

In certain aspects, the Fv3D polypeptide of the invention comprises a hybrid/fusion/chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 60, 62, 64, 66, 68, 70, 72, 74, 76, 78, and 79, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Fv3D (SEQ ID NO:58). In some aspects, the first β-glucosidase sequence comprises an N-terminal sequence of at least 200 amino acid residues of any one of SEQ ID NOs: 54, 56, 60, 62, 64, 66, 68, 70, 72, 74, 76, 78, and 79, or comprises one or more or all of polypeptide sequence motifs SEQ ID NOs: 164-169, and the second β-glucosidase sequence comprises a C-terminal sequence of at least 50 contiguous amino acid residues of SEQ ID NO:58.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an Fv3D polypeptide or a variant thereof. In some aspects, the N-terminal sequence comprises one or more or all of the polypeptide sequence motifs represented by SEQ ID NOs:136-148, or preferably sequence motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Fv3D, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Tr3A

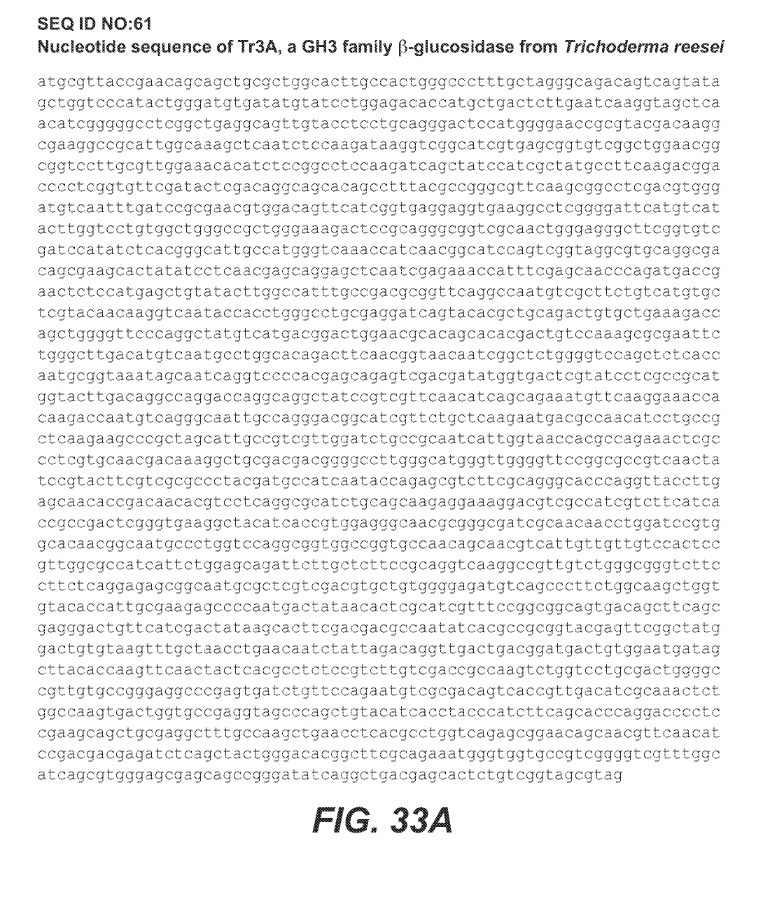

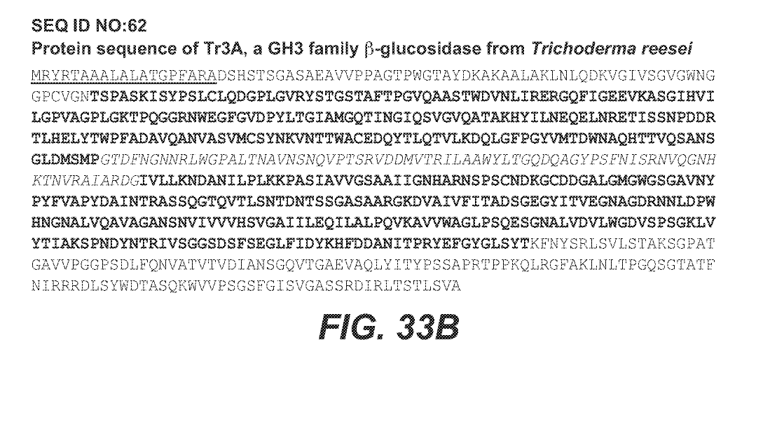

The amino acid sequence of Tr3A (SEQ ID NO:62) is shown in FIGS. 33B and 43. Tr3A is also known as T. reesei Bgl1. SEQ ID NO:62 is the sequence of the immature Tr3A. Tr3A has a predicted signal sequence corresponding to positions 1 to 19 of SEQ ID NO:62 (underlined); cleavage of the signal sequence is predicted to yield a mature protein having a sequence corresponding to positions 20 to 744 of SEQ ID NO:62. Signal sequence predictions were made with the SignalP-NN algorithm. The predicted conserved domain is in boldface type in FIG. 33B. Domain predictions were made based on the Pfam, SMART, or NCBI databases. Tr3A residues E472 and D267 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc (see, FIG. 43). As used herein, “a Tr3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, or 700 contiguous amino acid residues among residues 20 to 744 of SEQ ID NO:62. A Tr3A polypeptide preferably is unaltered, as compared to a native Tr3A, at residues E472 and D267. A Tr3A polypeptide is preferably unaltered in at least 70%, 80%, 90%, 95%, 98%, or 99% of the amino acid residues that are conserved among the herein described GH3 family β-glucosidases as shown in the alignment of FIG. 43. A Tr3A polypeptide suitably comprises the entire predicted conserved domains of native Tr3A shown in FIG. 33B. An exemplary Tr3A polypeptide comprises a sequence having at least 85%, 86%, 87%, 88%, 89%, 90%, 91%, 92%, 93%, 94%, 95%, 96%, 97%, 98%, 99%, or 100% identity to the mature Tr3A sequence shown in FIG. 33B. The Tr3A polypeptide of the invention preferably has β-glucosidase activity.

Accordingly a Tr3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:62, or to residues (i) 20-287, (ii) 22-611, (iii) 20-744, (iv) 362-611, or (v) 362-744 of SEQ ID NO:62. The polypeptide suitably has β-glucosidase activity.

In some aspects, a “Tr3A polypeptide” of the invention can also refer to a mutant Tr3A polypeptide. Amino acid substitutions can be introduced into the Tr3A polypeptide to improve the β-glucosidase activity of the molecule. For example, amino acid substitutions that increase the binding affinity of the Tr3A polypeptide for its substrate or that improve Tr3A's ability to catalyze the hydrolysis of terminal non-reducing residues in β-D-glucosides can be introduced into the Tr3A polypeptide. In some aspects, the mutant Tr3A polypeptides comprise one or more conservative amino acid substitutions. In some aspects, the mutant Tr3A polypeptides comprise one or more non-conservative amino acid substitutions. In some aspects, the one or more amino acid substitutions are in the Tr3A polypeptide CD. In some aspects, the one or more amino acid substitutions are in the Tr3A polypeptide CBM. In some aspects, the one or more amino acid substitutions are in both the CD and the CBM. In some aspects, the Tr3A polypeptide amino acid substitutions can take place at amino acids E472 and/or D267. In some aspects, the Tr3A polypeptide amino acid substitutions can take place at one or more of amino acids D92, R98, L141, R156, K189, H190, R200, M232, Y235, D267, W268, S415, and/or E472. The mutant Tr3A polypeptide(s) suitably have β-glucosidase activity.

In some aspects, the Tr3A polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Tr3A (SEQ ID NO:62), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 64, 68, 70, 72, 74, 76, 78, and 79, or comprises a polypeptide sequence motif SEQ ID NO:170. In some aspects, the first β-glucosidase sequence comprises an N-terminal sequence of at least 200 amino acid residues of SEQ ID NO:62, and the second β-glucosidase sequence comprising a C-terminal sequence of at least about 50 contiguous amino acid residues of any one of SEQ ID NOs:54, 56, 58, 60, 64, 66, 68, 70, 72, 74, 76, 78, and 79, or comprises a polypeptide sequence motif SEQ ID NO:170.

In certain aspects, the Tr3A polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 64, 66, 68, 70, 72, 74, 76, 78, and 79, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Tr3A (SEQ ID NO:62). In some aspects, the first β-glucosidase sequence comprises an N-terminal sequence of at least 200 amino acid residues of any one of SEQ ID NOs: 54, 56, 58, 60, 64, 66, 68, 70, 72, 74, 76, 78, and 79, or comprises one or more or all of polypeptide sequence motifs SEQ ID NOs: 164-169, and the second β-glucosidase sequence comprises a C-terminal sequence of at least 50 contiguous amino acid residues of SEQ ID NO:62.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Tr3A polypeptide or a variant thereof. In some aspects, the N-terminal sequence comprises one or more or all of the polypeptide sequence motifs represented by SEQ ID NOs:136-148, or preferably the sequence motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Tr3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The non-naturally occurring cellulase composition comprises β-glucosidase activity. The non-naturally occurring cellulase composition may further comprise one or more of xylanase, β-xylosidase, and/or L-α-arabinofuranosidase activities.

### Tr3B

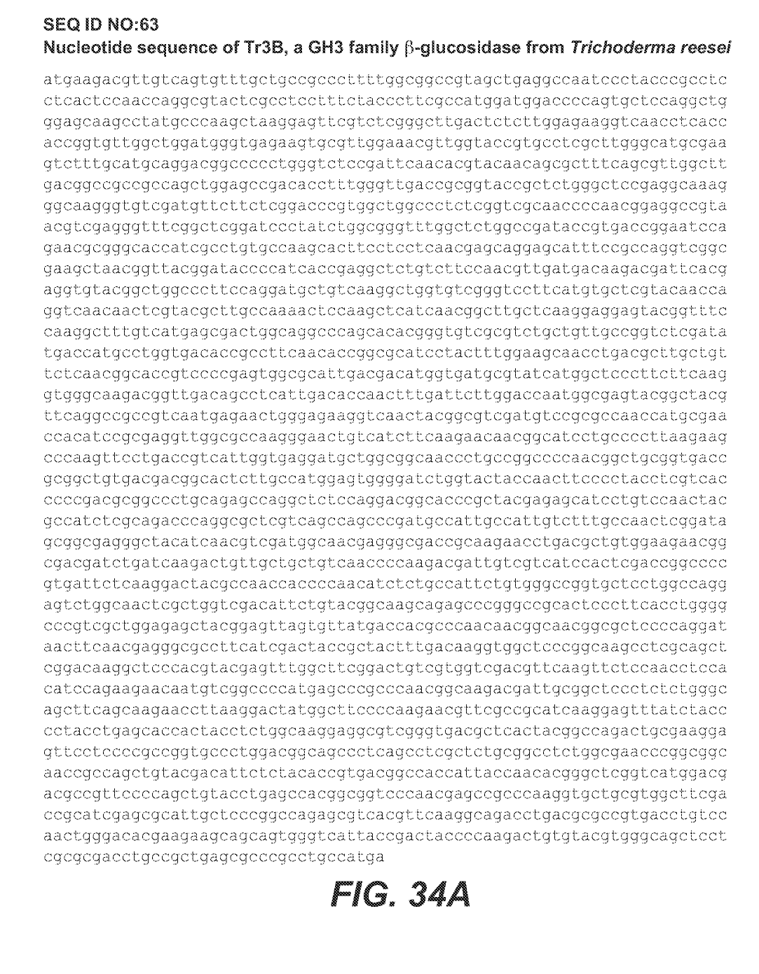

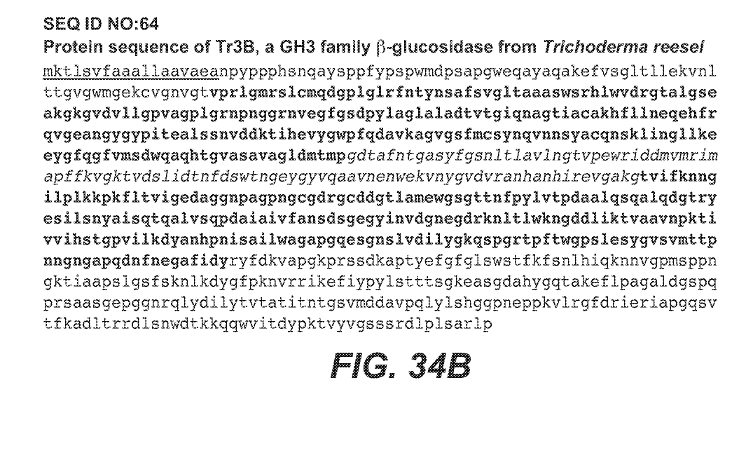

The amino acid sequence of Tr3B (SEQ ID NO:64) is shown in FIGS. 34B and 43. Tr3B is also known as “T. reesei Bgl3” or “T. reesei Cel3B.” SEQ ID NO:64 is the sequence of the immature Tr3B. Tr3B has a predicted signal sequence corresponding to positions 1 to 18 of SEQ ID NO:64 (underlined); cleavage of the signal sequence is predicted to yield a mature protein having a sequence corresponding to positions 19 to 874 of SEQ ID NO:64. Signal sequence predictions were made with the SignalP-NN algorithm. The predicted conserved domain is in boldface type in FIG. 34B. Domain predictions were made based on the Pfam, SMART, or NCBI databases. Tr3B residues E516 and D287 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Tr3B polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, 800, or 850 contiguous amino acid residues among residues 19 to 874 of SEQ ID NO:64. A Tr3B polypeptide preferably is unaltered, as compared to a native Tr3B, at residues E516 and D287. A Tr3B polypeptide is preferably unaltered in at least 70%, 80%, 90%, 95%, 98%, or 99% of the amino acid residues that are conserved among the herein described GH3 family β-glucosidases as shown in the alignment of FIG. 43. A Tr3B polypeptide suitably comprises the entire predicted conserved domains of native Tr3B shown in FIG. 34B. An exemplary Tr3A polypeptide comprises a sequence having at least 85%, 86%, 87%, 88%, 89%, 90%, 91%, 92%, 93%, 94%, 95%, 96%, 97%, 98%, 99%, or 100% identity to the mature Tr3B sequence shown in FIG. 34B. The Tr3B polypeptide of the invention preferably has β-glucosidase activity.

Accordingly a Tr3B polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:64, or to residues (i) 19-307, (ii) 19-640, (iii) 19-874, (iv) 407-640, or (v) 407-874 of SEQ ID NO:64. The polypeptide suitably has β-glucosidase activity.

In some aspects, a “Tr3B polypeptide” of the invention can also refer to a mutant Tr3B polypeptide. Amino acid substitutions can be introduced into the Tr3B polypeptide to improve the β-glucosidase activity of the molecule. For example, amino acid substitutions that increase the binding affinity of the Tr3B polypeptide for its substrate or that improve Tr3B's ability to catalyze the hydrolysis of terminal non-reducing residues in β-D-glucosides can be introduced into the Tr3B polypeptide. In some aspects, the mutant Tr3B polypeptides comprise one or more conservative amino acid substitutions. In some aspects, the mutant Tr3B polypeptides comprise one or more non-conservative amino acid substitutions. In some aspects, the one or more amino acid substitutions are in the Tr3B polypeptide CD. In some aspects, the one or more amino acid substitutions are in the Tr3B polypeptide CBM. In some aspects, the one or more amino acid substitutions are in both the CD and the CBM. In some aspects, the Tr3B polypeptide amino acid substitutions can take place at amino acids E516 and/or D287. In some aspects, the Tr3B polypeptide amino acid substitutions can take place at one or more of amino acids D99, R105, L148, R163, K196, H197, R207, M252, Y255, D287, W288, S457, and/or E516. The mutant Tr3B polypeptide(s) suitably have β-glucosidase activity.

In some aspects, the Tr3B polypeptide comprises a chimera/hybrid/fusion of two β-glucosidase sequences, wherein the first β-glucosidase sequence is at least about 200 amino acid residues in length and comprises about 60%, 65%, 70%, 75%, or 80% or more sequence identity to a sequence of equal length of Tr3B (SEQ ID NO:64) and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 66, 68, 70, 72, 74, 76, 78, and 79, or comprises the polypeptide sequence motif of SEQ ID NO:170. In some aspects, the first β-glucosidase sequence comprising an N-terminal sequence of at least 200 amino acid residues of SEQ ID NO:64, and the second β-glucosidase sequence comprising a C-terminal sequence of at least about 50 contiguous amino acid residues of any one of SEQ ID NOs:54, 56, 58, 60, 62, 68, 70, 72, 74, 76, 78, and 79, or comprises the polypeptide sequence motif of SEQ ID NO:170.

In certain aspects, the Tr3B polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 66, 68, 70, 72, 74, 76, 78, and 79, or comprises one or more polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Tr3B (SEQ ID NO:64). In some aspects, the first β-glucosidase sequence comprises an N-terminal sequence of at least 200 amino acid residues of any one of SEQ ID NOs:54, 56, 58, 60, 62, 66, 68, 70, 72, 74, 76, 78, and 79, or comprises one or more or all of polypeptide sequence motifs SEQ ID NOs:164-169, and the second β-glucosidase sequence comprises a C-terminal sequence of at least 50 contiguous amino acid residues of SEQ ID NO:64.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Tr3B polypeptide or a variant thereof. In some aspects, the N-terminal sequence comprises one or more or all of the polypeptide sequence motifs represented by SEQ ID NOs:136-148, or preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Tr3B, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in the rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Te3A

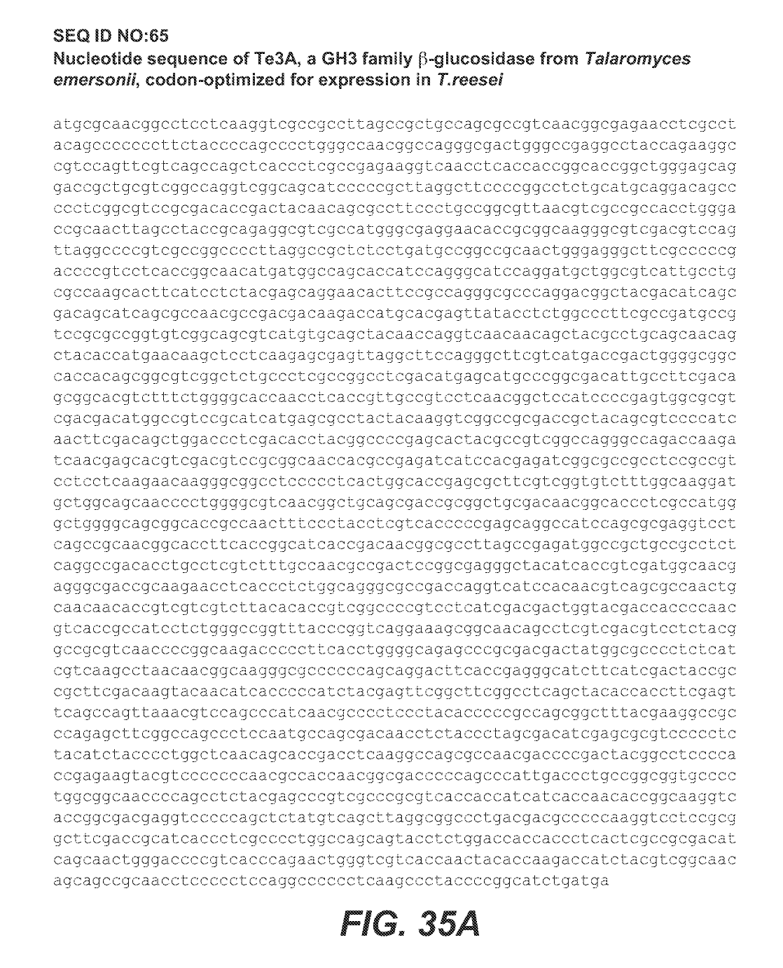

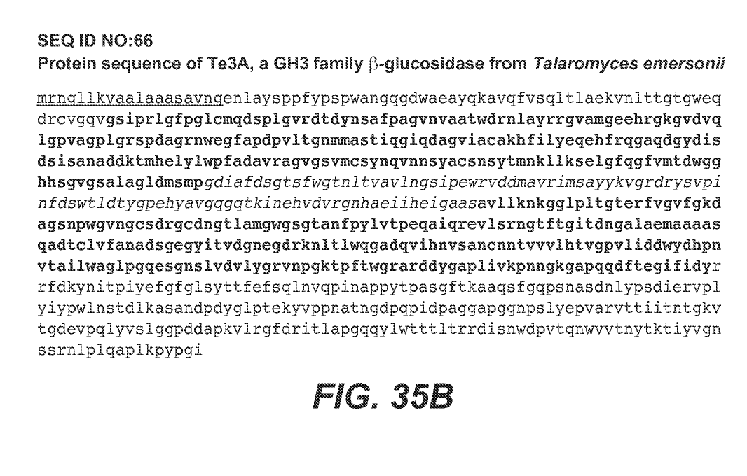

The amino acid sequence of Te3A (SEQ ID NO:66) is shown in FIGS. 35B and 43. Te3A is also known as “Abg2.” SEQ ID NO:66 is the sequence of the immature Te3A. Te3A has a predicted signal sequence corresponding to positions 1 to 19 of SEQ ID NO:66 (underlined); cleavage of the signal sequence is predicted to yield a mature protein having a sequence corresponding to positions 20 to 857 of SEQ ID NO:66. Signal sequence predictions were made with the SignalP-NN algorithm. The predicted conserved domain is in boldface type in FIG. 35B. Domain predictions were made based on the Pfam, SMART, or NCBI databases. Te3A residues E505 and D277 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07) etc. (see, FIG. 43). As used herein, “a Te3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, or 800 contiguous amino acid residues among residues 20 to 857 of SEQ ID NO:66. A Te3A polypeptide preferably is unaltered, as compared to a native Te3A, at residues E505 and D277. A Te3A polypeptide is preferably unaltered in at least 70%, 80%, 90%, 95%, 98%, or 99% of the amino acid residues that are conserved among the herein described GH3 family β-glucosidases as shown in the alignment of FIG. 43. A Te3A polypeptide suitably comprises the entire predicted conserved domains of native Te3A shown in FIG. 35B. An exemplary Te3A polypeptide comprises a sequence having at least 85%, 86%, 87%, 88%, 89%, 90%, 91%, 92%, 93%, 94%, 95%, 96%, 97%, 98%, 99%, or 100% identity to the mature Te3A sequence shown in FIG. 35B. The Te3A polypeptide of the invention preferably has β-glucosidase activity.

Accordingly a Te3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:66, or to residues (i) 20-297, (ii) 20-629, (iii) 20-857, (iv) 396-629, or (v) 396-857 of SEQ ID NO:66. The polypeptide suitably has β-glucosidase activity.

In some aspects, a “Te3A polypeptide” of the invention can also refer to a mutant Te3A polypeptide. Amino acid substitutions can be introduced into the Te3A polypeptide to improve the β-glucosidase activity of the molecule. For example, amino acid substitutions that increase the binding affinity of the Te3A polypeptide for its substrate or that improve Te3A's ability to catalyze the hydrolysis of terminal non-reducing residues in β-D-glucosides can be introduced into the Te3A polypeptide. In some aspects, the mutant Te3A polypeptides comprise one or more conservative amino acid substitutions. In some aspects, the mutant Te3A polypeptides comprise one or more non-conservative amino acid substitutions. In some aspects, the one or more amino acid substitutions are in the Te3A polypeptide CD. In some aspects, the one or more amino acid substitutions are in the Te3A polypeptide CBM. In some aspects, the one or more amino acid substitutions are in both the CD and the CBM. In some aspects, the Te3A polypeptide amino acid substitutions can take place at amino acids E505 and/or D277. In some aspects, the Te3A polypeptide amino acid substitutions can take place at one or more of amino acids D92, R98, L141, R156, K189, H190, R200, M242, Y245, D277, W278, S447, and/or E505. The mutant Te3A polypeptide(s) suitably have β-glucosidase activity.

In some aspects, the Te3A polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Te3A (SEQ ID NO:66), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 68, 70, 72, 74, 76, 78, and 79, or comprises the polypeptide sequence motif SEQ ID NO:170. In some aspects, the first β-glucosidase sequence comprising an N-terminal sequence of at least 200 amino acid residues of SEQ ID NO:66, and the second β-glucosidase sequence comprising a C-terminal sequence of at least about 50 contiguous amino acid residues of any one of SEQ ID NOs:54, 56, 58, 60, 62, 64, 68, 70, 72, 74, 76, 78, and 79, or comprises the polypeptide sequence motif SEQ ID NO:170.

In certain aspects, the Te3A polypeptide of the invention comprises a chimera/hybrid/fusion or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 68, 70, 72, 74, 76, 78, and 79, or comprises one or more or all of polypeptide sequence motifs SEQ ID NOs:164-169, whereas the second β-glucosidase sequence is at least about 50 amino acid residues in length and comprises about 60%, 65%, 70%, 75%, 80% or more sequence identity to sequence of equal length of Te3A (SEQ ID NO:66). In some aspects, the first β-glucosidase sequence comprises an N-terminal sequence of at least 200 amino acid residues of any one of SEQ ID NOs: 54, 56, 58, 60, 62, 64, 68, 70, 72, 74, 76, 78, and 79, or comprises one or more or all of polypeptide sequence motifs SEQ ID NOs:164-169, and the second β-glucosidase sequence comprises a C-terminal sequence of at least 50 contiguous amino acid residues of SEQ ID NO:66.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Te3A polypeptide or a variant thereof. In some aspects, the N-terminal sequence comprises one or more or all of the polypeptide sequence motifs represented by SEQ ID NOs:136-148, or preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Te3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### An3A

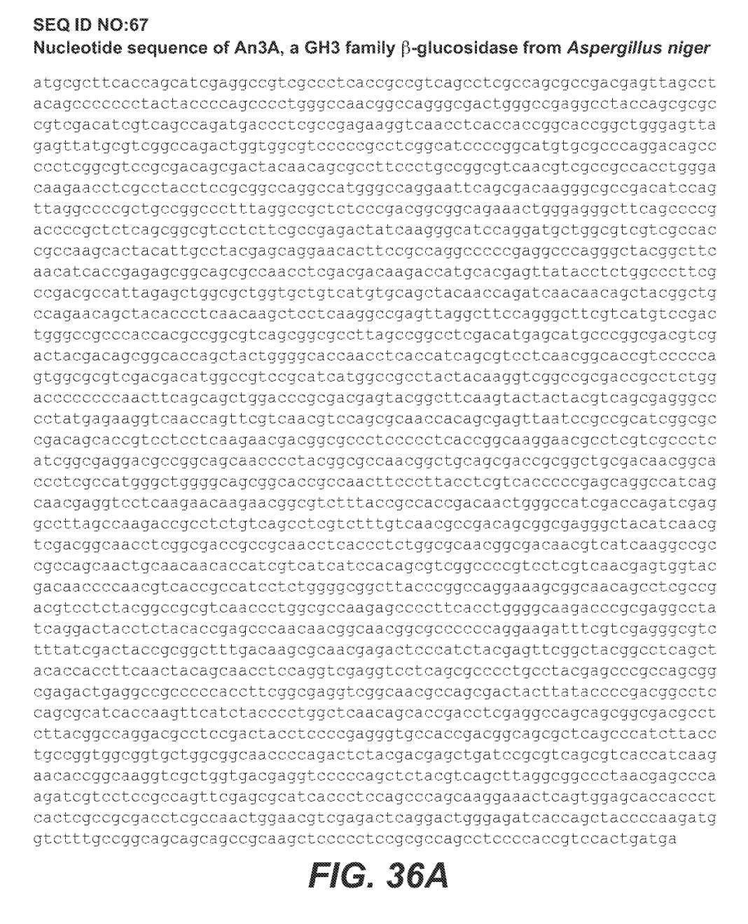

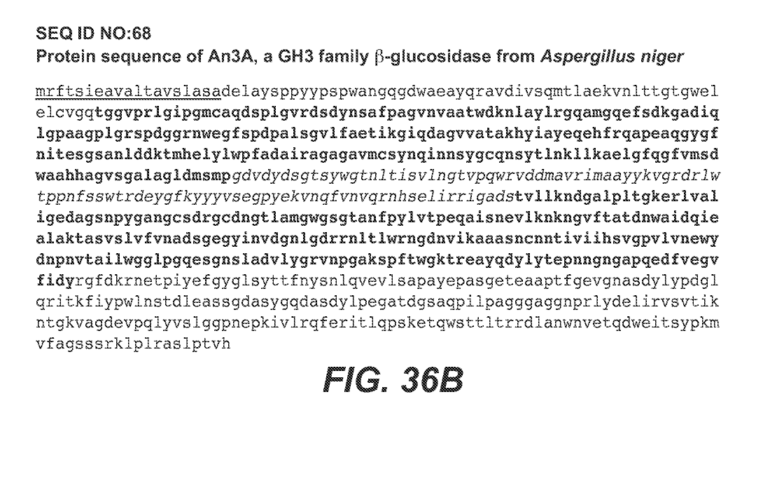

The amino acid sequence of An3A (SEQ ID NO:68) is shown in FIGS. 36B and 43. An3A is also known as “A .niger Bglu.” SEQ ID NO:68 is the sequence of the immature An3A. An3A has a predicted signal sequence corresponding to positions 1 to 19 of SEQ ID NO:68 (underlined); cleavage of the signal sequence is predicted to yield a mature protein having a sequence corresponding to positions 20 to 860 of SEQ ID NO:68. Signal sequence predictions were made with the SignalP-NN algorithm. The predicted conserved domain is in boldface type in FIG. 36B. Domain predictions were made based on the Pfam, SMART, or NCBI databases. An3A residues E509 and D277 are predicted to function as catalytic acid-base and nucleophile, respectively, based on a sequence alignment of the above-mentioned GH3 glucosidases from e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “an An3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, or 800 contiguous amino acid residues among residues 20 to 860 of SEQ ID NO:68. An An3A polypeptide preferably is unaltered, as compared to a native An3A, at residues E509 and D277. An An3A polypeptide is preferably unaltered in at least 70%, 80%, 90%, 95%, 98%, or 99% of the amino acid residues that are conserved among the herein described GH3 family β-glucosidases as shown in the alignment of FIG. 43. An An3A polypeptide suitably comprises the entire predicted conserved domains of native An3A shown in FIG. 36B. An exemplary An3A polypeptide comprises a sequence having at least 85%, 86%, 87%, 88%, 89%, 90%, 91%, 92%, 93%, 94%, 95%, 96%, 97%, 98%, 99%, or 100% identity to the mature An3A sequence shown in FIG. 36B. The An3A polypeptide of the invention preferably has β-glucosidase activity.

Accordingly an An3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:68, or to residues (i) 20-300, (ii) 20-634, (iii) 20-860, (iv) 400-634, or (v) 400-860 of SEQ ID NO:68. The polypeptide suitably has β-glucosidase activity.

In some aspects, an “An3A polypeptide” of the invention can also refer to a mutant An3A polypeptide. Amino acid substitutions can be introduced into the An3A polypeptide to improve the β-glucosidase activity of the molecule. For example, amino acid substitutions that increase the binding affinity of the An3A polypeptide for its substrate or that improve An3A's ability to catalyze the hydrolysis of terminal non-reducing residues in β-D-glucosides can be introduced into the An3A polypeptide. In some aspects, the mutant An3A polypeptides comprise one or more conservative amino acid substitutions. In some aspects, the mutant An3A polypeptides comprise one or more non-conservative amino acid substitutions. In some aspects, the one or more amino acid substitutions are in the An3A polypeptide CD. In some aspects, the one or more amino acid substitutions are in the An3A polypeptide CBM. In some aspects, the one or more amino acid substitutions are in both the CD and the CBM. In some aspects, the An3A polypeptide amino acid substitutions can take place at amino acids E509 and/or D277. In some aspects, the An3A polypeptide amino acid substitutions can take place at one or more of amino acids D92, R98, L141, R156, K189, H190, R200, M245, Y248, D277, W278, S451, and/or E509. The mutant An3A polypeptide(s) suitably have β-glucosidase activity.

In some aspects, the An3A polypeptide comprises a chimera/hybrid/fusion of two β-glucosidase sequences, wherein the first β-glucosidase sequence is at least about 200 amino acid residues in length and comprises about 60%, 65%, 70%, 75%, or 80% or more sequence identity to a sequence of equal length of An3A (SEQ ID NO:68), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 66, 70, 72, 74, 76, 78, and 79, or comprises a polypeptide sequence motif SEQ ID NO:170. In some aspects, the first β-glucosidase sequence comprising an N-terminal sequence of at least 200 amino acid residues of SEQ ID NO:68, and the second β-glucosidase sequence comprises a C-terminal sequence of at least about 50 contiguous amino acid residues of any one of SEQ ID NOs:54, 56, 58, 60, 62, 64, 66, 70, 72, 74, 76, 78, and 79, or comprises a polypeptide sequence motif SEQ ID NO:170.

In certain aspects, the An3A polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 66, 70, 72, 74, 76, 78, and 79, or comprises one or more or all of polypeptide sequence motifs SEQ ID NOs:164-169, whereas the second β-glucosidase sequence is at least about 50 amino acid residues in length and comprises about 60%, 65%, 70%, 75%, 80% or more sequence identity to a sequence of equal length of An3A (SEQ ID NO:68). In some aspects, the first β-glucosidase sequence comprises an N-terminal sequence of at least 200 amino acid residues of any one of SEQ ID NOs:54, 56, 58, 60, 62, 64, 66, 70, 72, 74, 76, 78, and 79, or comprises one or more or all of polypeptide sequence motifs SEQ ID NOs:164-169, and the second β-glucosidase sequence comprises a C-terminal sequence of at least 50 contiguous amino acid residues of SEQ ID NO:68.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an An3A polypeptide or a variant thereof. In some aspects, the N-terminal sequence comprises one or more or all of the polypeptide sequence motifs represented by SEQ ID NOs:136-148, preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including An3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Fo3A

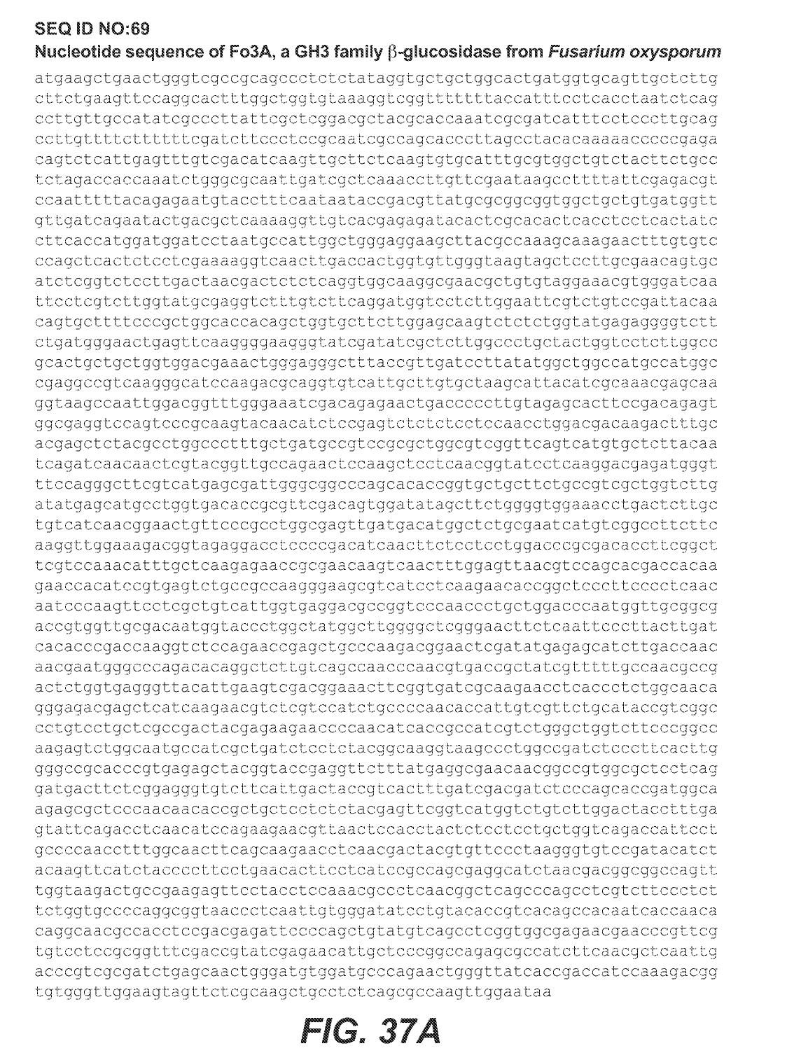

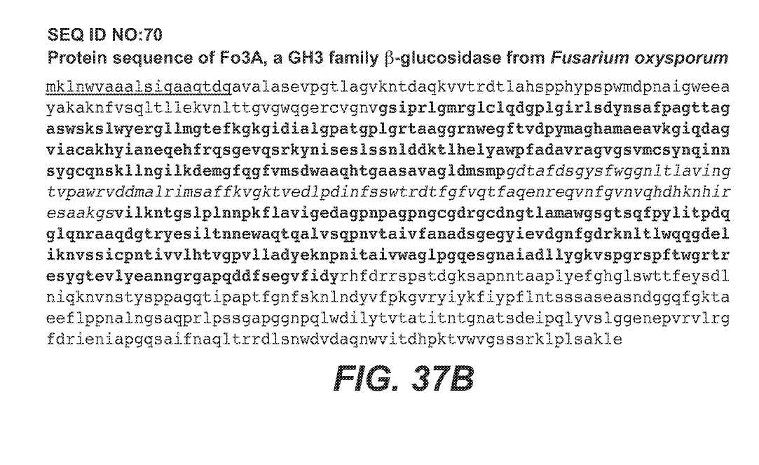

The amino acid sequence of Fo3A (SEQ ID NO:70) is shown in FIGS. 37B and 43. SEQ ID NO:70 is the sequence of the immature Fo3A. Fo3A has a predicted signal sequence corresponding to positions 1 to 19 of SEQ ID NO:70 (underlined); cleavage of the signal sequence is predicted to yield a mature protein having a sequence corresponding to positions 20 to 899 of SEQ ID NO:70. Signal sequence predictions were made with the SignalP-NN algorithm. The predicted conserved domain is in boldface type in FIG. 37B. Domain predictions were made based on the Pfam, SMART, or NCBI databases. Fo3A residues E536 and D307 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07) etc. (see, FIG. 43). As used herein, “an Fo3A polypeptide” refers, in some aspect, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, 800, or 850 contiguous amino acid residues among residues 20 to 899 of SEQ ID NO:70. An Fo3A polypeptide preferably is unaltered, as compared to a native Fo3A, at residues E536 and D307. An Fo3A polypeptide is preferably unaltered in at least 70%, 80%, 90%, 95%, 98%, or 99% of the amino acid residues that are conserved among the herein described GH3 family β-glucosidases as shown in the alignment of FIG. 43. An Fo3A polypeptide suitably comprises the entire predicted conserved domains of native Fo3A shown in FIG. 37B. An exemplary Fo3A polypeptide comprises a sequence having at least 85%, 86%, 87%, 88%, 89%, 90%, 91%, 92%, 93%, 94%, 95%, 96%, 97%, 98%, 99%, or 100% identity to the mature Fo3A sequence shown in FIG. 37B. The Fo3A polypeptide of the invention preferably has β-glucosidase activity.

Accordingly an Fo3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:70, or to residues (i) 20-327, (ii) 20-660, (iii) 20-899, (iv) 428-660, or (v) 428-899 of SEQ ID NO:70. The polypeptide suitably has β-glucosidase activity.

In some aspects, an “Fo3A polypeptide” of the invention can also refer to a mutant Fo3A polypeptide. Amino acid substitutions can be introduced into the Fo3A polypeptide to improve the β-glucosidase activity of the molecule. For example, amino acid substitutions that increase the binding affinity of the Fo3A polypeptide for its substrate or that improve Fo3A's ability to catalyze the hydrolysis of terminal non-reducing residues in β-D-glucosides can be introduced into the Fo3A polypeptide. In some aspects, the mutant Fo3A polypeptides comprise one or more conservative amino acid substitutions. In some aspects, the mutant Fo3A polypeptides comprise one or more non-conservative amino acid substitutions. In some aspects, the one or more amino acid substitutions are in the Fo3A polypeptide CD. In some aspects, the one or more amino acid substitutions are in the Fo3A polypeptide CBM. In some aspects, the one or more amino acid substitutions are in both the CD and the CBM. In some aspects, the Fo3A polypeptide amino acid substitutions can take place at amino acids E536 and/or D307. In some aspects, the Fo3A polypeptide amino acid substitutions can take place at one or more of amino acids D119, R125, L168, R183, K216, H217, R227, M272, Y275, D307, W308, S477, and/or E536. The mutant Fo3A polypeptide(s) suitably have β-glucosidase activity.

In some aspects, the Fo3A polypeptide comprises a chimera/hybrid/fusion of two β-glucosidase sequences, wherein the first β-glucosidase sequence is at least about 200 amino acid residues in length and comprises about 60%, 65%, 70%, 75%, or 80% or more sequence identity to a sequence of equal length of Fo3A (SEQ ID NO:70), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 66, 68, 72, 74, 76, 78, and 79, or comprises a polypeptide sequence motif SEQ ID NO:170. In some aspects, the first β-glucosidase sequence comprising an N-terminal sequence of at least 200 amino acid residues of SEQ ID NO:70, and the second β-glucosidase sequence comprising a C-terminal sequence of at least about 50 contiguous amino acid residues of any one of SEQ ID NOs:54, 56, 58, 60, 62, 64, 66, 68, 72, 74, 76, 78, and 79, or comprises a polypeptide sequence motif SEQ ID NO:170.

In certain aspects, the Fo3A polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 66, 68, 72, 74, 76, 78, and 79, or comprises one or more or all of polypeptide sequence motifs SEQ ID NOs:164-169, whereas the second β-glucosidase sequence is at least about 50 amino acid residues in length and comprises about 60%, 65%, 70%, 75%, 80% or more sequence identity to a sequence of equal length of Fo3A (SEQ ID NO:70). In some aspects, the first β-glucosidase sequence comprises an N-terminal sequence of at least 200 amino acid residues of any one of SEQ ID NOs: 54, 56, 58, 60, 62, 64, 66, 68, 72, 74, 76, 78, and 79, or comprises one or more or all of polypeptide sequence motifs SEQ ID NOs:164-169, and the second β-glucosidase sequence comprises a C-terminal sequence of at least 50 contiguous amino acid residues of SEQ ID NO:70.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an Fo3A polypeptide or a variant thereof. In some aspects, the N-terminal sequence comprises one or more or all of the polypeptide sequence motifs represented by SEQ ID NOs:136-148, preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Fo3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Gz3A

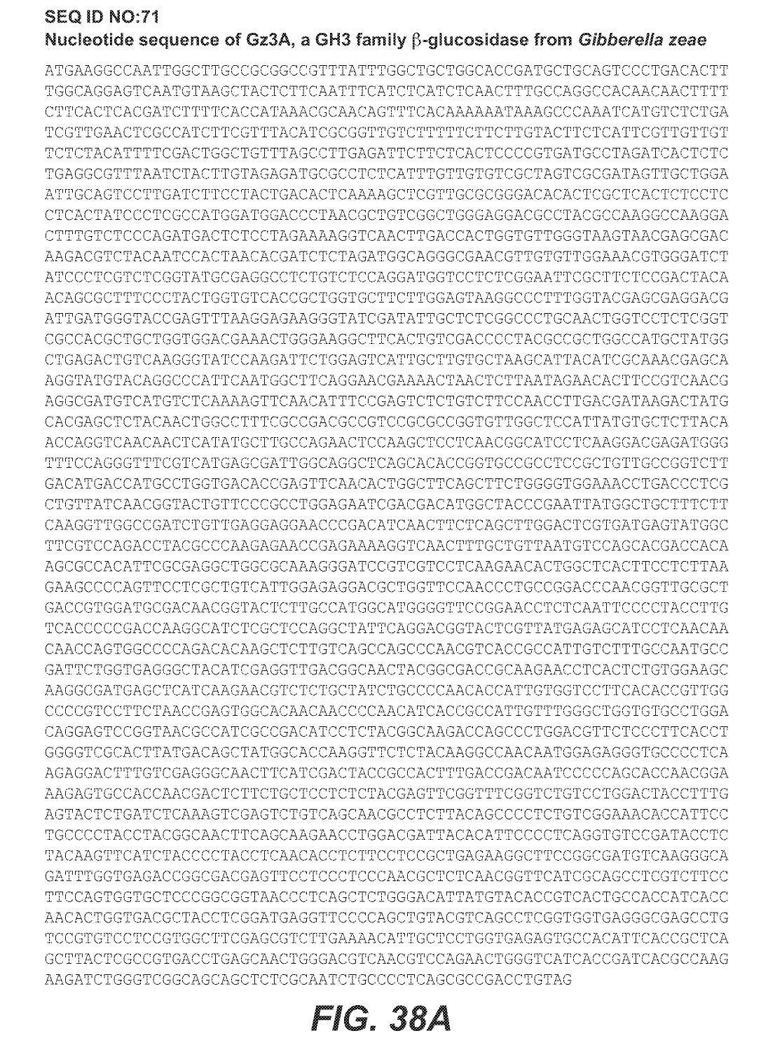

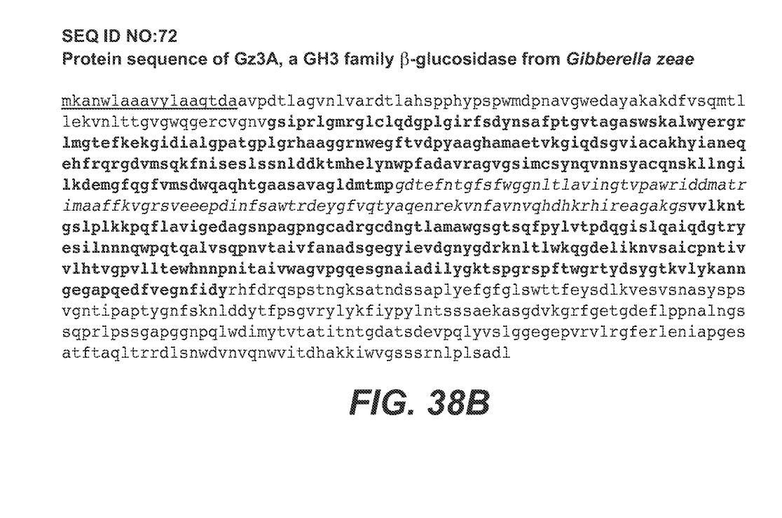

The amino acid sequence of Gz3A (SEQ ID NO:72) is shown in FIGS. 38B and 43. SEQ ID NO:72 is the sequence of the immature Gz3A. Gz3A has a predicted signal sequence corresponding to positions 1 to 18 of SEQ ID NO:72 (underlined); cleavage of the signal sequence is predicted to yield a mature protein having a sequence corresponding to positions 19 to 886 of SEQ ID NO:72. Signal sequence predictions were made with the SignalP-NN algorithm. The predicted conserved domain is in boldface type in FIG. 38B. Domain predictions were made based on the Pfam, SMART, or NCBI databases. Gz3A residues E523 and D294 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Gz3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, 800, or 850 contiguous amino acid residues among residues 19 to 886 of SEQ ID NO:72. A Gz3A polypeptide preferably is unaltered, as compared to a native Gz3A, at residues E536 and D307. A Gz3A polypeptide is preferably unaltered in at least 70%, 80%, 90%, 95%, 98%, or 99% of the amino acid residues that are conserved among the herein described GH3 family β-glucosidases as shown in the alignment of FIG. 43. A Gz3A polypeptide suitably comprises the entire predicted conserved domains of native Gz3A shown in FIG. 38B. An exemplary Gz3A polypeptide comprises a sequence having at least 85%, 86%, 87%, 88%, 89%, 90%, 91%, 92%, 93%, 94%, 95%, 96%, 97%, 98%, 99%, or 100% identity to the mature Gz3A sequence shown in FIG. 38B. The Gz3A polypeptide of the invention preferably has β-glucosidase activity.

Accordingly a Gz3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:72, or to residues (i) 19-314, (ii) 19-647, (iii) 19-886, (iv) 415-647, or (v) 415-886 of SEQ ID NO:72. The polypeptide suitably has β-glucosidase activity.

In some aspects, a “Gz3A polypeptide” of the invention can also refer to a mutant Gz3A polypeptide. Amino acid substitutions can be introduced into the Gz3A polypeptide to improve the β-glucosidase activity of the molecule. For example, amino acid substitutions that increase the binding affinity of the Gz3A polypeptide for its substrate or that improve Gz3A's ability to catalyze the hydrolysis of terminal non-reducing residues in β-D-glucosides can be introduced into the Gz3A polypeptide. In some aspects, the mutant Gz3A polypeptides comprise one or more conservative amino acid substitutions. In some aspects, the mutant Gz3A polypeptides comprise one or more non-conservative amino acid substitutions. In some aspects, the one or more amino acid substitutions are in the Gz3A polypeptide CD. In some aspects, the one or more amino acid substitutions are in the Gz3A polypeptide CBM. In some aspects, the one or more amino acid substitutions are in both the CD and the CBM. In some aspects, the Gz3A polypeptide amino acid substitutions can take place at amino acids E536 and/or D307. In some aspects, the Gz3A polypeptide amino acid substitutions can take place at one or more of amino acids D106, R112, L155, R170, K203, H204, R214, M259, Y262, D294, W295, S464, and/or E523. The mutant Gz3A polypeptide(s) suitably have β-glucosidase activity.

In some aspects, the Gz3A polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Gz3A (SEQ ID NO:72), and wherein the second β-glucosidase sequence is at least about 50 amino acid residues in length and comprises at least about 60%, 65%, 70%, 75%, 80% or more sequence identity to a sequence of equal of any one of SEQ ID NOs:54, 56, 58, 60, 62, 64, 66, 68, 70, 74, 76, 78, and 79, or comprises a polypeptide sequence motif SEQ ID NO:170. In some aspects, the first β-glucosidase sequence comprising an N-terminal sequence of at least 200 amino acid residues of SEQ ID NO:72, and the second β-glucosidase sequence comprising a C-terminal sequence of at least about 50 contiguous amino acid residues of any one of SEQ ID NOs:54, 56, 58, 60, 62, 64, 66, 68, 70, 74, 76, 78, and 79, or comprises a polypeptide sequence motif SEQ ID NO:170.

In certain aspects, the Gz3A polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 66, 68, 70, 74, 76, 78, and 79, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Gz3A (SEQ ID NO:72). In some aspects, the first β-glucosidase sequence comprises an N-terminal sequence of at least 200 amino acid residues of any one of SEQ ID NOs:54, 56, 58, 60, 62, 64, 66, 68, 70, 74, 76, 78, and 79, or comprises one or more or all of polypeptide sequence motifs SEQ ID NOs: 164-169, and the second β-glucosidase sequence comprises a C-terminal sequence of at least 50 contiguous amino acid residues of SEQ ID NO:72.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Gz3A polypeptide or a variant thereof. In some aspects, the N-terminal sequence comprises one or more or all of the polypeptide sequence motifs represented by SEQ ID NOs:136-148, preferably sequence motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Gz3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Nh3A

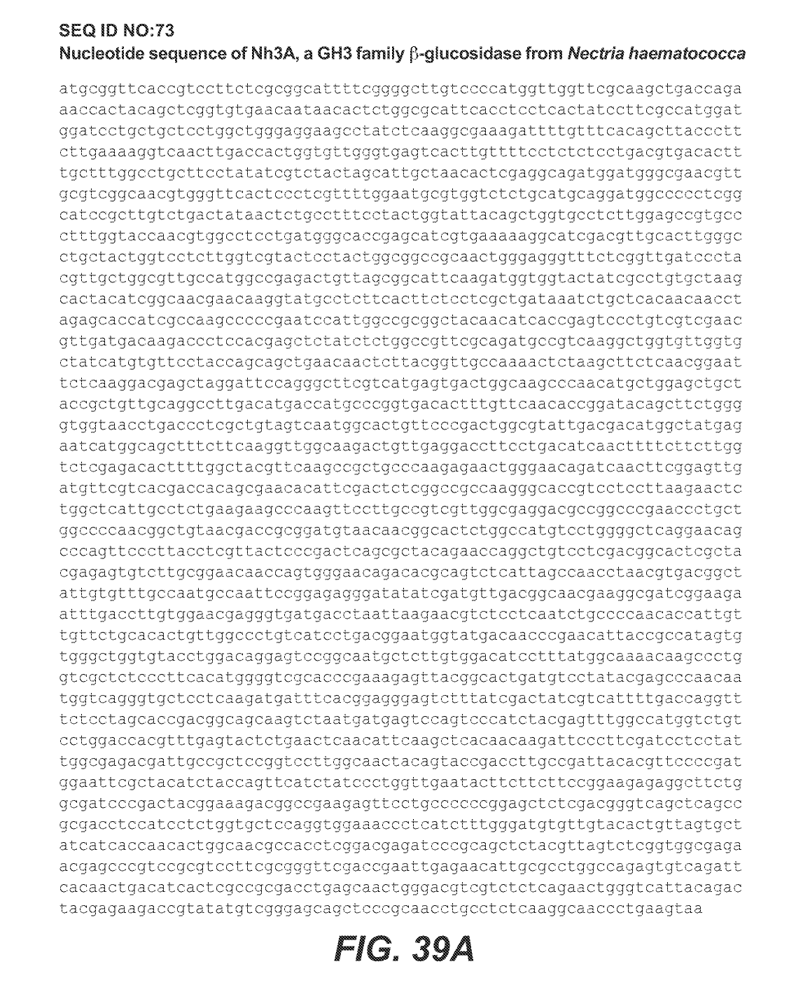

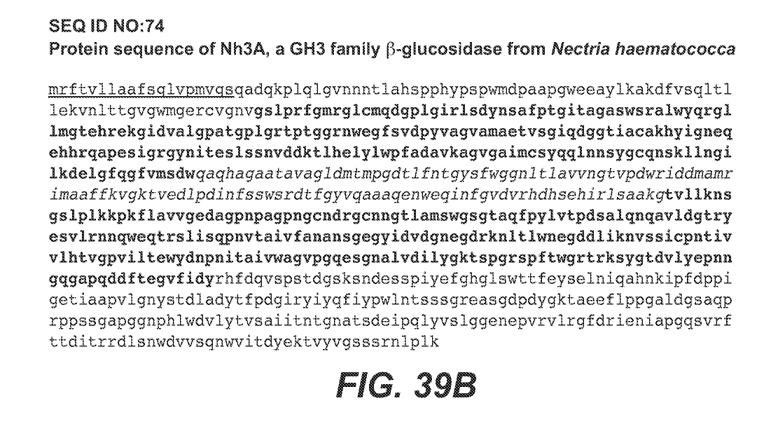

The amino acid sequence of Nh3A (SEQ ID NO:74) is shown in FIGS. 39B and 43. SEQ ID NO:74 is the sequence of the immature Nh3A. Nh3A has a predicted signal sequence corresponding to positions 1 to 19 of SEQ ID NO:74 (underlined); cleavage of the signal sequence is predicted to yield a mature protein having a sequence corresponding to positions 20 to 880 of SEQ ID NO:74. Signal sequence predictions were made with the SignalP-NN algorithm. The predicted conserved domain is in boldface type in FIG. 39B. Domain predictions were made based on the Pfam, SMART, or NCBI databases. Nh3A residues E523 and D294 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “an Nh3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, 800, or 850 contiguous amino acid residues among residues 20 to 880 of SEQ ID NO:74. An Nh3A polypeptide preferably is unaltered, as compared to a native Nh3A, at residues E523 and D294. An Nh3A polypeptide is preferably unaltered in at least 70%, 80%, 90%, 95%, 98%, or 99% of the amino acid residues that are conserved among the herein described GH3 family β-glucosidases as shown in the alignment of FIG. 43. An Nh3A polypeptide suitably comprises the entire predicted conserved domains of native Nh3A shown in FIG. 39B. An exemplary Nh3A polypeptide comprises a sequence having at least 85%, 86%, 87%, 88%, 89%, 90%, 91%, 92%, 93%, 94%, 95%, 96%, 97%, 98%, 99%, or 100% identity to the mature Nh3A sequence shown in FIG. 39B. The Nh3A polypeptide of the invention preferably has β-glucosidase activity.

Accordingly an Nh3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:74, or to residues (i) 20-295, (ii) 20-647, (iii) 20-880, (iv) 414-647, or (v) 414-880 of SEQ ID NO:74. The polypeptide suitably has β-glucosidase activity.

In some aspects, an “Nh3A polypeptide” of the invention can also refer to a mutant Nh3A polypeptide. Amino acid substitutions can be introduced into the Nh3A polypeptide to improve the β-glucosidase activity of the molecule. For example, amino acid substitutions that increase the binding affinity of the Nh3A polypeptide for its substrate or that improve Nh3A's ability to catalyze the hydrolysis of terminal non-reducing residues in β-D-glucosides can be introduced into the Nh3A polypeptide. In some aspects, the mutant Nh3A polypeptides comprise one or more conservative amino acid substitutions. In some aspects, the mutant Nh3A polypeptides comprise one or more non-conservative amino acid substitutions. In some aspects, the one or more amino acid substitutions are in the Nh3A polypeptide CD. In some aspects, the one or more amino acid substitutions are in the Nh3A polypeptide CBM. In some aspects, the one or more amino acid substitutions are in both the CD and the CBM. In some aspects, the Nh3A polypeptide amino acid substitutions can take place at amino acids E523 and/or D294. In some aspects, the Nh3A polypeptide amino acid substitutions can take place at one or more of amino acids D106, R112, L155, R170, K203, H204, R214, M259, Y262, D294, W295, S464, and/or E523. The mutant Nh3A polypeptide(s) suitably have β-glucosidase activity.

In some aspects, the Nh3A polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Nh3A (SEQ ID NO:74), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 66, 68, 70, 72, 76, 78, and 79, or comprises a polypeptide sequence motif SEQ ID NO:170. In some aspects, the first β-glucosidase sequence comprising an N-terminal sequence of at least 200 amino acid residues of SEQ ID NO:74, and the second β-glucosidase sequence comprising a C-terminal sequence of at least about 50 contiguous amino acid residues of any one of SEQ ID NOs:54, 56, 58, 60, 62, 64, 66, 68, 70, 72, 76, 78, and 79, or comprises a polypeptide sequence motif SEQ ID NO:170.

In certain aspects, the Nh3A polypeptide of the invention comprises a chimera or a chimeric construct of two β-glucosidase sequences, wherein the first β-glucosidase sequence is at least about 200 amino acid residues in length, and comprises about 60%, 65%, 70%, 75%, 80% or more sequence identity to a sequence of equal length of any one of SEQ ID NOs: 54, 56, 58, 60, 62, 64, 66, 68, 70, 72, 76, 78, and 79, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Nh3A (SEQ ID NO:74). In some aspects, the first β-glucosidase sequence comprises an N-terminal sequence of at least 200 amino acid residues of any one of SEQ ID NOs:54, 56, 58, 60, 62, 64, 66, 68, 70, 72, 76, 78, and 79, or comprises one or more or all of polypeptide sequence motifs SEQ ID NOs: 164-169, and the second β-glucosidase sequence comprises a C-terminal sequence of at least 50 contiguous amino acid residues of SEQ ID NO:74.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from an Nh3A polypeptide or a variant thereof. In some aspects, the N-terminal sequence comprises one or more or all of the polypeptide sequence motifs represented by SEQ ID NOs:136-148, preferably the sequence motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Nh3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in extent or rate of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Vd3A

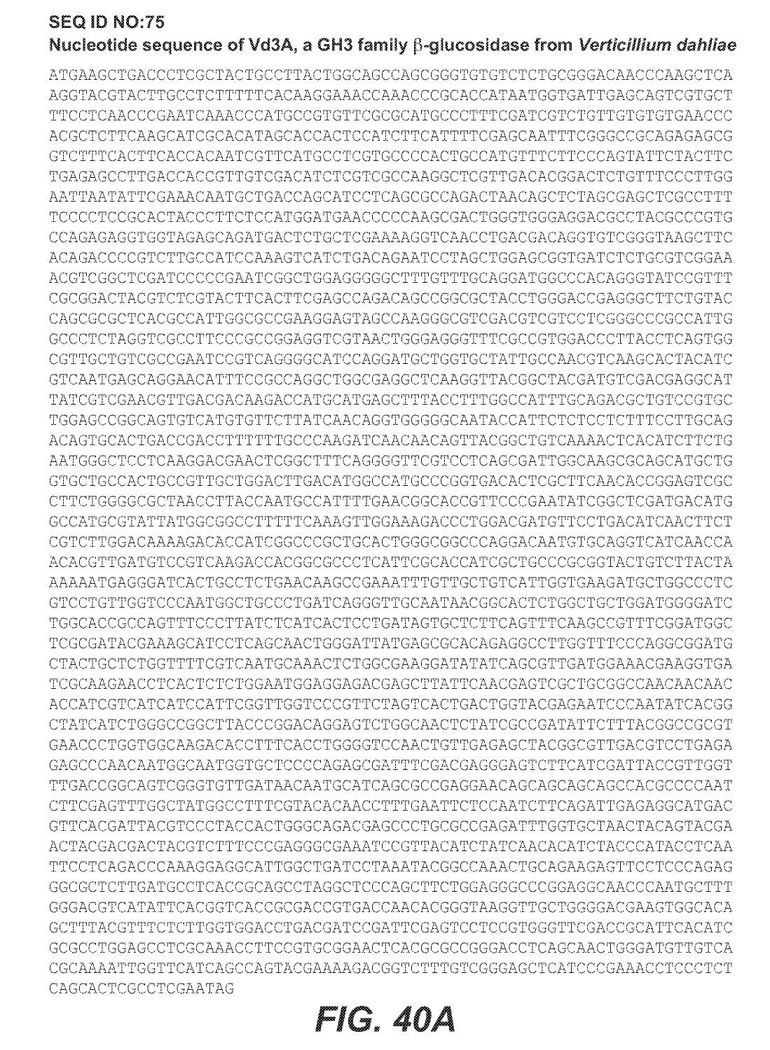

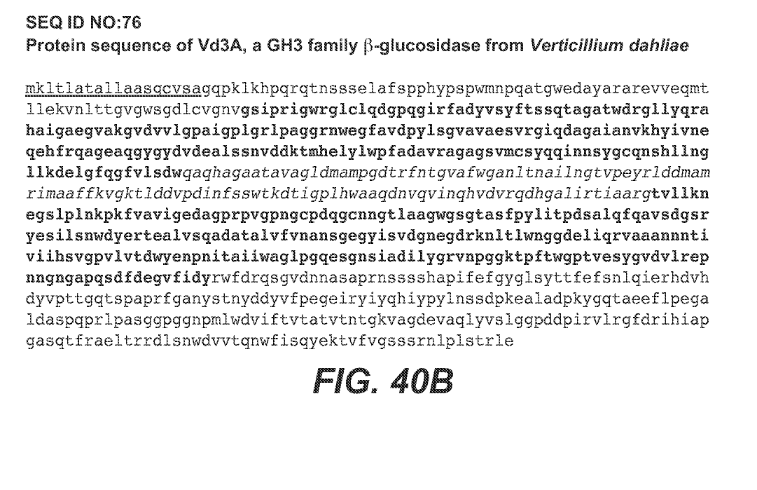

The amino acid sequence of Vd3A (SEQ ID NO:76) is shown in FIGS. 40B and 43. SEQ ID NO:76 is the sequence of the immature Vd3A. Vd3A has a predicted signal sequence corresponding to positions 1 to 18 of SEQ ID NO:76 (underlined); cleavage of the signal sequence is predicted to yield a mature protein having a sequence corresponding to positions 19 to 890 of SEQ ID NO:76. Signal sequence predictions were made with the SignalP-NN algorithm. The predicted conserved domain is in boldface type in FIG. 40B. Domain predictions were made based on the Pfam, SMART, or NCBI databases. Vd3A was shown to have β-glucosidase activity in, e.g., an enzymatic assay using cNPG and cellobiose, and in hydrolysis of dilute ammonia pretreated corncob as substrates. Vd3A residues E524 and D295 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Vd3A polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, 800, or 850 contiguous amino acid residues among residues 19 to 890 of SEQ ID NO:76. A Vd3A polypeptide preferably is unaltered, as compared to a native Vd3A, at residues E524 and D295. A Vd3A polypeptide is preferably unaltered in at least 70%, 80%, 90%, 95%, 98%, or 99% of the amino acid residues that are conserved among the herein described GH3 family β-glucosidases as shown in the alignment of FIG. 43. A Vd3A polypeptide suitably comprises the entire predicted conserved domains of native Vd3A shown in FIG. 40B. An exemplary Nh3A polypeptide comprises a sequence having at least 85%, 86%, 87%, 88%, 89%, 90%, 91%, 92%, 93%, 94%, 95%, 96%, 97%, 98%, 99%, or 100% identity to the mature Vd3A sequence shown in FIG. 40B. The Vd3A polypeptide of the invention preferably has β-glucosidase activity.

Accordingly a Vd3A polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:76, or to residues (i) 19-296, (ii) 19-649, (iii) 19-890, (iv) 415-649, or (v) 415-890 of SEQ ID NO:76. The polypeptide suitably has β-glucosidase activity.

In some aspects, a “Vd3A polypeptide” of the invention can also refer to a mutant Vd3A polypeptide. Amino acid substitutions can be introduced into the Vd3A polypeptide to improve the β-glucosidase activity of the molecule. For example, amino acid substitutions that increase the binding affinity of the Vd3A polypeptide for its substrate or that improve Vd3A's ability to catalyze the hydrolysis of terminal non-reducing residues in β-D-glucosides can be introduced into the Vd3A polypeptide. In some aspects, the mutant Vd3A polypeptides comprise one or more conservative amino acid substitutions. In some aspects, the mutant Vd3A polypeptides comprise one or more non-conservative amino acid substitutions. In some aspects, the one or more amino acid substitutions are in the Vd3A polypeptide CD. In some aspects, the one or more amino acid substitutions are in the Vd3A polypeptide CBM. In some aspects, the one or more amino acid substitutions are in both the CD and the CBM. In some aspects, the Vd3A polypeptide amino acid substitutions can take place at amino acids E524 and/or D295. In some aspects, the Vd3A polypeptide amino acid substitutions can take place at one or more of amino acids D107, R113, L156, R171, K204, H205, R215, M260, Y263, D295, W296, S465, and/or E524. The mutant Vd3A polypeptide(s) suitably have β-glucosidase activity.

In some aspects, the Vd3A polypeptide comprises a chimera/hybrid/fusion of two β-glucosidase sequences, wherein the first β-glucosidase sequence is at least about 200 amino acid residues in length and comprises about 60%, 65%, 70%, 75%, or 80% or more sequence identity to a sequence of equal length of Vd3A (SEQ ID NO:76), and wherein the second β-glucosidase sequence is at least about 50 amino acid residues in length and comprises about 60%, 65%, 70%, 75%, 80% or more sequence identity to a sequence of equal length of any one of SEQ ID NOs:54, 56, 58, 60, 62, 64, 66, 68, 70, 72, 74, 78, and 79, or comprises a polypeptide sequence motif SEQ ID NO: 170. In some aspects, the first β-glucosidase sequence comprising an N-terminal sequence of at least 200 amino acid residues of SEQ ID NO:76, and the second β-glucosidase sequence comprising a C-terminal sequence of at least about 50 contiguous amino acid residues of any one of SEQ ID NOs:54, 56, 58, 60, 62, 64, 66, 68, 70, 72, 74, 78, and 79, or comprises a polypeptide sequence motif SEQ ID NO: 170.

In certain aspects, the Vd3A polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs:54, 56, 58, 60, 62, 64, 66, 68, 70, 72, 74, 78, and 79, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Vd3A (SEQ ID NO:76). In some aspects, the first β-glucosidase sequence comprises an N-terminal sequence of at least 200 amino acid residues of any one of SEQ ID NOs: 54, 56, 58, 60, 62, 64, 66, 68, 70, 72, 74, 78, and 79, or comprises one or more or all of polypeptide sequence motifs SEQ ID NOs: 164-169, and the second β-glucosidase sequence comprises a C-terminal sequence of at least 50 contiguous amino acid residues of SEQ ID NO:76.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Vd3A polypeptide or a variant thereof. In some aspects, the N-terminal sequence comprises one or more or all of the polypeptide sequence motifs represented by SEQ ID NOs:136-148, or preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the sequence motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Vd3A, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Pa3G

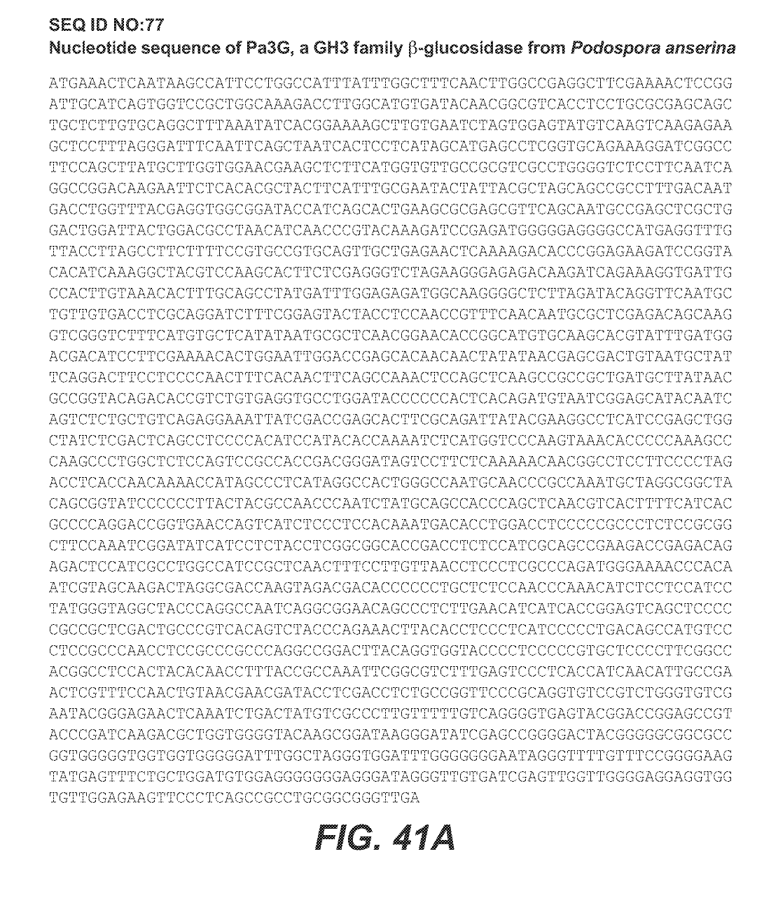

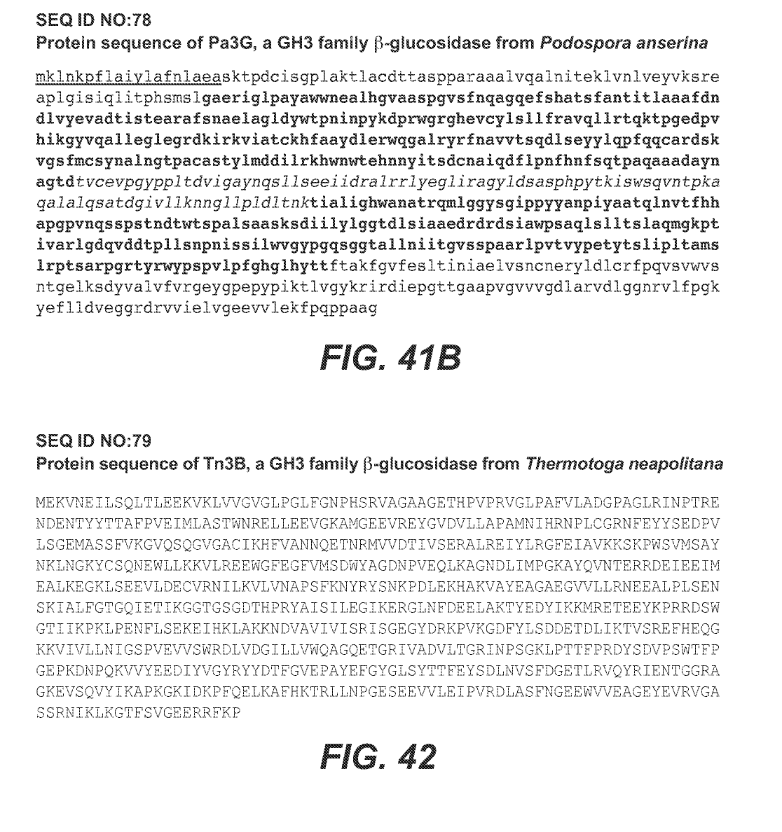

The amino acid sequence of Pa3G (SEQ ID NO:78) is shown in FIGS. 41B and 43. SEQ ID NO:78 is the sequence of the immature Pa3G. Pa3G has a predicted signal sequence corresponding to positions 1 to 19 of SEQ ID NO:78 (underlined); cleavage of the signal sequence is predicted to yield a mature protein having a sequence corresponding to positions 20 to 805 of SEQ ID NO:78. Signal sequence predictions were made with the SignalP-NN algorithm. The predicted conserved domain is in boldface type in FIG. 41B. Domain predictions were made based on the Pfam, SMART, or NCBI databases. Pa3G residues E517 and D289 are predicted to function as catalytic acid-base and nucleophile, respectively, based on a sequence alignment of the above-mentioned GH3 glucosidases from, e.g., P. anserina (Accession No. XP_{—}001912683), V. dahliae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Pa3G polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 20 to 805 of SEQ ID NO:78. A Pa3G polypeptide preferably is unaltered, as compared to a native Pa3G, at residues E517 and D289. A Pa3G polypeptide is preferably unaltered in at least 70%, 80%, 90%, 95%, 98%, or 99% of the amino acid residues that are conserved among the herein described GH3 family β-glucosidases as shown in the alignment of FIG. 43. A Pa3G polypeptide suitably comprises the entire predicted conserved domains of native Pa3G shown in FIG. 41B. An exemplary Pa3G polypeptide comprises a sequence having at least 85%, 86%, 87%, 88%, 89%, 90%, 91%, 92%, 93%, 94%, 95%, 96%, 97%, 98%, 99%, or 100% identity to the mature Pa3G sequence shown in FIG. 41B. The Pa3G polypeptide of the invention preferably has β-glucosidase activity.

Accordingly a Pa3G polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:78, or to residues (i) 20-354, (ii) 20-660, (iii) 20-805, (iv) 449-660, or (v) 449-805 of SEQ ID NO:78. The polypeptide suitably has β-glucosidase activity.

In some aspects, a “Pa3G polypeptide” of the invention can also refer to a mutant Vd3A polypeptide. Amino acid substitutions can be introduced into the Pa3G polypeptide to improve the β-glucosidase activity of the molecule. For example, amino acid substitutions that increase the binding affinity of the Pa3G polypeptide for its substrate or that improve its ability to catalyze the hydrolysis of terminal non-reducing residues in β-D-glucosides can be introduced into the Pa3G polypeptide. In some aspects, the mutant Pa3G polypeptides comprise one or more conservative amino acid substitutions. In some aspects, the mutant Pa3G polypeptides comprise one or more non-conservative amino acid substitutions. In some aspects, the one or more amino acid substitutions are in the Pa3G polypeptide CD. In some aspects, the one or more amino acid substitutions are in the Pa3G polypeptide CBM. In some aspects, the one or more amino acid substitutions are in both the CD and the CBM. In some aspects, the Pa3G polypeptide amino acid substitutions can take place at amino acids E517 and/or D289. In some aspects, the Pa3G polypeptide amino acid substitutions can take place at one or more of amino acids D101, R107, L150, R165, K199, H209, R215, M254, Y257, D289, W290, S458, and/or E517. The mutant Pa3G polypeptide(s) suitably have β-glucosidase activity.

In some aspects, the Pa3G polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Pa3G (SEQ ID NO:78), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 66, 68, 70, 72, 74, 76, and 79, or comprises a polypeptide sequence motif SEQ ID NO:170. In some aspects, the first β-glucosidase sequence comprising an N-terminal sequence of at least 200 amino acid residues of SEQ ID NO:78, and the second β-glucosidase sequence comprising a C-terminal sequence of at least about 50 contiguous amino acid residues of any one of SEQ ID NOs:54, 56, 58, 60, 62, 64, 66, 68, 70, 72, 74, 76, and 79, or comprises a polypeptide sequence motif SEQ ID NO:170.

In certain aspects, the Pa3G polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 66, 68, 70, 72, 74, 76, and 79, or comprises one or more or all of polypeptide sequence motifs SEQ ID NOs:164-169, whereas the second β-glucosidase sequence is at least about 50 amino acid residues in length and comprises about 60%, 65%, 70%, 75%, 80% or more sequence identity to a sequence of equal length Pa3G (SEQ ID NO:78). In some aspects, the first β-glucosidase sequence comprises an N-terminal sequence of at least 200 amino acid residues of any one of SEQ ID NOs:54, 56, 58, 60, 62, 64, 66, 68, 70, 72, 74, 76, and 79, or comprises one or more or all of polypeptide sequence motifs SEQ ID NOs:164-169, and the second β-glucosidase sequence comprises a C-terminal sequence of at least 50 contiguous amino acid residues of SEQ ID NO:78.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues, a sequence of FDRRSPG (SEQ ID NO:171), or of FD(R/K)YNIT (SEQ ID NO:172). In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Pa3G polypeptide or a variant thereof. In some aspects, the N-terminal sequence comprises one or more or all of the polypeptide sequence motifs represented by SEQ ID NOs:136-148, or preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Pa3G, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

### Tn3B

The amino acid sequence of Tn3B (SEQ ID NO:79) is shown in FIGS. 42 and 43. SEQ ID NO:79 is the sequence of the immature Tn3B. The SignalP-NN algorithm (http://www.cbs.dtu.dk) did not provide a predicted signal sequence. Tn3B residues E458 and D242 are predicted to function as catalytic acid-base and nucleophile, respectively, based on a sequence alignment of the above-mentioned GH3 glucosidases, e.g., P. anserina (Accession No. XP_{—}001912683), V. dahhae, N. haematococca (Accession No. XP_{—}003045443), G. zeae (Accession No. XP_{—}386781), F. oxysporum (Accession No. BGL FOXG_{—}02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. Q0GC07), etc. (see, FIG. 43). As used herein, “a Tn3B polypeptide” refers, in some aspects, to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues of SEQ ID NO:79. A Tn3B polypeptide preferably is unaltered, as compared to a native Tn3B, at residues E458 and D242. A Tn3B polypeptide is preferably unaltered in at least 70%, 80%, 90%, 95%, 98%, or 99% of the amino acid residues that are conserved among the herein described GH3 family β-glucosidases as shown in the alignment of FIG. 43. A Tn3B polypeptide suitably comprises the entire predicted conserved domains of native Tn3B shown in FIG. 43. An exemplary Tn3B polypeptide comprises a sequence having at least 85%, 86%, 87%, 88%, 89%, 90%, 91%, 92%, 93%, 94%, 95%, 96%, 97%, 98%, 99%, or 100% identity to the mature Tn3B sequence shown in FIG. 42. The Tn3B polypeptide of the invention preferably has β-glucosidase activity.

Accordingly a Tn3B polypeptide of the invention suitably comprise an amino acid sequence with at least 80%, 85%, 90%, 91%, 92%, 93%, 94%, 95%, 96%, 97%, 98%, 99% or 100% sequence identity to the amino acid sequence of SEQ ID NO:79. The polypeptide suitably has β-glucosidase activity.

In some aspects, a “Tn3B polypeptide” of the invention can also refer to a mutant Tn3B polypeptide. Amino acid substitutions can be introduced into the Tn3B polypeptide to improve the β-glucosidase activity of the molecule. For example, amino acid substitutions that increase the binding affinity of the Tn3B polypeptide for its substrate or that improve Tn3B's ability to catalyze the hydrolysis of terminal non-reducing residues in β-D-glucosides can be introduced into the Tn3B polypeptide. In some aspects, the mutant Tn3B polypeptides comprise one or more conservative amino acid substitutions. In some aspects, the mutant Tn3B polypeptides comprise one or more non-conservative amino acid substitutions. In some aspects, the one or more amino acid substitutions are in the Tn3B polypeptide CD. In some aspects, the one or more amino acid substitutions are in the Tn3B polypeptide CBM. In some aspects, the one or more amino acid substitutions are in both the CD and the CBM. In some aspects, the Tn3B polypeptide amino acid substitutions can take place at amino acids E458 and/or D242. In some aspects, the Tn3B polypeptide amino acid substitutions can take place at one or more of amino acids D58, R64, L116, R130, K163, H164, R174, M207, Y210, D242, W243, S370, and/or E458. The mutant Tn3B polypeptide(s) suitably have β-glucosidase activity.

In some aspects, the Tn3B polypeptide comprises a chimera/fusion/hybrid of two β-glucosidase sequences, wherein the first β-glucosidase sequence is at least about 200 amino acid residues in length and comprises about 60%, 65%, 70%, 75%, or 80% or more sequence identity to a sequence of equal length of Tn3B (SEQ ID NO:79), and wherein the second β-glucosidase sequence is at least about 50 amino acid residues in length and comprises at least about 60%, 65%, 70%, 75%, 80% or more sequence identity to a sequence of equal length of any one of SEQ ID NOs:54, 56, 58, 60, 62, 64, 66, 68, 70, 72, 74, 76, and 78, or comprises a polypeptide sequence motif SEQ ID NO:170. In some aspects, the first β-glucosidase sequence comprising an N-terminal sequence of at least 200 amino acid residues of SEQ ID NO:79, and the second β-glucosidase sequence comprising a C-terminal sequence of at least about 50 contiguous amino acid residues of any one of SEQ ID NOs:54, 56, 58, 60, 62, 64, 66, 68, 70, 72, 74, 76, and 78, or comprises a polypeptide sequence motif SEQ ID NO:170.

In certain aspects, the Tn3B polypeptide of the invention comprises a chimera or a chimeric construct of two β-glucosidase sequences, wherein the first β-glucosidase sequence is at least about 200 amino acid residues in length and comprises about 60%, 65%, 70%, 75%, 80% or more sequence identity to a sequence of equal length of any one of SEQ ID NOs: 54, 56, 58, 60, 62, 64, 66, 68, 70, 72, 74, 76, and 78, or comprises one or more or all of polypeptide sequence motifs SEQ ID NOs: 164-169, whereas the second β-glucosidase sequence is at least about 50 amino acid residues in length and comprises about 60%, 65%, 70%, 75%, 80% or more sequence identity to a sequence of equal length of Tn3B (SEQ ID NO:79). In some aspects, the first β-glucosidase sequence comprises an N-terminal sequence of at least 200 amino acid residues of any one of SEQ ID NOs:54, 56, 58, 60, 62, 64, 66, 68, 70, 72, 74, 76, and 78, or comprises one or more or all of polypeptide sequence motifs SEQ ID NOs: 164-169, and the second β-glucosidase sequence comprises a C-terminal sequence of at least 50 contiguous amino acid residues of SEQ ID NO:79.

In some aspects, the first β-glucosidase sequence is located at the N-terminal of the chimeric β-glucosidase polypeptide whereas the second β-glucosidase sequence is located at the C-terminal of the chimeric β-glucosidase polypeptide. In certain embodiments, the first, the second, or both of the β-glucosidase sequences further comprise one or more glycosylation sites. In certain embodiments, the first and second β-glucosidase sequences are immediately adjacent to each other or directly connected to each other. In other embodiments, the first and second β-glucosidase sequences are not immediately adjacent but are connected via a linker domain. In some aspects, the first or the second β-glucosidase sequence comprises a loop region or a sequence representing a loop-like structure, comprising about 3, 4, 5, 6, 7, 8, 9, 10, or 11 amino acid residues, comprising a sequence of FDRRSPG (SEQ ID NO:171), or of FD(R/K)YNIT (SEQ ID NO:172). In some aspects, neither the first nor the second β-glucosidase sequence comprises a loop sequence. In some embodiments, the linker domain comprises a loop region, which comprises about 3, 4, 5, 6, 7, 8, 9, 10, or 11 amino acid residues. In some embodiments, the linker domain connecting the first β-glucosidase sequence and the second β-glucosidase sequence are located centrally (i.e., not located at the N- or C-terminal of the chimeric polypeptide). In some aspects, the N-terminal sequence of the chimeric β-glucosidase comprises a sequence of at least 200, 250, 300, 350, 400, 450, 500, 550, or 600 residues in length derived from a Tn3B polypeptide or a variant thereof. In some aspects, the N-terminal sequence comprises one or more or all of the polypeptide sequence motifs represented by SEQ ID NOs:136-148, or preferably the motifs SEQ ID NOs:164-169. In some aspects, the C-terminal sequence comprises a sequence of at least 50, 75, 100, 125, 150, 175, or 200 amino acid residues in length derived from a β-glucosidase polypeptide or a variant thereof. In some aspects, the C-terminal sequence comprises one or more or all of the polypeptide sequence motifs represented by SEQ ID NOs:149-156, or preferably the motif SEQ ID NO:170. In certain embodiments, the β-glucosidase polypeptide, the variant thereof, or the hybrid or chimera thereof further comprises one or more glycosylation sites. The one or more glycosylation sites can be located either within the C-terminal sequence or within the N-terminal sequence, or within both.

In some aspects, the non-naturally occurring cellulase or hemicellulase composition of the invention further comprises one or more naturally occurring hemicellulases. In some aspects, the non-naturally occurring cellulase composition has improved stability over the native enzymes, including Tn3B, from which either the C-terminal or the N-terminal sequences of the chimeric β-glucosidase were derived. In some aspects, the improved stability comprises an improvement in proteolytic stability during storage, expression or production processes. In some aspects, the improved stability comprises an associated decrease in rate or extent of enzymatic activity loss during storage or production conditions, wherein the enzymatic activity loss is preferably less than about 50%, less than about 40%, less than about 20%, more preferably less than about 15%, or even more preferably less than about 10%. In some aspects, the N-terminal sequence or the C-terminal sequence can comprise a loop sequence, comprising about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). The N-terminal and C-terminal sequences can be immediately adjacent or directly connected to each other. In other aspects, the N-terminal sequence and the C-terminal sequence can be connected via a linker domain. In certain embodiments, the linker domain comprises a loop sequence of about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some aspects, the non-naturally occurring cellulase composition comprises β-glucosidase activity. In some aspects, the non-naturally occurring cellulase composition further comprises one or more of xylanase, β-xylosidase, and/or L-α-arabinofuranosidase activities.

Nucleic Acids

Exemplary β-glucosidase nucleic acids include nucleic acids that encode a polypeptide, fragment of a polypeptide, peptide, or fusion polypeptide that has at least one activity of a β-glucosidase polypeptide. Exemplary β-glucosidase polypeptides and nucleic acids include naturally-occurring polypeptides and nucleic acids from any of the source organisms described herein as well as mutant polypeptides and nucleic acids derived from any of the source organisms described herein. Exemplary β-glucosidase nucleic acids include, e.g., β-glucosidase isolated from, without limitation, one or more of the following organisms: Crinipellis scapella, Macrophomina phaseolina, Myceliophthora thermophila, Sordaria fimicola, Volutella colletotrichoides, Thielavia terrestris, Acremonium sp., Exidia glandulosa, Fomes fomentarius, Spongipellis sp., Rhizophlyctis rosea, Rhizomucor pusillus, Phycomyces niteus, Chaetostylum fresenii, Diplodia gossypina, Ulospora bilgramii, Saccobolus dilutellus, Penicillium verruculosum, Penicillium chrysogenum, Thermomyces verrucosus, Diaporthe syngenesia, Colletotrichum lagenarium, Nigrospora sp., Xylaria hypoxylon, Nectria pinea, Sordaria macrospora, Thielavia thermophila, Chaetomium mororum, Chaetomium virscens, Chaetomium brasiliensis, Chaetomium cunicolorum, Syspastospora boninensis, Cladorrhinum foecundissimum, Scytalidium thermophila, Gliocladium catenulatum, Fusarium oxysporum ssp. lycopersici, Fusarium oxysporum ssp. passiflora, Fusarium solani, Fusarium anguioides, Fusarium poae, Humicola nigrescens, Humicola grisea, Panaeolus retirugis, Trametes sanguinea, Schizophyllum commune, Trichothecium roseum, Microsphaeropsis sp., Acsobolus stictoideus spej., Poronia punctata, Nodulisporum sp., Trichoderma sp. (e.g., T. reesei) and Cylindrocarpon sp.

The disclosure provides isolated, synthetic or recombinant nucleic acids comprising a nucleic acid sequence having at least about 70%, e.g., at least about 71%, 72%, 73%, 74%, 75%, 76%, 77%, 78%, 79%, 80%, 81%, 82%, 83%, 84%, 85%, 86%, 87%, 88%; 89%, 90%, 91%, 92%, 93%, 94%, 95%, 96%, 97%, 98%, or 99%, or complete (100%) sequence identity to a nucleic acid of SEQ ID NO:1, 3, 5, 7, 9, 11, 13, 15, 17, 19, 21, 23, 25, 27, 29, 31, 33, 35, 37, 39, 41, 46, 47, 48, 49, 50, 51, 53, 57, 59, 61, 63, 65, 67, 69, 71, 73, 75, or 77, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 nucleotides. The present disclosure also provides nucleic acids encoding at least one polypeptide having a hemicellulolytic activity (e.g., a xylanase, β-xylosidase, and/or L-α-arabinofuranosidase activity). Furthermore, the present disclosure provides nucleic acids encoding polypeptides having celluloytic activities (e.g., β-glucosidase activity, or endoglucanase activity).

Nucleic acids of the disclosure also include isolated, synthetic or recombinant nucleic acids encoding an enzyme or a mature portion of an enzyme comprising the sequence of SEQ ID NO:2, 4, 6, 8, 10, 12, 14, 16, 18, 20, 22, 24, 26, 28, 30, 32, 34, 36, 38, 40, 42, 43, 44, 52, 54, 56, 58, 60, 62, 64, 66, 68, 70, 72, 74, 76, 78, or 79, or to a GH61 endoglucanase enzyme or a mature portion of that enzyme comprising the polypeptide sequence motifs: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91, and subsequences thereof (e.g., a conserved domain or carbohydrate binding domain (“CBM”), and variants thereof.

The disclosure specifically provides a nucleic acid encoding an Fv3A, a Pf43A, an Fv43E, an Fv39A, an Fv43A, an Fv43B, a Pa51A, a Gz43A, an Fo43A, an Af43A, a Pf51A, an AfuXyn2, an AfuXyn5, a Fv43D, a Pf43B, Fv43B, a Fv51A, a T. reesei Xyn3, a T. reesei Xyn2, a T. reesei Bxl1, a T. reesei Bgl1 (Tr3A), a T. reesei Eg4, a T. reesei Bgl3 (Tr3B), a Pa3D, an Fv3G, an Fv3D, an Fv3C, a Te3A, an An3A, an Fo3A, a Gz3A, an Nh3A, a Vd3A, a Pa3G or a Tn3B polypeptide, a variant, a mutant, or a hybrid or chimeric polypeptide thereof. In some aspects, the disclosure provides a nucleic acid encoding a chimeric or fusion enzyme comprising, e.g., a first β-glucosidase sequence and a second β-glucosidase sequence, wherein the first β-glucosidase sequence and the second β-glucosidase sequence are derived from different organisms. In certain aspect, the first β-glucosidase sequence is at the N-terminal, and the second β-glucosidase is at the C-terminal of the hybrid or chimera β-glucosidase polypeptide. In certain aspect, the first β-glucosidase sequence, or more specifically, the C-terminus of the first β-glucosidase sequence, is directly adjacent or connected to the second β-glucosidase sequence, or more specifically, to the N-terminus of the second β-glucosidase sequence. In some embodiments, the first β-glucosidase sequence and the second β-glucosidase are not directly adjacent or connected, but rather, the first β-glucosidase sequence is operably linked or connected to the second β-glucosidase sequence via a linker sequence or domain. In some examples, the first β-glucosidase sequence is at least about 200 amino acid residues in length, and comprises one or more or all of the polypeptide sequence motifs represented by SEQ ID NOs: 136-148, whereas the second β-glucosidase sequence is at least about 50 amino acid residues in length, and comprises one or more or all of the polypeptide sequence motifs represented by SEQ ID NOs: 149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In some aspects, the first β-glucosidase sequence and the second β-glucosidase sequence are directly connected or immediately adjacent to each other. In some aspect, the first β-glucosidase sequence is not directly connected or immediately adjacent to the second β-glucosidase sequence, but rather, the first and second β-glucosidase are connected via a linker sequence. In certain embodiments, the linker sequence is centrally located. In certain specific example, the first β-glucosidase sequence comprises a sequence, e.g., an N-terminal sequence of at least 200 amino acid residues in length of an Fv3C polypeptide. In some embodiments, the second β-glucosidase sequence comprises a sequence, e.g., a C-terminal sequence of at least 50 amino acid residues in length, of a T. reesei Bgl3 polypeptide. In a particular example, the β-glucosidase polypeptide is a hybrid or chimeric Fv3C polypeptide, or a T. reesei Bgl3 (Tr3B) polypeptide, and comprises an amino acid sequence of SEQ ID NO:159. In another example, the β-glucosidase polypeptide is a hybrid or chimeric Fv3C polypeptide, or a T. reesei Bgl3 polypeptide, optionally comprising a linker sequence derived from a third β-glucosidase polypeptide sequence, wherein the β-glucosidase polypeptide comprises an amino acid sequence of SEQ ID NO:135. The chimeric or fusion enzyme suitably also comprise a linker sequence in some aspects, and accordingly, the disclosure provides a nucleic acid encoding a chimeric enzyme, which can be deemed a β-glucosidase polypeptide from which any of the N-terminal sequence, C-terminal sequence, or subsequences thereof are derived. For example, a hybrid Fv3C/Bgl3 polypeptide can be deemed an Fv3C polypeptide, a variant thereof, a T. reesei Bgl3 polypeptide, a variant thereof, or a chimeric Fv3C/Bgl3 polypeptide or a variant thereof. In another example, a hybrid Fv3C/Te3A/Bgl3 polypeptide can be deemed an Fv3C polypeptide or a variant thereof, a T. reesei Bgl3 polypeptide or a variant thereof, a Te3A polypeptide or a variant thereof, or a chimeric Fv3C/Te3A/Bgl3/polypeptide or a variant thereof.

The term “variant,” when used in the context of a polynucleotide sequence, may encompass a polynucleotide sequence related to that of a gene or the coding sequence thereof. This definition may also include, e.g., “allelic,” “splice,” “species,” or “polymorphic” variants. A splice variant may have significant identity to a reference polynucleotide, but will generally have a greater or fewer number of residues due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other, as further detailed within. A polymorphic variant is a variation in the polynucleotide sequence of a particular gene between individuals of a given species.

For example, the disclosure provides an isolated nucleic acid molecule, wherein the nucleic acid molecule encodes:

(1) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:54, or to residues (i) 18-282, (ii) 18-601, (iii) 18-733, (iv) 356-601, or (v) 356-733 of SEQ ID NO:54; or
(2) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:56, or to residues (i) 22-292, (ii) 22-629, (iii) 22-780, (iv) 373-629, or (v) 373-780 of SEQ ID NO:56; or
(3) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:58, or to residues (i) 20-321, (ii) 20-651, (iii) 20-811, (iv) 423-651, or (v) 423-811 of SEQ ID NO:58; or
(4) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:60, or to residues (i) 20-327, (ii) 22-600, (iii) 20-899, (iv) 428-899, or (v) 428-660 of SEQ ID NO:60; or
(5) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:62, or to residues (i) 20-287, (ii) 22-611, (iii) 20-744, (iv) 362-611, or (v) 362-744 of SEQ ID NO:62; or
(6) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:64, or to residues (i) 19-307, (ii) 19-640, (iii) 19-874, (iv) 407-640, or (v) 407-874 of SEQ ID NO:64; or
(7) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:66, or to residues (i) 20-297, (ii) 20-629, (iii) 20-857, (iv) 396-629, or (v) 396-857 of SEQ ID NO:66; or
(8) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:68, or to residues (i) 20-300, (ii) 20-634, (iii) 20-860, (iv) 400-634, or (v) 400-860 of SEQ ID NO:68; or
(9) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:70, or to residues (i) 20-327, (ii) 20-660, (iii) 20-899, (iv) 428-660, or (v) 428-899 of SEQ ID NO:70; or
(10) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:72, or to residues (i) 19-314, (ii) 19-647, (iii) 19-886, (iv) 415-647, or (v) 415-886 of SEQ ID NO:72; or
(11) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:74, or to residues (i) 20-295, (ii) 20-647, (iii) 20-880, (iv) 414-647, or (v) 414-880 of SEQ ID NO:74; or
(121) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:76, or to residues (i) 19-296, (ii) 19-649, (iii) 19-890, (iv) 415-649, or (v) 415-890 of SEQ ID NO:76; or
(13) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:78, or to residues (i) 20-354, (ii) 20-660, (iii) 20-805, (iv) 449-660, or (v) 449-805 of SEQ ID NO:78; or
(14) a polypeptide comprising an amino acid sequence with at least 80%, 85%, 90%, 91%, 92%, 93%, 94%, 95%, 96%, 97%, 98%, 99% or 100% sequence identity to the amino acid sequence of SEQ ID NO:79.

The instant disclosure also provides:

(1) a nucleic acid having at least 90% (e.g., at least 90%, 91%, 92%, 93%, 94%, 95%, 96%, 97%, 98%, 99% or more) sequence identity to SEQ ID NO:53, or a nucleic acid that is capable of hybridizing under high stringency conditions to a complement of SEQ ID NO:53, or to a fragment thereof; or
(2 a nucleic acid having at least 90% (e.g., at least 90%, 91%, 92%, 93%, 94%, 95%, 96%, 97%, 98%, 99% or more) sequence identity to SEQ ID NO:55, or a nucleic acid that is capable of hybridizing under high stringency conditions to a complement of SEQ ID NO:55, or to a fragment thereof; or
(3) a nucleic acid having at least 90% (e.g., at least 90%, 91%, 92%, 93%, 94%, 95%, 96%, 97%, 98%, 99% or more) sequence identity to SEQ ID NO:57, or a nucleic acid that is capable of hybridizing under high stringency conditions to a complement of SEQ ID NO:57, or to a fragment thereof; or
(4) a nucleic acid having at least 90% (e.g., at least 90%, 91%, 92%, 93%, 94%, 95%, 96%, 97%, 98%, 99% or more) sequence identity to SEQ ID NO:59, or a nucleic acid that is capable of hybridizing under high stringency conditions to a complement of SEQ ID NO:59, or to a fragment thereof; or
(5) a nucleic acid having at least 90% (e.g., at least 90%, 91%, 92%, 93%, 94%, 95%, 96%, 97%, 98%, 99% or more) sequence identity to SEQ ID NO:61, or a nucleic acid that is capable of hybridizing under high stringency conditions to a complement of SEQ ID NO:61, or to a fragment thereof; or
(6) a nucleic acid having at least 90% (e.g., at least 90%, 91%, 92%, 93%, 94%, 95%, 96%, 97%, 98%, 99% or more) sequence identity to SEQ ID NO:63, or a nucleic acid that is capable of hybridizing under high stringency conditions to a complement of SEQ ID NO:63, or to a fragment thereof; or
(7) a nucleic acid having at least 90% (e.g., at least 90%, 91%, 92%, 93%, 94%, 95%, 96%, 97%, 98%, 99% or more) sequence identity to SEQ ID NO:65, or a nucleic acid that is capable of hybridizing under high stringency conditions to a complement of SEQ ID NO:65, or to a fragment thereof; or
(8) a nucleic acid having at least 90% (e.g., at least 90%, 91%, 92%, 93%, 94%, 95%, 96%, 97%, 98%, 99% or more) sequence identity to SEQ ID NO:67, or a nucleic acid that is capable of hybridizing under high stringency conditions to a complement of SEQ ID NO:67, or to a fragment thereof; or
(9) a nucleic acid having at least 90% (e.g., at least 90%, 91%, 92%, 93%, 94%, 95%, 96%, 97%, 98%, 99% or more) sequence identity to SEQ ID NO:69, or a nucleic acid that is capable of hybridizing under high stringency conditions to a complement of SEQ ID NO:69, or to a fragment thereof; or
(10) a nucleic acid having at least 90% (e.g., at least 90%, 91%, 92%, 93%, 94%, 95%, 96%, 97%, 98%, 99% or more) sequence identity to SEQ ID NO:71, or a nucleic acid that is capable of hybridizing under high stringency conditions to a complement of SEQ ID NO:71, or to a fragment thereof; or
(11) a nucleic acid having at least 90% (e.g., at least 90%, 91%, 92%, 93%, 94%, 95%, 96%, 97%, 98%, 99% or more) sequence identity to SEQ ID NO:73, or a nucleic acid that is capable of hybridizing under high stringency conditions to a complement of SEQ ID NO:73, or to a fragment thereof; or
(12) a nucleic acid having at least 90% (e.g., at least 90%, 91%, 92%, 93%, 94%, 95%, 96%, 97%, 98%, 99% or more) sequence identity to SEQ ID NO:75, or a nucleic acid that is capable of hybridizing under high stringency conditions to a complement of SEQ ID NO:75, or to a fragment thereof; or
(13) a nucleic acid having at least 90% (e.g., at least 90%, 91%, 92%, 93%, 94%, 95%, 96%, 97%, 98%, 99% or more) sequence identity to SEQ ID NO:77, or a nucleic acid that is capable of hybridizing under high stringency conditions to a complement of SEQ ID NO:77, or to a fragment thereof.
As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified

Example of Methods for Isolating Nucleic Acids

β-glucosidase and other nucleic acids of the present disclosure can be isolated using standard methods. Methods of obtaining desired nucleic acids from a source organism of interest (such as a bacterial genome) are common and well known in the art of molecular biology. Standard methods of isolating nucleic acids, including PCR amplification of known sequences, synthesis of nucleic acids, screening of genomic libraries, screening of cosmid libraries are described in International Publication No. WO 2009/076676 A2 and U.S. patent application Ser. No. 12/335,071.

Examples of Host Cells

The present disclosure provides host cells that are engineered to express one or more enzymes of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus lichenifonnis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thennophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

The disclosure further provides a recombinant host cell that is engineered to express one or more, two or more, three or more, four or more, or five or more of an Fv3A, a Pf43A, an Fv43E, an Fv39A, an Fv43A, an Fv43B, a Pa51A, a Gz43A, an Fo43A, an Af43A, a Pf51A, an AfuXyn2, an AfuXyn5, a Fv43D, a Pf43B, Fv43B, a Fv51A, a T. reesei Xyn3, a T. reesei Xyn2, a T. reesei Bxl1, a T. reesei Bgl1 (Tr3A), a GH61 endoglucanase, a T. reesei Eg4, a Pa3D, an Fv3G, an Fv3D, an Fv3C, a Tr3B, a Te3A, an An3A, an Fo3A, a Gz3A, an Nh3A, a Vd3A, a Pa3G or a Tn3B polypeptide, or a variant thereof.

In certain embodiments, recombinant host cell expressing hybrid or chimeric enzymes derived from two or more cellulase sequences and/or hemicellulase sequences are contemplated. In some aspects, the hybrid or chimeric enzyme comprises two or more β-glucosidase sequences. In some aspects, the first β-glucosidase sequence is at least about 200 amino acid residues in length, and comprises one or more or all of the polypeptide sequence motifs of SEQ ID NOs:136-148, and the second β-glucosidase sequence is at least about 50 amino acid residues in length and comprises one or more or all of the polypeptide sequence motifs selected from SEQ ID NOs: 149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In certain embodiments, the first β-glucosidase sequence is at the N-terminal and the second β-glucosidase sequence is at the C-terminal of the hybrid or chimeric polypeptide. In certain embodiments, the first and second β-glucosidase sequences are immediately adjacent or directly connected to each other. In other embodiments, the first and second β-glucosidase sequences are not immediately adjacent or directly connected, but rather are connected via a linker domain. In certain embodiments, the linker domain is centrally located. In certain aspects, either the first or the second β-glucosidase sequence comprises a loop sequence, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172), the modification of which improves the stability of the hybrid or chimeric polypeptide as compared to the unmodified counterpart polypeptide, or the polypeptides from which the chimeric parts of the hybrid or chimeric polypeptide are derived. In certain embodiments, neither the first nor the second β-glucosidase sequences comprise the loop sequence, but rather the linker domain comprises the loop sequence. In some embodiments, the modification of the loop sequence, e.g., shortening, lengthening, deleting, replacing, substituting, or otherwise modifying the sequence, lessens the cleavage of residues in the loop sequence. In other embodiments, the modification of the loop sequence lessens the cleavage of residues at sites outside of the loop sequence.

In certain embodiments, recombinant host cell expressing hybrid or chimeric enzymes derived from two or more cellulase sequences and/or hemicellulase sequences are contemplated. In some aspects, the hybrid or chimeric enzyme comprises two or more β-glucosidase sequences. In some embodiments, recombinant host cell expressing hybrid or chimeric enzymes comprising a first sequence is at least about 200 contiguous amino acid residues in length, and has least 60%, 70%, 80%, 90%, 91%, 92%, 93%, 94%, 95%, 96%, 97%, 98%, 99% or more sequence identity to an equal length sequence of SEQ ID NO:60; and a second sequence is at least about 50 contiguous amino acid residues in length and has at least about 60%, 70%, 80%, 90%, 91%, 92%, 93%, 94%, 95%, 96%, 97%, 98%, 99% or more sequence identity to a sequence of equal length of any one of SEQ ID NOs:54, 56, 58, 62, 64, 66, 68, 70, 72, 74, 76, 78, and 79 are contemplated. In alternative embodiments, recombinant host cell expressing hybrid or chimeric enzymes comprising a first sequence is at least about 200 contiguous amino acid residues in length, and has least 60%, 70%, 80%, 90%, 91%, 92%, 93%, 94%, 95%, 96%, 97%, 98%, 99% or more sequence identity to an equal length sequence of any one of SEQ ID NOs:54, 56, 58, 60, 62, 64, 66, 68, 70, 72, 74, 76, 78, and 79; and a second sequence is at least about 50 contiguous amino acid residues in length and has at least about 60%, 70%, 80%, 90%, 91%, 92%, 93%, 94%, 95%, 96%, 97%, 98%, 99% or more sequence identity to a sequence of SEQ ID NO:60 are contemplated. In certain embodiments, the first β-glucosidase sequence is at the N-terminal and the second β-glucosidase sequence is at the C-terminal of the hybrid or chimeric polypeptide. In certain embodiments, the first and second β-glucosidase sequences are immediately adjacent or directly connected to each other. In other embodiments, the first and second β-glucosidase sequences are not immediately adjacent or directly connected, but rather are connected via a linker domain. In certain embodiments, the linker domain is centrally located. In certain aspects, either the first or the second β-glucosidase sequence comprises a loop sequence, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172) the modification of which improves the stability of the hybrid or chimeric polypeptide as compared to the unmodified counterpart polypeptide, or the polypeptides from which the chimeric parts of the hybrid or chimeric polypeptide are derived. In certain embodiments, neither the first nor the second β-glucosidase sequences comprise the loop sequence, but rather the linker domain comprises the loop sequence. In some embodiments, the modification of the loop sequence, e.g., shortening, lengthening, deleting, replacing, substituting, or otherwise modifying the sequence, lessens the cleavage of residues in the loop sequence. In other embodiments, the modification of the loop sequence lessens the cleavage of residues at sites outside of the loop sequence.

In some aspects, the recombinant host cell expresses one or more chimeric enzyme, e.g., an Fv3C fusion enzyme, a T. reesei Bgl3 fusion enzyme, an Fv3C/Bgl3 fusion enzyme, a Te3A fusion enzyme, or an Fv3C/Te3A/Bgl3 fusion enzyme. For the disclosure herein, the terms “an XX fusion enzyme”, “an XX chimeric enzyme” and “an XX hybrid enzyme” are used interchangeably to refer to an enzyme having at least one chimeric part derived from an XX enzyme. For example, an Fv3C fusion or chimeric enzyme can refer to an Fv3C/Bgl3 hybrid enzyme (which is also a Bgl3 chimeric enzyme), or to an Fv3C/Te3A/Bgl3 hibrid enzyme (which is also a Te3A or Bgl3 chimeric enzyme).

The recombinant host cell is, e.g., a recombinant T. reesei host cell. In a particular example, the disclosure provides a recombinant fungus, such as a recombinant T. reesei, that is engineered to express 1 or more, 2 or more, 3 or more, 4 or more, or 5 or more of Fv3A, Pf43A, Fv43E, Fv39A, Fv43A, Fv43B, Pa51A, Gz43A, Fo43A, Af43A, Pf51A, AfuXyn2, AfuXyn5, Fv43D, Pf43B, Fv43B, Fv51A, T. reesei Xyn3, T. reesei Xyn2, a T. reesei Bxl1, T. reesei Bgl1(Tr3A), T. reesei Bgl3 (Tr3B), GH61 endoglucanase, T. reesei Eg4, Pa3D, Fv3G, Fv3D, Fv3C, Fv3C fusion/chimeric enzyme, Fv3C/Bgl3, Fv3C/Te3A/Bgl3 fusion/chimeric enzyme, Te3A, An3A, Fo3A, Gz3A, Nh3A, Vd3A, Pa3G or Tn3B polypeptide, or a variant or mutant thereof, including, e.g., a hybrid or chimeric polypeptide thereof.

The disclosure provides a host cell, e.g., a recombinant fungal host cell or a recombinant filamentous fungus, engineered to recombinantly express at least one xylanase, at least one β-xylosidase, and one L-α-arabinofuranosidase. The disclosure also provides a recombinant host cell, e.g., a recombinant fungal host cell or a recombinant filamentous fungus such as a recombinant T. reesei, that is engineered to express 1, 2, 3, 4, 5, or more of Fv3A, Pf43A, Fv43E, Fv39A, Fv43A, Fv43B, Pa51A, Gz43A, Fo43A, Af43A, Pf51A, AfuXyn2, AfuXyn5, Fv43D, Pf43B, Fv43B, Fv51A, Pa3D, Fv3G, Fv3D, Fv3C, Fv3C fusion enzyme, a T. reesei Bgl3 (Tr3B), a T. reesei Bgl3 fusion enzyme, an Fv3C/Bgl3 fusion enzyme, Tr3A, Te3A, a Te3A fusion enzyme, an Fv3C/Te3A/Bgl3 fusion enzyme, An3A, Fo3A, Gz3A, Nh3A, Vd3A, Pa3G or Tn3B polypeptide, in addition to one or more of a T. reesei Xyn3, a T. reesei Xyn2, a T. reesei Bxl1, a T. reesei Bgl1, a GH61 endoglucanase, a T. reesei Eg4, or a variant thereof. The recombinant host cell is, e.g., a T. reesei host cell.

The present disclosure also provides a recombinant host cell e.g., a recombinant fungal host cell or a recombinant organism, e.g., a filamentous fungus, such as a recombinant T. reesei, that is engineered to recombinantly express T. reesei Xyn3, T. reesei Bgl1, T. reesei Bgl3 (Tr3B), T. reesei Bgl3 fusion enzyme, Fv3A, Fv43D, and Fv51A polypeptides. For example, the recombinant host cell is suitably a T. reesei host cell. The recombinant fungus is suitably a recombinant T. reesei. The disclosure provides, e.g., a T. reesei host cell engineered to recombinantly express T. reesei Xyn3, T. reesei Bgl1, a T. reesei Bgl3 fusion enzyme, Fv3A, Fv43D, and Fv51A polypeptides

Examples of Promoters and Vectors

The disclosure also provides expression cassettes and/or vectors comprising the above-described nucleic acids. Suitably, the nucleic acid encoding an enzyme of the disclosure is operably linked to a promoter. Promoters are well known in the art. Any promoter that functions in the host cell can be used for expression of a β-glucosidase and/or any of the other nucleic acids of the present disclosure. Initiation control regions or promoters, which are useful to drive expression of a β-glucosidase nucleic acids and/or any of the other nucleic acids of the present disclosure in various host cells are numerous and familiar to those skilled in the art (see, e.g., WO 2004/033646 and references cited therein). Virtually any promoter capable of driving these nucleic acids can be used.

Specifically, where recombinant expression in a filamentous fungal host is desired, the promoter can be a filamentous fungal promoter. The nucleic acids can be, e.g., under the control of heterologous promoters. The nucleic acids can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or β-glucosidase promoter. A particularly suitable promoter can be, e.g., a T. reesei cellobiohydrolase, endoglucanase, or β-glucosidase promoter. For example, the promoter is a cellobiohydrolase I (cbh1) promoter. Non-limiting examples of promoters include a cbh1, cbh2, egl1, egl2, egl3, egl4, eg15, pki1, gpd1, xyn1, or xyn2 promoter. Additional non-limiting examples of promoters include a T. reesei cbh1, cbh2, egl1, egl2, egl3, egl4, eg15, pki1, gpd1, xyn1, or xyn2 promoter.

As used herein, the term “operably linked” means that selected nucleotide sequence (e.g., encoding a polypeptide described herein) is in proximity with a promoter to allow the promoter to regulate expression of the selected DNA. In addition, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

Any of the β-glucosidases and/or other nucleic acids described herein can be included in one or more vectors. Accordingly, also described herein are vectors with one more nucleic acids encoding any of the β-glucosidases and/or other nucleic acids of the present disclosure. In some aspects, the vector contains a nucleic acid under the control of an expression control sequence. In some aspects, the expression control sequence is a native expression control sequence. In some aspects, the expression control sequence is a non-native expression control sequence. In some aspects, the vector contains a selective marker or selectable marker. In some aspects, one or more β-glucosidase(s) integrates into a chromosome of the cells without a selectable marker.

Suitable vectors are those which are compatible with the host cell employed. Suitable vectors can be derived, e.g., from a bacterium, a virus (such as bacteriophage T7 or a M-13 derived phage), a cosmid, a yeast, or a plant. Suitable vectors can be maintained in low, medium, or high copy number in the host cell. Protocols for obtaining and using such vectors are known to those in the art (see, e.g., Sambrook et al., Molecular Cloning: A Laboratory Manual, 2^{nd} ed., Cold Spring Harbor, 1989).

In some aspects, the expression vector also includes a termination sequence. Termination control regions may also be derived from various genes native to the host cell. In some aspects, the termination sequence and the promoter sequence are derived from the same source.

A β-glucosidases nucleic acid can be incorporated into a vector, such as an expression vector, using standard techniques (Sambrook et al., Molecular Cloning: A Laboratory Manual, Cold Spring Harbor, 1982).

In some aspects, it may be desirable to over-express one or more β-glucosidase(s) and/or one or more of any other nucleic acid described in the present disclosure at levels far higher than currently found in naturally-occurring cells. In some embodiments, it may be desirable to under-express (e.g., mutate, inactivate, or delete) β-glucosidase(s) and/or one or more of any other nucleic acid described in the present disclosure at levels far below that those currently found in naturally-occurring cells.

Examples of Transformation Methods

β-glucosidase nucleic acids or vectors containing them can be inserted into a host cell (e.g., a plant cell, a fungal cell, a yeast cell, or a bacterial cell described herein) using standard techniques for introduction of a DNA construct or vector into a host cell, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (see, e.g., Current Protocols in Molecular Biology (F. M. Ausubel et al. (eds) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989). The introduced nucleic acids may be integrated into chromosomal DNA or maintained as extrachromosomal replicating sequences. Transformants can be selected by any method known in the art.

Examples of Cell Culture Media

Generally, the microorganism is cultivated in a cell culture medium suitable for production of the polypeptides described herein. The cultivation takes place in a suitable nutrient medium comprising carbon and nitrogen sources and inorganic salts, using procedures and variations known in the art. Suitable culture media, temperature ranges and other conditions for growth and cellulase production are known in the art. As a non-limiting example, a typical temperature range for the production of cellulases by Trichoderma reesei is 24° C. to 28° C.

Examples of Cell Culture Conditions

Materials and methods suitable for the maintenance and growth of bacterial cultures are well known in the art. Exemplary techniques may be found in Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In some aspects, the cells are cultured in a culture medium under conditions permitting the expression of one or more β-glucosidases polypeptides encoded by a nucleic acid inserted into the host cells. Standard cell culture conditions can be used to culture the cells. In some aspects, cells are grown and maintained at an appropriate temperature, gas mixture, and pH. In some aspects, cells are grown at in an appropriate cell medium.

### Compositions of the Invention

The present disclosure provides engineered enzyme compositions (e.g., cellulase compositions) or fermentation broths enriched with one or more of the above-described polypeptides. In some aspects, the composition is a cellulase composition. The cellulase composition can be, e.g., a filamentous fungal cellulase composition, such as a Trichoderma cellulase composition. In some aspects, the composition is a cell comprising one or more nucleic acids encoding one or more cellulase polypeptides. In some aspects, the composition is a fermentation broth comprising cellulase activity, wherein the broth is capable of converting greater than about 50% by weight of the cellulose present in a biomass sample into sugars. The term “fermentation broth” as used herein refers to an enzyme preparation produced by fermentation that undergoes no or minimal recovery and/or purification subsequent to fermentation. The fermentation broth can be a fermentation broth of a filamentous fungus, e.g., a Trichoderma, Humicola, Fusarium, Aspergillus, Neurospora, Penicillium, Cephalosporium, Achlya, Podospora, Endothia, Mucor, Cochliobolus, Pyricularia, or Chrysosporium fermentation broth. In particular, the fermentation broth can be, e.g., one of Trichoderma spp. such as a T. reesei, or Penicillium spp., such as a P. funiculosum. The fermentation broth can also suitably be a cell-free fermentation broth. In one aspect, any of the cellulase, cell, or fermentation broth compositions of the present invention can further comprise one or more hemicellulases. In one aspect, the fermentation broth comprises whole cellulase. In certain embodiments, the fermentation broth may be used with limited post-production processing, including, e.g., purification, ultrafiltration, filtration, or a cell kill step, and as such, the fermentation broth is said to be used in a whole broth formulation. In some aspects, the whole cellulase composition is expressed in T. reesei. In some aspects the whole cellulase composition is expressed in T. reesei integrated strain H3A. In some aspects the whole cellulase composition is expressed in T. reesei integrated strain H3A, wherein one or more components of the polypeptides expressed in the T. reesei integrated strain H3A have been deleted. In some aspects, the whole cellulase composition is expressed in A. niger or an engineered strain thereof. In some aspects, the cellulase composition is capable of achieving at least 0.1 to 0.4 fraction product as determined by the calcofluor assay. In some aspects, the cellulase composition comprises 0.1 to 25 wt. % of the total enzyme weight of the composition. In some aspects, the cellulase composition further comprises one or more hemicellulases. In some aspects, the cellulase composition is capable of converting greater than about 70%, 75%, 80%, 85%, 90%, of the weight of the cellulose present in biomass into sugars. In some aspects, the cellulase composition comprises a polypeptide, wherein the percent by weight of cellulose in a biomass sample that is converted to sugars is increased relative to a cellulase composition that does not comprise the polypeptide.

In some aspects, the composition is a cellulase composition comprising a polypeptide having at least about 60%, e.g., at least about 65%, 70%, 75%, 80%, 85%, 90%, 91%, 92%, 93%, 94%, 95%, 96%, 97%, 98%, or 99% sequence identity to any one of the amino acid sequences of SEQ ID NOs: 54, 56, 58, 60, 62, 64, 66, 68, 70, 72, 74, 76, 78, and 79. In some aspects, the cellulase composition comprises a polypeptide having at least about 60%, e.g., at least about 65%, 70%, 75%, 80%, 85%, 90%, 91%, 92%, 93%, 94%, 95%, 96%, 97%, 98%, or 99% sequence identity to any one of the amino acid sequences of SEQ ID NOs: 54, 56, 58, 60, 62, 64, 66, 68, 70, 72, 74, 76, 78, and 79, wherein the cellulase composition is capable of converting greater than about 30%, e.g., greater than about 40%, 45%, 50%, 55%, 60%, 65%, 70%, 75%, or 80% by weight of the cellulose present in a biomass substrate into sugars. In certain embodiments, the biomass substrate is a mixture, in a solid, a gel, a semi-liquid, or a liquid form, typically as a result of subjecting the biomass substrate to certain suitable pretreatment processes, such as those described herein. In some aspects, the cellulase composition, which comprises a polypeptide having at least about 60%, (e.g., at least about 65%, 70%, 75%, 80%, 85%, 90%, 91%, 92%, 93%, 94%, 95%, 96%, 97%, 98%, or 99%) sequence identity to the amino acid sequence of SEQ ID NO: 54, 56, 58, 60, 62, 64, 66, 68, 70, 72, 74, 76, 78, and 79, and which is capable of converting greater than about 30%, (e.g., greater than about 40%, 45%, 50%, 55%, 60%, 65%, 70%, 75%, or 80%) by weight of the cellulose present in a biomass sample into sugars, is a whole cell composition. In some aspects, the cellulase composition, which comprises a polypeptide having at least about 60% (e.g., at least about 65%, 70%, 75%, 80%, 85%, 90%, 91%, 92%, 93%, 94%, 95%, 96%, 97%, 98%, or 99%) sequence identity to the amino acid sequence of any one of SEQ ID NOs: 54, 56, 58, 60, 62, 64, 66, 68, 70, 72, 74, 76, 78, and 79, wherein the cellulase composition is capable of converting greater than about 30%, e.g., greater than about 40%, 45%, 50%, 55%, 60%, 65%, 70%, 75%, or 80% by weight of the cellulose present in a biomass sample into sugars, is a fermentation broth. In some aspects, the fermentation broth comprises whole cellulase. In some aspects, the fermentation broth is a cell-free fermentation broth. In some aspects, the cellulase composition comprising a polypeptide having at least about 60% (e.g., at least about 65%, 70%, 75%, 80%, 85%, 90%, 91%, 92%, 93%, 94%, 95%, 96%, 97%, 98%, or 99%) sequence identity to the amino acid sequence of SEQ ID NO: 54, 56, 58, 60, 62, 64, 66, 68, 70, 72, 74, 76, 78, and 79 is expressed in T. reesei. In some aspects the cellulase composition comprising a polypeptide having at least about 60% (e.g., at least about 65%, 70%, 75%, 80%, 85%, 90%, 91%, 92%, 93%, 94%, 95%, 96%, 97%, 98%, or 99%) sequence identity to any one of the amino acid sequences of SEQ ID NOs: 54, 56, 58, 60, 62, 64, 66, 68, 70, 72, 74, 76, 78, and 79 is expressed in T. reesei integrated strain H3A. In some aspects one or more components of the polypeptides expressed in the T. reesei integrated strain H3A have been deleted. In some aspects, the cellulase composition comprising a polypeptide having at least about 60% (e.g., at least about 65%, 70%, 75%, 80%, 85%, or 90%) sequence identity to at least one of the amino acid sequences of SEQ ID NOs: 54, 56, 58, 60, 62, 64, 66, 68, 70, 72, 74, 76, 78, and 79 is expressed in A. niger or an engineered strain thereof. In some aspects, the cellulase composition comprising a polypeptide having at least about 60% (e.g., at least about 65%, 70%, 75%, 80%, 85%, or 90%) sequence identity to any one of the amino acid sequences of SEQ ID NOs: 54, 56, 58, 60, 62, 64, 66, 68, 70, 72, 74, 76, 78, and 79 is capable of achieving at least 0.1 to 0.4 fraction product as determined by the calcofluor assay. In some aspects, the cellulase composition comprising a polypeptide having at least about 60% (e.g., at least about 65%, 70%, 75%, 80%, 85%, or 90%) sequence identity to at least one of the amino acid sequences of SEQ ID NOs: 54, 56, 58, 60, 62, 64, 66, 68, 70, 72, 74, 76, 78, and 79 comprises 0.1 to 25 wt. % (e.g., 0.5 to 22 wt. %, 1 to 20 wt. %, 5 to 19 wt. %, 7 to 18 wt. %, 9 to 17 wt. %, 10 to 15 wt. %) of the total weight of proteins of the composition. In some aspects, the cellulase composition comprising a polypeptide having at least about 60% (e.g., at least about 65%, 70%, 75%, 80%, 85%, or 90%) sequence identity to at least one of the amino acid sequences of SEQ ID NOs: 54, 56, 58, 60, 62, 64, 66, 68, 70, 72, 74, 76, 78, and 79 further comprises one or more hemicellulases. In some aspects, the cellulase composition comprising a polypeptide having at least about 60% (e.g., at least about 65%, 70%, 75%, 80%, 85%, or 90%) sequence identity to at least one of the amino acid sequences of SEQ ID NOs: 54, 56, 58, 60, 62, 64, 66, 68, 70, 72, 74, 76, 78, and 79 is capable of converting greater than about 50% (e.g., greater than about 55%, 60%, 65%, 70%, 75%, 80%, 85%, or 90%) of the weight of the cellulose present in biomass into sugars. In some aspects, the cellulase composition comprises a polypeptide having at least about 60% (e.g., at least about 65%, 70%, 75%, 80%, 85%, or 90%) sequence identity to at least one of the amino acid sequences of SEQ ID NOs: 54, 56, 58, 60, 62, 64, 66, 68, 70, 72, 74, 76, 78, and 79, wherein the percent by weight of cellulose in a biomass sample that is converted to sugars is increased relative to a cellulase composition that does not comprise the polypeptide.

In some aspects, the cellulase composition is a a non-naturally occurring cellulase composition, which comprises a chimera/hybrid/fusion of two or more β-glucosidase sequences, wherein the first β-glucosidase sequence is at least about 200 amino acid residues in length and comprises about 60% (e.g., about 65%, 70%, 75%, 80%) or more sequence identity to an equal length (to the first β-glucosidase sequence) contiguous sequence of Fv3C (SEQ ID NO:60) and wherein the second β-glucosidase sequence is at least about 50 amino acid residues in length and comprises at least 60% (e.g., at least about 65%, 70%, 75%, 80%) sequence identity to an equal length (to the second β-glucosidase sequence) contiguous sequence of any one of SEQ ID NOs:54, 56, 58, 62, 64, 66, 68, 70, 72, 74, 76, 78, and 79, or comprises a polypeptide sequence motif of SEQ ID NO:170. In some aspects, the first β-glucosidase sequence is at the N-terminal of the chimeric polypeptide whereas the second β-glucosidase sequence is at the C-terminal of the chimeric polypeptide. In some aspects, the cellulase composition is a whole cell composition. In some aspects, the cellulase composition is a fermentation broth. In some aspects, the fermentation broth comprises whole cellulase. In some aspects, the fermentation broth is a cell-free fermentation broth.

In some aspects, the cellulase composition is a a non-naturally occurring cellulase composition, which comprises a chimera or a hybrid of two or more β-glucosidase sequences, wherein the first β-glucosidase sequence is at least about 200 amino acid residues in length and comprises about 60% (e.g., about 65%, 70%, 75%, 80%) or more sequence identity to an equal length (to the first β-glucosidase sequence) contiguous sequence of any one of SEQ ID NOs:54, 56, 58, 62, 64, 66, 68, 70, 72, 74, 76, 78, and 79, or comprises one or more or all of polypeptide sequence motifs SEQ ID NOs: 164-169, and wherein the second β-glucosidase sequence is at least about 50 amino acid residues in length and comprises at least 60% (e.g., at least about 65%, 70%, 75%, 80%) sequence identity to an equal length (to the second β-glucosidase sequence) contiguous sequence of Fv3C (SEQ ID NO:60). In some aspects, the first β-glucosidase sequence is at the N-terminal of the chimeric polypeptide whereas the second β-glucosidase sequence is at the C-terminal of the chimeric polypeptide. In some aspects, the cellulase composition is a fermentation broth. In some aspects, the fermentation broth comprises whole cellulase. In some aspects, the fermentation broth is a cell-free fermentation broth.

In certain embodiments, the first β-glucosidase sequence and the second β-glucosidase sequence are directly adjacent or connected. In some embodiments, the first β-glucosidase sequence and the second β-glucosidase sequence are not directly adjacent but are connected via a linker domain. In certain embodiments, the linker domain is centrally located (i.e., not at either the N-terminal end or the C-terminal end) in the hybrid or chimeric β-glucosidase polypeptide. In certain embodiments, either the first β-glucosidase sequence or the second β-glucosidase sequence, or both of these sequences comprises one or more glycosylation sites. In certain embodiments, either the first β-glucosidase sequence or the second β-glucosidase sequence comprises a loop sequence, which is, e.g., about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the loop sequence provides the linker sequence linking the first and the second β-glucosidase sequences. In some aspects, the cellulase composition is a whole cell composition. In some aspects, the cellulase composition is a fermentation broth. In some aspects, the fermentation broth comprises whole cellulase. In some aspects, the fermentation broth is a cell-free fermentation broth.

In some aspects, the cellulase composition is a a non-naturally occurring cellulase composition, which comprises a chimera or a hybrid of two or more β-glucosidase sequences, wherein the first β-glucosidase sequence is at least about 200 amino acid residues in length and comprises about 60% (e.g., about 65%, 70%, 75%, 80%) or more sequence identity to an equal length (to the first β-glucosidase sequence) contiguous sequence of Fv3C (SEQ ID NO:60), and wherein the second β-glucosidase sequence is at least about 50 amino acid residues in length and comprises at least 60% (e.g., at least about 65%, 70%, 75%, 80%) sequence identity to an equal length (to the second β-glucosidase sequence) contiguous sequence of any one of SEQ ID NOs:54, 56, 58, 62, 64, 66, 68, 70, 72, 74, 76, 78, and 79, or comprises a polypeptide sequence motif SEQ ID NO:170. In some aspects, the first β-glucosidase sequence is at the N-terminal of the chimeric polypeptide whereas the second β-glucosidase sequence is at the C-terminal of the chimeric polypeptide. In certain embodiments, the first β-glucosidase sequence and the second β-glucosidase sequence are directly adjacent or connected. In some embodiments, the first β-glucosidase sequence and the second β-glucosidase sequence are not directly adjacent but are connected via a linker domain. In certain embodiments, the linker domain is centrally located (i.e., not at either the N-terminal end or the C-terminal end) in the hybrid or chimeric β-glucosidase polypeptide. In certain embodiments, either the first β-glucosidase sequence or the second β-glucosidase sequence, or both of these sequences comprises one or more glycosylation sites. In certain embodiments, either the first β-glucosidase sequence or the second β-glucosidase sequence comprises a loop sequence, which is, e.g., about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the loop sequence provides the linker sequence linking the first and the second β-glucosidase sequences. In some aspects, the cellulase composition is a whole cell composition. In some aspects, the cellulase composition is a fermentation broth. In some aspects, the fermentation broth comprises whole cellulase.

In some aspects, the fermentation broth is a cell-free fermentation broth. In some aspects, the cellulase composition is a a non-naturally occurring cellulase composition, which comprises a chimera or a hybrid of two or more β-glucosidase sequences, wherein the first β-glucosidase sequence is one of at least about 200 (e.g., at least about 250, 300, 350, 400, or 450) contiguous amino acid residues in length, comprising one or more or all of the amino acid sequence motifs of SEQ ID NOs:136-148; whereas the second β-glucosidase sequence is one of at least about 50 (e.g., at least about 50, 75, 100, 120, 150, 180, 200, 220, or 250) contiguous amino acid residues in length, comprising one or more or all of the amino acid sequence motifs of SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In some aspects, the first β-glucosidase sequence is at the N-terminal of the chimeric polypeptide whereas the second β-glucosidase sequence is at the C-terminal of the chimeric polypeptide. In certain embodiments, the first β-glucosidase sequence and the second β-glucosidase sequence are directly adjacent or connected. In some embodiments, the first β-glucosidase sequence and the second β-glucosidase sequence are not directly adjacent but are connected via a linker domain. In certain embodiments, the linker domain is centrally located (i.e., not at either the N-terminal end or the C-terminal end) in the hybrid or chimeric β-glucosidase polypeptide. In certain embodiments, either the first β-glucosidase sequence or the second β-glucosidase sequence, or both of these sequences comprises one or more glycosylation sites. In certain embodiments, either the first β-glucosidase sequence or the second β-glucosidase sequence comprises a loop sequence, which is, e.g., about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the loop sequence provides the linker sequence linking the first and the second β-glucosidase sequences. In some aspects, the cellulase composition is a whole cell composition. In some aspects, the cellulase composition is a fermentation broth. In some aspects, the fermentation broth comprises whole cellulase. In some aspects, the fermentation broth is a cell-free fermentation broth

Hemicellulase Compositions

In some aspects, any of the cellulase compositions of the present invention further comprise one or more hemicellulases. In that case, then, the cellulase compositions are also hemicellulase compositions. In some aspects, the hemicellulase composition of the invention comprises hemicellulases selected from xylanases, β-xylosidases, L-α-arabinofuranosidases, and combinations thereof. In some aspects, the hemicellulase composition of the invention comprises at least one xylanase. In some aspects, the at least one xylanase is selected from the group consisting of T. reesei Xyn2, a T. reesei Xyn3, an AfuXyn2, and an AfuXyn5. In some aspects, the hemicellulase composition of the invention comprises at least one β-xylosidase. In some aspects, the β-xylosidase comprises a group 1 β-xylosidase, selected from β-xylosidases such as, e.g., Fv3A and Fv43A. In some aspects, the β-xylosidase comprises a group 2 β-xylosidase, selected from β-xylosidases such as, e.g., Pf43A, Fv43D, Fv39A, Fv43E, Fo43E, Fv43B, Pa51A, Gz43A, and T. reesei Bxl1. In some aspects, the cellulase composition of the invention comprises a single β-xylosidase, selected from a β-xylosidase of either group 1 or group 2. In some aspects, the cellulase composition of the invention comprises two β-xylosidases, wherein one β-xylosidase is selected from group 1 and the other one selected from group 2. In some aspects, the hemicellulase composition of the invention comprises at least one L-α-arabinofuranosidases. In some aspects, the at least one L-α-arabinofuranosidases is selected from the group consisting of Af43A, Fv43B, Pf51A, Pa51A, and Fv51A.

Xylanases:

In some aspects, the cellulase compositions are hemicellulase compositions, comprising at least one suitable xylanase. In some aspects, the at least one xylanase is selected from the group consisting of T. reesei Xyn2, T. reesei Xyn3, AfuXyn2, and AfuXyn5.

Any xylanase (EC 3.2.1.8) can be used as the one or more xylanases. Suitable xylanases include, e.g., a Caldocellum saccharolyticum xylanase (Luthi et al. 1990, Appl. Environ. Microbiol. 56(9):2677-2683), a Thermatoga maritima xylanase (Winterhalter & Liebel, 1995, Appl. Environ. Microbiol. 61(5):1810-1815), a Thermatoga Sp. Strain FJSS-B.1 xylanase (Simpson et al. 1991, Biochem. J. 277, 413-417), a Bacillus circulans xylanase (BcX) (U.S. Pat. No. 5,405,769), an Aspergillus niger xylanase (Kinoshita et al. 1995, Journal of Fermentation and Bioengineering 79(5):422-428), a Streptomyces lividans xylanase (Shareck et al. 1991, Gene 107:75-82; Morosoli et al. 1986 Biochem. J. 239:587-592; Kluepfel et al. 1990, Biochem. J. 287:45-50), a Bacillus subtilis xylanase (Bernier et al. 1983, Gene 26(1):59-65), a Cellulomonas fimi xylanase (Clarke et al., 1996, FEMS Microbiology Letters 139:27-35), a Pseudomonas fluorescens xylanase (Gilbert et al. 1988, Journal of General Microbiology 134:3239-3247), a Clostridium thermocellum xylanase (Dominguez et al., 1995, Nature Structural Biology 2:569-576), a Bacillus pumilus xylanase (Nuyens et al. Applied Microbiology and Biotechnology 2001, 56:431-434; Yang et al. 1998, Nucleic Acids Res. 16(14B):7187), a Clostridium acetobutylicum P262 xylanase (Zappe et al. 1990, Nucleic Acids Res. 18(8):2179), or a Trichoderma harzianum xylanase (Rose et al. 1987, J. Mol. Biol. 194(4):755-756).

Xyn2:

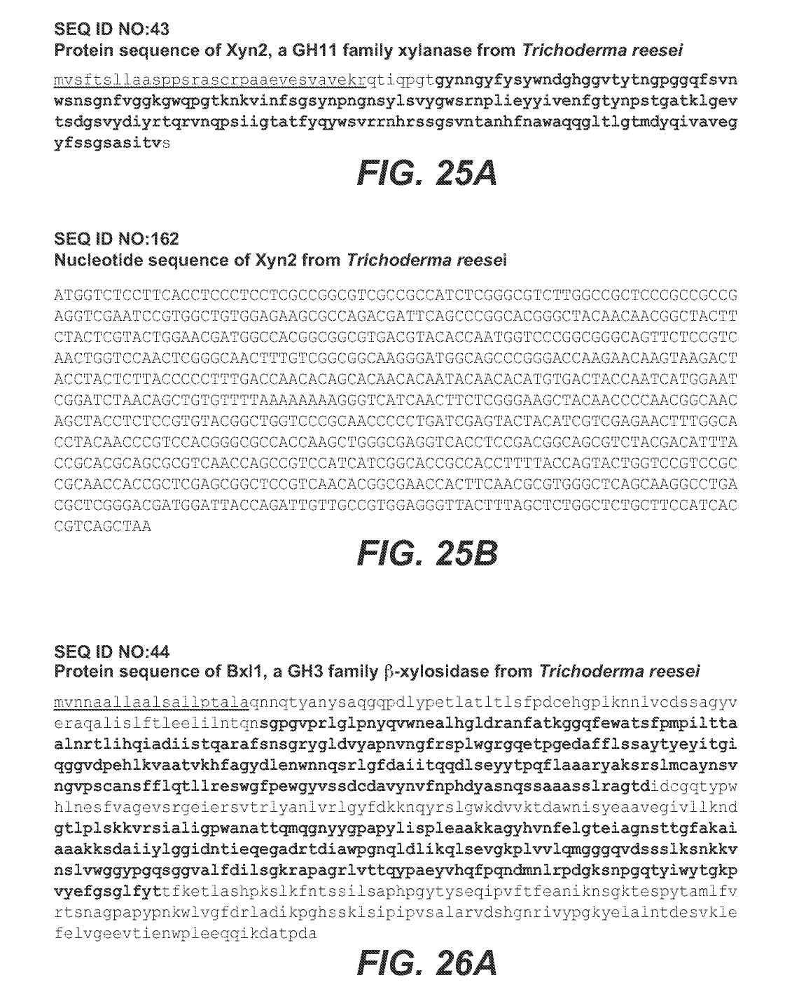

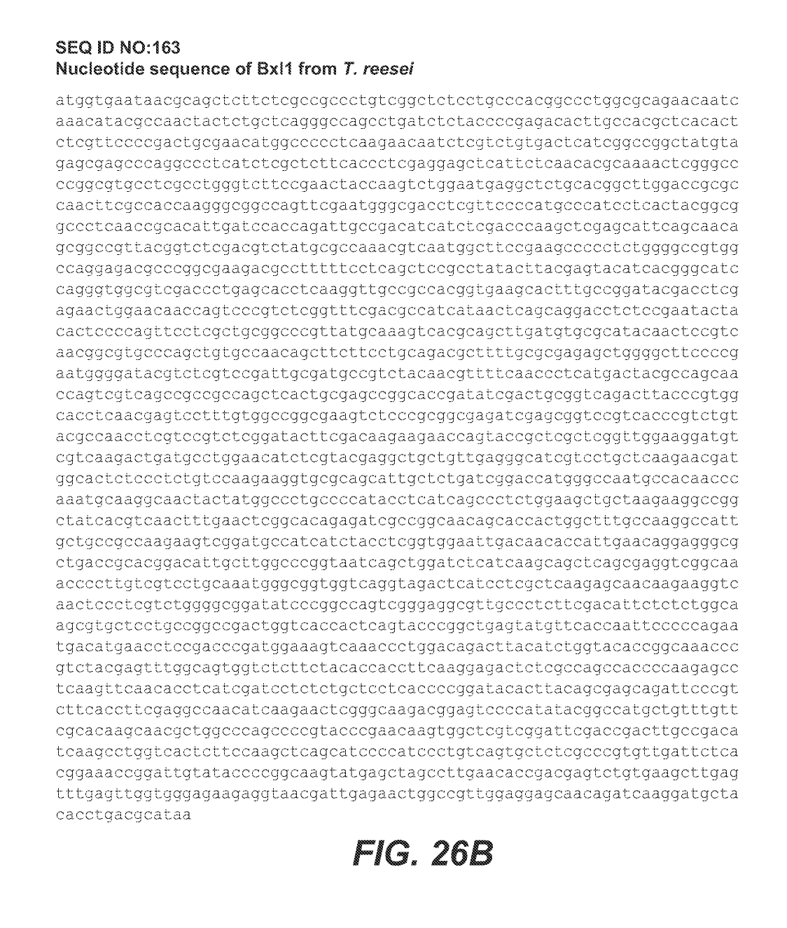

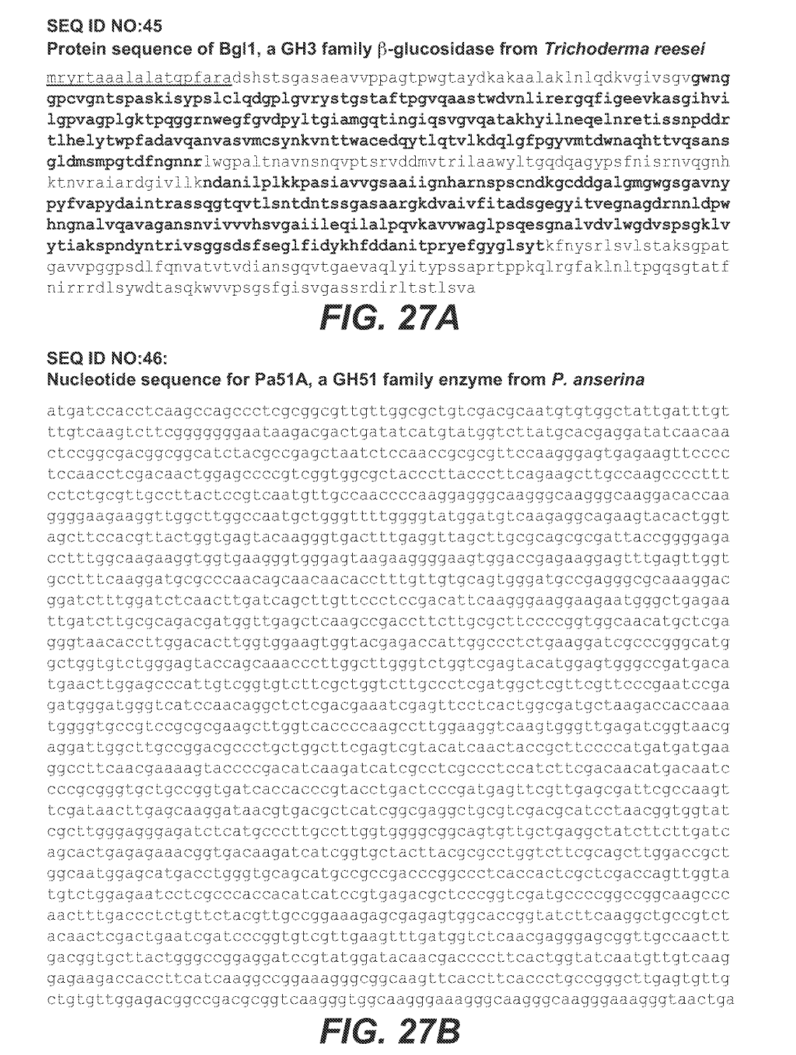

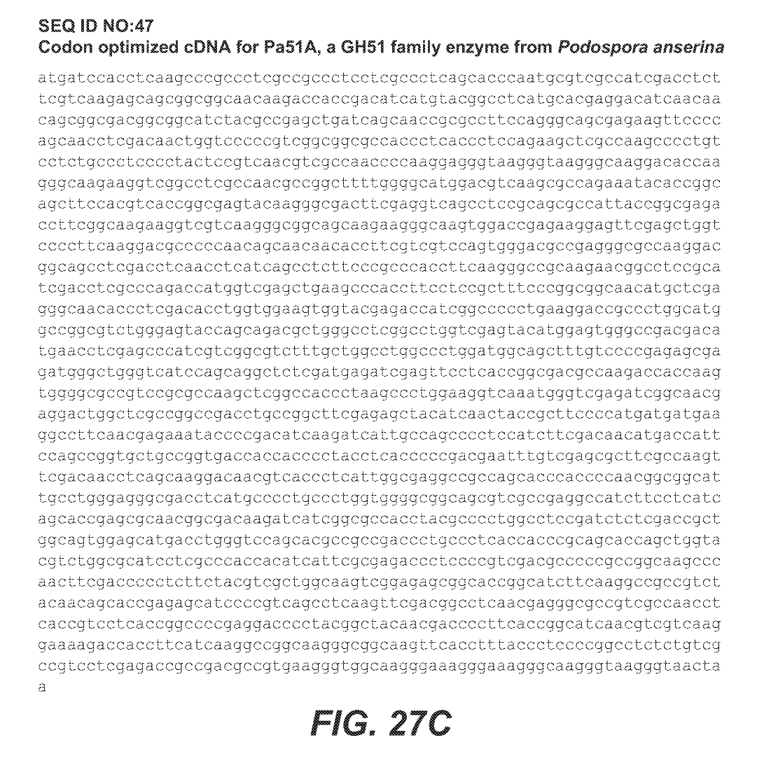

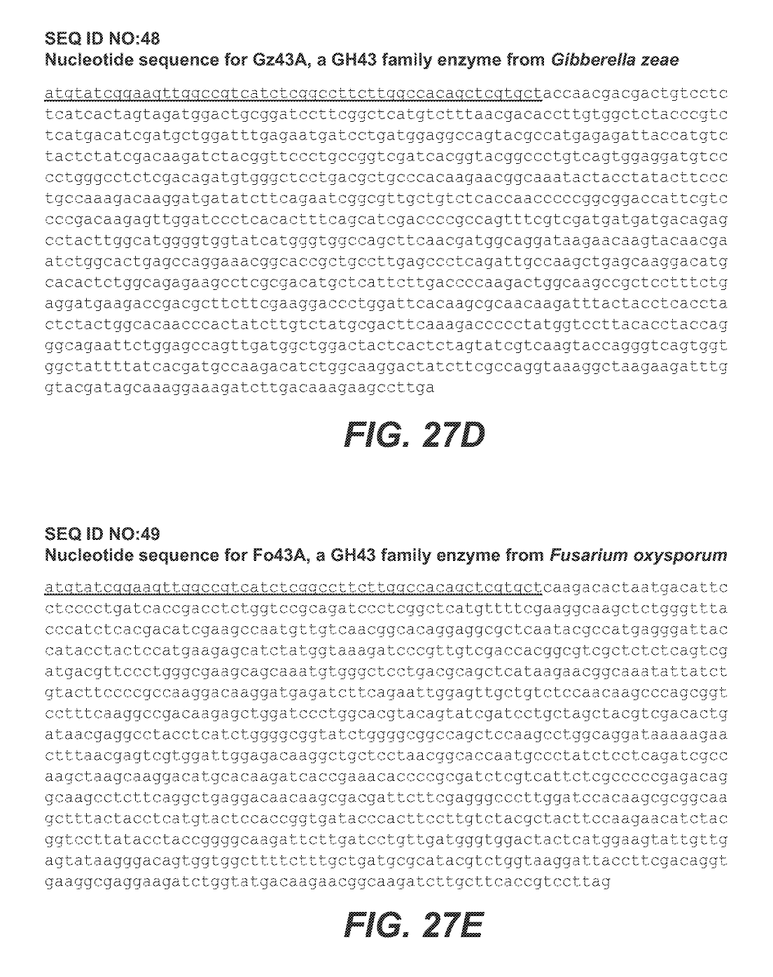

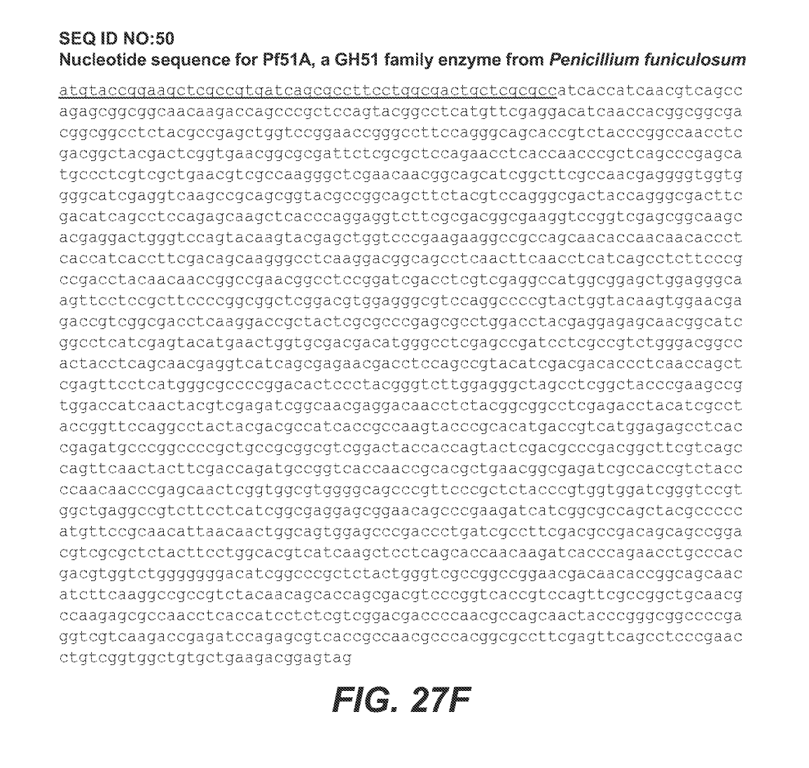

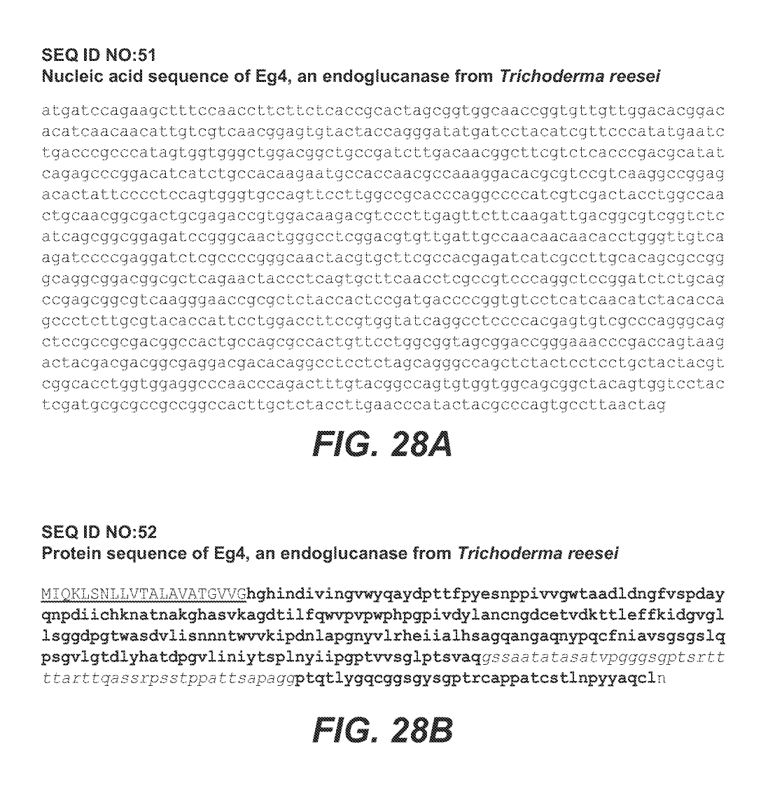

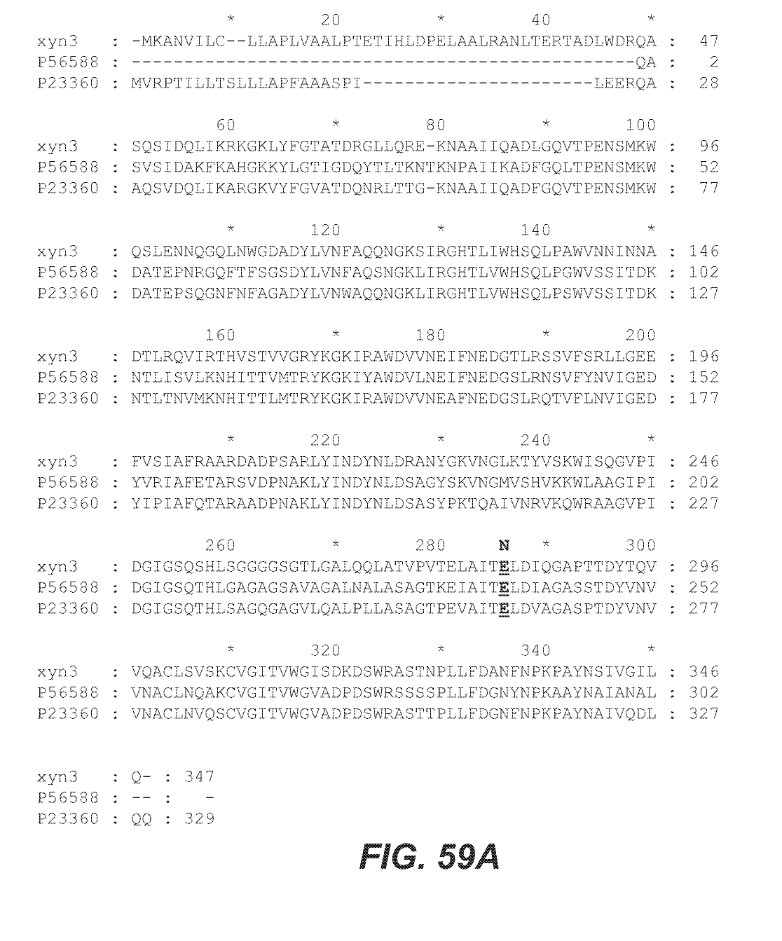

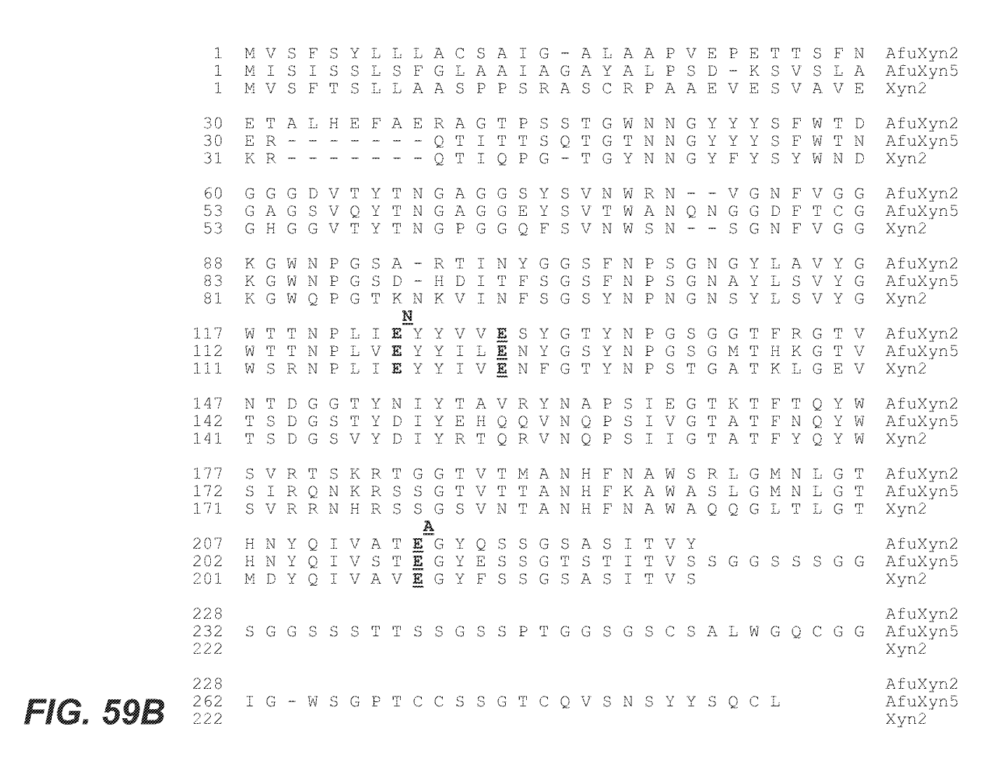

In some aspects, the cellulase compositions of the present invention further comprise Xyn2. The amino acid sequence of T. reesei Xyn2 (SEQ ID NO:43) is shown in FIGS. 25 and 59B. SEQ ID NO:43 is the sequence of the immature T. reesei Xyn2. T. reesei Xyn2 has a predicted prepropeptide sequence corresponding to residues 1 to 33 of SEQ ID NO:43 (underlined in FIG. 25); cleavage of the predicted signal sequence between positions 16 and 17 is predicted to yield a propeptide, which is processed by a kexin-like protease between positions 32 and 33, generating the mature protein having a sequence corresponding to residues 33 to 222 of SEQ ID NO:43. The predicted conserved domain is in boldface type in FIG. 25. T. reesei Xyn2 was shown to have endoxylanase activity indirectly by observation of its ability to catalyze an increased xylose monomer production in the presence of xylobiosidase when the enzymes act on pretreated biomass or on isolated hemicellulose. The conserved acidic residues include E118, E123, and E209. As used herein, “a T. reesei Xyn2 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, or 175 contiguous amino acid residues among residues 33 to 222 of SEQ ID NO:43. A T. reesei Xyn2 polypeptide preferably is unaltered, as compared to a native T. reesei Xyn2, at residues E118, E123, and E209. A T. reesei Xyn2 polypeptide is preferably unaltered in at least 70%, 80%, 90%, 95%, 98%, or 99% of the amino acid residues that are conserved among T. reesei Xyn2, AfuXyn2, and AfuXyn5, as shown in the alignment of FIG. 59B. A T. reesei Xyn2 polypeptide suitably comprises the entire predicted conserved domain of native T. reesei Xyn2 shown in FIG. 25. An exemplary T. reesei Xyn2 polypeptide comprises a sequence having at least 85%, 86%, 87%, 88%, 89%, 90%, 91%, 92%, 93%, 94%, 95%, 96%, 97%, 98%, 99%, or 100% identity to the mature T. reesei Xyn2 sequence shown in FIG. 25. The T. reesei Xyn2 polypeptide of the invention preferably has xylanase activity.

Xyn3:

In some aspects, the cellulase compositions of the present invention further comprise Xyn3. The amino acid sequence of T. reesei Xyn3 (SEQ ID NO:42) is shown in FIG. 24B. SEQ ID NO:42 is the sequence of the immature T. reesei Xyn3. T. reesei Xyn3 has a predicted signal sequence corresponding to residues 1 to 16 of SEQ ID NO:42 (underlined in FIG. 24B); cleavage of the signal sequence is predicted to yield a mature protein having a sequence corresponding to residues 17 to 347 of SEQ ID NO:42. The predicted conserved domain is in boldface type in FIG. 24B. T. reesei Xyn3 was shown to have endoxylanase activity indirectly by observation of its ability to catalyze increased xylose monomer production in the presence of xylobiosidase when the enzymes act on pretreated biomass or on isolated hemicellulose. The conserved catalytic residues include E91, E176, E180, E195, and E282, as determined by alignment with another GH10 family enzyme, the Xys1 delta from Streptomyces halstedii (Canals et al., 2003, Act Crystalogr. D Biol. 59:1447-53), which has 33% sequence identity to T. reesei Xyn3. As used herein, “a T. reesei Xyn3 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues among residues 17 to 347 of SEQ ID NO:42. A T. reesei Xyn3 polypeptide preferably is unaltered, as compared to native T. reesei Xyn3, at residues E91, E176, E180, E195, and E282. A T. reesei Xyn3 polypeptide is preferably unaltered in at least 70%, 80%, 90%, 95%, 98%, or 99% of the amino acid residues that are conserved between T. reesei Xyn3 and Xys1 delta. A T. reesei Xyn3 polypeptide suitably comprises the entire predicted conserved domain of native T. reesei Xyn3 shown in FIG. 24B. An exemplary T. reesei Xyn3 polypeptide comprises a sequence having at least 85%, 86%, 87%, 88%, 89%, 90%, 91%, 92%, 93%, 94%, 95%, 96%, 97%, 98%, 99%, or 100% identity to the mature T. reesei Xyn3 sequence shown in FIG. 24B. The T. reesei Xyn3 polypeptide of the invention preferably has xylanase activity.

AfuXyn2:

In some aspects, the cellulase compositions of the present invention further comprise AfuXyn2. The amino acid sequence of AfuXyn2 (SEQ ID NO:24) is shown in FIGS. 19B and 59B. SEQ ID NO:24 is the sequence of the immature AfuXyn2. AfuXyn2 has a predicted signal sequence corresponding to residues 1 to 18 of SEQ ID NO:24 (underlined in FIG. 19B); cleavage of the signal sequence is predicted to yield a mature protein having a sequence corresponding to residues 19 to 228 of SEQ ID NO:24. The predicted GH11 conserved domain is in boldface type in FIG. 19B. AfuXyn2 was shown to have endoxylanase activity indirectly by observing its ability to catalyze the increased xylose monomer production in the presence of xylobiosidase when the enzymes act on pretreated biomass or on isolated hemicellulose. The conserved catalytic residues include E124, E129, and E215. As used herein, “an AfuXyn2 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, or 200 contiguous amino acid residues among residues 19 to 228 of SEQ ID NO:24. An AfuXyn2 polypeptide preferably is unaltered, as compared to native AfuXyn2, at residues E124, E129 and E215. An AfuXyn2 polypeptide is preferably unaltered in at least 70%, 80%, 90%, 95%, 98%, or 99% of the amino acid residues that are conserved among AfuXyn2, AfuXyn5, and T. reesei Xyn2, as shown in the alignment of FIG. 59B. An AfuXyn2 polypeptide suitably comprises the entire predicted conserved domain of native AfuXyn2 shown in FIG. 19B. An exemplary AfuXyn2 polypeptide comprises a sequence having at least 85%, 86%, 87%, 88%, 89%, 90%, 91%, 92%, 93%, 94%, 95%, 96%, 97%, 98%, 99%, or 100% identity to the mature AfuXyn2 sequence shown in FIG. 19B. The AfuXyn2 polypeptide of the invention preferably has xylanase activity.

AfuXyn5:

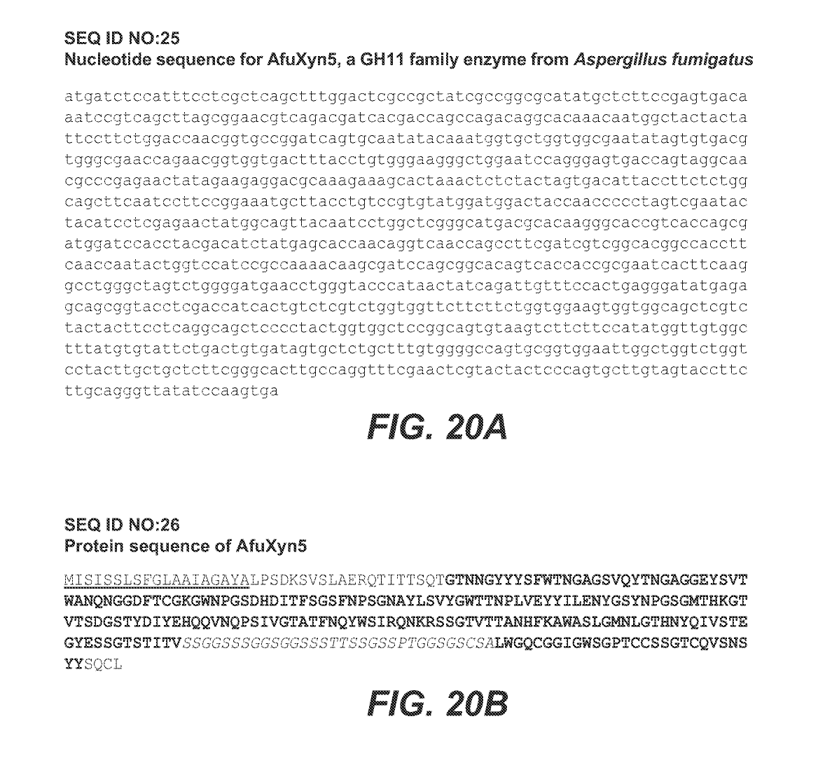

In some aspects, the cellulase compositions of the present invention further comprise AfuXyn5. The amino acid sequence of AfuXyn5 (SEQ ID NO:26) is shown in FIGS. 20B and 59B. SEQ ID NO:26 is the sequence of the immature AfuXyn5. AfuXyn5 has a predicted signal sequence corresponding to residues 1 to 19 of SEQ ID NO:26 (underlined in FIG. 20B); cleavage of the signal sequence is predicted to yield a mature protein having a sequence corresponding to residues 20 to 313 of SEQ ID NO:26. The predicted GH11 conserved domains are in boldface type in FIG. 20B. AfuXyn5 was shown to have endoxylanase activity indirectly by observing its ability to catalyze increased xylose monomer production in the presence of xylobiosidase when the enzymes act on pretreated biomass or on isolated hemicellulose. The conserved catalytic residues include E119, E124, and E210. The predicted CBM is near the C-terminal end, characterized by numerous hydrophobic residues and follows the long serine-, threonine-rich series of amino acids. The region is shown underlined in FIG. 59B. As used herein, “an AfuXyn5 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 275 contiguous amino acid residues among residues 20 to 313 of SEQ ID NO:26. An AfuXyn5 polypeptide preferably is unaltered, as compared to native AfuXyn5, at residues E119, E120, and E210. An AfuXyn5 polypeptide is preferably unaltered in at least 70%, 80%, 90%, 95%, 98%, or 99% of the amino acid residues that are conserved among AfuXyn5, AfuXyn2, and T. reesei Xyn2, as shown in the alignment of FIG. 59B. An AfuXyn5 polypeptide suitably comprises the entire predicted CBM of native AfuXyn5 and/or the entire predicted conserved domain of native AfuXyn5 (underlined) shown in FIG. 20B. An exemplary AfuXyn5 polypeptide comprises a sequence having at least 85%, 86%, 87%, 88%, 89%, 90%, 91%, 92%, 93%, 94%, 95%, 96%, 97%, 98%, 99%, or 100% identity to the mature AfuXyn5 sequence shown in FIG. 20B. The AfuXyn5 polypeptide of the invention preferably has xylanase activity.

The xylanase(s) suitably constitutes about 0.05 wt. % to about 50 wt. % of the cellulase compositions of the disclosure, wherein the wt. % represents the combined weight of xylanase(s) relative to the combined weight of all enzymes in a given composition. The xylanase(s) can be present in a range wherein the lower limit is 0.05 wt. %, 1 wt. %, 1.5 wt. %, 2 wt. %, 3 wt. %, 4 wt. %, 5 wt. %, 6 wt. %, 7 wt. %, 8 wt. %, 9 wt. %, 10 wt. %, 12 wt. %, 15 wt. %, 20 wt. %, 25 wt. %, 30 wt. %, 40 wt. %, or 45 wt. %, and the upper limit is 5 wt. %, 10 wt. %,15 wt. %, 20 wt. %, 25 wt. %, 30 wt. %, 35 wt. %, 40 wt. %, or 50 wt. %. Suitably, the combined weight of one or more xylanases in an enzyme composition of the invention can constitute, e.g., about 0.05 wt. % to about 50 wt. % (e.g., 0.05 wt. %, 1 wt. %, 2 wt. %, 3 wt. % to 50 wt. %, 3 wt. % to 40 wt. %, 3 wt. % to 30 wt. %, 3 wt. % to 20 wt. %, 5 wt. % to 20 wt. %, 10 wt. % to 30 wt. %, 15 wt. % to 35 wt. %, 20 wt. % to 40 wt. %, 20 wt. % to 50 wt. %, etc) of the total weight of all enzymes in the enzyme composition.

The xylanase can be produced by expressing an endogenous or exogenous gene encoding a xylanase. The xylanase can be, in some circumstances, overexpressed or underexpressed.

β-xylosidases:

In some aspects, the cellulase composition of the present invention comprises at least one β-xylosidase. In some aspects, the cellulase composition comprises at least one group 1 β-xylosidase, selected from the group consisting of, e.g., Fv3A and Fv43A. In some aspects, the cellulase composition comprises at least one group 2 β-xylosidase, selected from the group consisting of, e.g., Pf43A, Fv43D, Fv39A, Fv43E, Fo43E, Fv43B, Pa51A, Gz43A, and T. reesei Bxl1. In some aspects, the cellulase composition comprises a single β-xylosidase, and that β-xylosidase is selected from one of either group 1 or group 2. In some aspects, the cellulase composition comprises two β-xylosidases, wherein one β-xylosidase is selected from group 1 and the other selected from group 2.

Any β-xylosidase (EC 3.2.1.37) can be used as a suitable β-xylosidases. Suitable β-xylosidases include, e.g., a T. emersonii Bxl1 (Reen et al. 2003, Biochem Biophys Res Commun. 305(3):579-85), a G. stearothermophilus β-xylosidases (Shallom et al. 2005, Biochemistry 44:387-397), a S. thermophilum β-xylosidases (Zanoelo et al. 2004, J. Ind. Microbiol. Biotechnol. 31:170-176), a T. lignorum β-xylosidases (Schmidt, 1998, Methods Enzymol. 160:662-671), an A. awamori βxylosidases (Kurakake et al. 2005, Biochim. Biophys. Acta 1726:272-279), an A. versicolor β-xylosidases (Andrade et al. 2004, Process Biochem. 39:1931-1938), a Streptomyces sp. β-xylosidases (Pinphanichakarn et al. 2004, World J. Microbiol. Biotechnol. 20:727-733), a T. maritima β-xylosidases (Xue and Shao, 2004, Biotechnol. Lett. 26:1511-1515), a Trichoderma sp. SY β-xylosidases (Kim et al. 2004, J. Microbiol. Biotechnol. 14:643-645), an A. niger β-xylosidases (Oguntimein and Reilly, 1980, Biotechnol. Bioeng. 22:1143-1154), or a P. wortmanni β-xylosidases (Matsuo et al. 1987, Agric. Biol. Chem. 51:2367-2379). Suitable β-xylosidases can be produced endogenously by the host organism, or can be recombinantly cloned and/or expressed by the host organism. Furthermore, suitable β-xylosidases can be added to a cellulase composition in a purified or isolated form.

Fv3A:

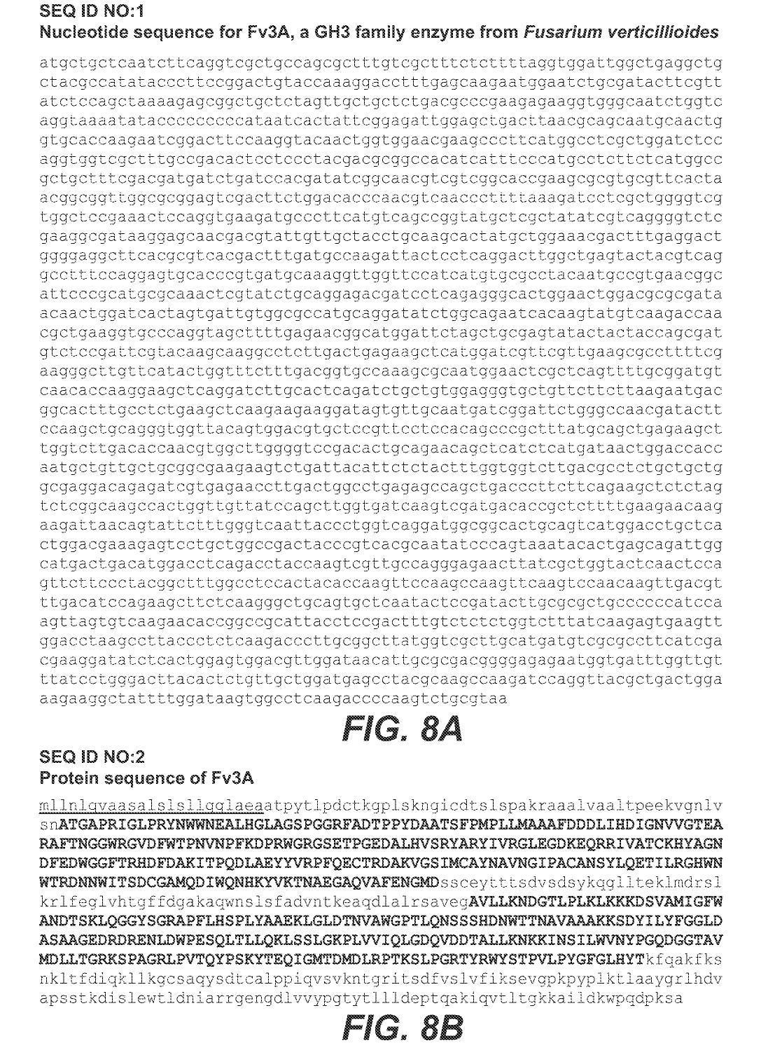

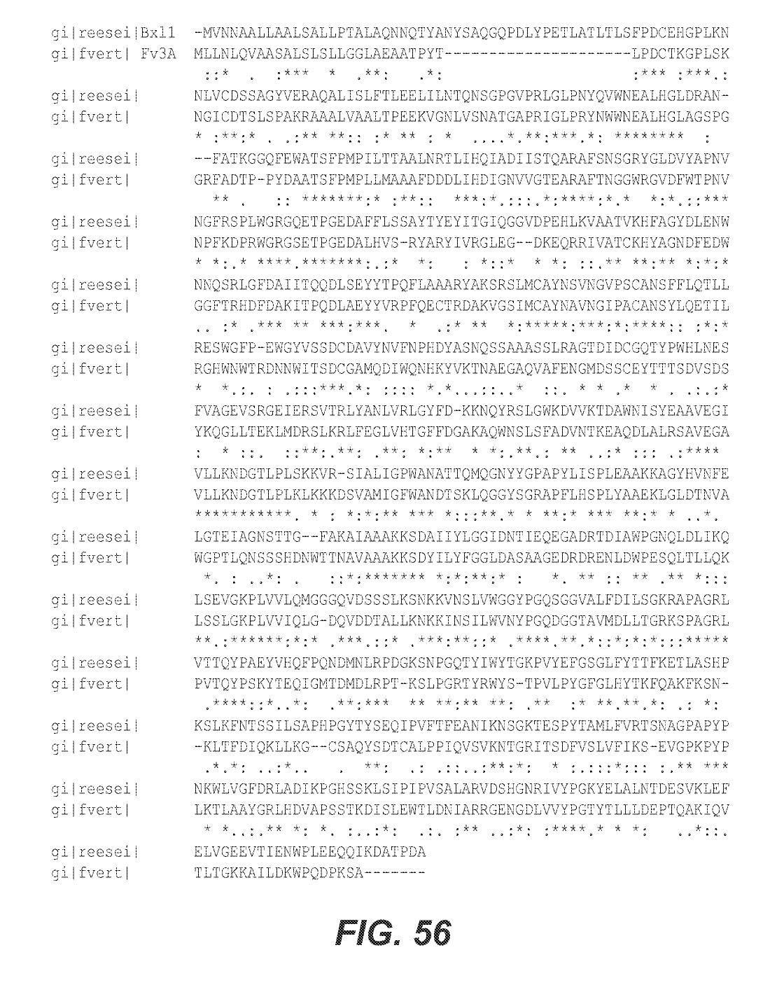

In some aspects, the cellulase composition of the present invention comprises an Fv3A polypeptide. The amino acid sequence of Fv3A (SEQ ID NO:2) is shown in FIGS. 8B and 56. SEQ ID NO:2 is the sequence of the immature Fv3A. Fv3A has a predicted signal sequence corresponding to residues 1 to 23 of SEQ ID NO:2 (underlined); cleavage of the signal sequence is predicted to yield a mature protein having a sequence corresponding to residues 24 to 766 of SEQ ID NO:2. The predicted conserved domains are in boldface type in FIG. 8B. Fv3A was shown to have β-xylosidase activity, e.g., in an enzymatic assay using p-nitophenyl-β-xylopyranoside, xylobiose, mixed linear xylo-oligomers, branched arabinoxylan oligomers from hemicellulose, or dilute ammonia pretreated corncob as substrates. The predicted catalytic residue is D291, while the flanking residues, S290 and C292, are predicted to be involved in substrate binding. E175 and E213 are conserved across other GH3 and GH39 enzymes and are predicted to have catalytic functions. As used herein, “an Fv3A polypeptide” refers to a polypeptide and/or to a variant thereof comprising a sequence having at least 85%, e.g., at least 86%, 87%, 88%, 89%, 90%, 91%, 92%, 93%, 94%, 95%, 96%, 97%, 98%, 99%, or 100% sequence identity to at least 50, e.g., at least 75, 100, 125, 150, 175, 200, 250, 300, 350, 400, 450, 500, 550, 600, 650, or 700 contiguous amino acid residues among residues 24 to 766 of SEQ ID NO:2. An Fv3A polypeptide preferably is unaltered as compared to native Fv3A in residues D291, S290, C292, E175, and E213. An Fv3A polypeptide is preferably unaltered in at least 70%, 75%, 80%, 85%, 90%, 95%, 98%, or 99% of the amino acid residues that are conserved between Fv3A, and Trichoderma reesei Bxl1, as shown in the alignment of FIG. 56. An Fv3A polypeptide suitably comprises the entire predicted conserved domain of native Fv3A as shown in FIG. 8B. An exemplary Fv3A polypeptide of the invention comprises a sequence having at least 85%, 86%, 87%, 88%, 89%, 90%, 91%, 92%, 93%, 94%, 95%, 96%, 97%, 98%, 99%, or 100% identity to the mature Fv3A sequence as shown in FIG. 8B. The Fv3A polypeptide of the invention preferably has β-xylosidase activity.

Accordingly an Fv3A polypeptide of the invention suitably comprises an amino acid sequence with at least 90%, 91%, 92%, 93%, 94%, 95%, 96%, 97%, 98%, 99%, or 100% sequence identity to the amino acid sequence of SEQ ID NO:2, or to residues (i) 24-766, (ii) 73-321, (iii) 73-394, (iv) 395-622, (v) 24-622, or (vi) 73-622 of SEQ ID NO:2. The polypeptide suitably has β-xylosidase activity.

Fv43A:

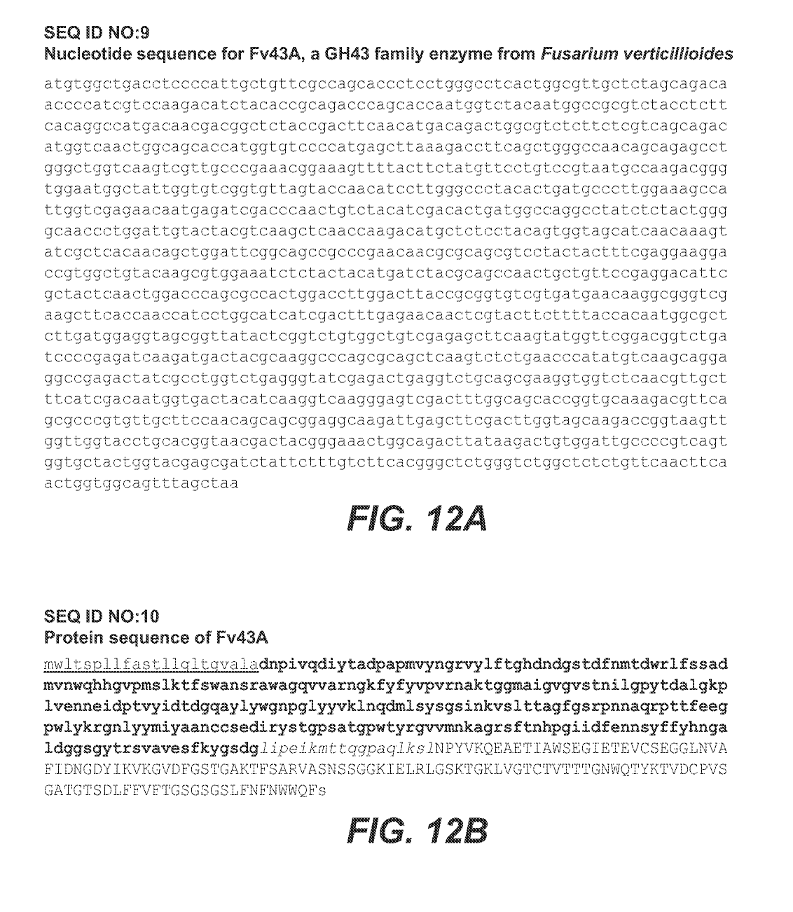

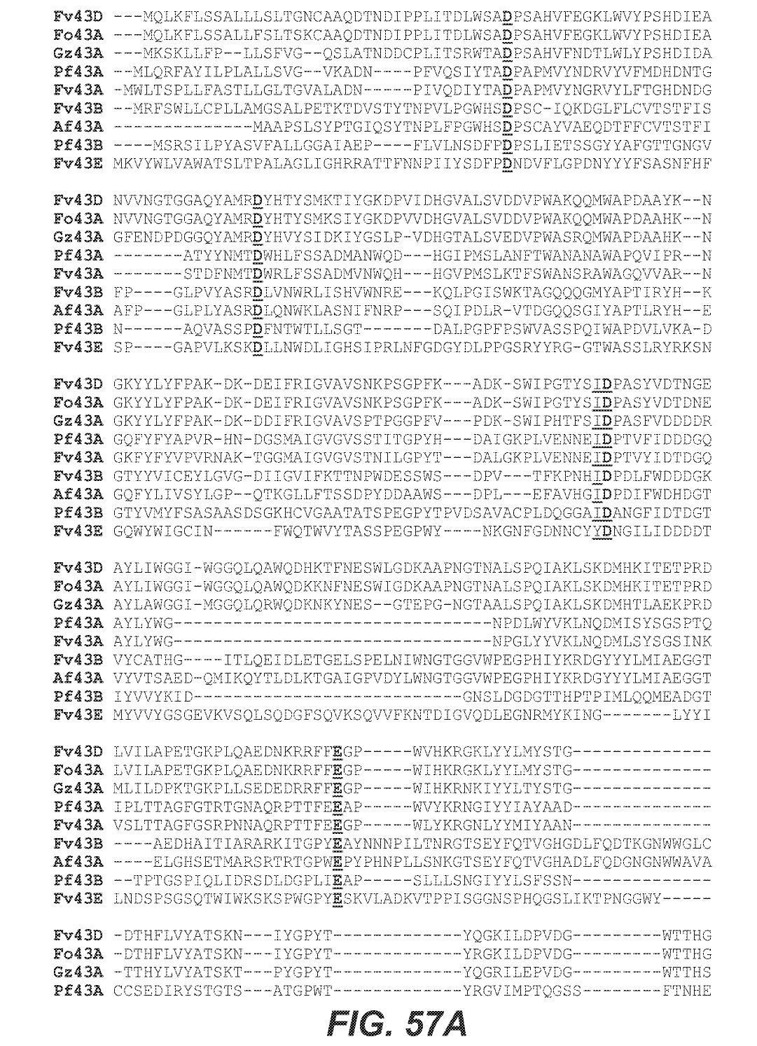

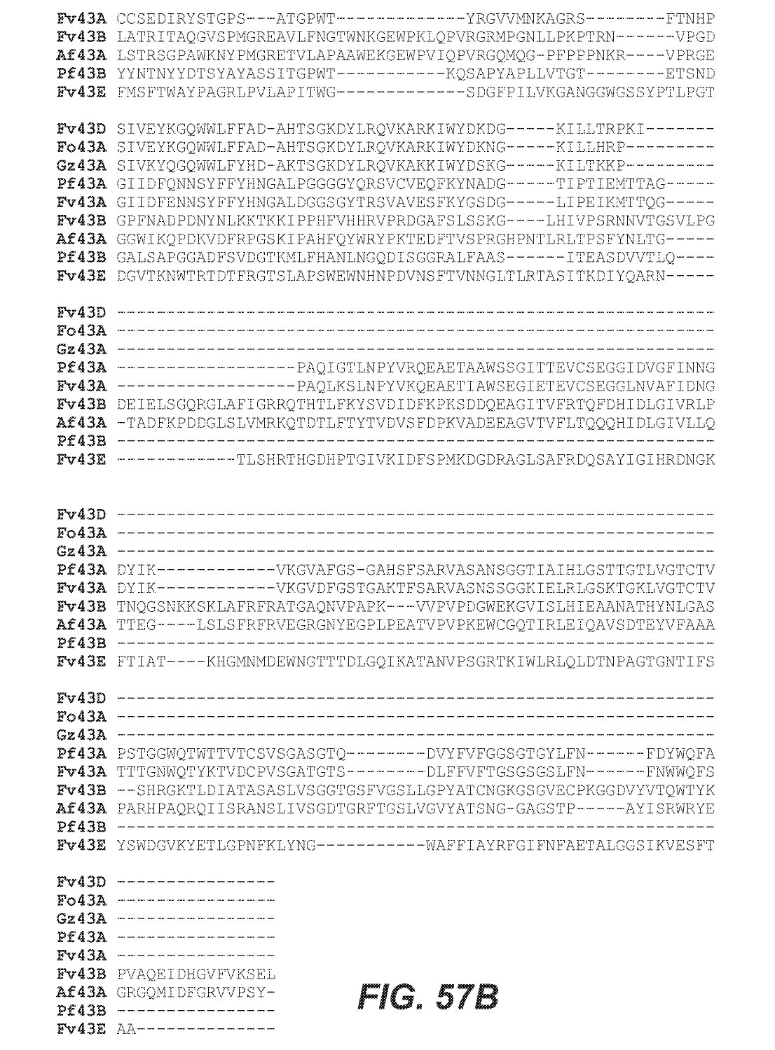

In some aspects, the cellulase composition of the present invention comprises an Fv43A polypeptide. The amino acid sequence of Fv43A (SEQ ID NO:10) is provided in FIGS. 12B and 57. SEQ ID NO:10 is the sequence of the immature Fv43A. Fv43A has a predicted signal sequence corresponding to residues 1 to 22 of SEQ ID NO:10 (underlined in FIG. 12B); cleavage of the signal sequence is predicted to yield a mature protein having a sequence corresponding to residues 23 to 449 of SEQ ID NO:10. In FIG. 12B, the predicted conserved domain is in boldface type, the predicted CBM is in uppercase type, and the predicted linker separating the CD and CBM is in italics. Fv43A was shown to have β-xylosidase activity in, e.g., an enzymatic assay using 4-nitophenyl-β-D-xylopyranoside, xylobiose, mixed, linear xylo-oligomers, branched arabinoxylan oligomers from hemicellulose, and/or linear xylo-oligomers as substrates. The predicted catalytic residues including either D34 or D62, D148, and E209. As used herein, “an Fv43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 23 to 449 of SEQ ID NO:10. An Fv43A polypeptide preferably is unaltered, as compared to native Fv43A, at residues D34 or D62, D148, and E209. An Fv43A polypeptide is preferably unaltered in at least 70%, 80%, 90%, 95%, 98%, or 99% of the amino acid residues that are conserved among a family of enzymes including Fv43A and 1, 2, 3, 4, 5, 6, 7, 8, or all 9 other amino acid sequences in the alignment of FIG. 57. An Fv43A polypeptide suitably comprises the entire predicted CBM of native Fv43A, and/or the entire predicted conserved domain of native Fv43A, and/or the linker of Fv43A as shown in FIG. 12B. An exemplary Fv43A polypeptide comprises a sequence having at least 85%, 86%, 87%, 88%, 89%, 90%, 91%, 92%, 93%, 94%, 95%, 96%, 97%, 98%, 99%, or 100% identity to the mature Fv43A sequence as shown in FIG. 12B. The Fv43A polypeptide of the invention preferably has β-xylosidase activity.

Accordingly an Fv43A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:10, or to residues (i) 23-449, (ii) 23-302, (iii) 23-320, (iv) 23-448, (v) 303-448, (vi) 303-449, (vii) 321-448, or (viii) 321-449 of SEQ ID NO:10. The polypeptide suitably has β-xylosidase activity.

Pf43A:

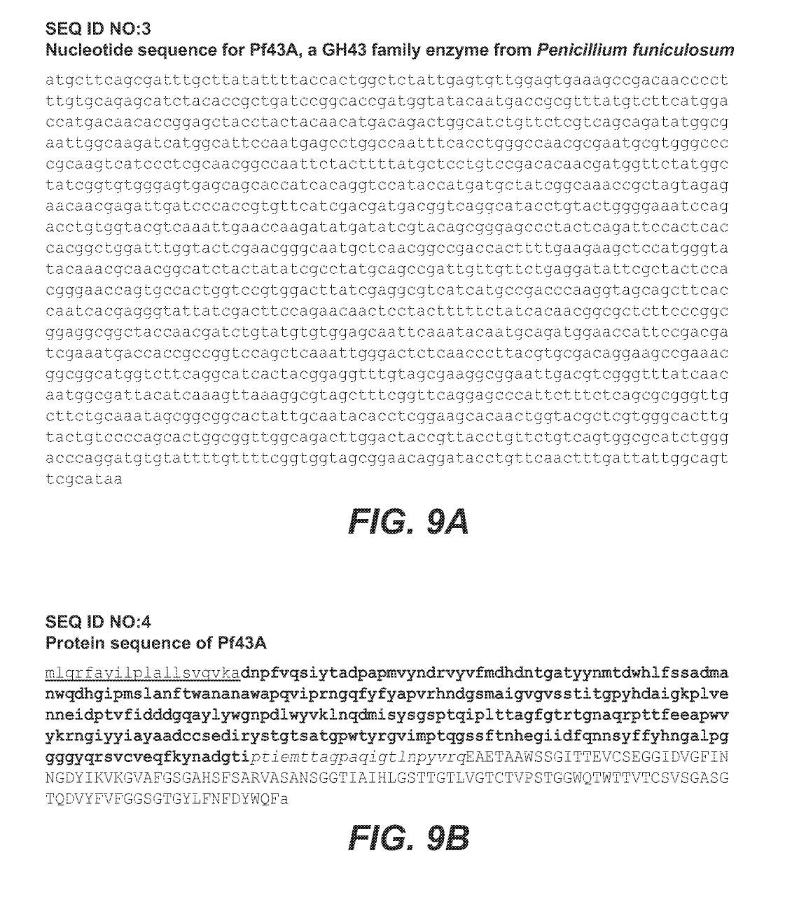

In some aspects, the cellulase composition of the present invention comprises a Pf43A polypeptide. The amino acid sequence of Pf43A (SEQ ID NO:4) is shown in FIGS. 9B and 57. SEQ ID NO:4 is the sequence of the immature Pf43A. Pf43A has a predicted signal sequence corresponding to residues 1 to 20 of SEQ ID NO:4 (underlined in FIG. 9B); cleavage of the signal sequence is predicted to yield a mature protein having a sequence corresponding to residues 21 to 445 of SEQ ID NO:4. The predicted conserved domain is in boldface type, the predicted CBM is in uppercase type, and the predicted linker separating the CD and CBM is in italics in FIG. 9B. Pf43A has been shown to have β-xylosidase activity, in, e.g., an enzymatic assay using p-nitophenyl-β-xylopyranoside, xylobiose, mixed linear xylo-oligomers, or dilute ammonia pretreated corncob as substrates. The predicted catalytic residues include either D32 or D60, D145, and E206. The C-terminal region underlined in FIG. 57 is the predicted CBM. As used herein, “a Pf43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 21 to 445 of SEQ ID NO:4. A Pf43A polypeptide preferably is unaltered as compared to the native Pf43A in residues D32 or D60, D145, and E206. A Pf43A is preferably unaltered in at least 70%, 80%, 90%, 95%, 98%, or 99% of the amino acid residues that are found conserved across a family of proteins including Pf43A and 1, 2, 3, 4, 5, 6, 7, or all 8 of other amino acid sequences in the alignment of FIG. 57. A Pf43A polypeptide of the invention suitably comprises two or more or all of the following domains: (1) the predicted CBM, (2) the predicted conserved domain, and (3) the linker of Pf43A as shown in FIG. 9B. An exemplary Pf43A polypeptide of the invention comprises a sequence having at least 85%, 86%, 87%, 88%, 89%, 90%, 91%, 92%, 93%, 94%, 95%, 96%, 97%, 98%, 99%, or 100% identity to the mature Pf43A sequence as shown in FIG. 9B. The Pf43A polypeptide of the invention preferably has β-xylosidase activity.

Accordingly a Pf43A polypeptide of the invention suitably comprises an amino acid sequence with at least 90%, 91%, 92%, 93%, 94%, 95%, 96%, 97%, 98%, 99%, or 100% sequence identity to the amino acid sequence of SEQ ID NO:4, or to residues (i) 21-445, (ii) 21-301, (iii) 21-323, (iv) 21-444, (v) 302-444, (vi) 302-445, (vii) 324-444, or (viii) 324-445 of SEQ ID NO:4. The polypeptide suitably has β-xylosidase activity.

Fv43D:

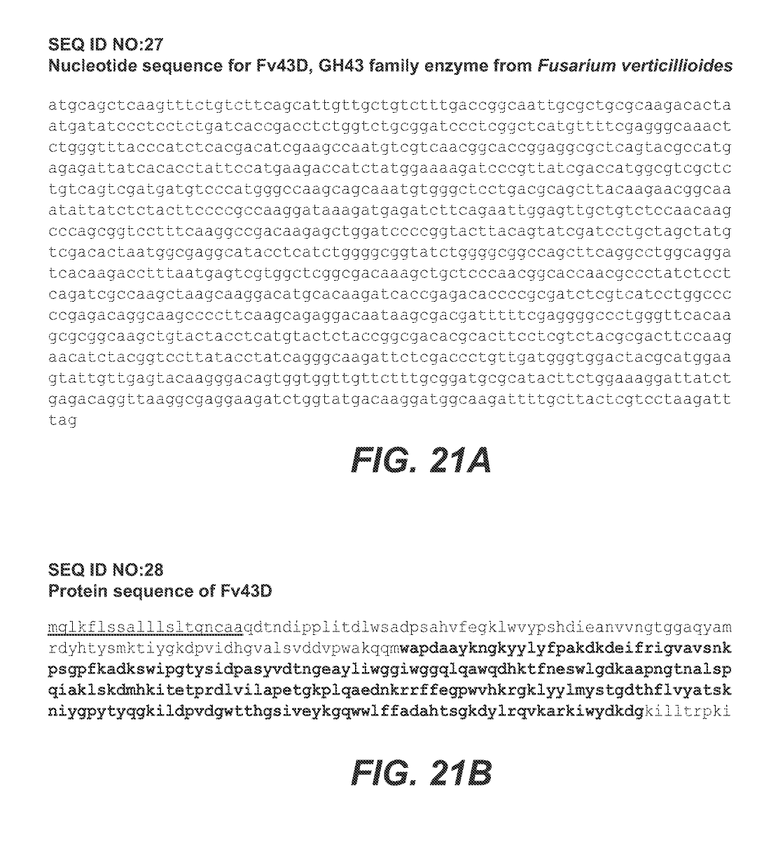

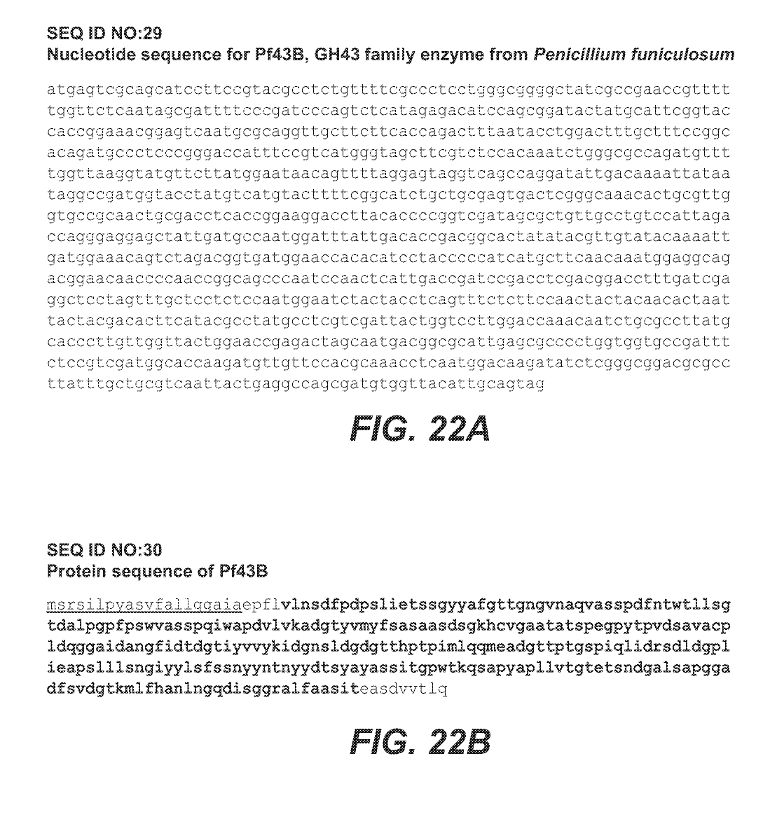

In some aspects, the cellulase composition of the present invention further comprises an Fv43D polypeptide. The amino acid sequence of Fv43D (SEQ ID NO:28) is shown in FIGS. 21B and 57. SEQ ID NO:28 is the sequence of the immature Fv43D. Fv43D has a predicted signal sequence corresponding to residues 1 to 20 of SEQ ID NO:28 (underlined in FIG. 21B); cleavage of the signal sequence is predicted to yield a mature protein having a sequence corresponding to residues 21 to 350 of SEQ ID NO:28. The predicted conserved domain is in boldface type in FIG. 21B. Fv43D was shown to have β-xylosidase activity in, e.g., an enzymatic assay using p-nitophenyl-β-xylopyranoside, xylobiose, and/or mixed, linear xylo-oligomers as substrates. The predicted catalytic residues include either D37 or D72, D159, and E251. As used herein, “an Fv43D polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, or 320 contiguous amino acid residues among residues 21 to 350 of SEQ ID NO:28. An Fv43D polypeptide preferably is unaltered, as compared to native Fv43D, at residues D37 or D72, D159, and E251. An Fv43D polypeptide is preferably unaltered in at least 70%, 80%, 90%, 95%, 98%, or 99% of the amino acid residues that are conserved among a group of enzymes including Fv43D and 1, 2, 3, 4, 5, 6, 7, 8, or all 9 other amino acid sequences in the alignment of FIG. 57. An Fv43D polypeptide suitably comprises the entire predicted CD of native Fv43D shown in FIG. 21B. An exemplary Fv43D polypeptide comprises a sequence having at least 85%, 86%, 87%, 88%, 89%, 90%, 91%, 92%, 93%, 94%, 95%, 96%, 97%, 98%, 99%, or 100% identity to the mature Fv43D sequence shown in FIG. 21B. The Fv43D polypeptide of the invention preferably has β-xylosidase activity.

Accordingly an Fv43D polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:28, or to residues (i) 20-341, (ii) 21-350, (iii) 107-341, or (iv) 107-350 of SEQ ID NO:28. The polypeptide suitably has O-xylosidase activity.

Fv39A:

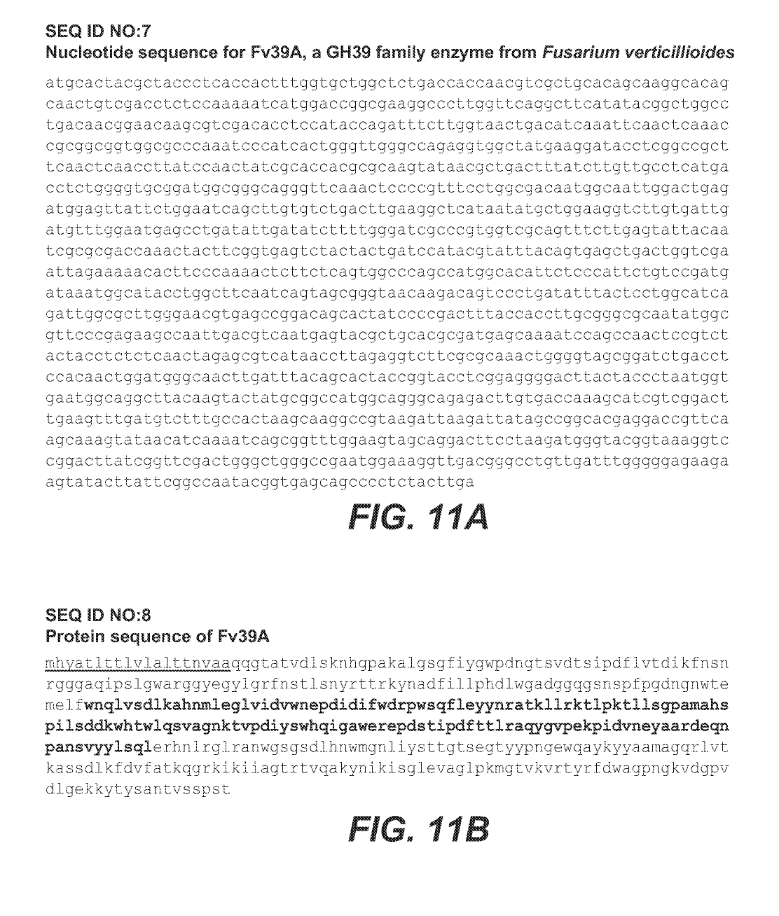

In some aspects, the cellulase composition of the present invention comprises an Fv39A polypeptide. The amino acid sequence of Fv39A (SEQ ID NO:8) is shown in FIG. 11B. SEQ ID NO:8 is the sequence of the immature Fv39A. Fv39A has a predicted signal sequence corresponding to residues 1 to 19 of SEQ ID NO:8 (underlined in FIG. 11B); cleavage of the signal sequence is predicted to yield a mature protein having a sequence corresponding to residues 20 to 439 of SEQ ID NO:8. The predicted conserved domain is shown in boldface type in FIG. 11B. Fv39A was shown to have β-xylosidase activity in, e.g., an enzymatic assay using p-nitophenyl-β-xylopyranoside, xylobiose or mixed, linear xylo-oligomers as substrates. Fv39A residues E168 and E272 are predicted to function as catalytic acid-base and nucleophile, respectively, based on a sequence alignment of the above-mentioned GH39 xylosidases from Thermoanaerobacterium saccharolyticum (Uniprot Accession No. P36906) and Geobacillus stearothermophilus (Uniprot Accession No. Q9ZFM2) with Fv39A. As used herein, “an Fv39A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 20 to 439 of SEQ ID NO:8. An Fv39A polypeptide preferably is unaltered as compared to native Fv39A in residues E168 and E272. An Fv39A polypeptide is preferably unaltered in at least 70%, 80%, 90%, 95%, 98%, or 99% of the amino acid residues that are conserved among a family or enzymes including Fv39A and xylosidases from Thermoanaerobacterium saccharolyticum and Geobacillus stearothermophilus (see above). An Fv39A polypeptide suitably comprises the entire predicted conserved domain of native Fv39A as shown in FIG. 11B. An exemplary Fv39A polypeptide comprises a sequence having at least 85%, 86%, 87%, 88%, 89%, 90%, 91%, 92%, 93%, 94%, 95%, 96%, 97%, 98%, 99%, or 100% identity to the mature Fv39A sequence as shown in FIG. 11B. The Fv39A polypeptide of the invention preferably has β-xylosidase activity.

Accordingly, an Fv39A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:8, or to residues (i) 20-439, (ii) 20-291, (iii) 145-291, or (iv) 145-439 of SEQ ID NO:8. The polypeptide suitably has β-xylosidase activity.

Fv43E:

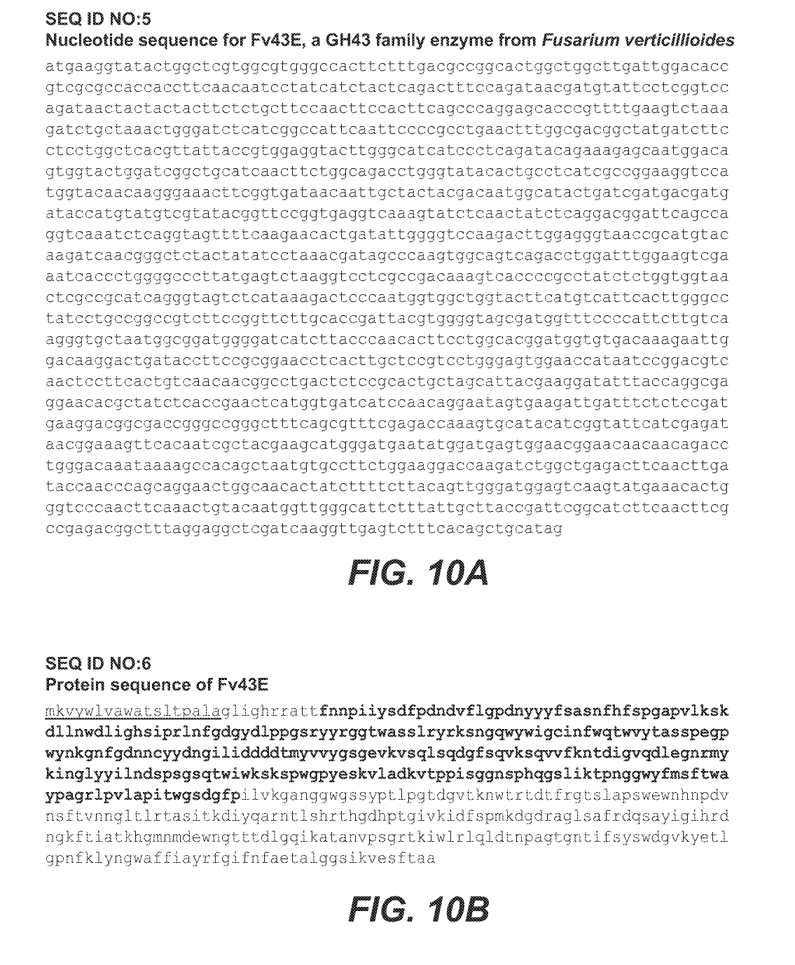

In some aspects, the cellulase composition of the present invention comprises an Fv43E polypeptide. The amino acid sequence of Fv43E (SEQ ID NO:6) is shown in FIGS. 10B and 57. SEQ ID NO:6 is the sequence of the immature Fv43E. Fv43E has a predicted signal sequence corresponding to residues 1 to 18 of SEQ ID NO:6 (underlined in FIG. 10B); cleavage of the signal sequence is predicted to yield a mature protein having a sequence corresponding to residues 19 to 530 of SEQ ID NO:6. The predicted conserved domain is marked in boldface type in FIG. 10B. Fv43E was shown to have β-xylosidase activity, in, e.g., enzymatic assay using 4-nitophenyl-β-D-xylopyranoside, xylobiose, and mixed, linear xylo-oligomers, or dilute ammonia pretreated corncob as substrates. The predicted catalytic residues include either D40 or D71, D155, and E241. As used herein, “an Fv43E polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, or 500 contiguous amino acid residues among residues 19 to 530 of SEQ ID NO:6. An Fv43E polypeptide preferably is unaltered as compared to the native Fv43E in residues D40 or D71, D155, and E241. An Fv43E polypeptide is preferably unaltered in at least 70%, 80%, 90%, 95%, 98%, or 99% of the amino acid residues that are found to be conserved among a family of enzymes including Fv43E, and 1, 2, 3, 4, 5, 6, 7, or all other 8 amino acid sequences in the alignment of FIG. 57. An Fv43E polypeptide suitably comprises the entire predicted conserved domain of native Fv43E as shown in FIG. 10B. An exemplary Fv43E polypeptide comprises a sequence having at least 85%, 86%, 87%, 88%, 89%, 90%, 91%, 92%, 93%, 94%, 95%, 96%, 97%, 98%, 99%, or 100% identity to mature Fv43E sequence as shown in FIG. 10B. The Fv43E polypeptide of the invention preferably has β-xylosidase activity.

Accordingly, an Fv43E polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:6, or to residues (i) 19-530, (ii) 29-530, (iii) 19-300, or (iv) 29-300 of SEQ ID NO:6. The polypeptide suitably has β-xylosidase activity.

Fv43B:

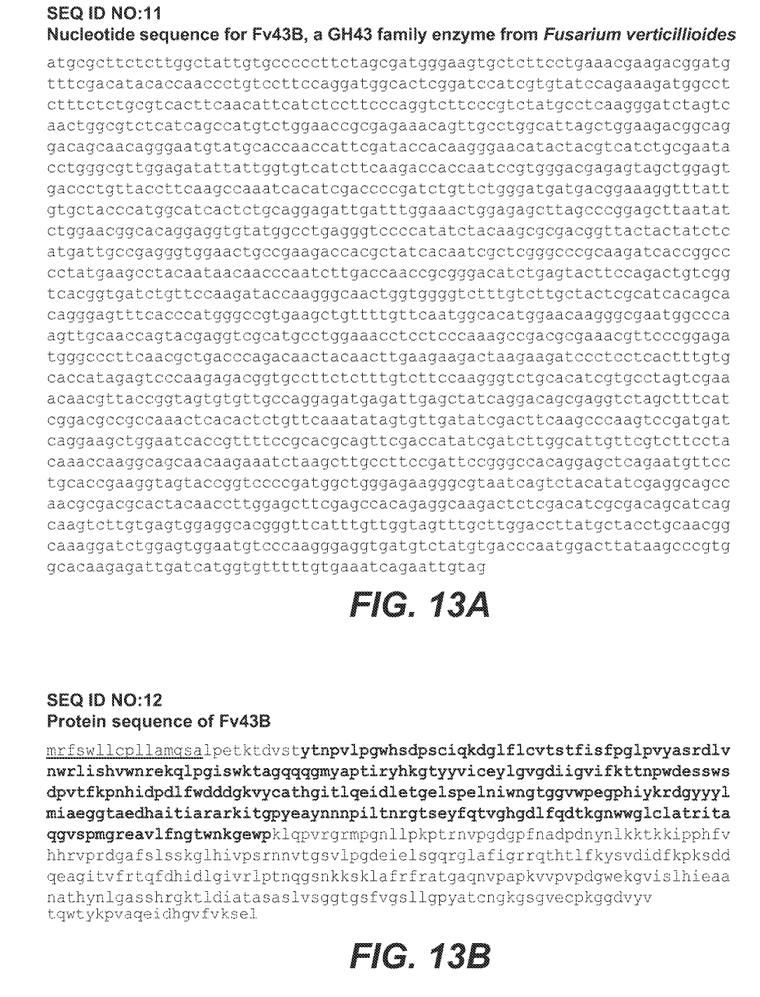

In some aspects, the cellulase composition of the present invention comprises an Fv43B polypeptide. The amino acid sequence of Fv43B (SEQ ID NO:12) is shown in FIGS. 13B and 57. SEQ ID NO:12 is the sequence of the immature Fv43B. Fv43B has a predicted signal sequence corresponding to residues 1 to 16 of SEQ ID NO:12 (underlined in FIG. 13B); cleavage of the signal sequence is predicted to yield a mature protein having a sequence corresponding to residues 17 to 574 of SEQ ID NO:12. The predicted conserved domain is in boldface type in FIG. 13B. Fv43B was shown to have both β-xylosidase and L-α-arabinofuranosidase activities, in, e.g., a first enzymatic assay using 4-nitophenyl-β-D-xylopyranoside and p-nitrophenyl-α-L-arabinofuranoside as substrates. It was shown, in a second enzymatic assay, to catalyze the release of arabinose from branched arabino-xylooligomers and to catalyze the increased xylose release from oligomer mixtures in the presence of other xylosidase enzymes. The predicted catalytic residues include either D38 or D68, D151, and E236. As used herein, “an Fv43B polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, or 550 contiguous amino acid residues among residues 17 to 574 of SEQ ID NO:12. An Fv43B polypeptide preferably is unaltered, as compared to native Fv43B, at residues D38 or D68, D151, and E236. An Fv43B polypeptide is preferably unaltered in at least 70%, 80%, 90%, 95%, 98%, or 99% of the amino acid residues that are conserved among a family of enzymes including Fv43B and 1, 2, 3, 4, 5, 6, 7, 8, or all 9 other amino acid sequences in the alignment of FIG. 57. An Fv43B polypeptide suitably comprises the entire predicted conserved domain of native Fv43B as shown in FIGS. 13B and 57. An exemplary Fv43B polypeptide comprises a sequence having at least 85%, 86%, 87%, 88%, 89%, 90%, 91%, 92%, 93%, 94%, 95%, 96%, 97%, 98%, 99%, or 100% identity to the mature Fv43B sequence as shown in FIG. 13B. The Fv43B polypeptide of the present invention preferably has β-xylosidase activity, L-α-arabinofuranosidase activity, or both β-xylosidase and L-α-arabinofuranosidase activities.

Accordingly, an Fv43B polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:12, or to residues (i) 17-574, (ii) 27-574, (iii) 17-303, or (iv) 27-303 of SEQ ID NO:12. The polypeptide suitably has 0-xylosidase activity, L-α-arabinofuranosidase activity, or both β-xylosidase and L-α-arabinofuranosidase activities.

Pa51A:

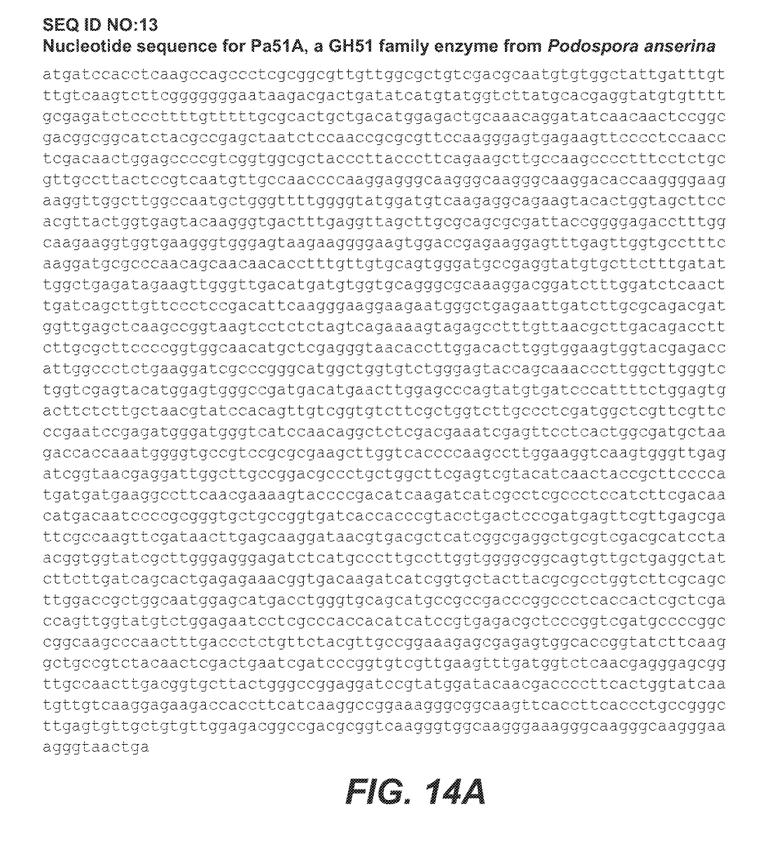

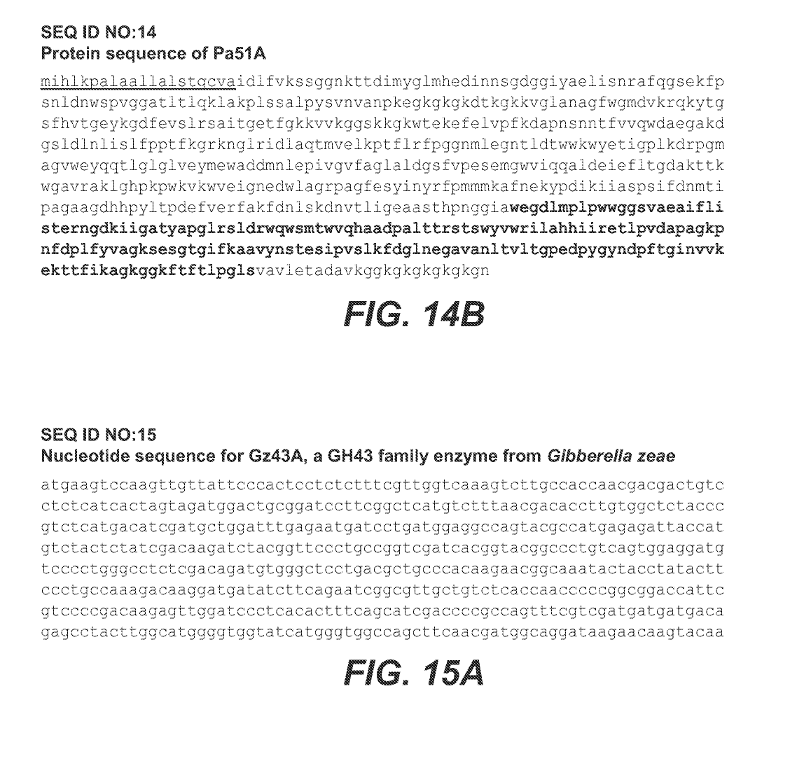

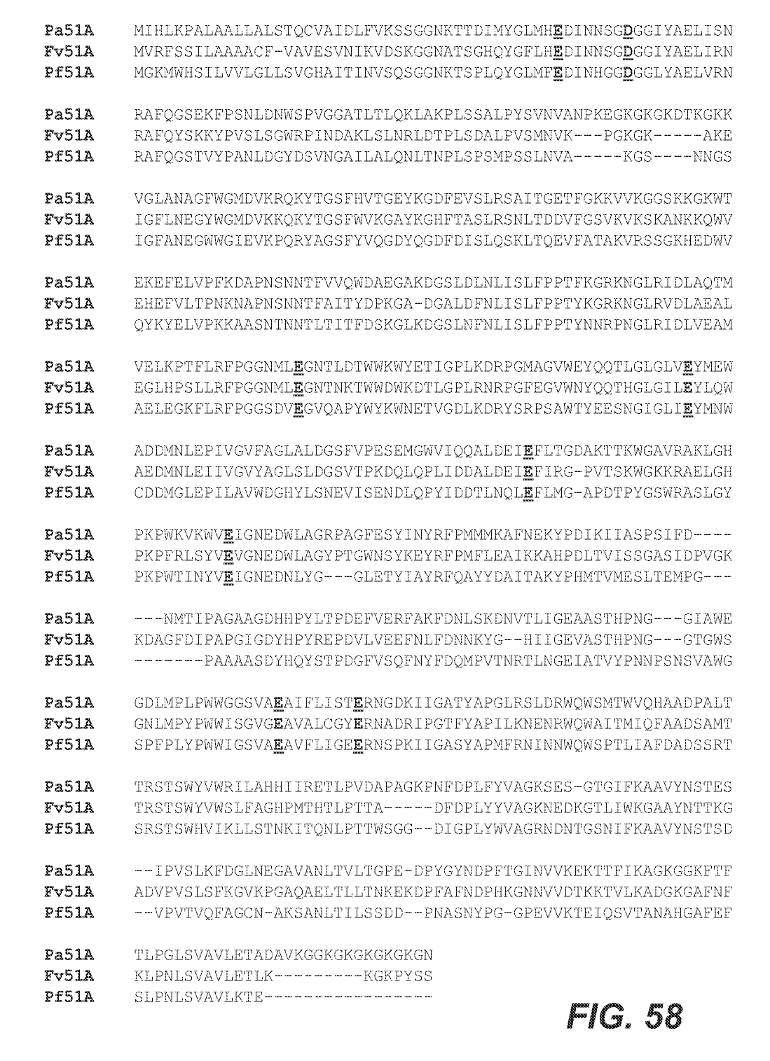

In some aspects, the cellulase composition of the present invention comprises a Pa51A polypeptide. The amino acid sequence of Pa51A (SEQ ID NO:14) is shown in FIGS. 14B and 58. SEQ ID NO:14 is the sequence of the immature Pa51A. Pa51A has a predicted signal sequence corresponding to residues 1 to 20 of SEQ ID NO:14 (underlined in FIG. 14B); cleavage of the signal sequence is predicted to yield a mature protein having a sequence corresponding to residues 21 to 676 of SEQ ID NO:14. The predicted L-α-arabinofuranosidase conserved domain is in boldface type in FIG. 14B. Pa51A was shown to have both β-xylosidase activity and L-α-arabinofuranosidase activity in, e.g., enzymatic assays using artificial substrates p-nitrophenyl-β-xylopyranoside and p-nitophenyl-α-L-arabinofuranoside. It was shown to catalyze the release of arabinose from branched arabino-xylo oligomers and to catalyze the increased xylose release from oligomer mixtures in the presence of other xylosidase enzymes. Conserved acidic residues include E43, D50, E257, E296, E340, E370, E485, and E493. As used herein, “a Pa51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, or 650 contiguous amino acid residues among residues 21 to 676 of SEQ ID NO:14. A Pa51A polypeptide preferably is unaltered, as compared to native Pa51A, at residues E43, D50, E257, E296, E340, E370, E485, and E493. A Pa51A polypeptide is preferably unaltered in at least 70%, 80%, 90%, 95%, 98%, or 99% of the amino acid residues that are conserved among a group of enzymes including Pa51A, Fv51A, and Pf51A, as shown in the alignment of FIG. 58. A Pa51A polypeptide suitably comprises the predicted conserved domain of native Pa51A as shown in FIG. 14B. An exemplary Pa51A polypeptide comprises a sequence having at least 85%, 86%, 87%, 88%, 89%, 90%, 91%, 92%, 93%, 94%, 95%, 96%, 97%, 98%, 99%, or 100% identity to the mature Pa51A sequence as shown in FIG. 14B. The Pa51A polypeptide of the invention preferably has β-xylosidase activity, L-α-arabinofuranosidase activity, or both β-xylosidase and L-α-arabinofuranosidase activities.

Accordingly, a Pa51A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:14, or to residues (i) 21-676, (ii) 21-652, (iii) 469-652, or (iv) 469-676 of SEQ ID NO:14. The polypeptide suitably has 0-xylosidase activity, L-α-arabinofuranosidase activity, or both β-xylosidase and L-α-arabinofuranosidase activities.

Gz43A:

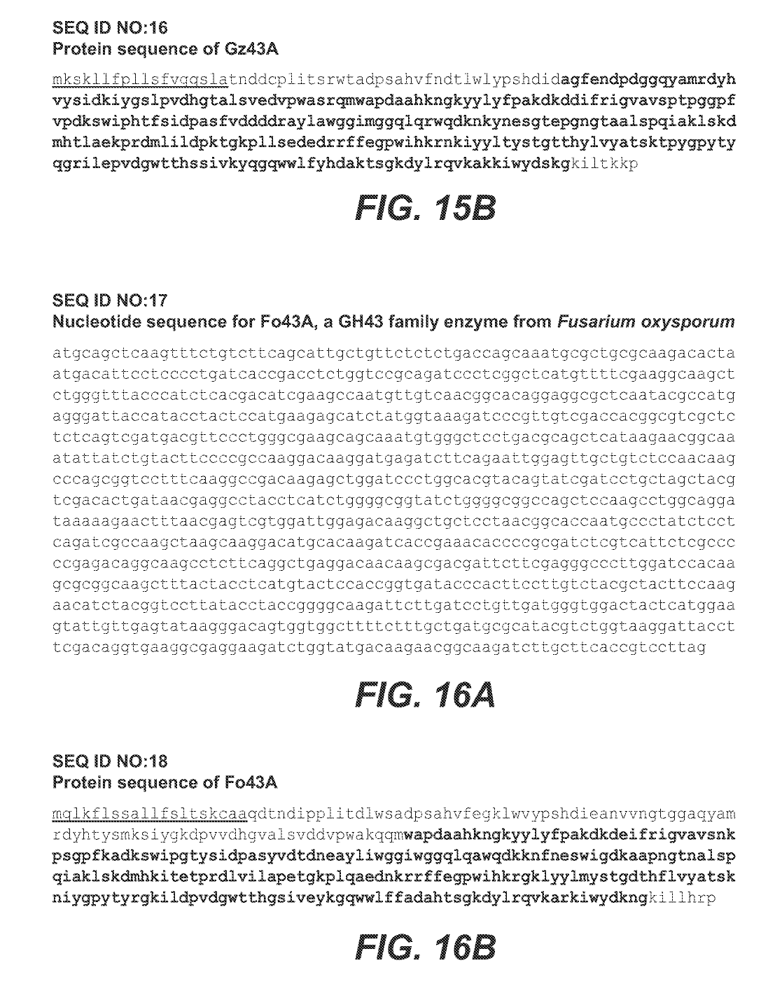

In some aspects, the cellulase composition of the present invention comprises a Gz43A polypeptide. The amino acid sequence of Gz43A (SEQ ID NO:16) is shown in FIGS. 15B and 57. SEQ ID NO:16 is the sequence of the immature Gz43A. Gz43A has a predicted signal sequence corresponding to residues 1 to 18 of SEQ ID NO:16 (underlined in FIG. 15B); cleavage of the signal sequence is predicted to yield a mature protein having a sequence corresponding to residues 19 to 340 of SEQ ID NO:16. The predicted conserved domain is in boldface type in FIG. 15B. Gz43A was shown to have β-xylosidase activity in, e.g., an enzymatic assay using p-nitophenyl-β-xylopyranoside, xylobiose or mixed, and/or linear xylo-oligomers as substrates. The predicted catalytic residues include either D33 or D68, D154, and E243. As used herein, “a Gz43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues among residues 19 to 340 of SEQ ID NO:16. A Gz43A polypeptide preferably is unaltered, as compared to native Gz43A, at residues D33 or D68, D154, and E243. A Gz43A polypeptide is preferably unaltered in at least 70%, 80%, 90%, 95%, 98%, or 99% of the amino acid residues that are conserved among a group of enzymes including Gz43A and 1, 2, 3, 4, 5, 6, 7, 8 or all 9 other amino acid sequences in the alignment of FIG. 57. A Gz43A polypeptide suitably comprises the predicted conserved domain of native Gz43A as shown in FIG. 15B. An exemplary Gz43A polypeptide comprises a sequence having at least 85%, 86%, 87%, 88%, 89%, 90%, 91%, 92%, 93%, 94%, 95%, 96%, 97%, 98%, 99%, or 100% identity to the mature Gz43A sequence as shown in FIG. 15B. The Gz43A polypeptide of the invention preferably has β-xylosidase activity.

Accordingly a Gz43A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:16, or to residues (i) 19-340, (ii) 53-340, (iii) 19-383, or (iv) 53-383 of SEQ ID NO:16. The polypeptide suitably has β-xylosidase activity.

The β-xylosidase(s) suitably constitutes about 0 wt. % to about 75 wt. % (e.g., about 0.1 wt. % to about 50 wt. %, about 1 wt. % to about 40 wt. %, about 2 wt. % to about 35 wt. %, about 5 wt. % to about 30 wt. %, about 10 wt. % to about 25 wt. %) of the total weight of enzymes in a cellulase or hemicellulase composition of the present invention. The ratio of any pair of proteins relative to each other can be readily calculated based on the disclosure herein. Compositions comprising enzymes in any weight ratio derivable from the weight percentages disclosed herein are contemplated. The β-xylosidase content can be in a range wherein the lower limit is about 0 wt. %, 0.05 wt. %, 0.5 wt. %, 1 wt. %, 2 wt. %, 3 wt. %, 4 wt. %, 5 wt. %, 6 wt. % 7 wt. %, 8 wt. %, 9 wt. %, 10 wt. %, 12 wt. %, 15 wt. %, 20 wt. %, 25 wt. %, 30 wt. %, 40 wt. %, 45 wt. %, or 50 wt. % of the total weight of enzymes in the blend/composition, and the upper limit is about 10 wt. %, 15 wt. %, 20 wt. %, 25 wt. %, 30 wt. %, 35 wt. %, 40 wt. %, 50 wt. %, 55 wt. %, 60 wt. %, 65 wt. % or 70 wt. % of the total weight of enzymes in the composition. For example, the β-xylosidase(s) suitably represent about 2 wt. % to about 30 wt. %; about 10 wt. % to about 20 wt. %; about 3 wt. % to about 10 wt. %, or about 5 wt. % to about 9 wt. % of the total weight of enzymes in the composition

The β-xylosidase can be produced by expressing an endogenous or exogenous gene encoding a β-xylosidase. The β-xylosidase can be, in some circumstances, overexpressed or underexpressed. Alternatively, the β-xylosidase can be heterologous to the host organism, which is recombinantly expressed by the host organism. Furthermore, the β-xylosidase can be added to a cellulase or hemicellulase composition of the invention in a purified or isolated form.

L-α-arabinofuranosidases:

In some aspects, the cellulase composition of the present invention comprises at least one L-α-arabinofuranosidase. In some aspects, the at least one L-α-arabinofuranosidase is selected from the group consisting of Af43A, Fv43B, Pf51A, Pa51A, and Fv51A. In some aspects, Pa51A, Fv43A have both L-α-arabinofuranosidase and β-xylosidase activity.

L-α-arabinofuranosidases (EC 3.2.1.55) from any suitable organism can be used as the one or more L-α-arabinofuranosidases. Suitable L-α-arabinofuranosidases include, e.g., an L-α-arabinofuranosidases of A. oryzae (Numan & Bhosle, J. Ind. Microbiol. Biotechnol. 2006, 33:247-260), A. sojae (Oshima et al. J. Appl. Glycosci. 2005, 52:261-265), B. brevis (Numan & Bhosle, J. Ind. Microbiol. Biotechnol. 2006, 33:247-260), B. stearothermophilus (Kim et al., J. Microbiol. Biotechnol. 2004, 14:474-482), B. breve (Shin et al., Appl. Environ. Microbiol. 2003, 69:7116-7123), B. longum (Margolles et al., Appl. Environ. Microbiol. 2003, 69:5096-5103), C. thermocellum (Taylor et al., Biochem. J. 2006, 395:31-37), F. oxysporum (Panagiotou et al., Can. J. Microbiol. 2003, 49:639-644), F. oxysporum f. sp. dianthi (Numan & Bhosle, J. Ind. Microbiol. Biotechnol. 2006, 33:247-260), G. stearothermophilus T-6 (Shallom et al., J. Biol. Chem. 2002, 277:43667-43673), H. vulgare (Lee et al., J. Biol. Chem. 2003, 278:5377-5387), P. chrysogenum (Sakamoto et al., Biophys. Acta 2003, 1621:204-210), Penicillium sp. (Rahman et al., Can. J. Microbiol. 2003, 49:58-64), P. cellulosa (Numan & Bhosle, J. Ind. Microbiol. Biotechnol. 2006, 33:247-260), R. pusillus (Rahman et al., Carbohydr. Res. 2003, 338:1469-1476), S. chartreusis, S. thermoviolacus, T. ethanolicus, T. xylanilyticus (Numan & Bhosle, J. Ind. Microbiol. Biotechnol. 2006, 33:247-260), T. fusca (Tuncer and Ball, Folia Microbiol. 2003, (Praha) 48:168-172), T. maritima (Miyazaki, Extremophiles 2005, 9:399-406), Trichoderma sp. S Y (Jung et al. Agric. Chem. Biotechnol. 2005, 48:7-10), A. kawachii (Koseki et al., Biochim. Biophys. Acta 2006, 1760:1458-1464), F. oxysporum f. sp. dianthi (Chacon-Martinez et al., Physiol. Mol. Plant. Pathol. 2004, 64:201-208), T. xylanilyticus (Debeche et al., Protein Eng. 2002, 15:21-28), H. insolens, M. giganteus (Sorensen et al., Biotechnol. Prog. 2007, 23:100-107), or R. sativus (Kotake et al. J. Exp. Bot. 2006, 57:2353-2362). Suitable L-α-arabinofuranosidases can be produced endogenously by the host organism, or can be recombinantly cloned and/or expressed by the host organism. Furthermore, suitable L-α-arabinofuranosidases can be added to a cellulase composition in a purified or isolated form.

Af43A:

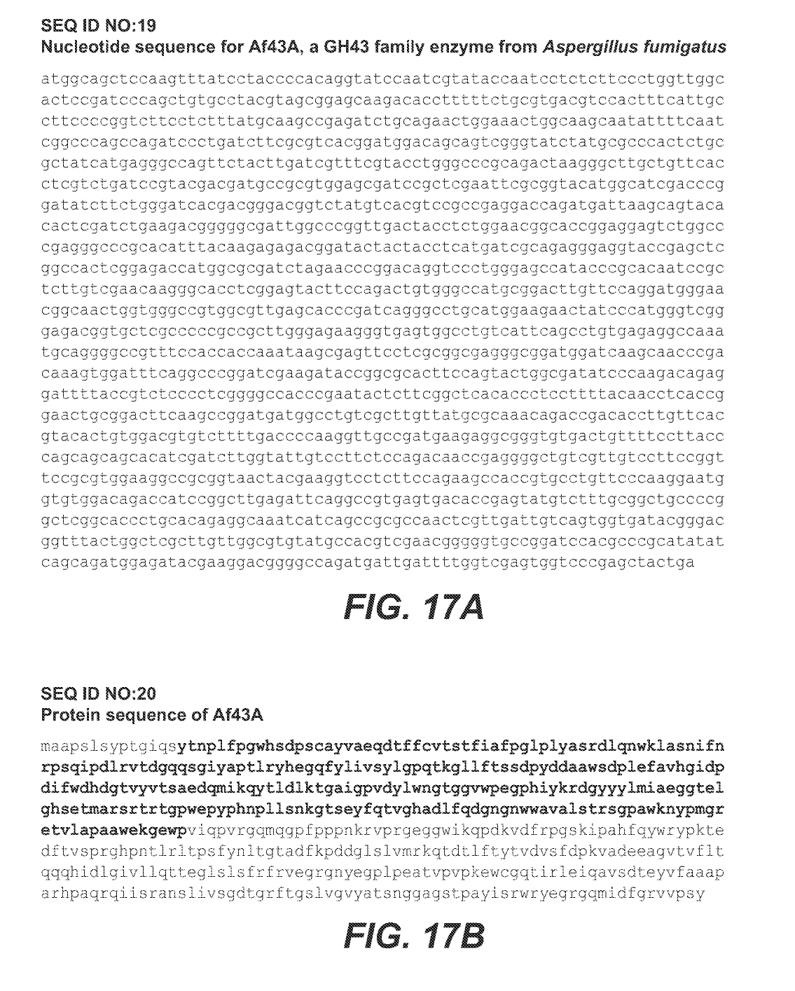

In some aspects, the cellulase composition of the present invention comprises an Af43A polypeptide. The amino acid sequence of Af43A (SEQ ID NO:20) is shown in FIGS. 17B and 57. SEQ ID NO:20 is the sequence of the immature Af43A. The predicted conserved domain is in boldface type in FIG. 17B. Af43A was shown to have L-α-arabinofuranosidase activity in, e.g., an enzymatic assay using p-nitophenyl-α-L-arabinofuranoside as a substrate. Af43A was shown to catalyze the release of arabinose from the set of oligomers released from hemicellulose via the action of endoxylanase. The predicted catalytic residues include either D26 or D58, D139, and E227. As used herein, “an Af43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues of SEQ ID NO:20. An Af43A polypeptide preferably is unaltered, as compared to native Af43A, at residues D26 or D58, D139, and E227. An Af43A polypeptide is preferably unaltered in at least 70%, 80%, 90%, 95%, 98%, or 99% of the amino acid residues that are conserved among a group of enzymes including Af43A and 1, 2, 3, 4, 5, 6, 7, 8, or all 9 other amino acid sequences in the alignment of FIG. 57. An Af43A polypeptide suitably comprises the predicted conserved domain of native Af43A as shown in FIG. 17B. An exemplary Af43A polypeptide comprises a sequence having at least 85%, 86%, 87%, 88%, 89%, 90%, 91%, 92%, 93%, 94%, 95%, 96%, 97%, 98%, 99%, or 100% sequence identity to SEQ ID NO:20. The Af43A polypeptide of the invention preferably has L-α-arabinofuranosidase activity.

Accordingly an Af43A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:20, or to residues (i) 15-558, or (ii) 15-295 of SEQ ID NO:20. The polypeptide suitably has L-α-arabinofuranosidase activity.

Pf51A:

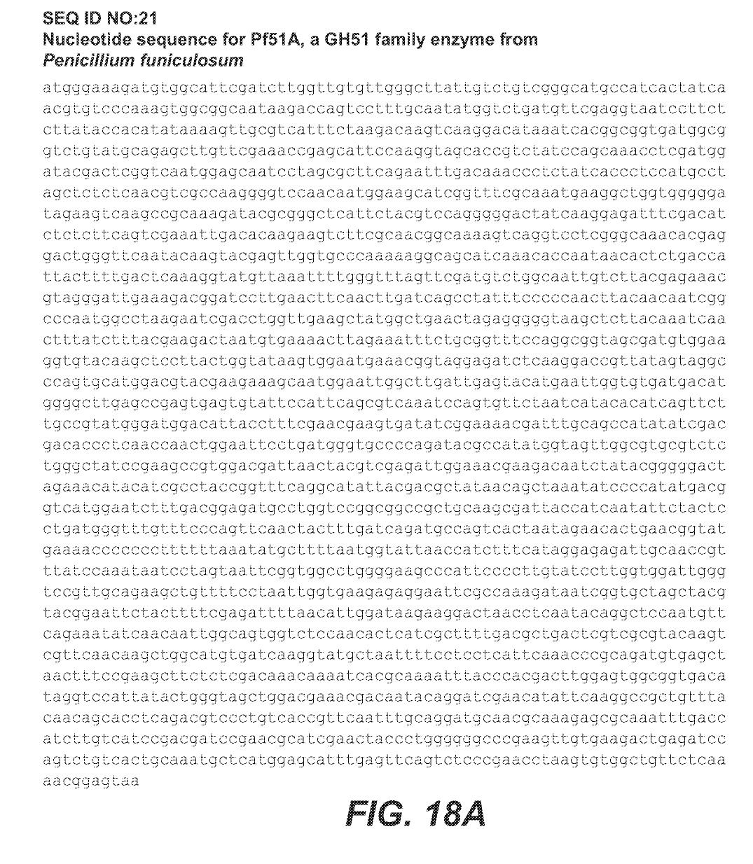

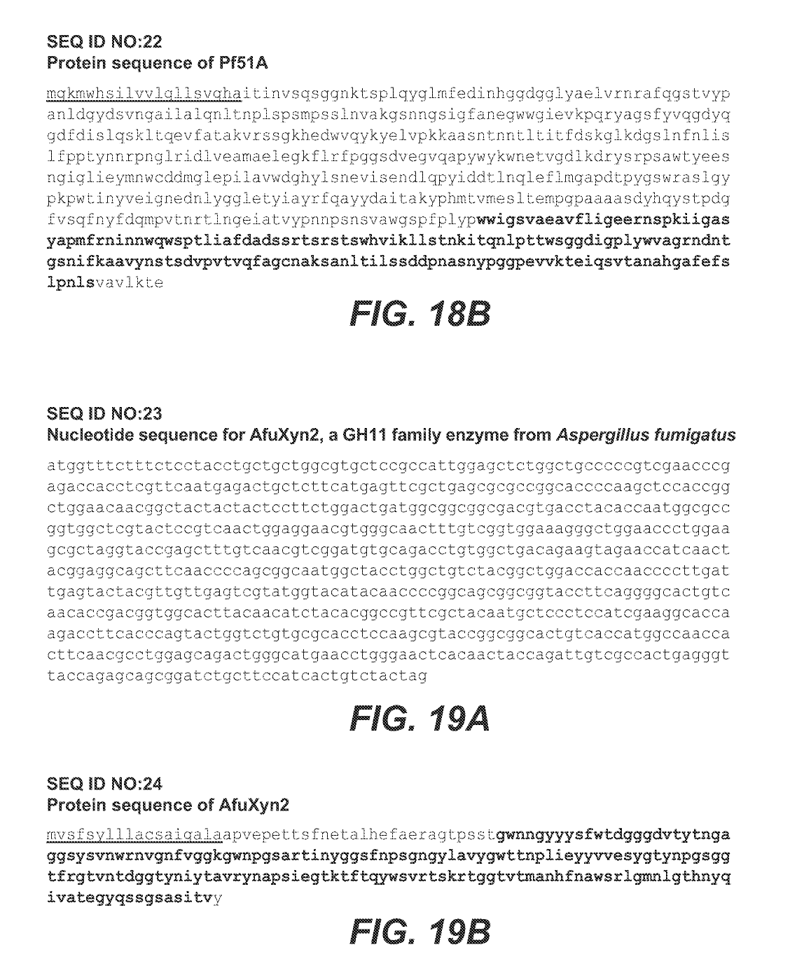

In some aspects, the cellulase composition of the present invention comprises a Pf51A polypeptide. The amino acid sequence of Pf51A (SEQ ID NO:22) is shown in FIGS. 18B and 58. SEQ ID NO:22 is the sequence of the immature Pf51A. Pf51A has a predicted signal sequence corresponding to residues 1 to 20 of SEQ ID NO:22 (underlined in FIG. 18B); cleavage of the signal sequence is predicted to yield a mature protein having a sequence corresponding to residues 21 to 642 of SEQ ID NO:22. The predicted L-α-arabinofuranosidase conserved domain is in boldface type in FIG. 18B. Pf51A was shown to have L-α-arabinofuranosidase activity in, e.g., an enzymatic assay using 4-nitrophenyl-α-L-arabinofuranoside as a substrate. Pf51A was shown to catalyze the release of arabinose from the set of oligomers released from hemicellulose via the action of endoxylanase. The predicted conserved acidic residues include E43, D50, E248, E287, E331, E360, E472, and E480. As used herein, “a Pf51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, or 600 contiguous amino acid residues among residues 21 to 642 of SEQ ID NO:22. A Pf51A polypeptide preferably is unaltered, as compared to native Pf51A, at residues E43, D50, E248, E287, E331, E360, E472, and E480. A Pf51A polypeptide is preferably unaltered in at least 70%, 80%, 90%, 95%, 98%, or 99% of the amino acid residues that are conserved among Pf51A, Pa51A, and Fv51A, as shown in in the alignment of FIG. 58. A Pf51A polypeptide suitably comprises the predicted conserved domain of native Pf51A shown in FIG. 18B. An exemplary Pf51A polypeptide comprises a sequence having at least 85%, 86%, 87%, 88%, 89%, 90%, 91%, 92%, 93%, 94%, 95%, 96%, 97%, 98%, 99%, or 100% identity to the mature Pf51A sequence shown in FIG. 18B. The Pf51A polypeptide of the invention preferably has L-α-arabinofuranosidase activity.

Accordingly a Pf51A polypeptide of the invention suitably comprises an amino acid sequence with at least 90%, 91%, 92%, 93%, 94%, 95%, 96%, 97%, 98%, 99% or 100% sequence identity to the amino acid sequence of SEQ ID NO:22, or to residues (i) 21-632, (ii) 461-632, (iii) 21-642, or (iv) 461-642 of SEQ ID NO:22. The polypeptide has L-α-arabinofuranosidase activity.

Fv51A:

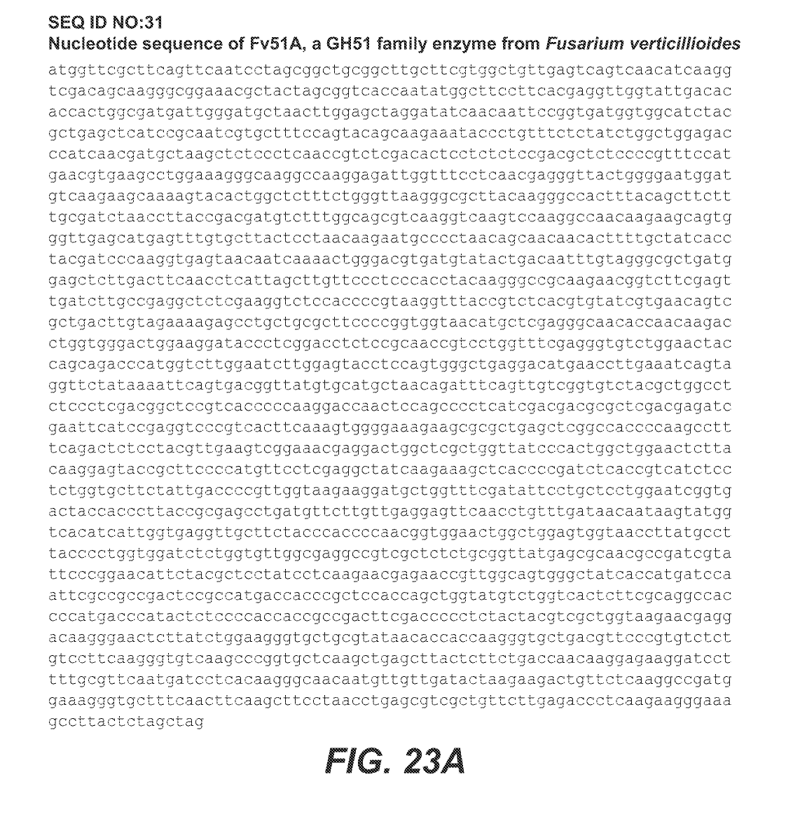

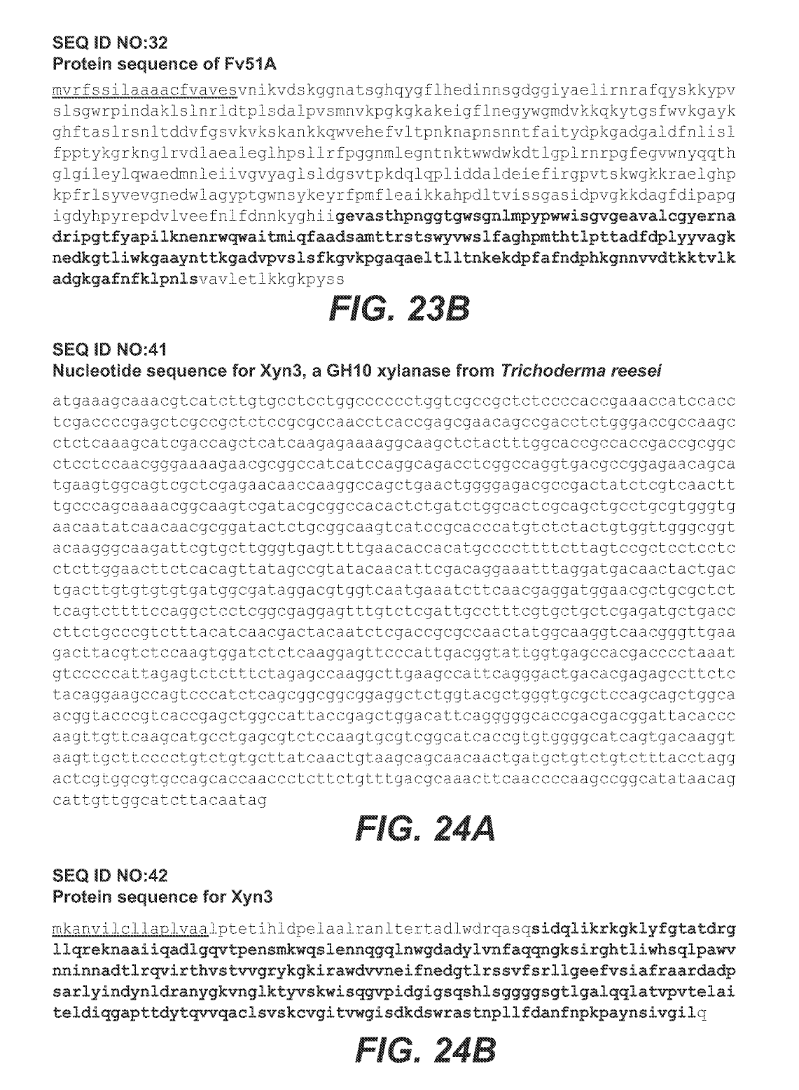

In some aspects, the cellulase composition of the present invention comprises an Fv51A polypeptide. The amino acid sequence of Fv51A (SEQ ID NO:32) is shown in FIGS. 23B and 58. SEQ ID NO:32 is the sequence of the immature Fv51A. Fv51A has a predicted signal sequence corresponding to residues 1 to 19 of SEQ ID NO:32 (underlined in FIG. 23B); cleavage of the signal sequence is predicted to yield a mature protein having a sequence corresponding to residues 20 to 660 of SEQ ID NO:32. The predicted L-α-arabinofuranosidase conserved domain is in boldface type in FIG. 23B. Fv51A was shown to have L-α-arabinofuranosidase activity in, e.g., an enzymatic assay using 4-nitrophenyl-α-L-arabinofuranoside as a substrate. Fv51A was shown to catalyze the release of arabinose from the set of oligomers released from hemicellulose via the action of endoxylanase. Conserved residues include E42, D49, E247, E286, E330, E359, E479, and E487. As used herein, “an Fv51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, or 625 contiguous amino acid residues among residues 20 to 660 of SEQ ID NO:32. An Fv51A polypeptide preferably is unaltered, as compared to native Fv51A, at residues E42, D49, E247, E286, E330, E359, E479, and E487. An Fv51A polypeptide is preferably unaltered in at least 70%, 80%, 90%, 95%, 98%, or 99% of the amino acid residues that are conserved among Fv51A, Pa51A, and Pf51A, as shown in the alignment of FIG. 58. An Fv51A polypeptide suitably comprises the predicted conserved domain of native Fv51A shown in FIG. 23B. An exemplary Fv51A polypeptide comprises a sequence having at least 85%, 86%, 87%, 88%, 89%, 90%, 91%, 92%, 93%, 94%, 95%, 96%, 97%, 98%, 99%, or 100% identity to the mature Fv51A sequence shown in FIG. 23B. The Fv51A polypeptide of the invention preferably has L-α-arabinofuranosidase activity.

Accordingly an Fv51A polypeptide of the invention suitably comprise an amino acid sequence with at least 90%, 91%, 92%, 93%, 94%, 95%, 96%, 97%, 98%, 99% or 100% sequence identity to the amino acid sequence of SEQ ID NO:32, or to residues (i) 21-660, (ii) 21-645, (iii) 450-645, or (iv) 450-660 of SEQ ID NO:32. The polypeptide suitably has L-α-arabinofuranosidase activity.

The L-α-arabinofuranosidase(s) suitably constitutes about 0.05% wt. % to about 30 wt. % (e.g., about 0.1 wt. % to about 25 wt. %, about 0.5 wt. % to about 20 wt. %, about 1 wt. % to about 10 wt. %) of the total amount of enzymes in a cellulase or hemicellulase composition of the disclosure, wherein the wt. % represents the combined weight of L-α-arabinofuranosidase(s) relative to the combined weight of all enzymes in a given composition. The L-α-arabinofuranosidase(s) can be present in a range wherein the lower limit is 0.05 wt. %, 0.5 wt., 1 wt. %, % 2 wt. %, 3 wt. %, 4 wt. %, 5 wt. %, 6 wt. % 7 wt. %, 8 wt. %, 9 wt. %, 10 wt. %, 12 wt. %, 15 wt. %, 20 wt. %, 25 wt. %, or 28 wt. %, and the upper limit is 5 wt. %, 10 wt. %, 15 wt. %, 20 wt. %, 25 wt. %, or 30 wt. %. For example, the one or more L-α-arabinofuranosidase(s) can suitably constitute about 2 wt. % to about 30 wt. % (e.g., about 2 wt. % to about 30 wt. %, about 5 wt. % to about 30 wt. %, about 5 wt. % to about 10 wt. %, about 10 wt. % to about 30 wt. %, about 20 wt. % to about 30 wt. %, about 25 wt. % to about 30 wt. %, about 2 wt. % to about 10 wt. %, about 5 wt. % to about 15 wt. %, about 10 wt. % to about 25 wt. %, about 20 wt. % to about 30 wt. %, etc) of the total weight of enzymes in a cellulase or hemicellulase composition of the invention.

The L-α-arabinofuranosidase can be produced by expressing an endogenous or exogenous gene encoding an L-α-arabinofuranosidase. The L-α-arabinofuranosidase can be, in some circumstances, overexpressed or underexpressed. Alternatively, the L-α-arabinofuranosidase can be heterologous to the host organism, which is recombinantly expressed by the host organism. Furthermore, the L-α-arabinofuranosidase can be added to a cellulase or hemicellulase composition of the invention in a purified or isolated form.

Cell Compositions

In some aspects, the present invention contemplates cells a nucleic acid encoding a polypeptide having cellulase activity. In some aspects, the cells are T. reesei cells. In some aspects, the cells are A. niger cells. In some aspects, the cells include cells of any microorganism (e.g., cells of a bacterium, a protist, an alga, a fungus (e.g., a yeast or filamentous fungus), or other microbe), and are preferably cells of a bacterium, a yeast, or a filamentous fungus. Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans. Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma. Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus,Scytaldium, Schizophyllum, Sporotrichum, Talaromyces, Thermoascus, Thielavia, Tolypocladium, Trametes, and Trichoderma. Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride. In some aspects, the cells are T. reesei cells. In some aspects, the cells are A. niger cells. In some aspects the cells further comprise one or more nucleic acids encoding one or more hemicellulase. In some aspects, the cells comprise a non-naturally occurring cellulase composition comprising a beta-glucosidase enzyme, which is a chimera of at least two beta-glucosidases.

In some aspects, the invention contemplates cells comprising a nucleic acid encoding a polypeptide having at least about 60% (e.g., at least about 65%, 70 wt. %, 75%, 80 wt. %, 85%, 90%, 91 wt. %, 92 wt. %, 93 wt. %, 94 wt. %, 95 wt. %, 96 wt. %, 97 wt. %, 98 wt. %, 99 wt. %) sequence identity to any one of SEQ ID NOs:60, 54, 56, 58, 62, 64, 66, 68, 70, 72, 74, 76, 78, and 79. In some aspects, the cells further comprises a nucleic acid encoding a polypeptide having at least one hemicellulase activity, such as, e.g., β-xylosidase, L-α-arabinofuranosidase, or xylanase activity. In some aspects, the present invention also contemplates cells comprising a chimera of two or more β-glucosidase sequences, wherein the first β-glucosidase sequence is at least about 200 amino acid residues in length, and comprises about 60% (e.g., about 65%, about 70%, about 75%, or about 80%) or more sequence identity to a contiguous stretch of SEQ ID NO:60 of equal length, and wherein the second β-glucosidase sequence is at least about 50 amino acid residues in length and comprises about 60%, (e.g., about 65%, about 65%, about 70%, about 75%, about 80%) or more sequence identity to a contiguous stretch of the equal length of one of the amino acid sequences selected form SEQ ID NOs:54, 56, 58, 62, 64, 66, 68, 70, 72, 74, 76, 78, and 79. In certain aspects, the present invention contemplates cells comprising a chimera or a hybrid of two or more β-glucosidase sequences, wherein the first β-glucosidase sequence is at least about 200 amino acid residues in length, and comprises about 60%, (e.g., about 65%, about 65%, about 70%, about 75%, about 80%) or more sequence identity to a contiguous stretch of the equal length of one of the amino acid sequences selected form SEQ ID NOs:54, 56, 58, 62, 64, 66, 68, 70, 72, 74, 76, 78, and 79, or comprises one or more or all of polypeptide sequence motifs SEQ ID NOs:164-169, and the second β-glucosidase sequence is at least about 50 amino acid residues in length, and comprises about 60%, (e.g., about 65%, about 65%, about 70%, about 75%, about 80%) or more sequence identity to a contiguous stretch of the equal length of SEQ ID NO:60. In certain embodiments, the first β-glucosidase sequence, the second β-glucosidase sequence, or both the first and the second β-glucosidase sequences comprises one or more glycosylation sites. In certain embodiments, the β-glucosidase sequence or the second β-glucosidase sequence comprises a loop region, or a sequence encoding a loop-like structure, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the first β-glucosidase sequence and the second β-glucosidase sequence are directly adjacent or connected. In some embodiments, the first β-glucosidase sequence and the second β-glucosidase sequence are not directly adjacent but rather are connected via a linker domain. In certain embodiments, the linker domain can comprise the loop region, wherein the loop region is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the linker domain is centrally located (i.e., not located at or near the N-terminal end or at or near the C-terminal end of the chimeric molecule).

In certain aspects, the invention contemplates cells comprising a chimera or hybrid of two or more β-glucosidase sequences, wherein the first β-glucosidase sequence is at least about 200 amino acid residues in length (e.g., about 250, 300, 350 or 400 amino acid residues in length) and comprises one or more or all of the amino acid sequence motifs of SEQ ID NOs:136-148, whereas the second β-glucosidase sequence is at least about 50 amino acid residues in length (e.g., about 120, 150, 170, 200, or 220 amino acid residues in length) and comprises one or more or all of the amino acid sequence motifs of SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In certain embodiments, the first β-glucosidase sequence, the second β-glucosidase sequence, or both the first and the second β-glucosidase sequences comprises one or more glycosylation sites. In certain embodiments, the β-glucosidase sequence or the second β-glucosidase sequence comprises a loop region, or a sequence encoding a loop-like structure, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the first β-glucosidase sequence and the second β-glucosidase sequence are directly adjacent or connected. In some embodiments, the first β-glucosidase sequence and the second β-glucosidase sequence are not directly adjacent but rather are connected via a linker domain. In certain embodiments, the linker domain can comprise the loop region, wherein the loop region is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the linker domain is centrally located (i.e., not located at or near the N-terminal end or at or near the C-terminal end of the chimeric molecule).

Fermentation Broth Compositions

In some aspects, the present invention contemplates a fermentation broth comprising one or more cellulase activities, wherein the broth is capable of converting greater than about 50 wt. % of the cellulose present in a biomass sample into fermentable sugars. In some aspects, the fermentation broth is capable of converting greater than about 55 wt. % (e.g., great than about 60 wt. %, 65 wt. %, 70 wt. %, 75 wt. %, 80 wt. %, 85 wt. %, or 90 wt. %) of the cellulose present in a biomass sample into fermentable sugars. In some aspects, the fermentation broth can further comprises one or more hemicellulase activities. In certain aspects, the present invention contemplates a fermentation broth comprising at least one β-glucosidase polypeptide having at least about 60% (e.g., at least about 65%, 70%, 75%, 80%, 85%, 90%, 91% 92%, 83%, 94%, 95%, 96%, 97%, 98%, 99%) sequence identity to any one of SEQ ID NOs:54, 56, 58, 60, 62, 64, 66, 68, 70, 72, 74, 76, 78, and 79. In certain aspects, the present invention contemplates a fermentation broth comprising a hybrid or chimeric β-glucosidase, which is a chimera of at least two β-glucosidase sequences.

In some aspects, the invention contemplates a fermentation broth comprising at least one β-glucosidase activity, wherein the fermentation broth is capable of converting greater than about 50 wt. % (e.g., about 55 wt. %, 60 wt. %, 65 wt. %, 70 wt. %, 75 wt. % or 80 wt. %) of the cellulose present in a biomass sample into fermentable sugars. In certain embodiments, the fermentation broth comprises an Fv3C cellulase activity, a Pa3D cellulase activity, an Fv3G activity, an Fv3D activity, a Tr3A activity, a Tr3B activity, a Te3A activity, an An3A activity, an Fo3A activity, a Gz3A activity, an Nh3A activity, a Vd3A activity, a Pa3G activity, and/or a Tn3B activity, wherein the broth is capable of converting greater than about 50 wt. % (e.g., greater than about 55 wt. %, 60 wt. %, 65 wt. %, 70 wt. %, 75 wt. %, or even 80 wt. %) of the cellulose present in a biomass sample into sugars.

In some aspects, the invention contemplates a fermentation broth comprising a chimera or hybrid of two β-glucosidase sequences, wherein the first β-glucosidase sequence is at least 200 amino acid residues in length and comprises about 60% (e.g., about 65%, about 70%, about 75%, or about 80%) or more sequence identity to a sequence of equal length of SEQ ID NO:60, and wherein the second β-glucosidase sequence is at least 50 amino acid residues in length and comprises at least about 60% (e.g., about 65%, about 70%, about 75%, or about 80%) or more sequence identity to a sequence of equal length of one of SEQ ID NOs: 54, 56, 58, 62, 64, 66, 68, 70, 72, 74, 76, 78, and 79. In some aspects, the invention contemplates a fermentation broth comprising a chimera or hybrid of two β-glucosidase sequences, wherein the first β-glucosidase sequence is at least 200 amino acid residues in length and comprises about 60% (e.g., about 65%, about 70%, about 75%, or about 80%) or more sequence identity to a sequence of equal length of one of SEQ ID NOs: 54, 56, 58, 62, 64, 66, 68, 70, 72, 74, 76, 78, and 79, and wherein the second β-glucosidase sequence is at least 50 amino acid residues in length and comprises at least about 60% (e.g., about 65%, about 70%, about 75%, or about 80%) or more sequence identity to a sequence of equal length of SEQ ID NO:60. In certain embodiments, the first β-glucosidase sequence, the second β-glucosidase sequence, or both the first and the second β-glucosidase sequences comprises one or more glycosylation sites. In certain embodiments, the β-glucosidase sequence or the second β-glucosidase sequence comprises a loop region, or a sequence encoding a loop-like structure, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the first β-glucosidase sequence and the second β-glucosidase sequence are directly adjacent or connected. In some embodiments, the first β-glucosidase sequence and the second β-glucosidase sequence are not directly adjacent but rather are connected via a linker domain. In certain embodiments, the linker domain can comprise the loop region, wherein the loop region is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the linker domain is centrally located (i.e., not located at or near the N-terminal end or the C-terminal end of the chimeric molecule).

### Methods of the Invention

In some aspects, provided herein are methods of creating chimeric enzyme backbones (e.g., cellulases such as endoglucanases, cellobiohydrolases, and β-glucosidases, and hemicellulases such as xylanases, α-arabinofuranosidases, β-xylosidases) to improve stability. In some aspects, the improved stability is an improved proteolytic stability, in that the resulting enzyme is less susceptible to proteolytic cleavage under certain standard conditions under which the enzyme is suitably or typically used. In some aspects, the proteolytic stability is for stability during storage, while in other aspects, the proteolytic stability is for stability during expression and production, which allows the more effective production of enzymes. As such, the improved stability is a reduced level of proteolytic cleavage under standard storage conditions, or under standard expression or production conditions, as compared to an unmodified enzyme that is the source enzyme for the chimeric enzyme (i.e., the enzyme whose sequence or a variant sequence thereof constitutes a part of the chimeric enzyme). In some aspects, the improved stability is reflected in both improved storage stability and improved proteolytic stability during expression and production. As such, the improved stability is a reduced level of proteolytic cleavage under standard conditions for storage as well as for expression and production.

In some aspects, provided herein are methods for converting biomass to sugars, the method comprising contacting the biomass with an amount of any of the compositions disclosed herein effective to convert biomass to fermentable sugars. In some aspects, provided herein is a a saccharification process comprising treating a biomass with a polypeptide, wherein the polypeptide has cellulase activity and wherein the process results in at least about 50 wt. % (e.g., at least about 55 wt. %, at least about 60 wt. %, at least about 65 wt. %, at least about 70 wt. %, at least about 75 wt. %, or at least about 80 wt. %) conversion of biomass to fermentable sugars. In some aspects, provided herein are methods of marketing any of the compositions disclosed herein, wherein the compositions are supplied or sold to ethanol refineries or other biochemical or biomaterial manufacturers and optionally wherein the compositions are manufactured in a manufacturing facility located at or in the vicinity of said ethanol refineries or other biochemical or biomaterial manufacturers.

Methods for Creating Chimeric Backbones

In some aspects, the invention provides for improved stability of certain β-glucosidase polypeptides. In certain aspects, the improved stability is an improved proteolytic stability, reflected in, e.g., a lesser degree of proteolytic degradation or cleavage of the β-glucosidase polypeptides under standard conditions wherein the β-glucosidase polypeptides are typically used. In some aspects, the improved proteolytic stability is an improved stability during storage, expression and/or production. As such, the improved proteolytic stability is reflected in a lesser level (e.g., as reflected in a reduced extent or level of activity loss) of proteolytic cleavage under standard storage, expression and/or production conditions where the β-glucosidase polypeptides are typically used or applied.

Not unlikely other heterologously expressed proteins, certain β-glucosidases are prone to proteolytic cleavage during production and storage by exogenase proteases, by proteases expressed by bacterial or fungal host cells, or by other external forces during the production and storage processes. Conventionally, such proteolytic degredation can be reduced by identifying known proteolytic consensus sequences or sites of cleavage in the primary amino acid sequence of a protein and mutating those amino acids so that a protease can no longer cleave the protein at that site. This approach has the disadvantage in that the polypeptide might be subject to proteolytic cleavage by more than one protease or that the cleavage might not be a result of enzymatic proteolysis. This approach is also insufficient to address situations where the proteolytic cleavage occurs at multiple sites, with tiered preference levels for the multiple sites. For example, the original protein, e.g., a β-glucosidase polypeptide of interest, may be initially cleaved at a certain site via a proteolytic cleavage mechanism. But once that initial cleavage site is identified, modified or mutated and is not longer susceptible to the same proteolytic cleavage mechanism, the same enzyme is then found to be cleaved via the same or a somewhat different proteolytic cleavage mechanism at a site that is distinct from the initial cleavage site. Of course the second site can also be identified, modified, or mutated to be no longer susceptible to proteolytic cleavage, but the enzyme can still be subject to proteolytic cleavage by the same or different mechanism as those described above, at yet anther site.

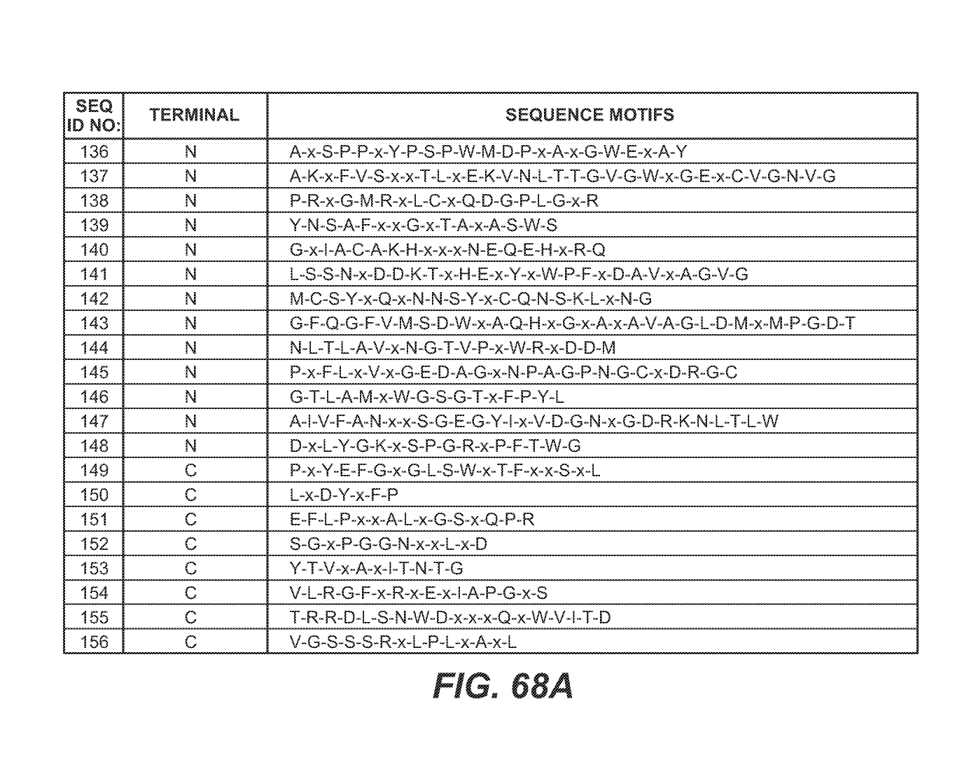

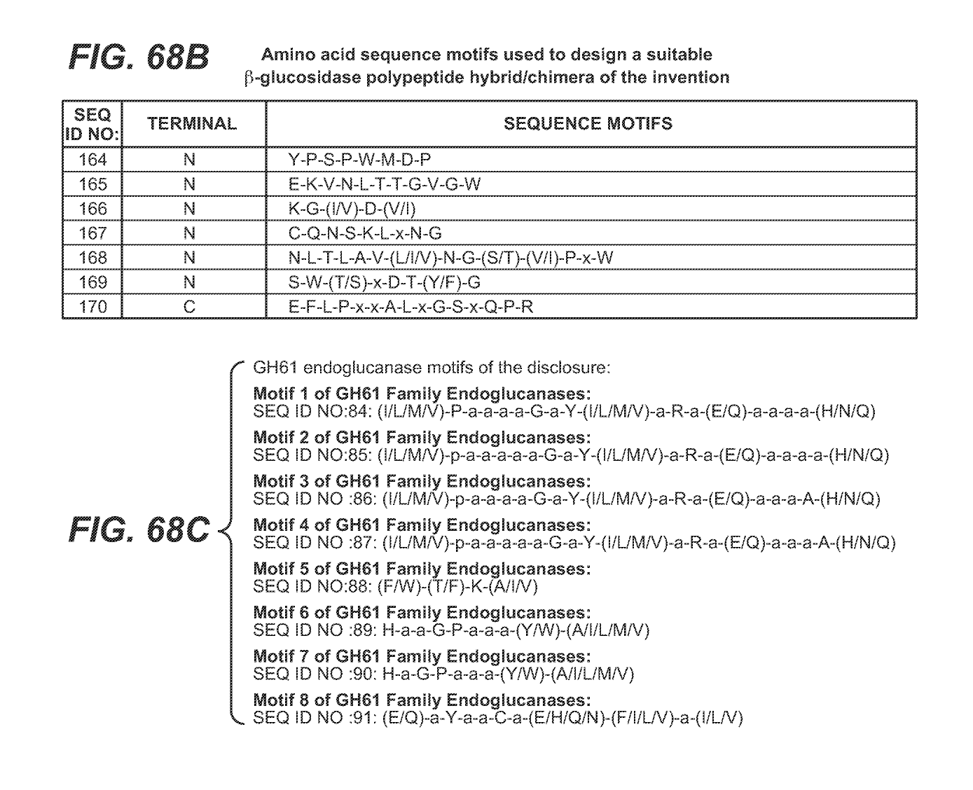

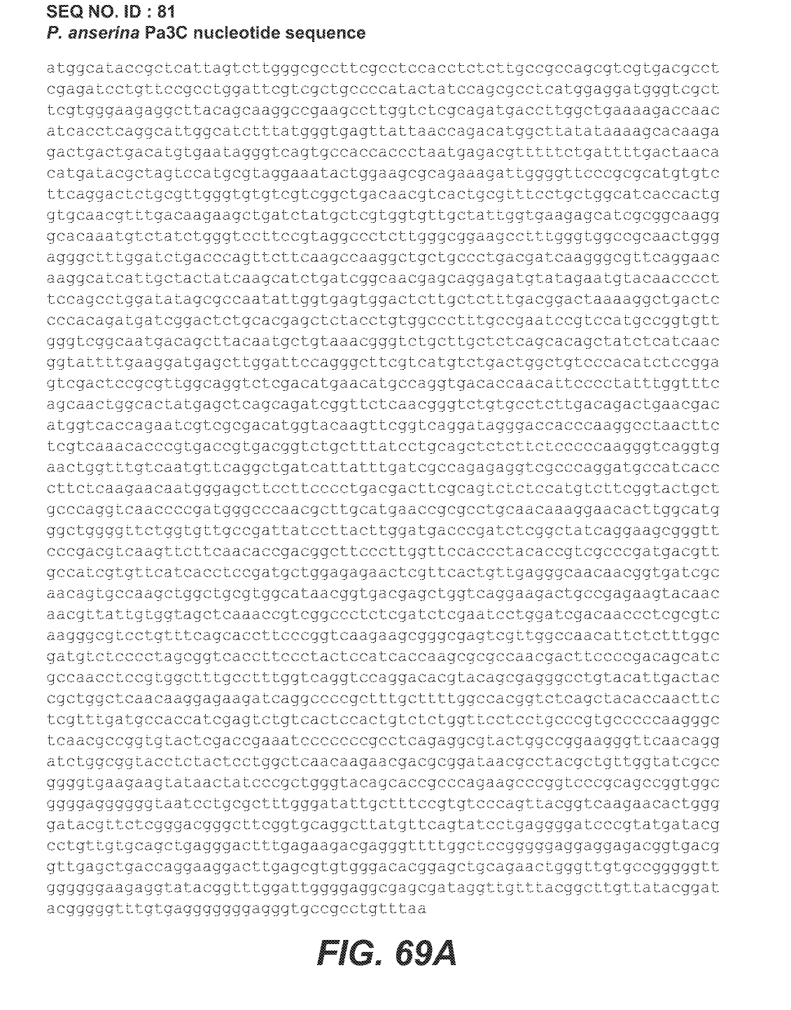

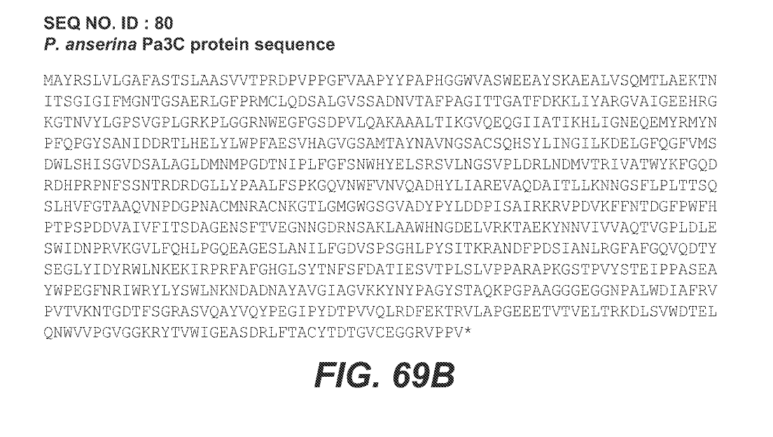

Applicants have discovered that sites of cleavage on heterologously expressed polypeptides can be identified on the basis of comparisons between the secondary structures of evolutionarily related enzymes. Comparing the amino acid sequences and predicted secondary structures of related enzymes that are not subject to cleavage during heterologous expression, production, and/or storage can lead to the identification of loop sequences present in the secondary structure of a protein. The loop sequences, however, may or may not be where the cleavage occurs. In some embodiments, the actual proteolytic cleavage can occur downstream or upstream of the loop sequences. Rather than mutating individual amino acids, and/or mutating individual amino acid residues or residues in the vicinity of the cleavage sites, as with the conventional approach, the present invention is drawn to modifying a loop domain, e.g., replacing such a loop domain, or otherwise modifying the length and/or sequence of the loop domain to achieve a polypeptide with superior stability during expression, production, and/or storage. In certain embodiments, modification can include, e.g., removing, lengthening, shortening, or replacing a loop identified in reference to evolutionarily related enzymes that are not subject to cleavage. Moreover, multiple heterologously expressed polypeptides may be subjected to this method and then fused into a single chimeric backbone possessing overall superior proteolytic stability in comparison to chimeric polypeptides which have not been altered to remove cleavage-prone secondary structures. It was determined that certain of the amino acid sequence motifs, e.g., those listed in FIG. 68A may be important to constructing a fully active and highly performing β-glucosidase hybrid/chimera/fusion molecules.

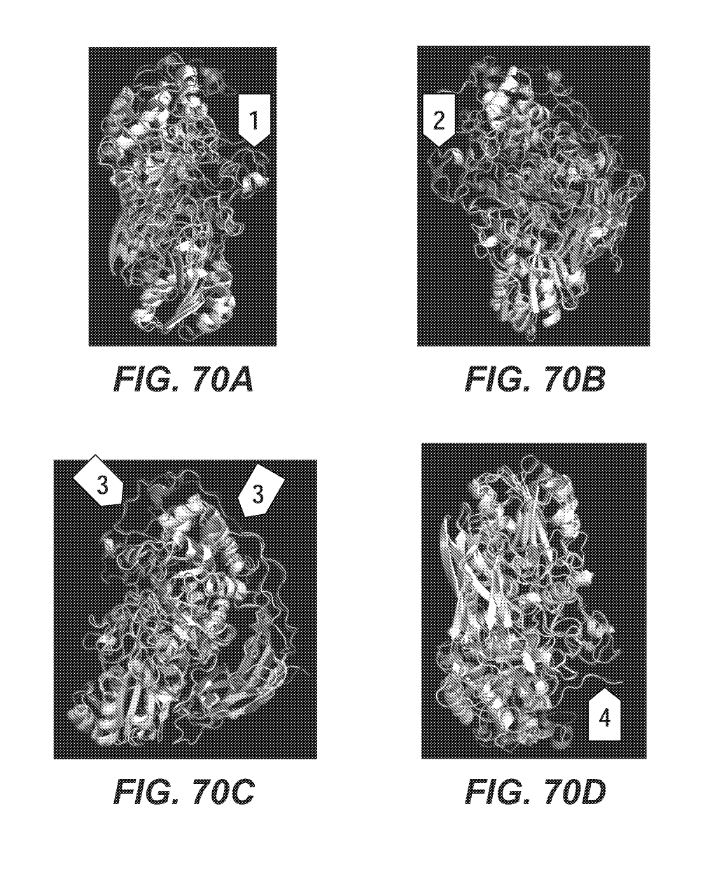

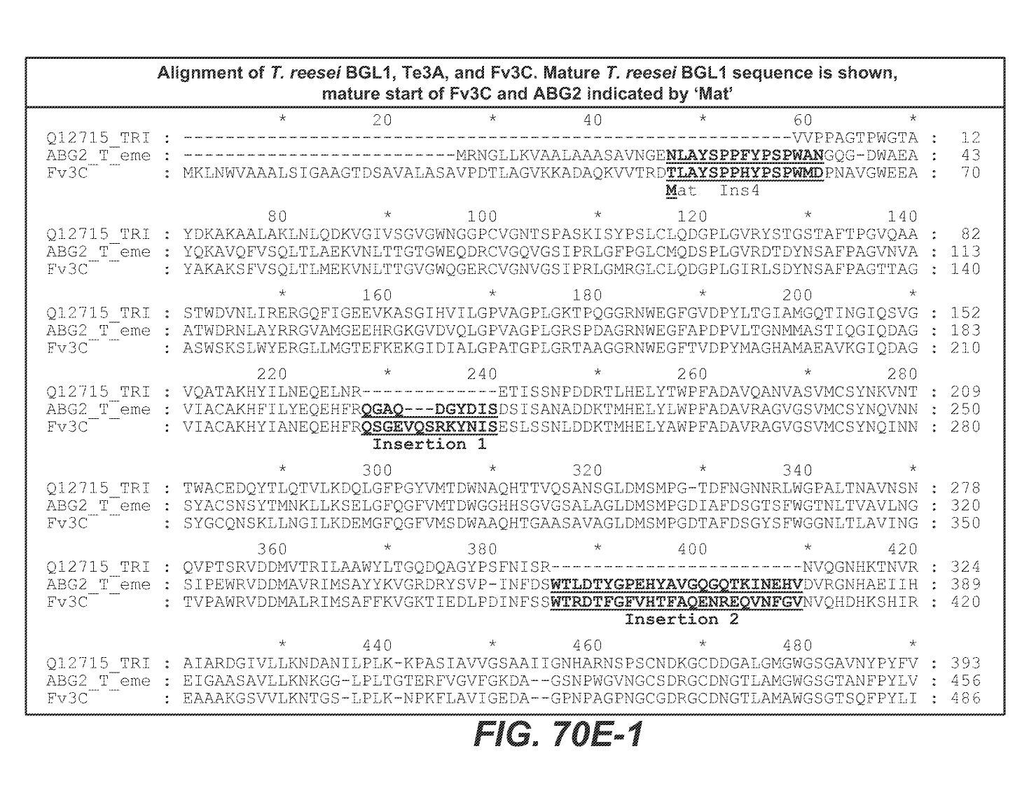

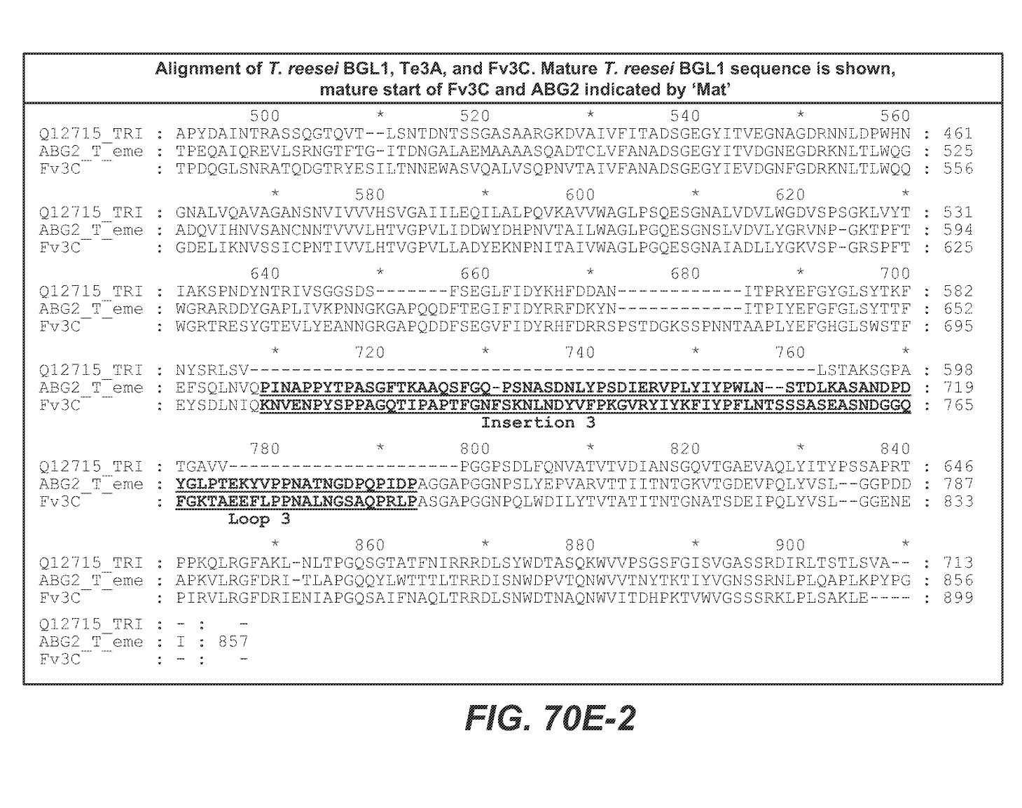

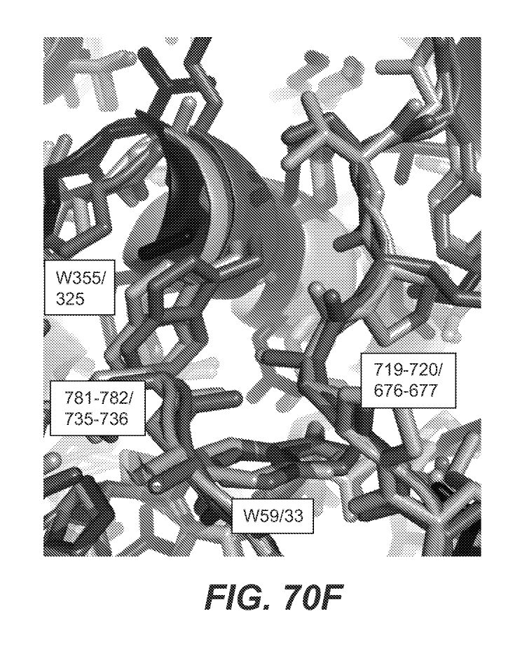

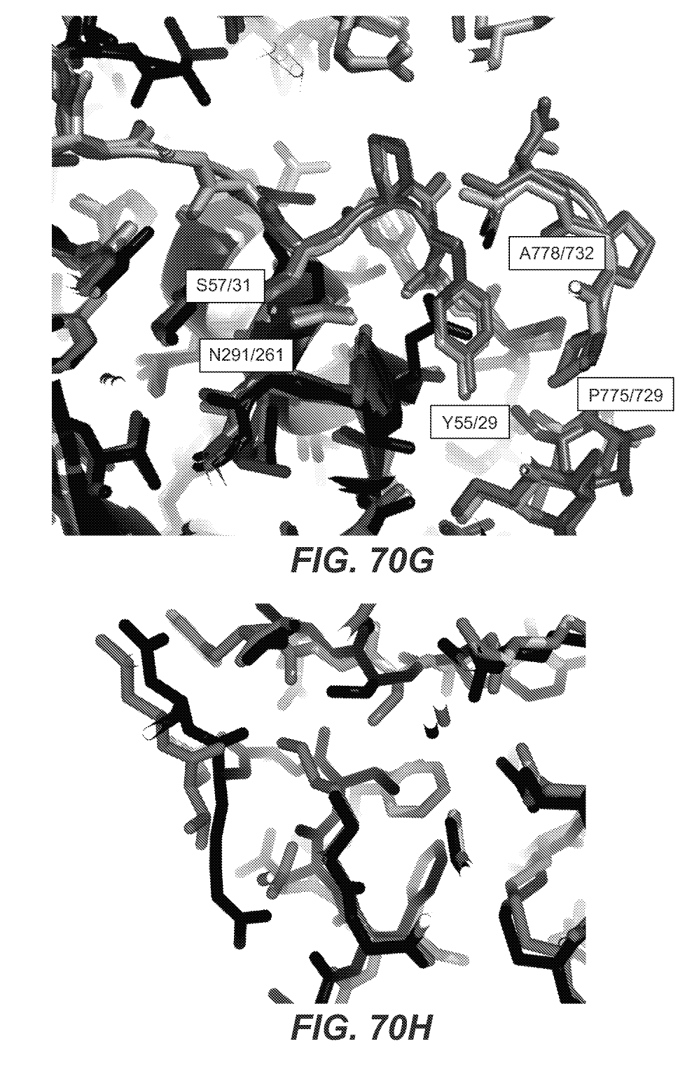

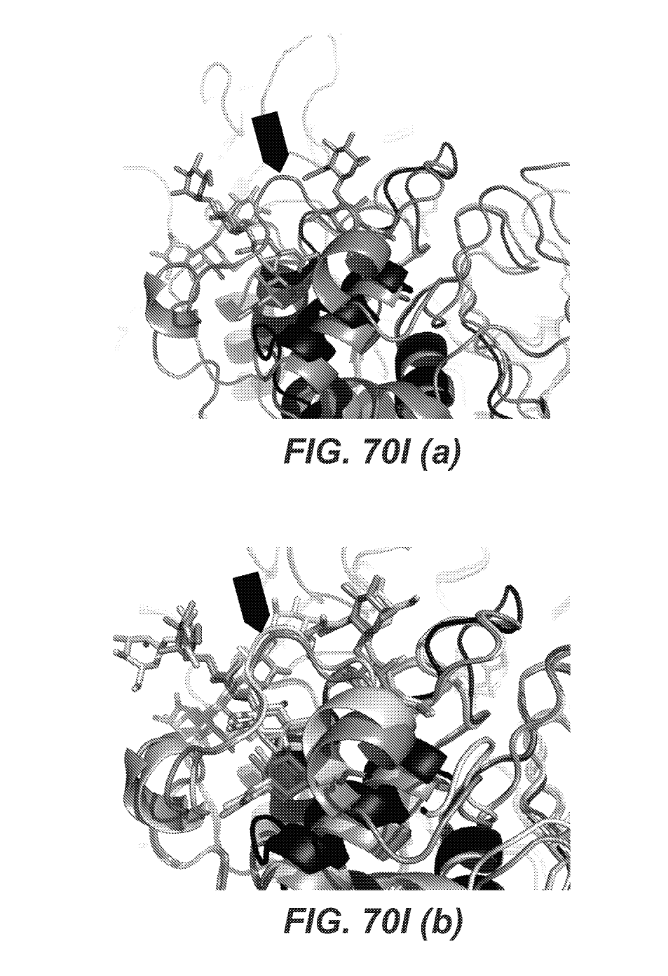

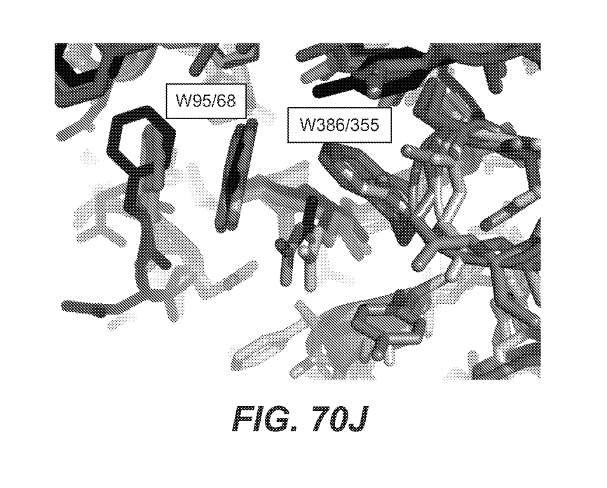

Applicants further compared the known 3-D structures of certain GH3 family β-glucosidases that are susceptible to clipping and resistant to clipping, and using conventional 3-D enzyme structure tools such as a modeling method named “Coot,” as described in e.g., Acta Cryst. (2010) D66, 486-501. For example, it was discovered that both Fv3C and Te3A had better β-glucosidase activity and performance on a number of cellulosic substrates than T. reesei Bgl1. It was also found that Fv3C is subject to proteolytic cleavage under standard storage or production conditions, rendering it less effective or desirable to be included as a component of a commercial or industrial enzyme composition. Using modeling techniques such as Coot, the shared features of Te3A, Fv3C as compared to T. reesei Bgl1 were interrogated, and four insertions were found, as indicated in FIG. 70E. From those insertions, residues and amino acid sequence motifs were further found to indicate conserved interactions (e.g., hydrogen bonding, glycosylation sites, that are present in Fv3C and Te3A, but not in T. reesei Bgl1, as indicated in FIGS. 70E-J. It was therefore determined that certain of the amino acid sequence motifs, including those listed in FIG. 68B are key to determining whether a given naturally-occurring β-glucosidase, or a mutant thereof, or a hybrid/chimera/fusion molecule thereof would have improved performance/activity as well as stability.

Without being bound by theory, improved protein stability may decrease enzyme activity. The decrease in enzymatic activity is preferably less than 20%, more preferably less than 15%, and even more preferably less than 10%. Accordingly, provided herein are methods for improving protein stability by modifying a loop sequence in an enzyme, e.g., a cellulase enzyme or a hemicellulase enzyme. In certain embodiments, the loop sequence is itself susceptible to proteolytic cleavage. In other embodiments, the loop sequence is not itself susceptible to proteolytic cleavage, but modification of the loop sequence can affect cleavage of at a site upstream or downstream of from the loop sequence in the enzyme.

In certain embodiments, the loop sequence is present in a hybrid or chimeric enzyme, e.g., a hybrid or chimeric β-glucosidase, which comprises two or more β-glucosidase sequences, each deriving from a different β-glucosidase. For example, the hybrid or chimeric β-glucosidase can comprises two β-glucosidase sequences, wherein the first β-glucosidase sequence is at least 200 amino acid residues in length, and is at least about 60% (e.g., at least about 65%, 70%, 75%, 80%, 85%, 90%, 91%, 92%, 93%, 94%, 95%, 96%, 97%, 98%, 99%) sequence identity to a sequence of equal length of SEQ ID NO:60, wherein the second β-glucosidase is at least 50 amino acid residues in length, and is at least about 60% (e.g., at least about 65%, 70%, 75%, 80%, 85%, 90%, 91%, 92%, 93%, 94%, 95%, 96%, 97%, 98%, 99%) sequence identity to a sequence of equal length of any one of SEQ ID NOs:54, 56, 58, 62, 64, 66, 68, 70, 72, 74, 76, 78, or 79. In another example, the hybrid or chimeric β-glucosidase can comprises two β-glucosidase sequences, wherein the first β-glucosidase sequence is at least 200 amino acid residues in length, and is at least about 60% (e.g., at least about 65%, 70%, 75%, 80%, 85%, 90%, 91%, 92%, 93%, 94%, 95%, 96%, 97%, 98%, 99%) sequence identity to a sequence of equal length of any one of SEQ ID NOs:54, 56, 58, 62, 64, 66, 68, 70, 72, 74, 76, 78, or 79, wherein the second β-glucosidase is at least about 50 amino acid residues in length, and is at least about 60% (e.g., at least about 65%, 70%, 75%, 80%, 85%, 90%, 91%, 92%, 93%, 94%, 95%, 96%, 97%, 98%, 99%) sequence identity to a sequence of equal length of SEQ ID NO:60. In some embodiments, the first β-glucosidase sequence of at least about 200 amino acid residues in length is at the N-terminal of the hybrid enzyme whereas the second β-glucosidase sequence of at least about 50 amino acid residues in length is at the C-terminal of the hybrid enzyme. In certain embodiments, either the N-terminal or the C-terminal β-glucosidase sequence comprises a loop sequence. In some embodiments, the loop sequence is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the N-terminal and the C-terminal β-glucosidase sequences are immediately adjacent or directly connected to each other. In other embodiments, the N-terminal and the C-terminal β-glucosidase sequences are not immediately adjacent to each other, but rather are connected via a linker domain. In certain embodiments, the linker domain is centrally located. In some embodiments, the linker domain comprises the loop sequence. In certain embodiments, the modification of the loop sequence, including, e.g., lengthening, shortening, mutating, deleting (in the entirety or partially), or replacing the loop sequence renders the resulting hybrid or chimeric enzyme less susceptible to proteolytic cleavage. As such, the resulting polypeptide or chimeric polypeptide desirably achieves an improved stability over their native counterparts (e.g., in the case of a chimeric polypeptide, the native counterparts refer to the native enzyme from which each of the chimeric part is derived). The improved stability can be reflected by a reduction or lesser level of breakdown products during standard storage, expression, production, or use conditions.

Improved stability of the heterologously expressed polypeptides and chimeric polypeptides can be determined by testing for an improvement in proteolytic stability during storage, expression or other production processes, as well as in processes where such polypeptides are used.

In certain embodiments, the loop sequence is present in a hybrid or chimeric enzyme, e.g., a hybrid or chimeric β-glucosidase, which comprises two or more β-glucosidase sequences, each deriving from a different β-glucosidase. For example, the hybrid or chimeric β-glucosidase can comprises two β-glucosidase sequences, wherein the first β-glucosidase sequence is at least 200 amino acid residues in length, and comprises one or more or all of the amino acid sequences SEQ ID NOs:136-148, wherein the second β-glucosidase is at least about 50 amino acid residues in length, and comprises one or more or all of the amino acid sequence motifs SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs:164-169, and the second of the two or more β-glucosidase is at least 50 amino acid residues in length and comprises SEQ ID NO:170. In some embodiments, the first β-glucosidase sequence of at least about 200 amino acid residues in length is at the N-terminal of the hybrid enzyme whereas the second β-glucosidase sequence of at least about 50 amino acid residues in length is at the C-terminal of the hybrid enzyme. In certain embodiments, either the N-terminal or the C-terminal β-glucosidase sequence comprises a loop sequence. In some embodiments, the loop sequence is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the N-terminal and the C-terminal β-glucosidase sequences are immediately adjacent or directly connected to each other. In other embodiments, the N-terminal and the C-terminal β-glucosidase sequences are not immediately adjacent to each other, but rather are connected via a linker domain. In certain embodiments, the linker domain is centrally located. In some embodiments, the linker domain comprises the loop sequence. In certain embodiments, the modification of the loop sequence, including, e.g., lengthening, shortening, mutating, deleting (in the entirety or partially), or replacing the loop sequence renders the resulting hybrid or chimeric enzyme less susceptible to proteolytic cleavage. As such, the resulting polypeptide or chimeric polypeptide desirably achieves an improved stability over their native counterparts (e.g., in the case of a chimeric polypeptide, the native counterparts refer to the native enzyme from which each of the chimeric part is derived). The improved stability can be reflected by a reduction or lesser level of breakdown products during standard storage, expression, production, or use conditions.

In some aspects, the loop sequence is present in a hybrid or chimeric enzyme, e.g., a hybrid or chimeric β-glucosidase, which comprises two or more enzyme sequences, wherein at least one is a β-glucosidase sequence, whereas another is not a sequence of another enzyme, and not one of a β-glucosidase. For example, the non-β-glucosidase sequence from which at least one chimeric part of a chimeric enzyme may be selected from other hemicellulases or cellulases, e.g., xylanases, endoglucanases, xylosidases, arabinofuranosidases, and others. The N-terminal domains and the C-terminal domains of the chimeric polypeptides can be directly adjacent to one another. Alternatively, the N-terminal domains and the C-terminal domains are not directly adjacent or connected, but rather are connected via a linker sequence. In certain embodiments, either the N-terminal or the C-terminal β-glucosidase sequence comprises a loop sequence. In some embodiments, the loop sequence is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In certain embodiments, the linker domain is centrally located. In some embodiments, the linker domain comprises the loop sequence. In certain embodiments, the modification of the loop sequence, including, e.g., lengthening, shortening, mutating, deleting (in the entirety or partially), or replacing the loop sequence renders the resulting hybrid or chimeric enzyme less susceptible to proteolytic cleavage. As such, the resulting polypeptide or chimeric polypeptide desirably achieves an improved stability over their native counterparts (e.g., in the case of a chimeric polypeptide, the native counterparts refer to the native enzyme from which each of the chimeric part is derived). The improved stability can be reflected by a reduction or lesser level of breakdown products during standard storage, expression, production, or use conditions. In certain embodiments, a chimeric or hybrid polypeptide can have dual cellulase and/or hemicellulase activities. For example, a chimeric or hybrid polypeptide of the invention can have both a β-glucosidase activity and a xylanase activity. In some embodiments, the chimeric or hybrid polypeptide can have improved stability over the native counterparts of its chimeric parts. For example, a chimeric β-glucosidase-xylanase polypeptide comprising a modified loop sequence can have improved stability, e.g., improved proteolytic stability under standard storage, expression, production or use conditions over the β-glucosidase and xylanase form which the chimeric polypeptide derived its β-glucosidase sequence and its xylanase sequence.

In some aspects, the invention pertains to a method of improving the stability of a cellulase or hemicellulase enzyme wherein the stability is improved by, e.g., 5% or more, 10% or more, 15% or more, 20% or more, 25% or more, or even 30% or more under standard storage, expression, production, or use conditions. The stability improvement can be measured by determining the amount of such enzyme that is cleaved after a certain period of time at certain standard storage, expression, production or use conditions. For example, the stability improvement can be measured by the amount of cleavage product at, e.g., about 1 (e.g., about 1, 2, 3, 4, 5, 6, 8, 10, 12, 15, 18, 20, 24) hrs or longer under the standard storage conditions, e.g., at ambient temperature or at an elevated temperature of about 40° C., 45° C., 50° C., or at an even higher temperature. In certain embodiments, the stability improvement can be measured by detecting and determining the amount of remaining intact product at, e.g., about 1 (e.g., about 1, 2, 3, 4, 5, 6, 8, 10, 12, 15, 18, 20, 24) hrs or longer under standard production conditions, e.g., at a temperature of over 50° C. (e.g., over 50° C., over 55° C., over 60° C., or even over 65° C.).

Methods for Converting Biomass to Sugars

In some aspects, provided herein are methods for converting biomass to sugars, the method comprising contacting the biomass with an amount of any of the compositions disclosed herein effective to convert biomass to fermentable sugars. In some aspects, the method further comprises pretreating the biomass with acid and/or base. In some aspects the acid comprises phosphoric acid. In some aspects, the base comprises sodium hydroxide or ammonia.

Biomass:

The disclosure provides methods and processes for biomass saccharification, using the cellulase or non-naturally occurring hemicellulase compositions of the disclosure. The term “biomass,” as used herein, refers to any composition comprising cellulose and/or hemicellulose (optionally also lignin in lignocellulosic biomass materials). As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure provides methods of saccharification comprising contacting a composition comprising a biomass material, e.g., a material comprising xylan, hemicellulose, cellulose, and/or a fermentable sugar, with a polypeptide of the disclosure, or a polypeptide encoded by a nucleic acid of the disclosure, or any one of the cellulase or non-naturally occurring hemicellulase compositions, or products of manufacture of the disclosure.

The scarified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, e.g., be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, e.g., also be made into a commodity chemical (e.g., ascorbic acid, isoprene, 1,3-propanediol), lipids, amino acids, proteins, and enzymes, via fermentation and/or chemical synthesis.

Pretreatment:

Prior to saccharification, biomass (e.g., lignocellulosic material) is preferably subject to one or more pretreatment step(s) in order to render xylan, hemicellulose, cellulose and/or lignin material more accessible or susceptible to enzymes and thus more amenable to hydrolysis by the enzyme(s) and/or the cellulase or non-naturally occurring hemicellulase compositions of the disclosure.

In an exemplary embodiment, the pretreatment entails subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

Another exemplary pretreatment method entails hydrolyzing biomass by subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

A further exemplary method involves processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

Another exemplary pretreatment method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369.

Further pretreatment methods can involve the use of hydrogen peroxide H₂O₂. See Gould, 1984, Biotech, and Bioengr. 26:46-52.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34.

Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia is used, e.g., in a preferred pretreatment method. Such a pretreatment method comprises subjecting a biomass material to low ammonia concentration under conditions of high solids. See, e.g., U.S. Patent Publication No. 20070031918 and PCT publication WO 06110901.

Saccharification Process

In some aspects, provided herein is a saccharification process comprising treating biomass with a polypeptide, wherein the polypeptide has cellulase activity and wherein the process results in at least about 50 wt. % (e.g., at least about 55 wt. %, 60 wt. %, 65 wt. %, 70 wt. %, 75 wt. %, or 80 wt. %) conversion of biomass to fermentable sugars. In some aspects, the biomass comprises lignin. In some aspects the biomass comprises cellulose. In some aspects the biomass comprises hemicellulose. In some aspects, the biomass comprising cellulose further comprises one or more of xylan, galactan, or arabinan. In some apects, the biomas comprises, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like), potatoes, soybean (e.g., rapeseed), barley, rye, oats, wheat, beets, and sugar cane bagasse. In some aspects, the material comprising biomass is treated with an acid and/or base prior to treatment with the polypeptide. In some aspects, the acid is phosphoric acid. In some aspects, the base is ammonia or sodium hydroxide. In some aspects, the saccharification process further comprises treating the biomass with a cellulase and/or a hemicellulase. In some aspects, the biomass is treated with whole cellulase. In some aspects, the saccharification process results in at least about 50%, 55%, 60%, 65%, 70%, 75%, 80%, 85%, or 90% by weight conversion of biomass to sugars. In some aspects, the cellulase composition or hemicellulase composition comprises a polypeptide that is a hybrid or chimeric β-glucosidase enzyme, which is a chimera of at least two β-glucosidase sequences.

In some aspects, provided is a saccharification process comprising treating biomass with a composition comprising a polypeptide, wherein the polypeptide has at least about 60% (e.g., at least about 65%, 70%, 75%, 80%, 85%, 90%, 91%, 92%, 93%, 94%, 95%, 96%, 97%, 98%, 99%) sequence identity to any one of SEQ ID NOs:60, 54, 56, 58, 62, 64, 66, 68, 70, 72, 74, 76, 78, and 79, and wherein the process results in at least about 50% (e.g., at least about 55%, 60%, 65%, 70%, 75%, 80%, 85%, or 90%) by weight conversion of biomass to fermentable sugars. In some aspects, the saccharification process comprising treating biomass with a polypeptide, wherein the polypeptide has at least about 60% (e.g., at least about 65%, 70%, 75%, 80%, 85%, 90%, 91%, 92%, 93%, 94%, 95%, 96%, 97%, 98%, 99%) sequence identity to any one of SEQ ID NOs:60, 54, 56, 58, 62, 64, 66, 68, 70, 72, 74, 76, 78, and 79, and results in at least about 60%, 70%, 75%, 80%, 85%, or 90% by weight conversion of biomass to sugars. In some aspects, the material comprising the biomass is treated with an acid and/or base prior to treatment with the polypeptide having at least 80%, at least 90%, at least 95%, or at least 97% sequence identity to any one of SEQ ID NOs:60, 54, 56, 58, 62, 64, 66, 68, 70, 72, 74, 76, 78, and 79. In some aspects, the acid is phosphoric acid.

In some aspects, provided is a saccharification process comprising treating biomass with a non-naturally occurring cellulase composition or hemicellulase composition comprising a β-glucosidase, which is a chimera or hybrid of at least two β-glucosidase sequences.

In some aspects, the saccharification process comprises treating biomass with a non-naturally occurring cellulase composition or hemicellulase composition comprising a chimera of at least two β-glucosidase sequences, wherein the first β-glucosidase sequence is at least about 200 amino acid residues in length, and comprises about 60% (e.g., about 65%, 70%, 75%, or 80%) or more sequence identity to a sequence of equal length of the amino acid sequence of Fv3C (SEQ ID NO: 60), and wherein the second β-glucosidase sequence is at least about 50 amino acid residues in length, and comprises at least about 60% (e.g., at least about 65%, 70%, 75%, or 80%) sequence identity to a sequence of equal length of one of the amino acid sequences selected from SEQ ID NOs:54, 56, 68, 62, 64, 66, 68, 70, 72, 74, 76, 78, or 79. In some aspects, the saccharification process comprises treating biomass with a non-naturally occurring cellulase composition or hemicellulase composition comprising a chimera of at least two β-glucosidase sequences, wherein the first β-glucosidase sequence is at least about 200 amino acid residues in length, and comprises about 60% (e.g., about 65%, 70%, 75%, or 80%) or more sequence identity to a sequence of equal length of the amino acid sequence of any one of the amino acid sequences selected from SEQ ID NOs:54, 56, 68, 62, 64, 66, 68, 70, 72, 74, 76, 78, or 79, and wherein the second β-glucosidase sequence is at least about 50 amino acid residues in length, and comprises at least about 60% (e.g., at least about 65%, 70%, 75%, or 80%) sequence identity to a sequence of equal length of SEQ ID NO:60. In some aspects, the saccharification process comprises treating biomass with a non-naturally occurring cellulase composition or hemicellulase composition comprising a chimera of at least two β-glucosidase sequences, wherein the first β-glucosidase sequence is at least about 200 amino acid residues in length, and comprises one or more or all of the amino acid sequence motifs SEQ ID NOs:136-148, and wherein the second β-glucosidase sequence is at least about 50 amino acid residues in length, and comprises one or more or all of the amino acid sequence motifs of SEQ ID NOs:149-156. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 164-169, and the second of the two or more β-glucosidase is at least 50 amino acid residues in length and comprises SEQ ID NO:170. In some embodiments, the first β-glucosidase sequence is at the N-terminal of the hybrid or chimeric polypeptide and the second β-glucosidase sequence is at the C-terminal of the hybrid or chimeric polypeptide. In certain embodiments, the first and the second β-glucosidase sequences are immediately adjacent or directly connected to each other. In other embodiments, the first and the second β-glucosidase sequences are not immediately adjacent, but rather are connected via a linker domain. In certain aspects, either the first or the second β-glucosidase sequence comprises a loop sequence, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some embodiments, the loop sequence is modified such that the hybrid or chimeric enzyme is less susceptible to proteolytic cleavage at a site in the loop sequence, or at residues that are outside of the loop sequence. In certain embodiments, neither the first nor the second β-glucosidase comprises the loop sequence, but rather the linker domain comprises the loop sequence. In some embodiments, the linker domain is centrally located in the hybrid or chimeric polypeptide. In some aspects, the material comprising the biomass is treated with an acid and/or base prior to treatment with the non-naturally occurring cellulase composition or hemicellulase composition comprising a chimera of at least two β-glucosidases. In some aspects, the acid is phosphoric acid. In some aspects, the base is ammonia or sodium hydroxide. In some aspects, the saccharification process further comprises treating the biomass with a hemicellulase. In some aspects, the biomass is treated with a whole cellulase. In some aspects, the saccharification process comprising treating biomass with a non-naturally occurring cellulase composition or a hemicellulase composition comprising a chimera or hybrid of at least two β-glucosidase sequences, wherein the first β-glucosidase sequence is at least about 200 amino acid residues in length and comprises about 60% (e.g., about 65%, about 70%, about 75%, or about 80%) or more sequence identity to a sequence of equal length of SEQ ID NO: 60, and wherein the second β-glucosidase sequence is at least about 50 amino acid residues in length and comprises at least about 60% (e.g., at least about 65%, 70%, 75%, or 80%) sequence identity to a sequence of equal length of any one of the amino acid sequences selected from SEQ ID NOs: 54, 56, 58, 62, 64, 66, 68, 70, 72, 74, 76, 78, and 79, results in at least about 50%, 60%, 70%, 75%, 80%, 85%, or 90% by weight conversion of the biomass to sugars. In some aspects, the saccharification process comprising treating biomass with a non-naturally occurring cellulase composition or a hemicellulase composition comprising a chimera or hybrid of at least two β-glucosidase sequences, wherein the first β-glucosidase sequence is at least about 200 amino acid residues in length and comprises about 60% (e.g., about 65%, about 70%, about 75%, or about 80%) or more sequence identity to a sequence of equal length of any one of the amino acid sequences selected from SEQ ID NOs: 54, 56, 58, 62, 64, 66, 68, 70, 72, 74, 76, 78, and 79, and wherein the second β-glucosidase sequence is at least about 50 amino acid residues in length and comprises at least about 60% (e.g., at least about 65%, 70%, 75%, or 80%) sequence identity to a sequence of equal length of SEQ ID NO:60, results in at least about 50%, 60%, 70%, 75%, 80%, 85%, or 90% by weight conversion of the biomass to sugars. In some aspects, the saccharification process comprising treating biomass with a non-naturally occurring cellulase composition or a hemicellulase composition comprising a chimera or hybrid of at least two β-glucosidase sequences, wherein the first β-glucosidase sequence is at least about 200 amino acid residues in length and comprises one or more or all of the amino acid sequence motifs of SEQ ID NOs:136-148, or preferably the motifs SEQ ID NOs: 164-169, and wherein the second β-glucosidase sequence is at least about 50 amino acid residues in length and comprises one or more or all of the amino acid sequence motifs of SEQ ID NOs:149-156, or preferably the sequence motif SEQ ID NO:170, results in at least about 50%, 60%, 70%, 75%, 80%, 85%, or 90% by weight conversion of the biomass to sugars. In some aspects, the first β-glucosidase sequence is at the N-terminal and the second β-glucosidase sequence is at the C-terminal of the chimeric or hybrid β-glucosidase polypeptide. In certain embodiments, the first and second β-glucosidase sequences are immediately adjacent or are directly connected. In other embodiments, the first and second β-glucosidase sequences are not immediately adjacent, but rather are connected via a linker domain. In some aspects, either the first or the second β-glucosidase sequence comprises a loop sequence, wherein the loop sequence comprises about 3, 4, 5, 6, 7, 8, 9, 10, or 11 amino acid residues, comprising a sequence of FDRRSPG (SEQ ID NO:171), or of FD(R/K)YNIT (SEQ ID NO:172), and wherein the modification of the loop sequence resulting in an improved stability, which may be reflected by a lesser extent of cleavage or breakdown of the hybrid or chimeric polypeptide. In certain embodiments, the improved stability is reflected by reduced or elimination of cleavage at a loop sequence residue. In some embodiments, the improved stability is reflected by reduced or elimination of cleavage at a residue outside the loop region. In certain embodiments, neither the first or second β-glucosidase sequence comprises the loop region, whereas the linker domain comprises the loop sequence, which is about 3, 4, 5, 6, 7, 8, 9, 10, or 11 amino acid residues in length, comprising a sequence of FDRRSPG (SEQ ID NO:171), or of FD(R/K)YNIT (SEQ ID NO:172). In some embodiments, the saccharification process results in at least about 50%, 60%, 70%, 75%, 80%, 85%, or 90% by weight conversion of the biomass to sugars.

Business Methods

The cellulase and/or hemicellulase compositions of the disclosure can be further used in an industrial and/or commercial settings. Accordingly a method or a method of manufacturing, marketing, or otherwise commercializing the instant cellulase and non-naturally occurring hemicellulase compositions is also contemplated.

In a specific embodiment, the cellulase and non-naturally occurring hemicellulase compositions of the invention can be supplied or sold to certain ethanol (bioethanol) refineries or other bio-chemical or bio-material manufacturers. In a first example, the non-naturally occurring cellulase and/or hemicellulase compositions can be manufactured in an enzyme manufacturing facility that is specialized in manufacturing enzymes at an industrial scale. The non-naturally occurring cellulase and/or hemicellulase compositions can then be packaged or sold to customers of the enzyme manufacturer. This operational strategy is termed the “merchant enzyme supply model” herein.

In another operational strategy, the non-naturally occurring cellulase and/or hemicellulase compositions of the invention can be produced in a state of the art enzyme production system that is built by the enzyme manufacturer at a site that is located at or in the vicinity of the bioethanol refineries or the bio-chemical/biomaterial manufacturers (“on-site”). In some embodiments, an enzyme supply agreement is executed by the enzyme manufacturer and the bioethanol refinery or the bio-chemical/biomaterial manufacturer. The enzyme manufacturer designs, controls and operates the enzyme production system on site, utilizing the host cell, expression, and production methods as described herein to produce the non-naturally-occurring cellulase and/or hemicellulase compositions. In certain embodiments, suitable biomass, preferably subject to appropriate pretreatments as described herein, can be hydrolyzed using the saccharification methods and the enzymes and/or enzyme compositions herein at or near the bioethanol refineries or the bio-chemical/biomaterial manufacturing facilities. The resulting fermentable sugars can then be subject to fermentation at the same facilities or at facilities in the vicinity. This operational strategy is termed the “on-site biorefinery model” herein.

The on-site biorefinery model provides certain advantages over the merchant enzyme supply model, including, e.g., the provision of a self-sufficient operation, allowing minimal reliance on enzyme supply from merchant enzyme suppliers. This in turn allows the bioethanol refineries or the bio-chemical/biomaterial manufacturers to better control enzyme supply based on real-time or nearly real-time demand. In certain embodiments, it is contemplated that an on-site enzyme production facility can be shared between two or among two or more bioethanol refineries and/or the bio-chemical/biomaterial manufacturers who are located near to each other, reducing the cost of transporting and storing enzymes. Moreover, this allows more immediate “drop-in” technology improvements at the enzyme production facility on-site, reducing the time lag between the improvements of enzyme compositions to a higher yield of fermentable sugars and ultimately, bioethanol or biochemicals.

The on-site biorefinery model has more general applicability in the industrial production and commercialization of bioethanols and biochemicals, in that it can be used to manufacture, supply, and produce not only the cellulase and non-naturally occurring hemicellulase compositions of the present disclosure but also those enzymes and enzyme compositions that process starch (e.g., corn) to allow for more efficient and effective direct conversion of starch to bioethanol or bio-chemicals. The starch-processing enzymes can, in certain embodiments, be produced in the on-site biorefinery, then quickly and easily integrated into the bioethanol refinery or the biochemical/biomaterial manufacturing facility in order to produce bioethanol.

Thus in certain aspects, the invention also pertains to certain business methods of applying the enzymes (e.g., cellulases, hemicellulases), cells, compositions and processes herein in the manufacturing and marketing of certain bioethanol, biofuel, biochemicals or other biomaterials. In some embodiments, the invention pertains to the application of such enzymes, cells, compositions and processes in an on-site biorefinery model. In other embodiments, the invention pertains to the application of such enzymes, cells, compositions and processes in a merchant enzyme supply model.

Relatedly, the disclosure provides the use of the enzymes and/or the enzyme compositions of the invention in a commercial setting. For example, the enzymes and/or enzyme compositions of the disclosure can be sold in a suitable market place together with instructions for typical or preferred methods of using the enzymes and/or compositions. Accordingly the enzymes and/or enzyme compositions of the disclosure can be used or commercialized within a merchant enzyme supplier model, where the enzymes and/or enzyme compositions of the disclosure are sold to a manufacturer of bioethanol, a fuel refinery, or a biochemical or biomaterials manufacturer in the business of producing fuels or bio-products. In some aspects, the enzyme and/or enzyme composition of the disclosure can be marketed or commercialized using an on-site bio-refinery model, wherein the enzyme and/or enzyme composition is produced or prepared in a facility at or near to a fuel refinery or biochemical/biomaterial manufacturer's facility, and the enzyme and/or enzyme composition of the invention is tailored to the specific needs of the fuel refinery or biochemical/biomaterial manufacturer on a real-time basis. Moreover, the disclosure relates to providing these manufacturers with technical support and/or instructions for using the enzymes and.or enzyme compositions such that the desired bio-product (e.g., biofuel, bio-chemicals, bio-materials, etc) can be manufactured and marketed.

The invention can be further understood by reference to the following examples, which are provided by way of illustration and are not meant to be limiting.

### EXAMPLES

### Example 1

### Assays/Methods

The following assays/methods were generally used in the Examples described below. Any deviations from the protocols provided below are indicated in specific Examples.

A. Pretreatment of Biomass Substrates

Corncob, corn stover and switch grass were pretreated prior to enzymatic hydrolysis according to the methods and processing ranges described in WO06110901A (unless otherwise noted). These references for pretreatment are also included in the disclosures of US-2007-0031918-A1, US-2007-0031919-A1, US-2007-0031953-A1, and/or US-2007-0037259-A1.

Ammonia fiber explosion treated (AFEX) corn stover was obtained from Michigan Biotechnology Institute International (MBI). The composition of the corn stover was determined by MBI (Teymouri, F et al. Applied Biochemistry and Biotechnology, 2004, 113:951-963) using the National Renewable Energy Laboratory (NREL) procedure, (NREL LAP-002). NREL procedures are available at: http://www.nrel.gov/biomass/analytical_procedures.html.

B. Compositional Analysis of Biomass

The 2-step acid hydrolysis method described in Determination of structural carbohydrates and lignin in the biomass (National Renewable Energy Laboratory, Golden, Colo. 2008 http://www.nrel.gov/biomass/pdfs/42618.pdf) was used to measure the composition of biomass substrates. Using this method, enzymatic hydrolysis results were reported herein in terms of percent conversion with respect to the theoretical yield from the starting cellulose and xylan content of the substrate.

C. Total Protein Assay

The BCA protein assay is a colorimetric assay that measures protein concentration with a spectrophotometer. The BCA Protein Assay Kit (Pierce Chemical) was used according to the manufacturer's suggestion. Enzyme dilutions were prepared in test tubes using 50 mM sodium acetate pH 5 buffer. Diluted enzyme solutions (each 0.1 mL) were individually added to a 2 mL Eppendorf centrifuge tube containing 1 mL 15% tricholoroacetic acid (TCA). The tubes were vortexed and placed in an ice bath for 10 min. The tubes were centrifuged at 14,000 rpm for 6 min. The supernatants were discarded, the pellets were individually re-suspended in 1 mL 0.1 N NaOH, and the tubes were again vortexed until the pellet dissolved. BSA standard solutions were prepared from a stock solution of 2 mg/mL. A BCA working solution was prepared by mixing 0.5 mL Reagent B with 25 mL Reagent A of the BCA Protein Assay Kit. The resuspended enzyme samples were added to 3 Eppendorf centrifuge tubes at a volume of 0.1 mL each. Two (2) mL Pierce BCA working solution was added to the tube of each sample and the BSA standards. The tubes were incubated in a 37° C. waterbath for 30 min. The samples were cooled to room temperature (15 min) and the absorbance at 562 nm of each sample was measured.

Average values for the protein absorbance for each standard were calculated. The average protein standard was plotted, absorbance on x-axis and concentration (mg/mL) on the y-axis. The points were fit to a linear equation: y=mx+b. The raw concentration of the enzyme samples was calculated by substituting the absorbance for the x-value. The total protein concentration was calculated by multiplying with the dilution factor.

The total protein of purified samples was determined by A280 (Pace, C N, et al. Protein Science, 1995, 4:2411-2423).

The total protein content of fermentation products was sometimes measured as total nitrogen by combustion, capture and measurement of released nitrogen, either using the Kjeldahl method (rtech laboratories) or using the DUMAS method (TruSpec CN) (Sader, A. P. O. et al., Archives of Veterinary Science, 2004, 9(2):73-79). For complex samples, e.g., fermentation broths, an average 16% N content, and the conversion factor of 6.25 for nitrogen to protein was used for calculation. In some cases, to account for interfering non-protein nitrogen, total precipitable protein was measured. In those cases, a 12.5% TCA concentration was used for the measurements, and the protein-containing TCA pellets were re-suspended in 0.1 M NaOH.

In some cases, Coomassie Plus, also known as the Better Bradford Assay (Thermo Scientific, Rockford, Ill.) was used according to manufacturer recommendation. In other cases total protein was measured using the Biuret method as modified by Weichselbaum and Gornall using Bovine Serum Albumin as a calibrator (Weichselbaum, T. Amer. J. Clin. Path. 1960, 16:40; Gornall, A. et al. J. Biol. Chem. 1949, 177:752).

D. Glucose Determination Using ABTS

The ABTS (2,2′-azino-bis(3-ethylenethiazoline-6)-sulfonic acid) assay for glucose determination was based on the principle that in the presence of O₂, glucose oxidase catalyzes the oxidation of glucose while producing stoichiometric amounts of hydrogen peroxide (H₂O₂). This reaction is followed by a horse radish peroxidase (HRP)-catalyzed oxidation of ABTS, which linearly correlates to the concentration of H₂O₂. The emergence of oxidized ABTS is indicated by the evolution of a green color, which is quantified at an OD of 405 nm. A mixture of 2.74 mg/mL ABTS powder (Sigma), 0.1 U/mL HRP (Sigma) and 1 U/mL Glucose Oxidase, (OxyGO® HP L5000, Genencor, Danisco USA) was prepared in a 50 mM sodium acetate buffer, pH 5.0, and kept in the dark. Glucose standards (at 0, 2, 4, 6, 8, 10 nmol) were prepared in 50 mM sodium acetate Buffer, pH 5.0. Ten (10) μL of the standards was added individually to a 96-well flat bottom micro titer plate in triplicate. Ten (10) μL of serially diluted samples were also added to the plate. One hundred (100) μL of ABTS substrate solution was added to each well and the plate was placed on a spectrophotometric plate reader. Oxidation of ABTS was read for 5 min at 405 nm.

Alternately, absorbance at 405 nm was measured after 15-30 min of incubation followed by quenching of the reaction using a quenching mix containing 50 mM sodium acetate buffer, pH 5.0, and 2% SDS.

E. Sugar Analysis by HPLC

Samples from cob saccharification hydrolysis were prepared by removing insoluble material using centrifugation, filtration through a 0.22 μm nylon Spin-X centrifuge tube filter (Corning, Corning, N.Y.), and dilution to the desired concentrations of soluble sugars using distilled water. Monomer sugars were determined on a Shodex Sugar SH-G SH1011, 8×300 mm with a 6×50 mm SH-1011P guard column (www.shodex.net). The solvent used was 0.01 NH₂SO₄, and the chromatography run was performed at a flow rate of 0.6 mL/min. The column temperature was maintained at 50° C., and detection was by refractive index. Alternately, the amounts of sugar were analyzed using a Biorad Aminex HPX-87H column with a Waters 2410 refractive index detector. The analysis time was about 20 min, the injection volume was 20 μL, the mobile phase was a 0.01 N sulfuric acid, which was filtered through a 0.2 μm filter and degassed, the flow rate was 0.6 mL/min, and the column temperature was maintained at 60° C. External standards of glucose, xylose, and arabinose were run with each sample set.

Size exclusion chromatography was used to separate and identify oligomeric sugars. A Tosoh Biosep G2000PW column 7.5 mm×60 cm was used. Distilled water was used to elute the sugars. A flow rate of 0.6 mL/min was used, and the column was run at room temperature. Six carbon sugar standards included stachyose, raffinose, cellobiose and glucose; five carbon sugar standards included xylohexose, xylopentose, xylotetrose, xylotriose, xylobiose and xylose. Xylo-oligomer standards were purchased (Megazyme). Detection was by refractive index. Either peak area units or relative peak area by percent was used to report the results.

Total soluble sugars were determined by hydrolysis of the centrifuged and filter-clarified samples (above). The clarified sample was diluted 1:1 using 0.8 NH₂SO₄. The resulting solution was autoclaved in a capped vial for 1 h at 121° C. Results are reported without correction for loss of monomer sugar during hydrolysis.

F. Oligomer Preparation from Cob and Enzyme Assays

Oligomers from T. reesei Xyn3 hydrolysis of corncobs were prepared by incubating 8 mg T. reesei Xyn3 per g Glucan+Xylan with 250 g dry weight of dilute ammonia pretreated corncob in a 50 mM pH 5.0 sodium acetate buffer. The reaction proceeded for 72 h at 48° C., with rotary shaking at 180 rpm. The supernatant was centrifuged 9,000×G, then filtered through 0.22 μm Nalgene filters to recover the soluble sugars.

G. Biomass Saccharification Assay

For typical examples herein, corncob saccharification assays were performed in a micro titer plate format in accordance with the following procedures, unless a particular example indicated specific variations. The biomass substrate, e.g., the dilute ammonia pretreated corncob, was diluted in water and pH-adjusted with sulfuric acid to create a pH 5, 7% cellulose slurry that was used without further processing in the assay. Enzyme samples were loaded based on mg total protein per g of cellulose, or per g of xylan, or per g of cellulose and xylan combined (as determined using conventional compositional analysis methods, supra) in the corncob substrate. The enzymes were diluted in 50 mM sodium acetate, pH 5.0, to obtain the desired loading concentrations. Forty (40) μL of enzyme solution were added to 70 mg of dilute-ammonia pretreated corncob at 7% cellulose per well (equivalent to 4.5% cellulose final per well). The assay plates were then covered with aluminum plate sealers, mixed at room temperature, and incubated at 50° C., 200 rpm, for 3 d. At the end of the incubation period, the saccharification reaction was quenched by the addition to each well of 100 μL of 100 mM glycine buffer, pH10.0, and the plate was centrifuged for 5 min at 3,000 rpm. Ten (10) μL of the supernatant was added to 200 μL of MilliQ water in a 96-well HPLC plate and the soluble sugars were measured by HPLC.

H. Microtiter Plate Saccharification Assay

Purified cellulases and whole cellulase strain cell-free products were introduced into the saccharification assay in an amount based on the total protein (in mg) per g cellulose in the substrate. Purified hemicellulases were loaded based on the xylan content of the substrate. Biomass substrates, including, e.g., dilute acid-pretreated cornstover (PCS), ammonia fiber expanded (AFEX) cornstover, dilute ammonia pretreated corncob, sodium hydroxide (NaOH) pretreated corncob, and dilute ammonia switchgrass, were mixed at the indicated % solids levels and the pH of the mixtures was adjusted to 5.0. The plates were covered with aluminum plate sealers and placed in a 50° C. incubator. Incubation took place with shaking, for 2 d. The reactions were terminated by adding 100 μL 100 mM glycine, pH 10 to individual wells. After thorough mixing, the plates were centrifuged and the supernatants were diluted 10 fold into an HPLC plate containing 100 μL 10 mM glycine buffer, pH 10. The concentrations of soluble sugars produced were measured using HPLC as described for the Cellobiose hydrolysis assay (below). The percent glucan conversion is defined as [mg glucose+(mg cellobiose×1.056+mg cellotriose×1.056)]/[mg cellulose in substrate×1.111]; % xylan conversion is defined as [mg xylose+(mg xylobiose×1.06)]/[mg xylan in substrate×1.136].

I. Cellobiose Hydrolysis Assay

Cellobiase activity was determined using the method of Ghose, T. K. Pure and Applied Chemistry, 1987, 59(2), 257-268. Cellobiose units (derived as described in Ghose) are defined as 0.815 divided by the amount of enzyme required to release 0.1 mg glucose under the assay conditions.

J. Chloro-Nitro-Phenyl-Glucoside (CNPG) Hydrolysis Assay

Two hundred (200) μL of a 50 mM sodium acetate buffer, pH 5 was added to individual wells of a microtiter plate. The plate was covered and allowed to equilibrate at 37° C. for 15 min in an Eppendorf Thermomixer. Five (5) μL of enzyme, diluted in 50 mM sodium acetate buffer, pH 5, was also added to individual wells. The plate was covered again, and allowed to equilibrate at 37° C. for 5 min. Twenty (20) μL of 2 mM 2-Chloro-4-nitrophenyl-beta-D-Glucopyranoside (CNPG, Rose Scientific Ltd., Edmonton, Calif.) prepared in Millipore water was added to individual wells and the plate was quickly transferred to a spectrophotometer (SpectraMax 250, Molecular Devices). A kinetic read was performed at OD 405 nm for 15 min and the data recorded as Vₘₐₓ. The extinction coefficient for CNP was used to convert Vₘₐₓ from units of OD/sec to μM CNP/sec. Specific activity (μM CNP/sec/mg Protein) was determined by dividing μM CNP/sec by the mg of enzyme protein used in the assay.

K. Calcofluor Assay

All chemicals used were of analytical grade. Avicel PH-101 was purchased from FMC BioPolymer (Philadelphia, Pa.). Cellobiose and calcofluor white were purchased from Sigma (St. Louise, Mo.). Phosphoric acid swollen cellulose (PASC) was prepared from Avicel PH-101 using an adapted protocol of Walseth, TAPPI 1971, 35:228 and Wood, Biochem. J. 1971, 121:353-362. In short, Avicel was solubilized in concentrated phosphoric acid then precipitated using cold deionized water. After the cellulose is collected and washed with more water to neutralize the pH, it was diluted to 1% solids in 50 mM sodium acetate pH5.

All enzyme dilutions were made into 50 mM sodium acetate buffer, pH5.0. GC220 Cellulase (Danisco US Inc., Genencor) was diluted to 2.5, 5, 10, and 15 mg protein/G PASC, to produce a linear calibration curve. Samples to be tested were diluted to fall within the range of the calibration curve, i.e. to obtain a response of 0.1 to 0.4 fraction product. 150 μL of cold 1% PASC was added to 20 μL of enzyme solution in 96-well microtiter plates. The plate was covered and incubated for 2 h at 50° C., 200 rpm in an Innova incubator/shaker. The reaction was quenched with 100 μL of 50 μg/mL Calcofluor in 100 mM Glycine, pH10. Fluorescence was read on a fluorescence microplate reader (SpectraMax M5 by Molecular Devices) at excitation wavelength Ex=365 nm and emission wavelength Em=435 nm. The result is expressed as the fraction product according to the equation:

FP=1−(Fl sample−Fl buffer w/cellobiose)/(Fl zero enzyme−Fl buffer w/cellobiose),

wherein FP is fraction product, and Fl=fluorescence units

### Example 2

### Construction of an Integrated Expression Strain of Trichoderma reesei

An integrated expression strain of Trichoderma reesei was constructed that co-expressed five genes: T. reesei β-glucosidase gene bgl1, T. reesei endoxylanase gene xyn3, F. verticillioides β-xylosidase gene fv3A, F. verticillioides β-xylosidase gene fv43D, and F. verticillioides α-arabinofuranosidase gene fv51A.

The construction of the expression cassettes for these different genes and the transformation of T. reesei strain are described below.

A. Construction of the β-Glucosidase Expression Vector

The N-terminal portion of the native T. reesei β-glucosidase gene bgl1 was codon optimized (DNA 2.0, Menlo Park, Calif.). This synthesized portion comprised the first 447 bases of the coding region of this enzyme. This fragment was then amplified by PCR using primers SK943 and SK941 (below). The remaining region of the native bgl1 gene was PCR amplified from a genomic DNA sample extracted from T. reesei strain RL-P37 (Sheir-Neiss, G et al. Appl. Microbiol. Biotechnol. 1984, 20:46-53), using the primers SK940 and SK942 (below). These two PCR fragments of the bgl1 gene were fused together in a fusion PCR reaction, using primers SK943 and SK942:

Forward Primer SK943:
(SEQ ID NO: 92)
(5′-CACCATGAGATATAGAACAGCTGCCGCT-3′)
Reverse Primer SK941:
(SEQ ID NO: 93)
(5′-CGACCGCCCTGCGGAGTCTTGCCCAGTGGTCCCGCGACAG-3′)
Forward Primer (SK940):
(SEQ ID NO: 94)
(5′-CTGTCGCGGGACCACTGGGCAAGACTCCGCAGGGCGGTCG-3′)
Reverse Primer (SK942):
(SEQ ID NO: 95)
(5′-CCTACGCTACCGACAGAGTG-3′)

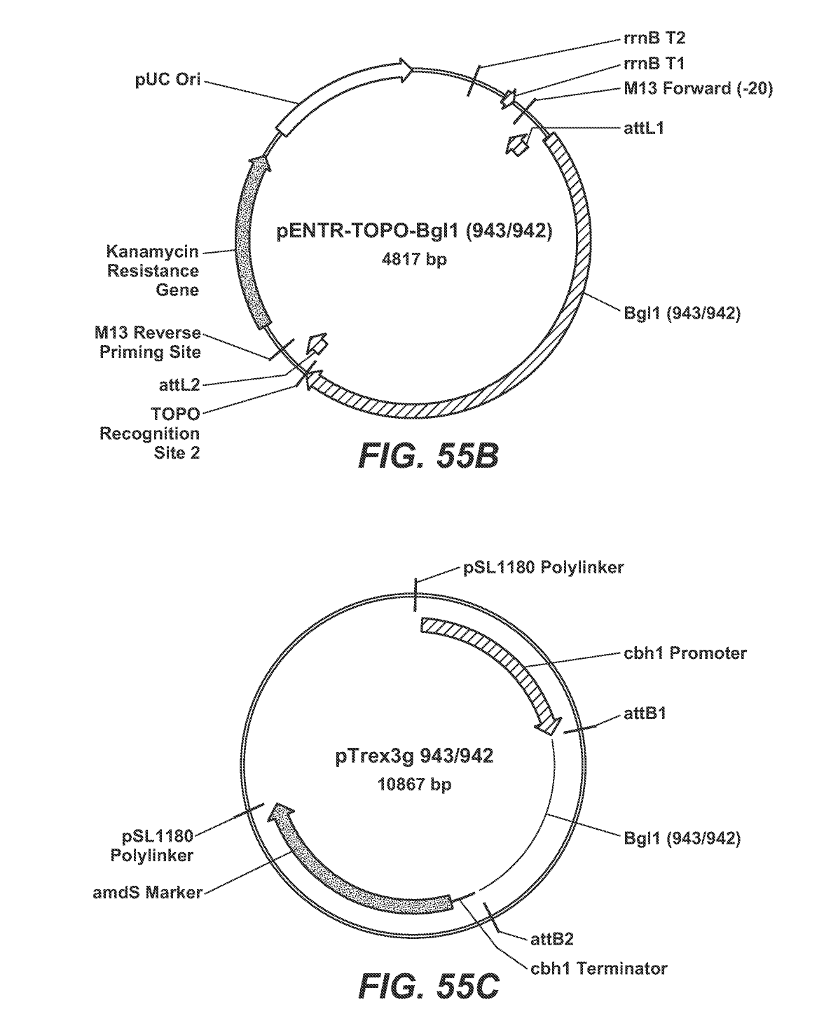

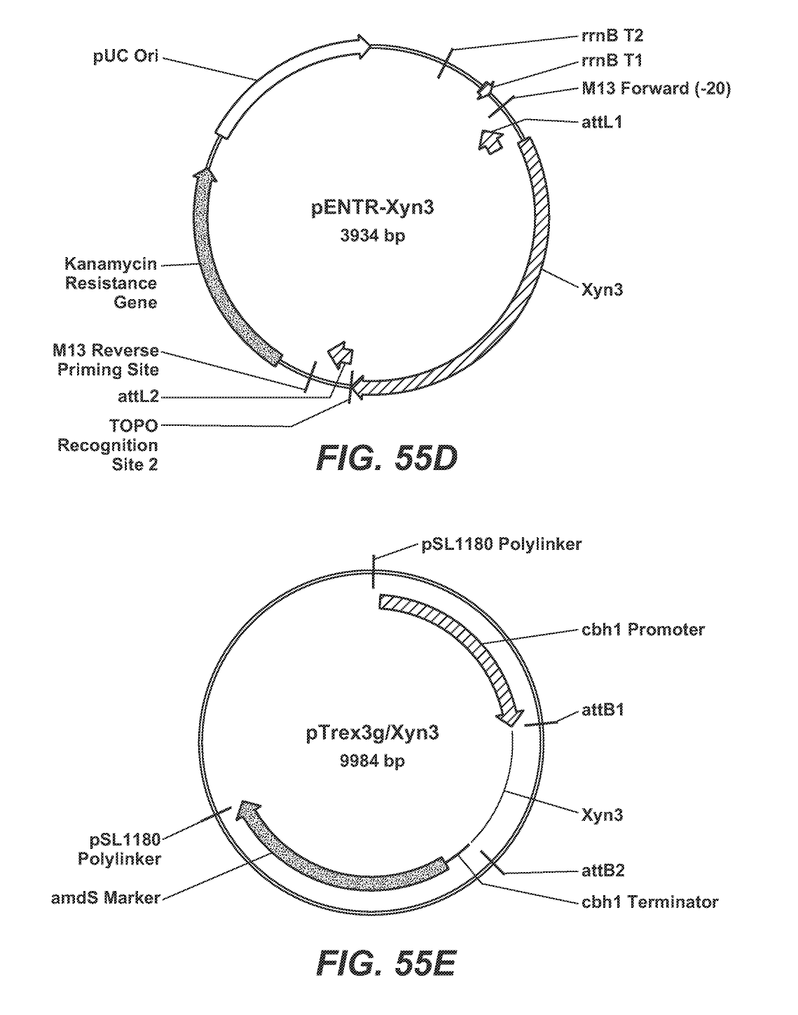

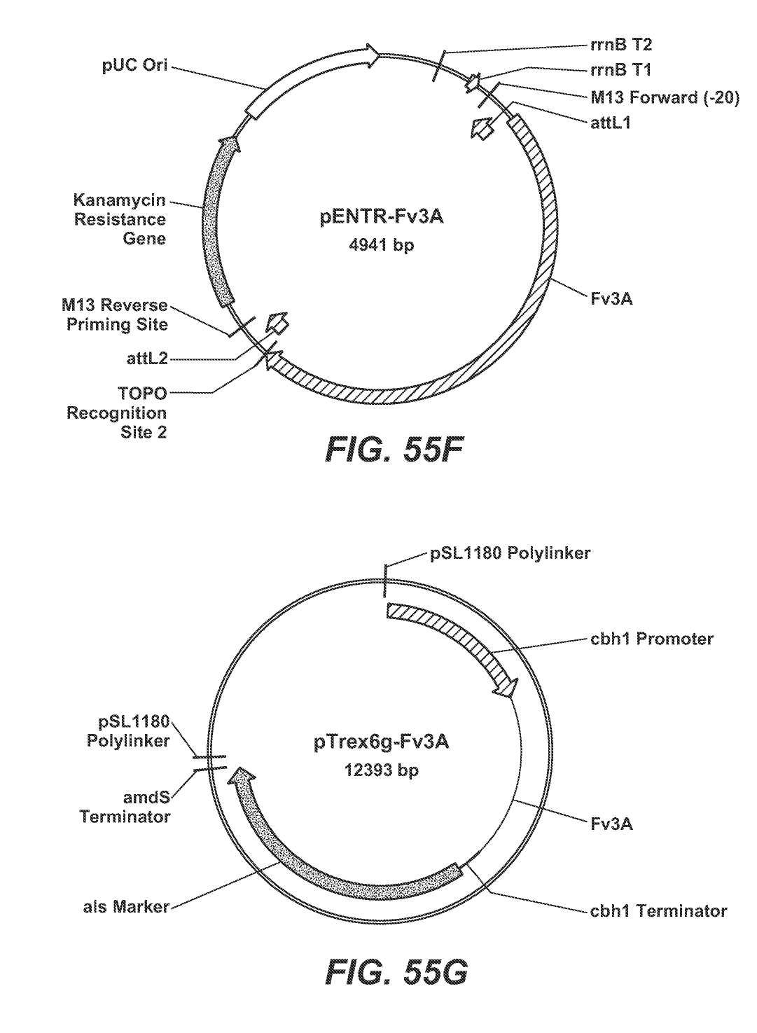

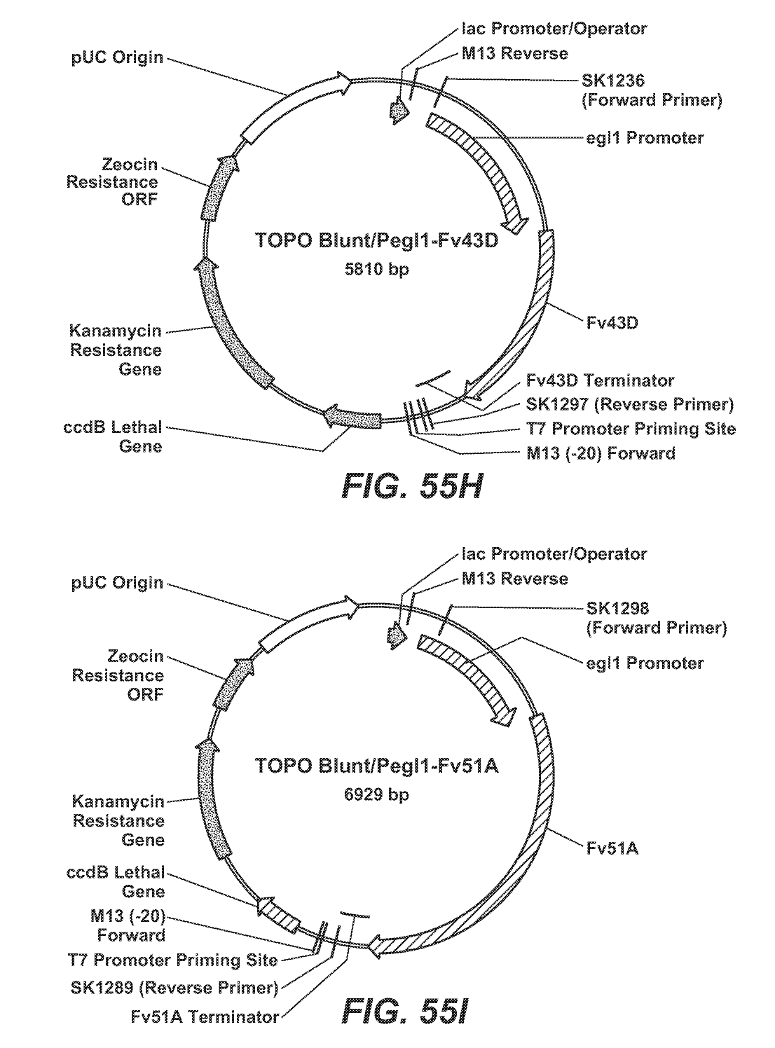

The resulting fusion PCR fragments were cloned into the Gateway® Entry vector pENTR™/D-TOPO®, and transformed into E. coli One Shot® TOP10 Chemically Competent cells (Invitrogen) resulting in the intermediate vector, pENTR TOPO-Bgl1(943/942) (FIG. 55B). The nucleotide sequence of the inserted DNA was determined. The pENTR-943/942 vector with the correct bgl1 sequence was recombined with pTrex3g using a LR Clonase® reaction (see, protocols outlined by Invitrogen). The LR clonase reaction mixture was transformed into E. coli One Shot® TOP10 Chemically Competent cells (Invitrogen), resulting in the expression vector, pTrex3g 943/942 (map see, FIG. 55C). The vector also contained the Aspergillus nidulans amdS gene, encoding acetamidase, as a selectable marker for transformation of T. reesei. The expression cassette was PCR amplified with primers SK745 and SK771 (below) to generate the product for transformation.

Forward Primer SK771:
(SEQ ID NO: 96)
(5′- GTCTAGACTGGAAACGCAAC -3′)
Reverse Primer SK745:
(SEQ ID NO: 97)
(5′- GAGTTGTGAAGTCGGTAATCC -3′)

### 1) Construction of the Endoxylanase Expression Cassette

The native T. reesei endoxylanase gene xyn3 was PCR amplified from a genomic DNA sample extracted from T. reesei, using primers xyn3F-2 and xyn3R-2.

Forward Primer xyn3F-2:
(SEQ ID NO: 98)
(5′-CACCATGAAAGCAAACGTCATCTTGTGCCTCCTGG-3′)
Reverse Primer xyn3R-2:
(SEQ ID NO: 99)
(5′-CTATTGTAAGATGCCAACAATGCTGTTATATGCCG
GCTTGGGG-3′)

The resulting PCR fragments were cloned into the Gateway® Entry vector pENTR™/D-TOPO®, and transformed into E. coli One Shot® TOP10 Chemically Competent Cells, resulting in a vector as shown in FIG. 55D. The nucleotide sequence of the inserted DNA was determined. The pENTR/Xyn3 vector with the correct xyn3 sequence was recombined with pTrex3g using a LR Clonase® reaction protocol (Invitrogen). The LR Clonase® reaction mixture was than transformed into E. coli One Shot® TOP10 Chemically Competent cells (Invitrogen), resulting in the final expression vector, pTrex3g/Xyn3 (see, FIG. 55E). The vector also contains the Aspergillus nidulans amdS gene, encoding acetamidase, as a selectable marker for transformation of T. reesei. The expression cassette was PCR amplified with primers SK745 and SK822 (below) to generate product for transformation.

(SEQ ID NO: 100)
Forward Primer SK745: (5′-GAGTTGTGAAGTCGGTAATCC-3′)
(SEQ ID NO: 101)
Reverse Primer SK822: (5′-CACGAAGAGCGGCGATTC-3′)

### 2) Construction of the β-Xylosidase Fv3A Expression Vector

The F. verticillioides β-xylosidase fv3A gene was amplified from a F. verticilloides genomic DNA sample using the primers MH124 and MH125.

Forward Primer MH124:
(SEQ ID NO: 102)
(5′-CACCCATGCTGCTCAATCTTCAG-3′)
Reverse Primer MH125:
(SEQ ID NO: 103)
(5′-TTACGCAGACTTGGGGTCTTGAG-3′)

The PCR fragments were cloned into the Gateway® Entry vector pENTR™/D-TOPO®, and transformed into E. coli One Shot® TOP10 Chemically Competent cells (Invitrogen) resulting in the intermediate vector, pENTR-Fv3A (see, FIG. 55F). The nucleotide sequence of the inserted DNA was determined. The pENTR-Fv3A vector with the correct fv3A sequence was recombined with pTrex6g using the LR Clonase® reaction protocol (Invitrogen). The LR Clonase® reaction mixture was transformed into E. coli One Shot® TOP10 Chemically Competent cells (Invitrogen), resulting in the final expression vector, pTrex6g/Fv3A (see, FIG. 55G). The vector also contained a chlorimuron ethyl resistant mutant of the native T. reesei acetolactate synthase (als) gene, alsR, which was used together with its native promoter and terminator as a selectable marker for transformation of T. reesei in accordance with the method described in International Publication WO2008/039370 A1. The expression cassette was PCR amplified using primers SK1334, SK1335 and SK1299 (below) to generate product for transformation.

Forward Primer SK1334:
(SEQ ID NO: 104)
(5′-GCTTGAGTGTATCGTGTAAG-3′)
Forward Primer SK1335:
(SEQ ID NO: 105)
(5′-GCAACGGCAAAGCCCCACTTC-3′)
Reverse Primer SK1299:
(SEQ ID NO: 106)
(5′-GTAGCGGCCGCCTCATCTCATCTCATCCATCC-3′)

### 3) Construction of the 1-Xylosidase Fv43D Expression Cassette

For the construction of the F. verticillioides β-xylosidase Fv43D expression cassette, the fv43D gene product was amplified from a F. verticillioides genomic DNA sample using the primers SK1322 and SK1297 (below). A region of the promoter of the endoglucanase gene egl1 was PCR amplified from a T. reesei genomic DNA sample extracted from strain RL-P37, using the primers SK1236 and SK1321 (below). These PCR amplified DNA fragments were subsequently fused in a fusion PCR reaction using the primers SK1236 and SK1297 (below). The resulting fusion PCR fragment was cloned into pCR-Blunt II-TOPO vector (Invitrogen) to produce the plasmid TOPO Blunt/Pegl1-Fv43D (see, FIG. 55H). This plasmid was then used to transform E. coli One Shot® TOP10 Chemically Competent cells (Invitrogen). The plasmid DNA was extracted from several E. coli clones and their sequences were confirmed by restriction digests.

Forward Primer SK1322:
(SEQ ID NO: 107)
(5′-CACCATGCAGCTCAAGTTTCTGTC-3′)
Reverse Primer SK1297:
(SEQ ID NO: 108)
(5′-GGTTACTAGTCAACTGCCCGTTCTGTAGCGAG-3′)
Forward Primer SK1236:
(SEQ ID NO: 109)
(5′-CATGCGATCGCGACGTTTTGGTCAGGTCG-3′)
Reverse Primer SK1321:
(SEQ ID NO: 110)
(5′-GACAGAAACTTGAGCTGCATGGTGTGGGACAACAAGAAGG-3′)

The expression cassette was PCR amplified from the TOPO Blunt/Pegl1-Fv43D using primers SK1236 and SK1297 (above) to generate the product for transformation.

### 4) Construction of the α-Arabinofuranosidase Expression Cassette

For the construction of the F. verticillioides α-arabinofuranosidase gene fv51A expression cassette, the fv51A gene product was amplified from a F. verticillioides genomic DNA sample using the primers SK1159 and SK1289 (below). A region of the promoter of the endoglucanase gene egl1 was PCR amplified from a T. reesei genomic DNA sample extracted from strain RL-P37 (supra), using the primers SK1236 and SK1262 (below). The PCR amplified DNA fragments were then fused in a fusion PCR reaction using the primers SK1236 and SK1289 (below). The resulting fusion PCR fragment was cloned into pCR-Blunt II-TOPO vector (Invitrogen) to produce the plasmid TOPO Blunt/Pegl1-Fv51A (see, FIG. 55I) and E. coli One Shot® TOP10 Chemically Competent cells (Invitrogen) were transformed using this plasmid.

Forward Primer SK1159:
(SEQ ID NO: 111)
(5′-CACCATGGTTCGCTTCAGTTCAATCCTAG-3′)
Reverse Primer SK1289:
(SEQ ID NO: 112)
(5′-GTGGCTAGAAGATATCCAACAC-3′)
Forward Primer SK1236:
(SEQ ID NO: 113)
(5′-CATGCGATCGCGACGTTTTGGTCAGGTCG-3′)
Reverse Primer SK1262:
(SEQ ID NO: 114)
(5′-GAACTGAAGCGAACCATGGTGTGGGACAACAAGAAGGAC-3′)

The expression cassette was PCR amplified with primers SK1298 and SK1289 (above) to generate the product for transformation.

Forward Primer SK1298:
(SEQ ID NO: 115)
(5′-GTAGTTATGCGCATGCTAGAC-3′)
Reverse Primer SK1289:
(SEQ ID NO: 112)
(5′-GTGGCTAGAAGATATCCAACAC-3′)

5) Co-Transformation of T. Reesei with the β-Glucosidase and Endoxylanase Expression Cassettes

A Trichoderma reesei mutant strain, derived from RL-P37 (Sheir-Neiss, G et al. Appl. Microbiol. Biotechnol. 1984, 20:46-53.) and selected for high cellulase production was co-transformed with the β-glucosidase expression cassette (cbh1 promoter, T. reesei beta-glucosidase1 gene, cbh1 terminator, and amdS marker), and the endoxylanase expression cassette (cbh1 promoter, T. reesei xyn3, and cbh1 terminator) using a PEG-mediated transformation method (see, Penttila, M et al. Gene 1987, 61(2):155-64). A number of transformants were isolated and examined for β-glucosidase and endoxylanase production. One transformant called T. reesei strain #229 was selected for transformation with the other expression cassettes.

6) Co-Transformation of T. Reesei Strain #229 with Two β-Xylosidase and α-Arabinofuranosidase Expression Cassettes

T. reesei strain #229 was co-transformed with the β-xylosidase fv3A expression cassette (cbh1 promoter, fv3A gene, cbh1 terminator, and alsR marker), the β-xylosidase fv43D expression cassette (egl1 promoter, fv43D gene, native fv43D terminator), and the fv51A α-arabinofuranosidase expression cassette (egl1 promoter, fv51A gene, fv51A native terminator) using electroporation in accordance with, e.g., International Publication WO2008153712A2. Transformants were selected on Vogels agar plates containing chlorimuron ethyl (80 ppm).

| | | |
|---|---|---|
| 50 x Vogels Stock Solution (recipe) | 20 | mL |
| BBL Agar | 20 | g |
| With deionized H₂O bring to | 980 | mL |
| post-sterile addition: 50% Glucose | 20 | mL |
| 50 x Vogels Stock Solution, per liter: | | |
| In 750 mL deionized H2O, dissolve successively: | | |
| Na₃Citrate*2H₂O | 125 | g |
| KH₂PO₄ (Anhydrous) | 250 | g |
| NH₄NO₃ (Anhydrous) | 100 | g |
| MgSO₄*7H₂O | 10 | g |
| CaCl₂*2H₂O | 5 | g |

| | | |
|---|---|---|
| Vogels Trace Element Solution (recipe below) | 5 | mL |
| d-Biotin | 0.1 | g |
| With deionized H₂O, | bring to 1 | L |
| Vogels Trace Element Solution: | | |
| Citric Acid | 50 | g |
| ZnSO₄•*7H₂O | 50 | g |
| Fe(NH₄)2SO₄•*6H₂O | 10 | g |
| CuSO₄•5H₂O | 2.5 | g |
| MnSO₄•4H₂O | 0.5 | g |
| H₃BO₃ | 0.5 | g |
| Na₂MoO₄•2H₂O | 0.5 | g |

A number of transformants were isolated and examined for β-xylosidase and L-α-arabinofuranosidase production. Transformants were also screened for biomass conversion performance according to the cob saccharification assay as described in Example 1. Examples of T. reesei integrated expression strains described herein are selected from H3A, 39A, A10A, 11A, and G9A, which expressed the T. reesei genes encoding beta-glucosidase 1, Xyn3, and Fusarium genes encoding Fv3A, Fv51A, and Fv43D, at different ratios. A particular H3A strain, #5 (“H3A-5”) expressed a lower level of T. reesei Bgl1 as compared with the other H3A strains, was used in an experiment described herein below. Another H3A strain expressing a reduced level of T. reesei Bgl1 was used in the experiment described in Example 5. Among others, one T. reesei strain lacked overexpressed T. reesei Xyn3; another lacked Fv51A, and two lacked Fv3A, as determined by Western Blot.

7) Composition of T. reesei Integrated Strain H3A

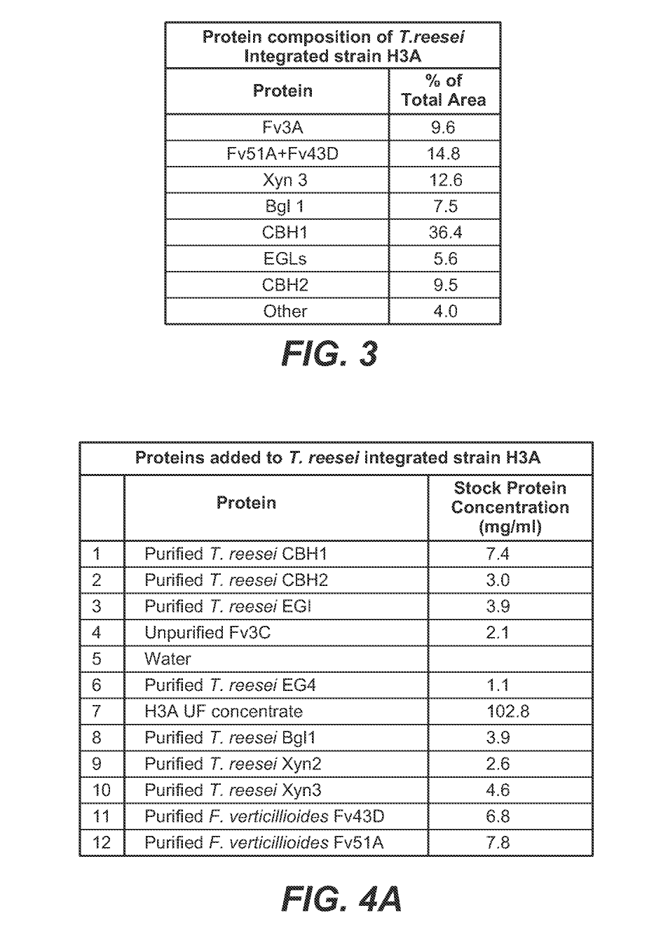

Fermentation of the T. reesei integrated strain H3A and compositional determination identified the existence of the following gene products: T. reesei Xyn3, T. reesei Bgl 1, Fv3A, Fv51A, and Fv43D, at ratios shown in FIG. 3 herein.

### 8) Protein Analysis by HPLC

Liquid chromatography (LC) and mass spectroscopy (MS) were performed to separate and quantify the enzymes contained in fermentation broths. Enzyme samples were first treated with a recombinantly expressed endoH glycosidase from S. plicatus (e.g., NEB P0702L). EndoH was used at an amount of 0.01-0.0314 endoH per 1 μg of total protein in the sample. The mixtures were incubated for 3 h at 37° C., pH 4.5-6.0 to enzymatically remove N-linked gycosylation prior to HPLC analysis. About 5014 of protein was then subject to hydrophobic interaction chromatography (Agilent 1100 HPLC) using an HIC-phenyl column and a high-to-low salt gradient over 35 min. The gradient was achieved using high salt buffer A: 4 M ammonium sulphate containing 20 mM potassium phosphate, pH 6.75; and low salt buffer B: 20 mM potassium phosphate, pH 6.75. Peaks were detected at UV 222 nm. Fractions were collected and analyzed using mass spectroscopy. Protein ratios are reported as the percent of each peak area relative to the total integrated area of the sample.

9) Effect of Addition of Purified Proteins to the Fermentation Broth of T. reesei Integrated Strain H3A on Saccharification of Dilute Ammonia Pretreated Corncob

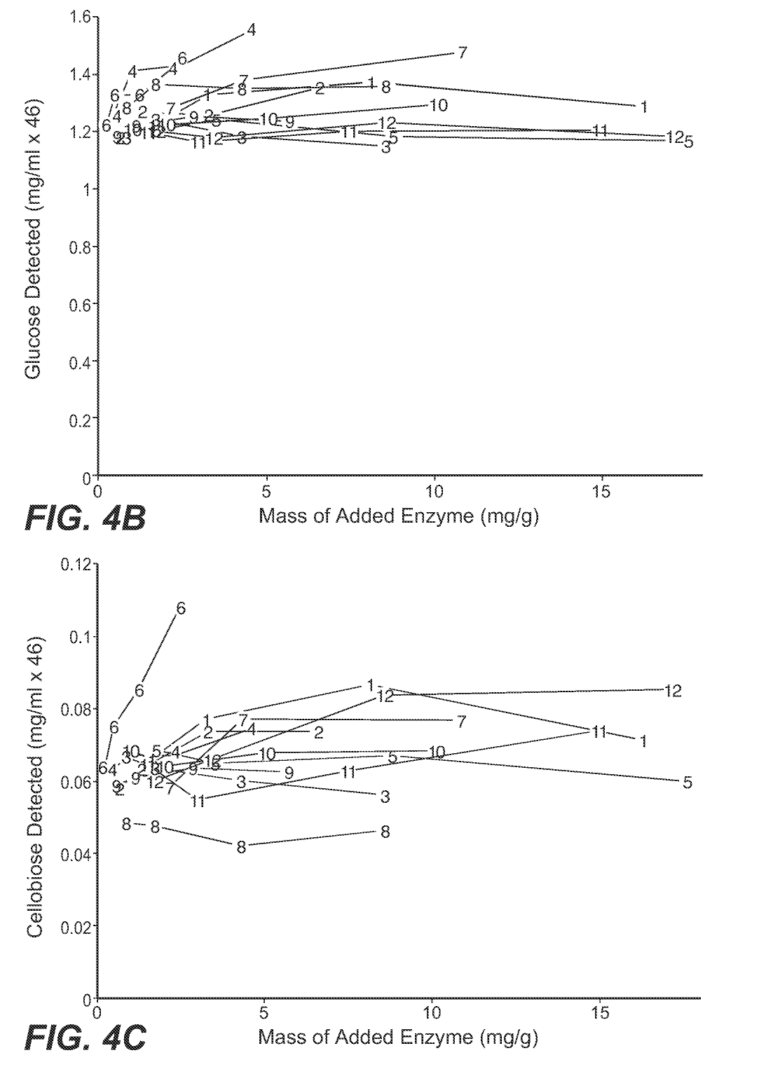

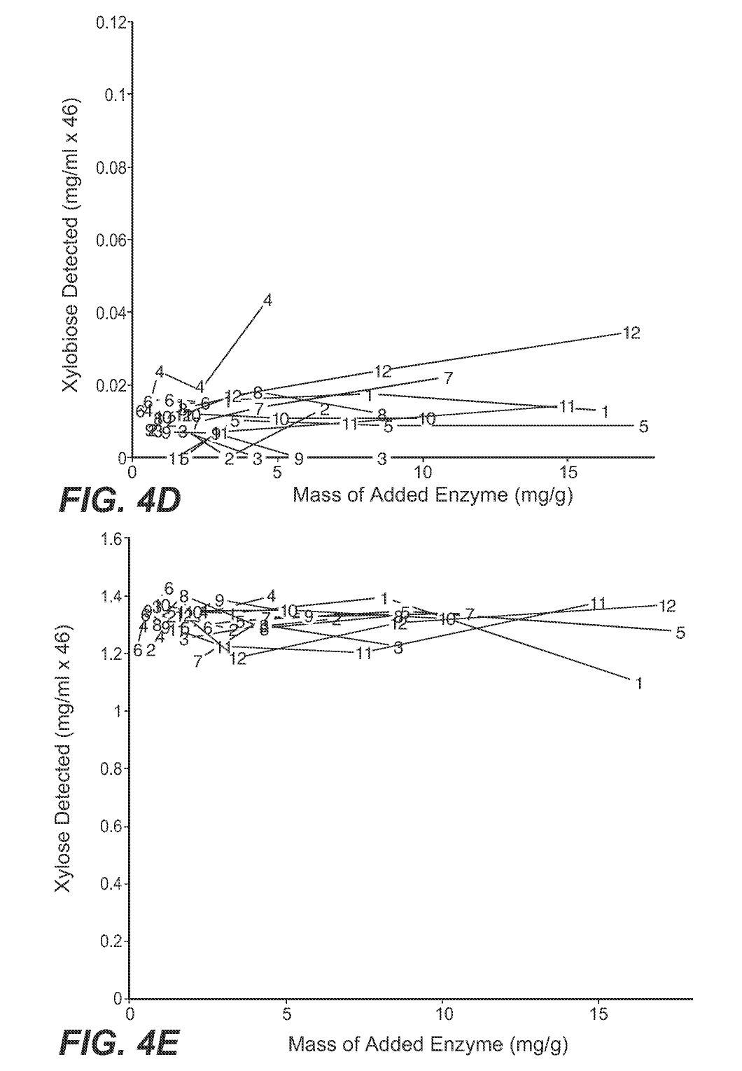

This experiment assessed the benefits conferred by various enzymes (mostly purified but also an unpurified enzyme) to the saccharification of pretreated biomass. Purified proteins and one unpurified protein were serially diluted from the stock solution and added to a fermentation broth of T. reesei integrated strain H3A. Dilute ammonia pretreated corncob was loaded into 96-well microtiter plate wells at 20% solids (w/w) (−5 mg of cellulose per well), pH 5. An H3A fermentation broth was added to each well at 20 mg protein/g cellulose. Volumes of 10, 5, 2, and 1 μL of each of the diluted proteins (FIG. 4A) were added into individual wells, and water was also added such that the liquid addition to an individual well totaled 10 μL. The reference wells included additions of either 10 μL water or dilutions of additional H3A. The microtiter plates were sealed with foil and incubated at 50° C., shaking at a rate of 200 rpm in an Innova incubator shaker for 3 d. The samples were quenched with 100 μL of 100 mM glycine pH 10. The plate was then covered with a plastic seal and centrifuged at 3,000 rpm for 5 min at 4° C. An aliquot of 5 μL of the quenched reaction mixture was diluted using 100 μL of water. The concentration of glucose produced in the reactions was determined using HPLC. The glucose yield was measured as a function of the protein concentration added to the 20 mg/g of H3A. Results are shown in FIGS. 4B-4E.

### Example 3

### Cloning, Expression and Purification of Fv3C

A. Cloning and Expression of Fv3C

Fv3C sequence (SEQ ID NO:60) was obtained by searching for GH3 β-glucosidase homologs in the Fusarium verticillioides genome in the Broad Institute database (http://www.broadinstitute.org/) The Fv3C open reading frame was amplified by PCR using purified genomic DNA from Fusarium verticillioides as the template. The PCR thermocycler used was DNA Engine Tetrad 2 Peltier Thermal Cycler (Bio-Rad Laboratories). The DNA polymerase used was PfuUltra II Fusion HS DNA Polymerase (Stratagene). The primers used to amplify the open reading frame were as follows:

(SEQ ID NO: 116)
Forward primer MH234 (5′-CACCATGAAGCTGAATTGGGTCGC-3′)
(SEQ ID NO: 117)
Reverse primer MH235 (5′-TTACTCCAACTTGGCGCTG-3′)

The forward primers included four additional nucleotides (sequences—CACC) at the 5′-end to facilitate directional cloning into pENTR/D-TOPO (Invitrogen, Carlsbad, Calif.). The PCR conditions for amplifying the open reading frames were as follows: Step 1: 94° C. for 2 min. Step 2: 94° C. for 30 sec. Step 3: 57° C. for 30 sec. Step 4: 72° C. for 60 sec. Steps 2, 3 and 4 were repeated for an additional 29 cycles. Step 5: 72° C. for 2 min. The PCR product of the Fv3C open reading frame was purified using a Qiaquick PCR Purification Kit (Qiagen). The purified PCR product was initially cloned into the pENTR/D-TOPO vector, transformed into TOP10 Chemically Competent E. coli cells (Invitrogen) and plated on LA plates containing 50 ppm kanamycin. Plasmid DNA was obtained from the E. coli transformants using a QIAspin plasmid preparation kit (Qiagen). Sequence confirmation for the DNA inserted in the pENTR/D-TOPO vector was obtained using M13 forward and reverse primers and the following additional sequencing primers:

| | |
|---|---|
| MH255 (5′-AAGCCAAGAGCTTTGTGTCC-3′) | (SEQ ID NO: 118) |
| MH256 (5′-TATGCACGAGCTCTACGCCT-3′) | (SEQ ID NO: 119) |
| MH257 (5′-ATGGTACCCTGGCTATGGCT-3′) | (SEQ ID NO: 120) |
| MH258 (5′-CGGTCACGGTCTATCTTGGT-3′) | (SEQ ID NO: 121) |

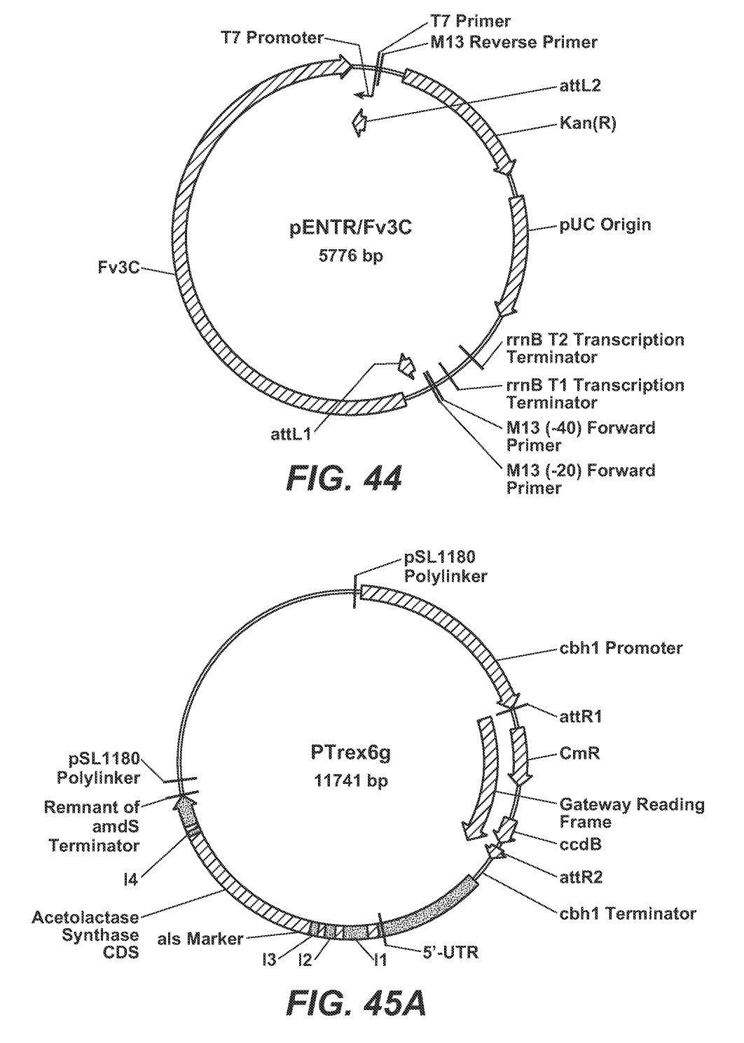

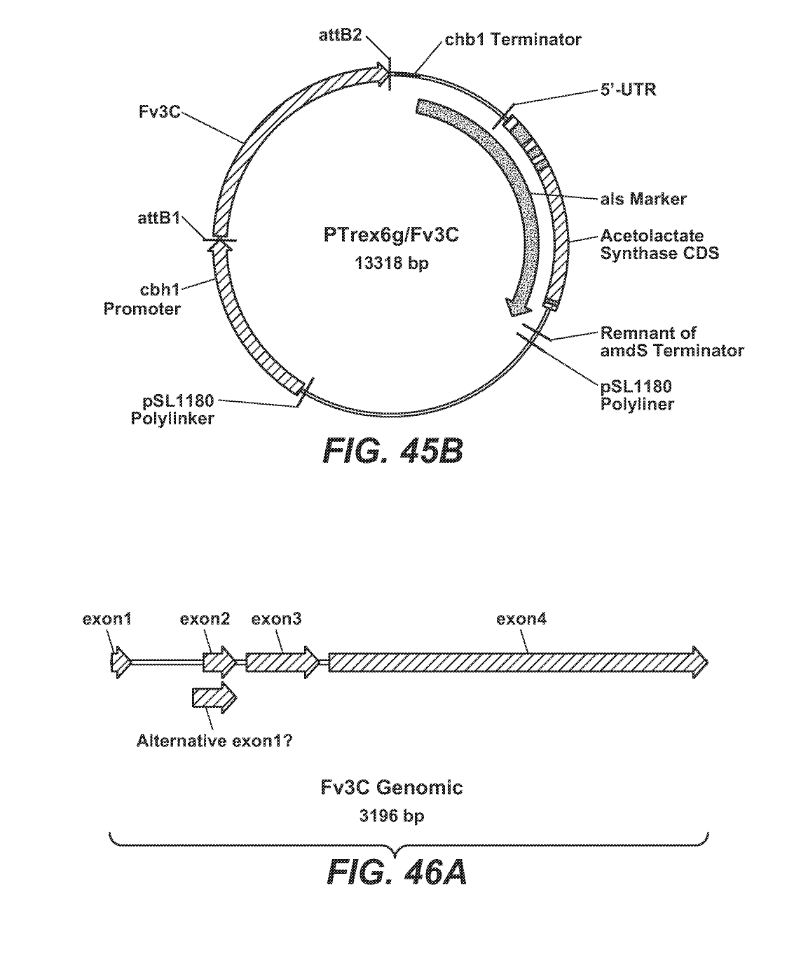

A pENTR/D-TOPO vector with the correct DNA sequence of the Fv3C open reading frame (FIG. 44) was recombined with the pTrex6g (FIG. 45A) destination vector using LR Clonase® reaction mixture (Invitrogen).

The product of the LR Clonase® reaction was subsequently transformed into TOP10 Chemically Competent E. coli cells (Invitrogen), which were then plated onto LA plates containing 50 ppm carbenicillin. The resulting pExpression construct was pTrex6g/Fv3C (FIG. 45B) containing the Fv3C open reading frame and the T. reesei mutated acetolactate synthase selection marker (als). DNA of the pExpression construct containing the Fv3C open reading frame was isolated using a Qiagen miniprep kit and used for biolistic transformation of T. reesei spores.

Biolistic transformation of T. reesei with the pTrex6g expression vector containing the appropriate Fv3C open reading frame was performed. Specifically, a T. reesei strain wherein cbh1, cbh2, eg1, eg2, eg3, and bgl1 have been deleted (i.e., the hexa-delete strain, see, International Publication WO 05/001036) was transformed by helium-bombardment using a Biolistic® PDS-1000/he Particle Delivery System (Bio-Rad) following the manufacturer's instructions (see US 2006/0003408). Transformants were transferred to fresh chlorimuron ethyl selection plates. Stable transformants were inoculated into filter microtiter plates (Corning), containing 200 μL/well of a glycine minimal medium (containing 6.0 g/L glycine; 4.7 g/L (NH₄)₂SO₄; 5.0 g/L KH₂PO₄; 1.0 g/L MgSO₄.7H₂O; 33.0 g/L PIPPS, pH 5.5) with post sterile addition of ˜2% glucose/sophorose mixture as the carbon source, 10 mL/L of 100 g/L of CaCl₂, 2.5 mL/L of a 400× T. reesei trace elements solution containing: 175 g/L Citric acid anhydrous; 200 g/L FeSO₄.7H₂O; 16 g/L ZnSO₄.7H₂O; 3.2 g/L CuSO₄.5H₂O; 1.4 g/L MnSO₄.H₂O; 0.8 g/L H₃BO₃. Transformants were grown in the liquid culture for five days in an O₂-rich chamber housed in a 28° C. incubator. The supernatant samples from the filter microtiter plate were collected on a vacuum manifold. Supernatant samples were run on 4-12% NuPAGE gels and stained using the Simply Blue stain (Invitrogen).

### B. Purification of Fv3C

Fv3C, from shake flask concentrate, was dialyzed overnight against a 25 mM TES buffer, pH 6.8. The dialyzed enzyme solution was loaded on a SEC HiLoad Superdex 200 Prep Grade cross-linked agarose and dextran column (GE Healthcare) at a flow rate of 1 mL/min, which had been pre-equilibrated with 25 mM TES, 0.1 M sodium chloride at pH 6.8. SDS-PAGE was used to identify and ascertain the presence of Fv3C in the fractions from the SEC separation. Fractions containing Fv3C were pooled and concentrated. The SEC purification was also used to separate Fv3C from low and high molecular mass contaminants. The purity of the enzyme preparation was determined using Coomassie blue stained SDS/PAGE. The SDS/PAGE showed a single major band at 97 kDa.

### C. Alternative Translation of Fv3C

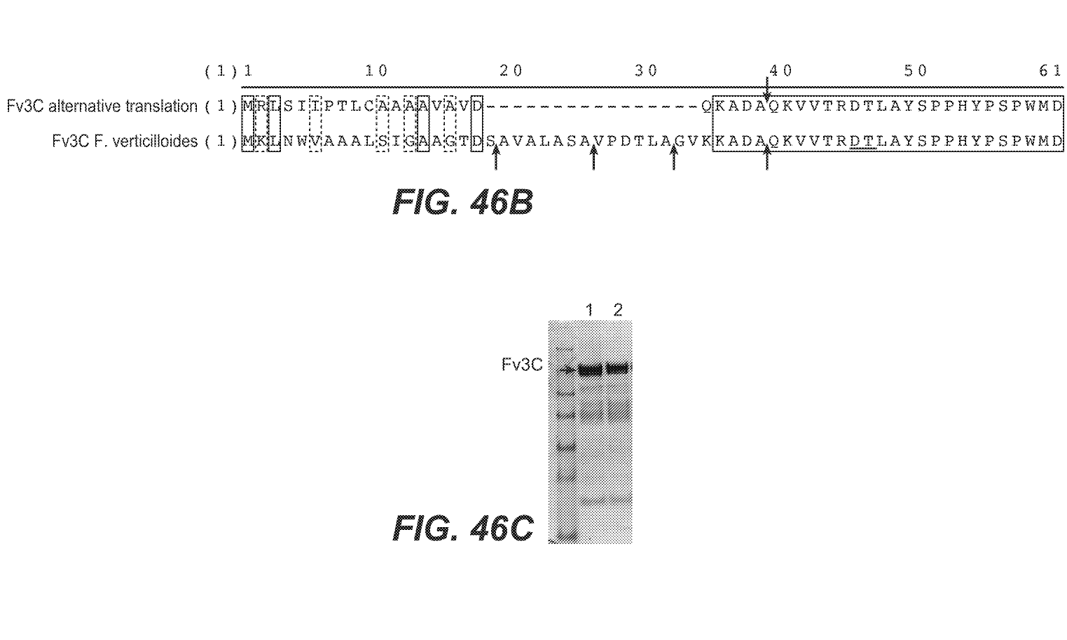

For expression of the Fv3C gene, the genomic sequence containing the ORF as annotated in the Fusarium database was used. http://www.broadinstitute.org/annotation/genome/fusarium_group/MultiHome.html. The predicted coding region contains 3 introns, with the first intron interrupting the signal peptide sequence (FIG. 46A).

However, at its 3′ part, the first intron contained an alternative ORF, in frame with the mature sequence, which is also predicted to code for a signal peptide (FIG. 46B). In both translations, the start site for the mature protein (underlined in FIG. 46B), as determined by N-terminal sequence analysis, started downstream from both putative signal peptide cleavage sites (shown by arrows). It was shown that Fv3C could be effectively expressed by using either of the ATGs as putative starts of translation (FIG. 46C).

### Example 4

### β-Glucosidase Activity on Cellobiose and Cnpg

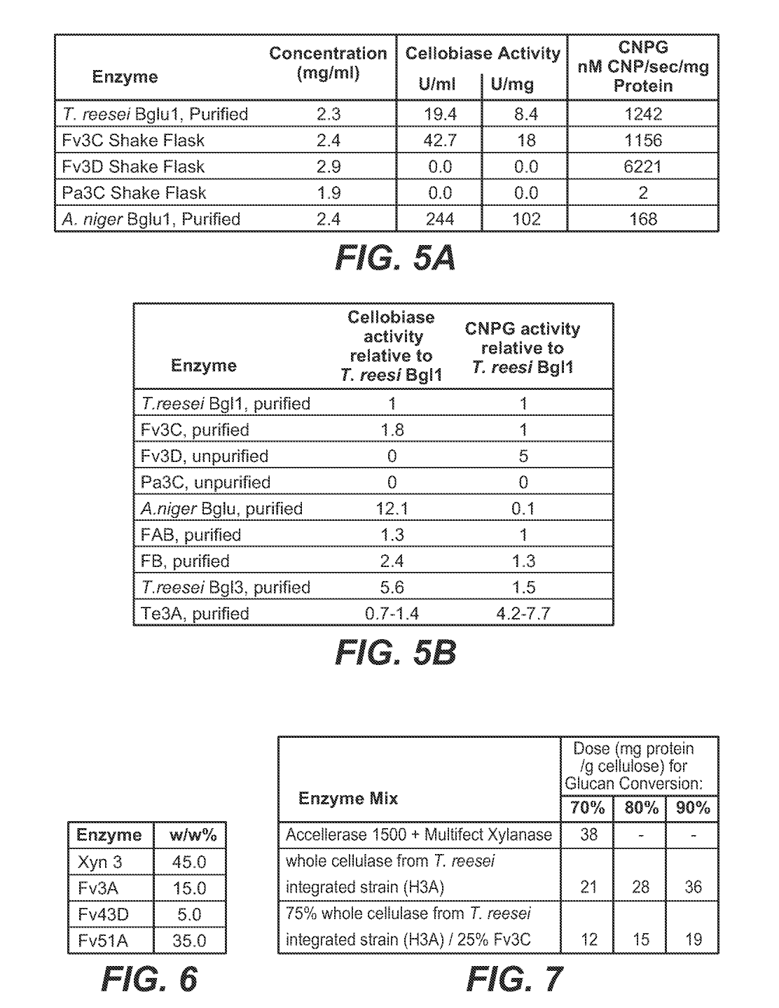

In this experiment, the β-glucosidase activities of T. reesei Bgl1, A. niger Bglu (An3A) (Megazyme International Ireland Ltd., Wicklow, Ireland), Fv3C (SEQ ID NO:60), Fv3D (SEQ ID NO:58), and Pa3C (SEQ ID NO:80) on cellobiose and CNPG were tested. T. reesei Bgl1, A. niger Bglu (“An3A”), Fv3C, Fv3C/Te3A/Bgl3 (FAB) chimera, Fv3C/Bgl3 (FB) chimera, T. reesei Bgl3, and Te3A were purified proteins. Fv3D and Pa3C were not purified proteins. They were expressed in a T. reesei hexa-delete strain (as defined above), but some background protein activities were still present. As shown in FIG. 5A, Fv3C was found to have about twice the activity of T. reesei Bgl1 on cellobiose, whereas A. niger Bglu was found to be about 12 times more active than T. reesei Bgl1.

Activity of Fv3C on the CNPG substrate was about equal to that of T. reesei Bgl1, but the activity of A. niger Bglu was about 14% of the activity of T. reesei Bgl1 (FIG. 5A). Fv3D, another Fusarium verticillioides beta-glucosidase expressed similarly to Fv3C, had no measurable cellobiase activity, yet its activity on CNPG was about 5 times that of T. reesei Bgl1. In addition, a similarly produced P. anserina beta-glucosidase homolog Pa3C had no measurable activity on cellobiose or CNPG substrate. These studies demonstrate that the activities of Fv3C on cellobiose and CNPG were due to the molecule itself and were not due to background protein activities.

### Example 5

### Fv3C Saccharification on Various Biomass Substrates

### A. Fv3C Saccharification Performance on PASC

In this experiment, the ability of T. reesei Bgl1, Fv3C, and several Fv3C homologs to enhance PASC saccharification was tested. Twenty (20) μL of each beta-glucosidase was added in an amount of 5 mg protein/g cellulose to a 10 mg protein/g cellulose loading of whole cellulase from a T. reesei bgl1-reduced strain, in a 96-well HPLC plate. One hundred and fifty (150) μL of a 0.7% solids slurry of PASC was added to each well and the plates were covered with aluminum plate sealers and placed in an incubator set at 50° C. for 2 h with shaking. The reaction was terminated by adding 100 μL of a 100 mM glycine buffer, pH10 to individual wells. After thorough mixing, the plates were centrifuged and the supernatants were diluted 10 fold into another HPLC plate, which contained 100 μL of 10 mM glycine, pH 10 in individual wells. The concentrations of soluble sugars produced were measured using HPLC (FIG. 47).

It was observed that the Fv3C-containing mixture yielded a higher proportion of glucose than the T. reesei Bgl1-containing mixture under the same conditions. This indicated that Fv3C has a higher cellobiase activity than T. reesei Bgl1 (see also FIG. 5B). Fv3G, Pa3D and Pa3G had no observable effect on PASC hydrolysis, which indicated the lack of contribution from the hexa-delete background (in which the various Fv3C homologs were cloned and expressed) on PASC hydrolysis.

### B. Fv3C Saccharification Performance on Dilute Acid Pretreated Cornstover (PCS)

In this experiment, the abilities of T. reesei Bgl1, Fv3C, and several Fv3C homologs to enhance PCS saccharification at 13% solids was tested using the method described in the Microtiter plate Saccharification assay (supra). For each enzyme tested, 5 mg protein/g cellulose of beta-glucosidase was added to 10 mg protein/g cellulose of a whole cellulase derived from a T. reesei-Bgl1 reduced strain.

Specifically, 5 mg protein/g cellulose of each of the beta-glucosidases (Bgl1, Fv3C, and homologs) was added to 10 mg protein/g cellulose of a whole cellulase derived from a T.

reesei Bgl1 reduced strain, or to 8 mg protein/g cellulose of a purified hemicellulase mixture (the components of which are indicated in FIG. 6). The % glucan conversion was measured after the enzymatic mixtures were incubated with the substrate for 2 d at 50° C.

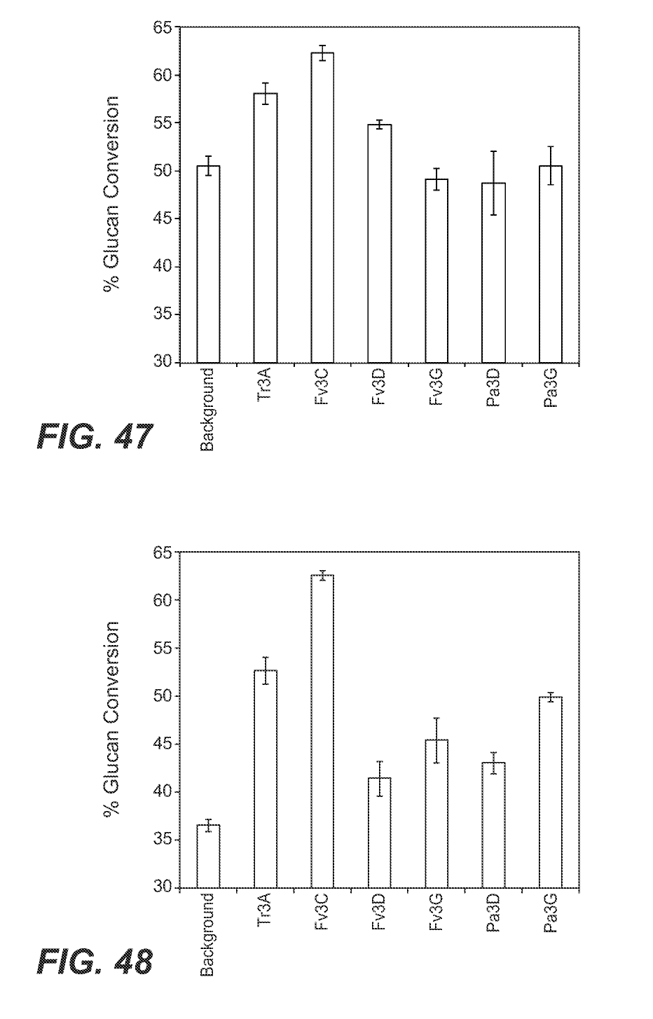

Results are shown in FIG. 48. It has also been observed that Fv3C imparted a clear benefit in terms of % glucan conversion as compared to T. reesei Bgl1. In addition, Fv3C also promoted higher glucose and total sugar yields than T. reesei Bgl1.

The results indicated limited if any contribution from host cell background proteins.

### C. Fv3C Saccharification Performance on Dilute Ammonia Pretreated Corncob

In this experiment, the ability of T. reesei Bgl1, Fv3C, and A. niger Bglu (An3A) to enhance saccharification of ammonia pre-treated corncob at 20% solids was tested in accordance with the method described in the Microtiter Plate Saccharification assay (supra). Specifically, 5 mg protein/g cellulose of beta-glucosidases (e.g., T. reesei Bgl1, Fv3C, and homologs) were added to the dilute ammonia pretreated corncob substrate, and 10 mg protein/g cellulose of whole cellulase derived from a T. reesei Bgl1-reduced strain was also added. In addition, 8 mg protein/g cellulose of a purified hemicellulase mix (FIG. 6) containing Xyn3, Fv3A, Fv43D and Fv51A was also added to the mixture. The % glucan conversion was measured after the enzyme mixtures were incubated with the substrate for 2 d at 50° C.

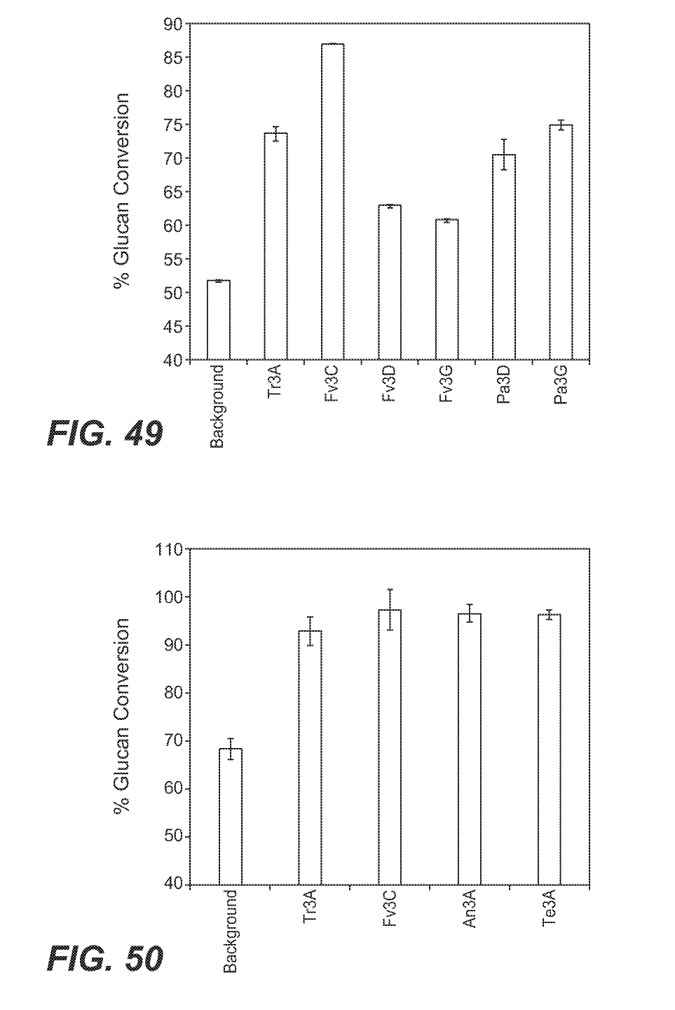

Results are shown in FIG. 49. It was also observed that Fv3C appeared to have performed better than the other beta-glucosidases, including T. reesei Bgl1 (Tr3A). It was additionally observed that A. niger Bglu (An3A) additions to the enzyme mixture to a level above 2.5 mg/g cellulose impeded saccharification.

### D. Fv3C Saccharification Performance on Sodium Hydroxide (NaOH) Pretreated Corncob

To test the effect of various substrate pretreatment methods on Fv3C performance, the ability of T. reesei Bgl1 (also termed Tr3A), Fv3C, and A. niger Bglu (An3A) to enhance saccharification of NaOH pre-treated corncob at 12% solids was measured in accordance with the method described in the Microtiter plate Saccharification assay (supra). Sodium hydroxide pretreatment of corncob was performed as follows: 1,000 g of corncob was milled to about 2 mm in size, and was then suspended in 4 L of 5% aqueous sodium hydroxide solution, and heated to 110° C. for 16 h. The dark brown liquid was filtered hot under laboratory vacuum. The solid residue on the filter was washed with water until no more color eluted. The solid was dried under laboratory vacuum for 24 h. One hundred (100) g of the sample was suspended in 700 mL water and stirred. The pH of the solution was measured to be 11.2. Aqueous citric acid solution (10%) was added to lower the pH to 5.0 and the suspension was stirred for 30 min. The solid was then filtered, washed with water, and dried under vacuum at room temperature for 24 h. After drying, 86.2 g of polysaccharide enriched biomass was obtained. The moisture content of this material was about 7.3 wt %. Glucan, xylan, lignin and total carbohydrate content were measured before and after sodium hydroxide treatment, as determined by the NREL methods for carbohydrate analysis. The pretreatment resulted in delignification of the biomass while maintaining a glucan/xylan weight ration within 15% of that for the untreated biomass.

About 5 mg protein/g cellulose of beta-glucosidases (Fv3C and homologs) were added to the NaOH pretreated substrate, in addition to the inclusion of 8.7 mg protein/g cellulose of a whole cellulase derived from an integrated T. reesei strain H3A specifically selected for its low level of Bgl1 expression (“the H3A-5 strain”). No additional purified hemicellulases (e.g., the mixture of FIG. 6) were added to the whole cellulase background in this experiment. The % glucan conversion was measured after the enzyme mixtures were incubated with the substrate for 2 d at 50° C.

The results are shown in FIG. 50. It was observed that Fv3C appeared to have performed somewhat better than the other beta-glucosidases, including T. reesei Bgl1 (Tr3A), An3A, and Te3A. It has also been observed that additions of A. niger Bglu (An3A) to the level above 4 mg/g cellulose resulted in lower conversion.

### E. Fv3C Saccharification Performance on Dilute Ammonia Pretreated Switchgrass

In this experiment, the ability of T. reesei Bgl1, Fv3C, and A. niger Bglu (An3A) to enhance saccharification of dilute ammonia pretreated switchgrass at 17% solids was tested in accordance with the method described in the Microtiter Plate Saccharification assay (supra). Dilute ammonia pretreated switchgrass was obtained from DuPont. The composition was determined using the National Renewable Energy Laboratory (NREL) procedure, (NREL LAP-002), available at: http://www.nrel.gov/biomass/analytical_procedures.html.

The composition based on dry weight was glucan (36.82%), xylan (26.09%), arabinan (3.51%), lignin-acid insoluble (24.7%), and acetyl (2.98%). This raw material was knife milled to pass a 1 mm screen. The milled material was pretreated at ˜160° C. for 90 min in the presence of 6 wt % (of dry solids) ammonia. Initial solids loading was about 50% dry matter. The treated biomass was stored at 4° C. before use.

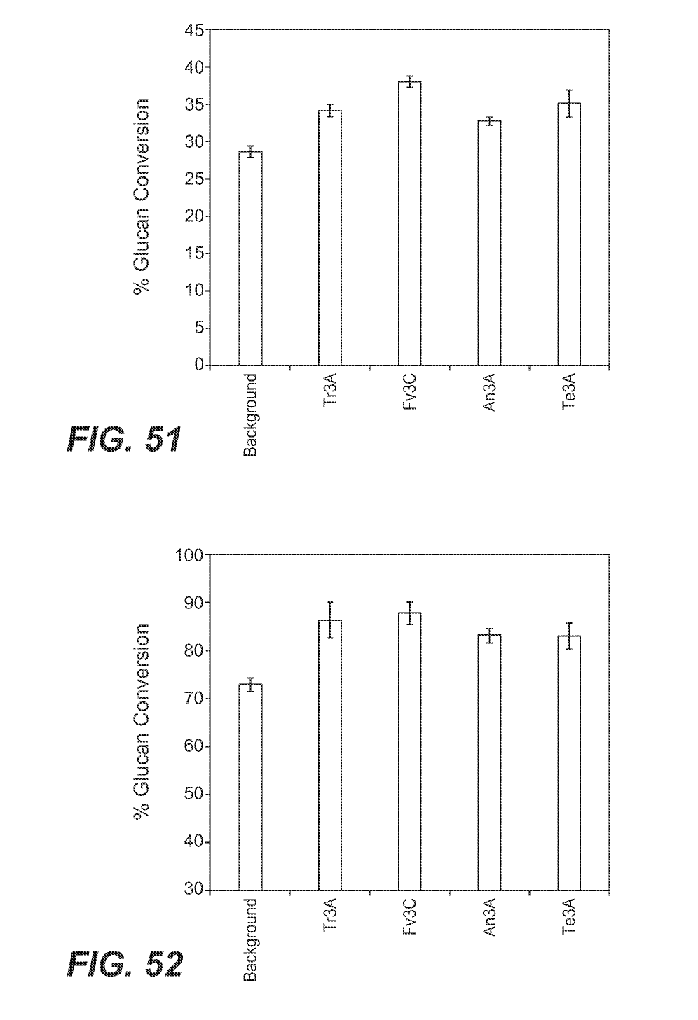

In this experiment, 5 mg protein/g cellulose of beta-glucosidases (e.g., T. reesei Bgl1, Fv3C, and homologs) were added to the dilute ammonia pretreated switchgrass, in the presence of 10 mg protein/g cellulose of a whole cellulase derived from an integrated T. reesei strain (H3A) selected for low β-glucosidase expression. The % glucan conversion was measured after the enzyme mixtures were incubated with the substrate for 2 d at 50° C. and the results are indicated in FIG. 51.

It appeared that Fv3C performed better than the T. reesei Bgl1 and the A. niger Bglu with the switchgrass substrate.

### F. Fv3C Saccharification Performance on AFEX Cornstover

In this experiment, the ability of T. reesei Bgl1, Fv3C, and A. niger Bglu to enhance saccharification of AFEX cornstover at 14% solids was tested in accordance to the method described in the Microtiter Plate Saccharification assay (supra). AFEX pretreated corn stover was obtained from Michigan Biotechnology Institute International (MBI). The composition of the corn stover was determined using the National Renewable Energy Laboratory (NREL) procedure LAP-002, available at: http://www.nrel.gov/biomass/analytical_procedures.html.

The composition based on dry weight was glucan (31.7%), xylan (19.1%), galactan (1.83%), and arabinan (3.4%). This raw material was AFEX treated in a 5 gallon pressure reactor (Parr) at 90° C., 60% moisture content, 1:1 biomass to ammonia loading, and for 30 min. The treated biomass was removed from the reactor and left in a fume hood to evaporate the residual ammonia. The treated biomass was stored at 4° C. before use.

In this experiment, about 5 mg protein/g cellulose of beta-glucosidases (Fv3C and homologs) were added to the pretreated substrate, in the presence of 10 mg protein/g cellulose of whole cellulase derived from a low β-glucosidase expressing integrated T. reesei strain (see FIG. 3). The % glucan conversion was measured after the enzyme mixtures were incubated with the substrate for 2 d at 50° C., and the results were indicated in FIG. 52.

It was observed that Fv3C performed better than T. reesei Bgl1 at glucan conversion. It was also noted that 10 mg/g cellulose of Fv3C and 10 mg/g cellulose of H3A whole cellulase under the above conditions resulted in a complete or an apparently complete glucan conversion. At levels below 1 mg/g cellulose, the A. niger Bglu (An3A) appeared to give higher glucose and total glucan conversions than that of Fv3C and T. reesei Bgl1, but at levels above 2.5 mg/g cellulose, it was observed that Fv3C and T. reesei Bgl1 had higher glucose and glucan conversion than A. niger Bglu.

### Example 6

### Optimization of Fv3C to Whole Cellulase Ratio for Dilute Ammonia Pretreated Corncob Saccharification

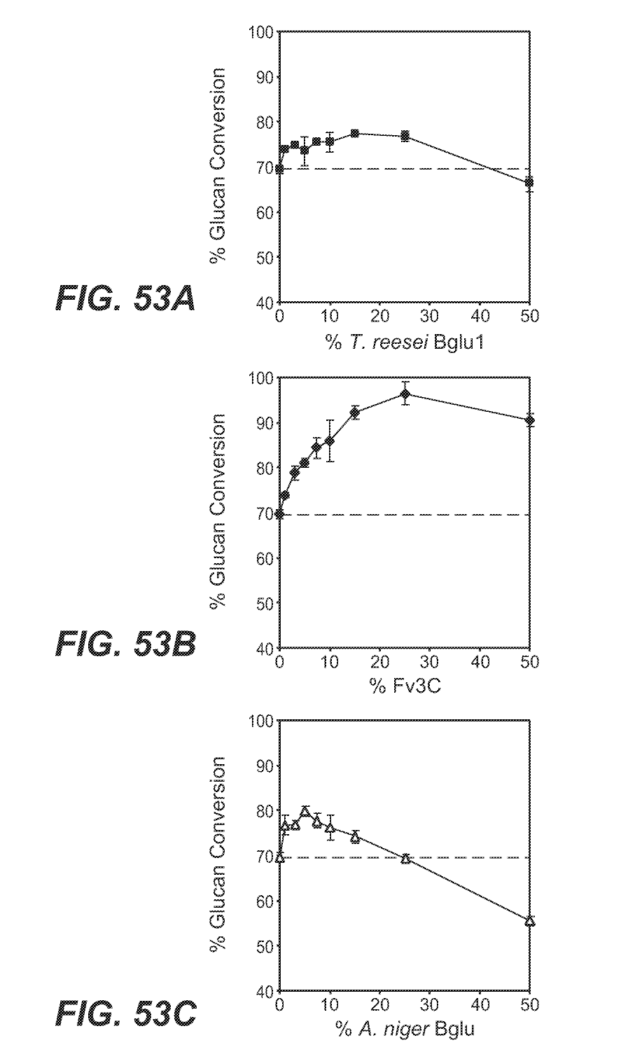

In this experiment, the ratio of Fv3C to whole cellulase was varied to determine the optimal ratio of Fv3C to whole cellulase in a hemicellulase composition. Dilute ammonia pretreated corncob was used as substrate. The ratio of beta-glucosidases (e.g., T. reesei Bgl1, Fv3C, A. niger Bglu) to the whole cellulase derived from T. reesei integrated strain (H3A) was varied from 0 to 50% in the hemicellulase composition. The mixtures were added to hydrolyze ammonia pre-treated corncob at 20% solids at 20 mg protein/g cellulose. The results are shown in FIGS. 53A-53C.

The optimal ratio of T. reesei Bgl1 to whole cellulase was broad, centering at about 10%, with the 50% mixture yielding similar performance to the same loading of whole cellulase alone. In contrast, the A. niger Bglu reached optimum at about 5%, and the peak was sharper. At the peak/optimum level, A. niger Bglu gave higher conversion than the optimal mix comprising T. reesei Bglu.

The optimal ratio of Fv3C to whole cellulase was determined to be about 25%, with the mixture yielding over 96% glucan conversion at 20 mg total protein/g cellulose. Thus, 25% of the enzymes in whole cellulase can be replaced with a single enzyme, Fv3C, resulting in improved saccharification performance.

### Example 7

### Saccharification of Ammonia Pretreated Corncob by Different Enzyme Blends

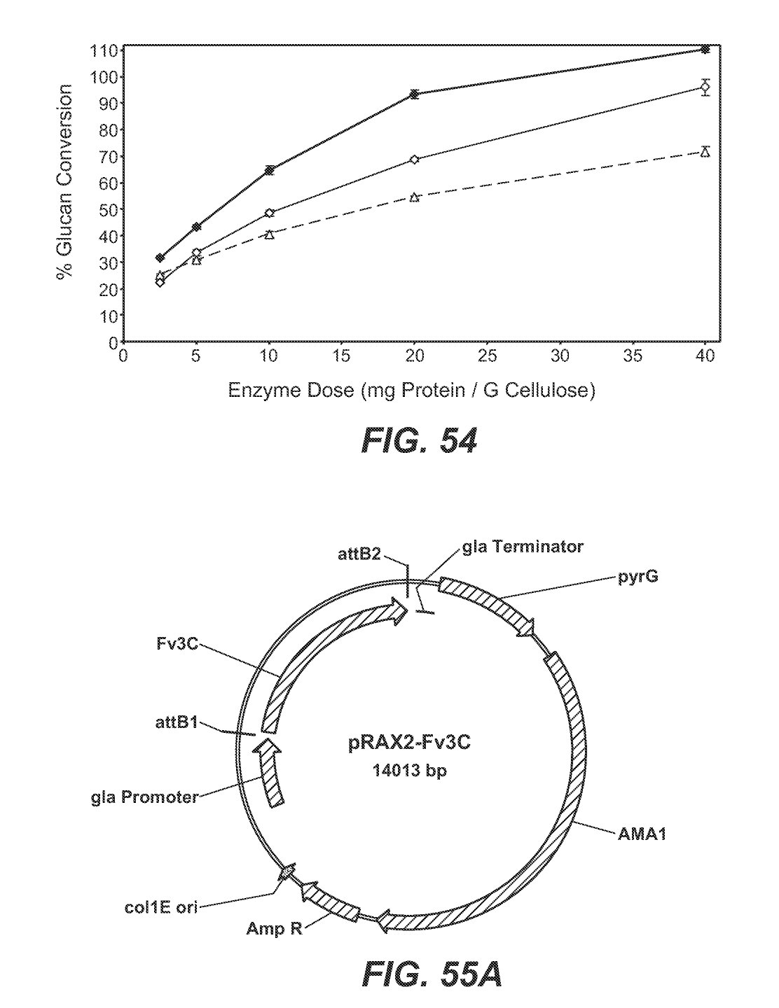

A 25% Fv3C/75% whole cellulase from T. reesei integrated strain (H3A) mixture was compared with other high performing cellulase mixtures in a dose response experiment. Whole cellulase from T. reesei integrated strain (H3A) alone, 25% Fv3C/75% whole cellulase from T. reesei integrated strain (H3A) mixture, and Accellerase® 1500+Multifect® Xylanase were compared for their saccharification performances on dilute ammonia pre-treated corncob at 20% solids. The enzyme blends were dosed from 2.5 to 40 mg protein/g cellulose in the reaction. Results are shown in FIG. 54.

The 25% Fv3C/75% whole cellulase from T. reesei integrated strain (H3A) mixture performed dramatically better than the Accellerase® 1500+Multifect® Xylanase blend, and showed a substantial improvement over the whole cellulase from T. reesei integrated strain (H3A). The dose required for 70, 80 or 90% glucan conversion from each enzyme mix are listed in FIG. 7. At 70% glucan conversion, the 25% Fv3C/75% whole cellulase from T. reesei integrated strain (H3A) mixture gave a 3.2 fold dose reduction when compared to the Accellerase® 1500+Multifect® Xylanase blend. At 70, 80 or 90% glucan conversion, the 25% Fv3C/75% whole cellulase from T. reesei integrated strain (H3A) mixture required about 1.8-fold less enzyme than the whole cellulase from T. reesei integrated strain (H3A) alone.

### Example 8

### Expression of Fv3C in Aspergillus Niger Strain

To express Fv3C in A. niger, the pENTR-Fv3C plasmid was recombined with a destination vector pRAXdest2, as described in U.S. Pat. No. 7,459,299, using the Gateway LR recombination reaction (Invitrogen). The expression plasmid contained the Fv3C genomic sequence under the control of the A. niger glucoamylase promoter and terminator, the A. nidulans pyrG gene as a selective marker, and the A. nidulans amal sequence for autonomous replication in fungal cells. Recombination products generated were transformed into E. coli Max Efficiency DH5a (Invitrogen), and clones containing the expression construct pRAX2-Fv3C (FIG. 55A) were selected on 2×YT agar plates, prepared with 16 g/L Bacto Tryptone (Difco), 10 g/L Bacto Yeast Extract (Difco), 5 g/L NaCl, 16 g/L Bacto Agar (Difco), and 100 μg/mL ampicillin.

About 50-100 mg of the expression plasmid was transformed into an A. niger var awamori strain (see, U.S. Pat. No. 7,459,299). The endogenous glucoamylase glaA gene was deleted from this strain, and it carried a mutation in the pyrG gene, which allowed for selection of transformants for uridine prototrophy. A. niger transformants were grown on MM medium (the same minimal medium as was used for T. reesei transformation but 10 mM NH₄Cl was used instead of acetamide as a nitrogen source) for 4-5 d at 37° C., and a total population of spores (about 10⁶ spores/mL) from different transformation plates was used to inoculate shake flasks containing production medium (per 1L): 12 g trypton; 8 g soyton; 15 g (NH₄)₂SO₄; 12.1 g NaH₂PO₄×H₂O; 2.19 g Na₂HPO₄×2H₂O; 1 g MgSO₄×7H₂O; 1 mL Tween 80; 150 g Maltose; pH 5.8. After 3 d of fermentation at 30° C. and shaking at 200 rpm, the expression of Fv3C in transformants was confirmed by SDS-PAGE.

### Example 9

### Performance of T. reesei Bgl3 (Tr3B)

A. Saccharification Using Whole Cellulase/T. reesei Bgl3 Blends on PASC and PCS

A clarified whole cellulase fermentation broth from a Trichoderma reesei mutant strain, derived from RL-P37 (Sheir-Neiss, G. et al. Appl. Microbiol. Biotechnol. 1984, 20:46-53) and selected for high cellulase production was used in the background of these experiments. The whole cellulase and purified T. reesei Bgl3 (Tr3B) were loaded into the saccharification assay based on mg total protein per g cellulose in the substrate. Purified T. reesei Bgl3 was blended with whole cellulase at a level of 0-100% Bgl3. The mixtures were loaded at 20 mg protein/g cellulose. Each sample was tested in triplicates.

Phosphoric acid swollen cellulose (PASC) was prepared from Avicel PH-101 using an adapted protocol of Walseth, TAPPI 1971, 35:228 and Wood, Biochem. J. 1971, 121:353-362. In short, 25 Avicel was solubilized in concentrated phosphoric acid followed by precipitating using cold deionized water. After the cellulose was collected and washed with more water toneutralize the pH, it was diluted to 1% solids in a 50 mM Sodium Acetate buffer, pH 5.0. Twenty (20) μL of the diluted enzyme mixture was added to individual wells of a flat bottom microtiter plate. Using a repeater pipette, 150 μL of substrate was added per well and the plate covered with 2 aluminum plate sealers.

The dilute acid pre-treated corn stover (supra) was diluted to 7% cellulose in a 50 mM Sodium Acetate pH 5 buffer, and the pH of the mixture adjusted to 5.0. Using a repeater pipette, 150 μL of substrate was added to individual wells of a flat bottom microtiter plate. Twenty (20) μL of the diluted enzyme mixture was added to individual wells and the plate covered with 2 aluminum plate sealers.

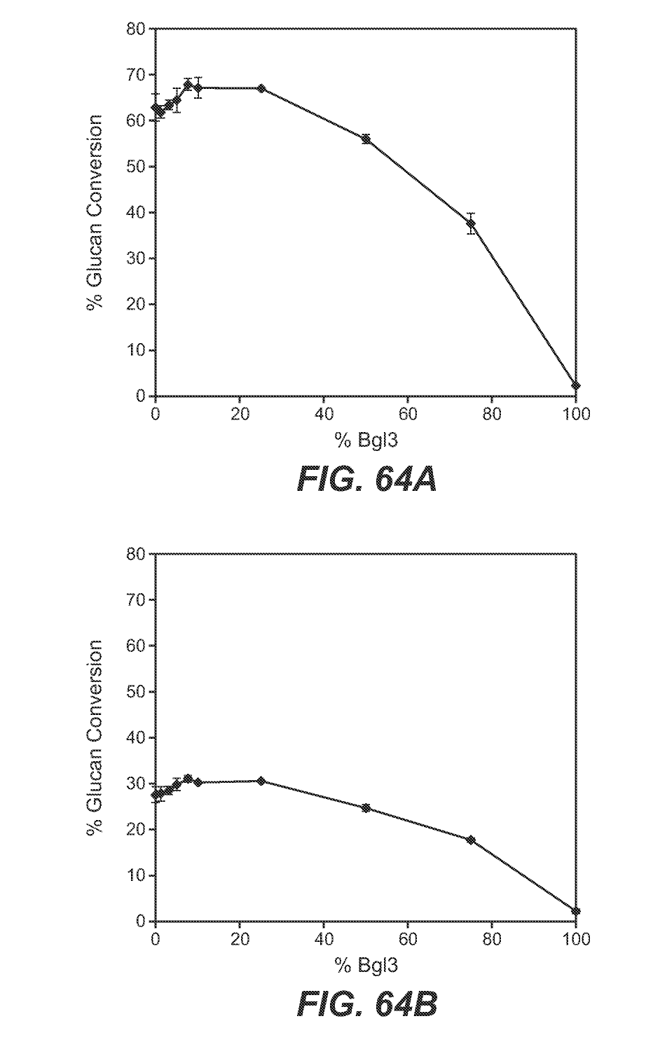

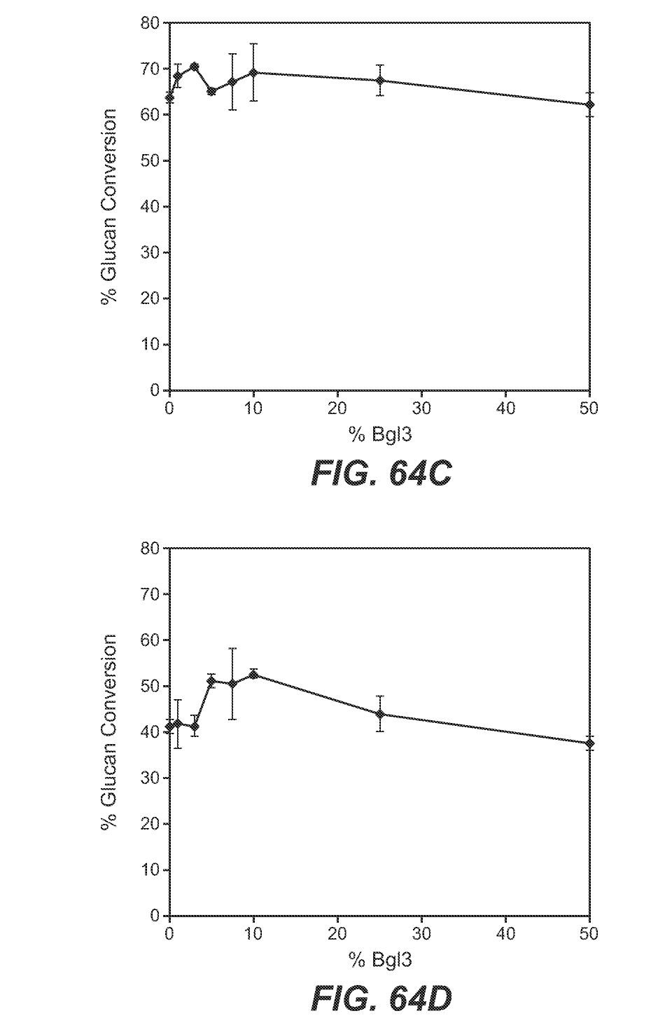

These plates were incubated at 37° C. or 50° C., with mixing at 700 rpm. The PASC was incubated for 2 h and the PCS plates for 48 h. The reactions were terminated by adding 100 μL of a 100 mM Glycine buffer, pH 10 to individual wells. After thorough mixing, the contents of the plates were filtered and the supernatant diluted 6-fold into an HPLC plate containing 100 μL of 10 mM Glycine, pH 10. The concentrations of soluble sugars produced were then measured using HPLC (Agilent 1100 series, equipped with a de-ashing/guard column (Biorad #125-0118)) and an Aminex HPX-87P carbohydrate column, which were maintained at 85° C. The mobile phase was water having a 0.6 mL/min flow rate. Percent glucan conversion is defined here as 100×[mg glucose+(mg cellobiose×1.056)]/[mg cellulose in substrate×1.111]. Accordingly, the % conversions were corrected for water of hydrolysis. Performance results of whole cellulase: T. reesei Bgl3 mixtures in saccharification of PASC at 50° C. are shown in FIG. 64A. Performance results of whole cellulase: T. reesei Bgl3 mixtures in saccharification of PASC at 37° C. are shown in FIG. 64B. Performance of whole cellulase: T. reesei Bgl3 mixtures in saccharification of acid re-treated cornstover at 50° C. are shown in FIG. 64C. Performance of whole cellulase: T. reesei Bgl3 mixtures in saccharification of acid re-treated cornstover at 37° C. are shown in FIG. 64D.

B. Dose Response of Bgl3 with Whole Cellulase Background on PASC

A clarified whole cellulase fermentation broth from a T. reesei mutant strain, derived from RL-P37 (Sheir-Neiss, G et al. Appl. Microbiol. Biotechnol. 1984, 20:46-53) and selected for high cellulase production was used in the background of these experiments.

Whole cellulase and purified T. reesei Bgl3 were loaded into the saccharification assay based on mg total protein per g cellulose in the substrate. Purified T. reesei Bgl3 was loaded in amounts of 0-10 mg protein/g cellulose. A constant level of 10 mg whole cellulase protein/g cellulose was also added to each sample. Each sample was tested in triplicates.

The phosphoric acid swollen cellulose substrate was diluted to 1% cellulose in a 50 mM Sodium Acetate pH 5 buffer, and the pH was adjusted to 5.0. Twenty (20) μL of the diluted enzyme mixture was added to individual wells of a flat bottom microtiter plate. Using a repeater pipette, 150 μL of substrate was added to individual wells and the plate was covered with 2 aluminum plate sealers. The plates were then incubated at 50° C. with mixing at 700 rpm for 1 h.

The reactions were terminated by adding 100 μL of a 100 mM glycine buffer, pH 10 to individual wells. After thorough mixing, the contents of the plates were filtered and the supernatant diluted 6-fold into an HPLC plate containing 100 μL of 10 mM Glycine, pH 10. The concentrations of soluble sugars produced were then measured using HPLC (Agilent 1100 series, equipped with a de-ashing/guard column (Biorad #125-0118)) and an Aminex HPX-87P carbohydrate column, which were maintained at 85° C. The mobile phase was water having a 0.6 mL/min flow rate.

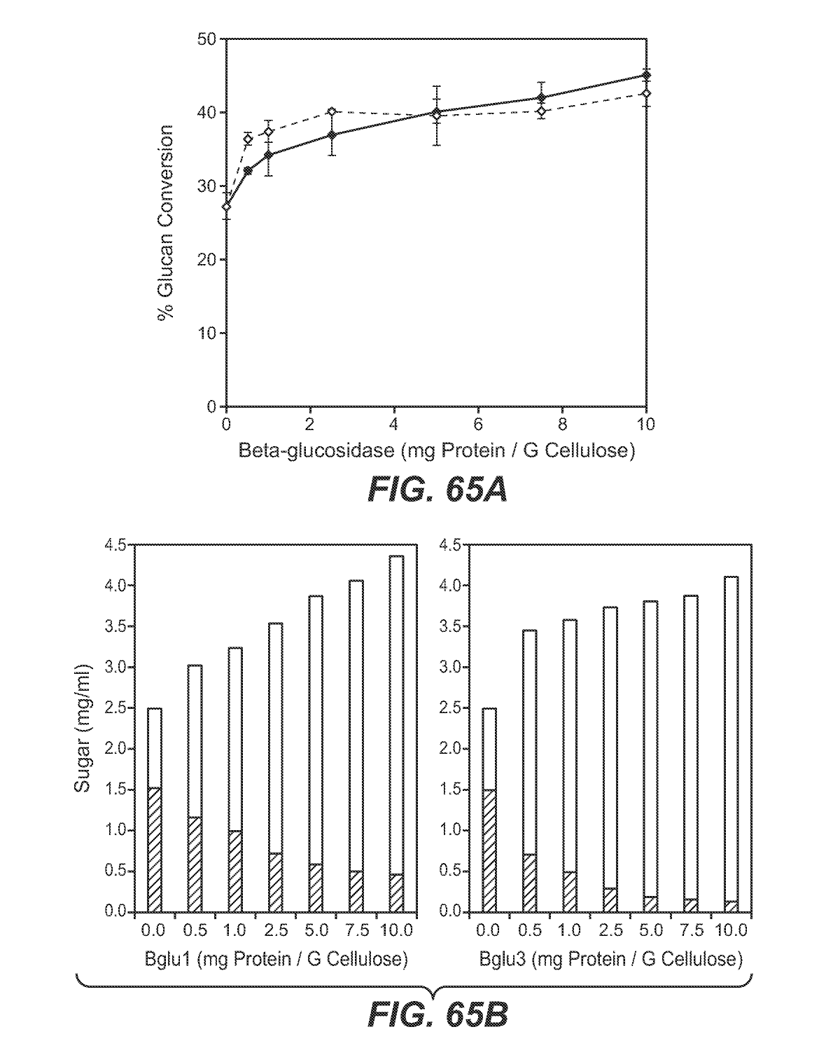

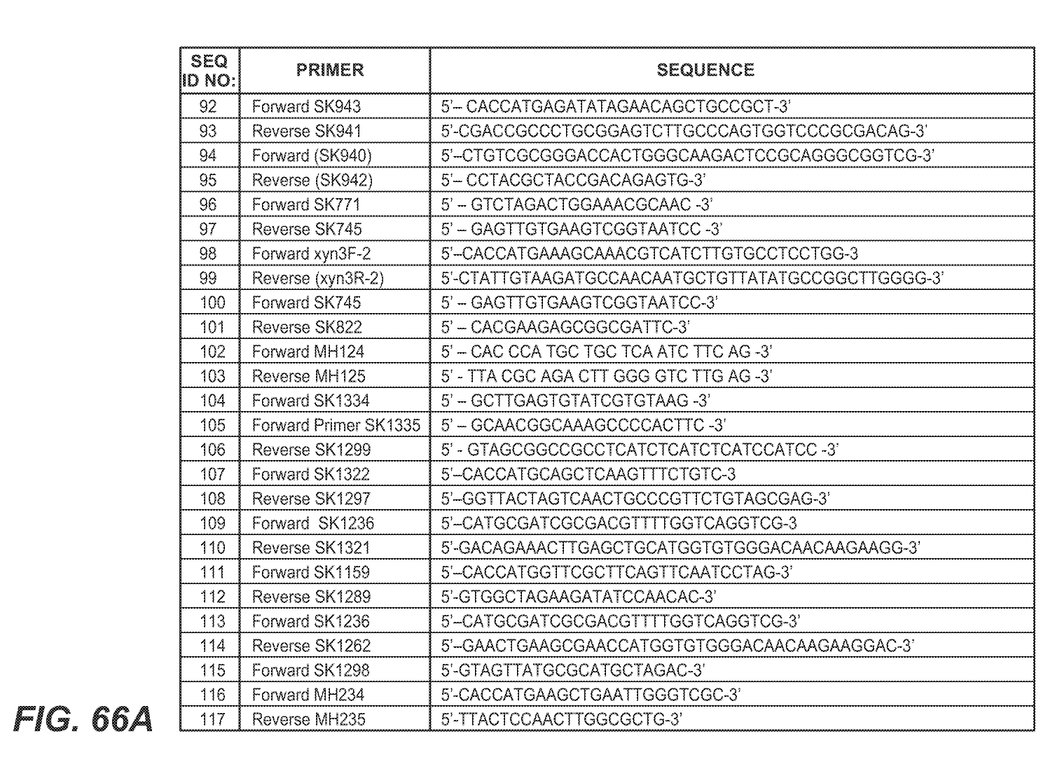

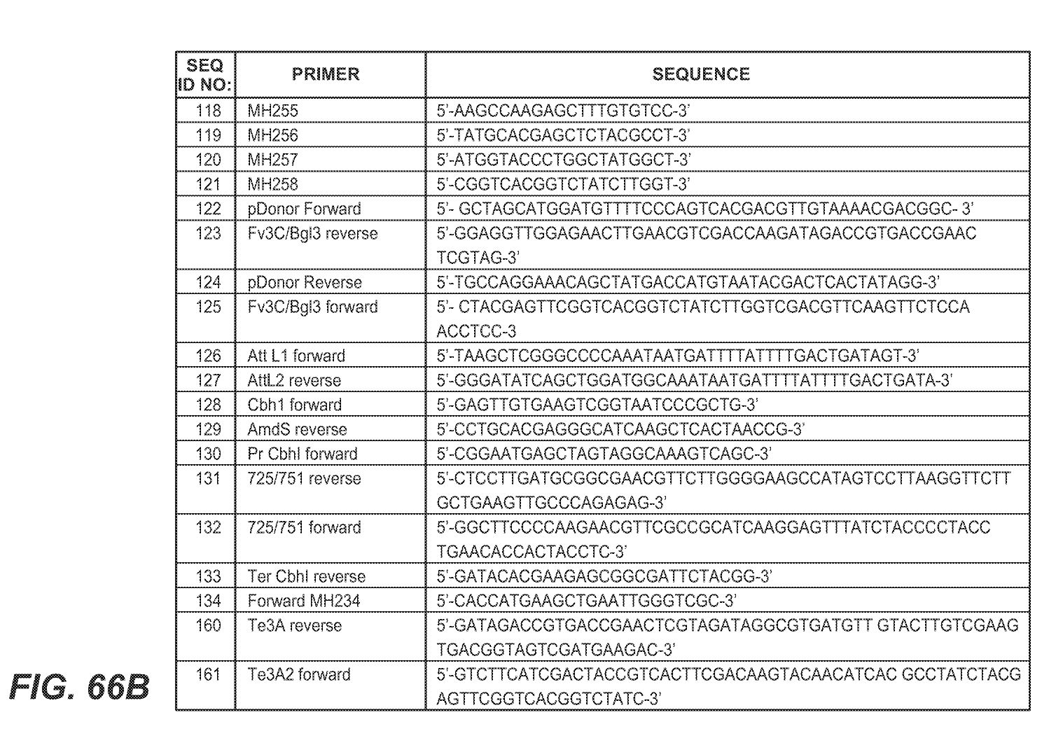

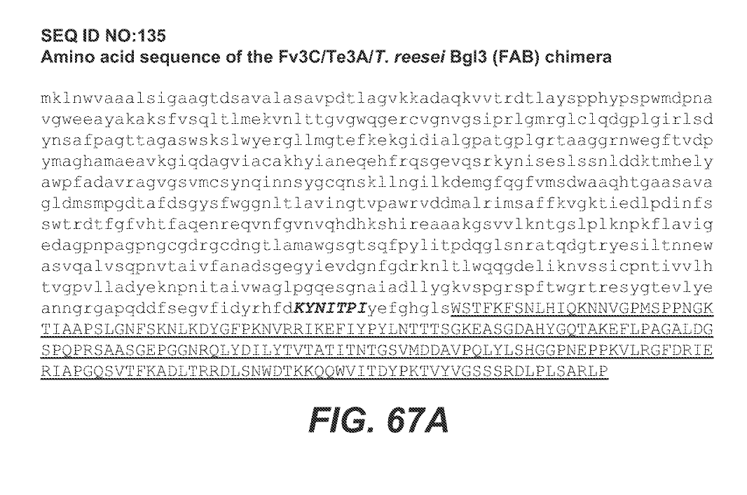

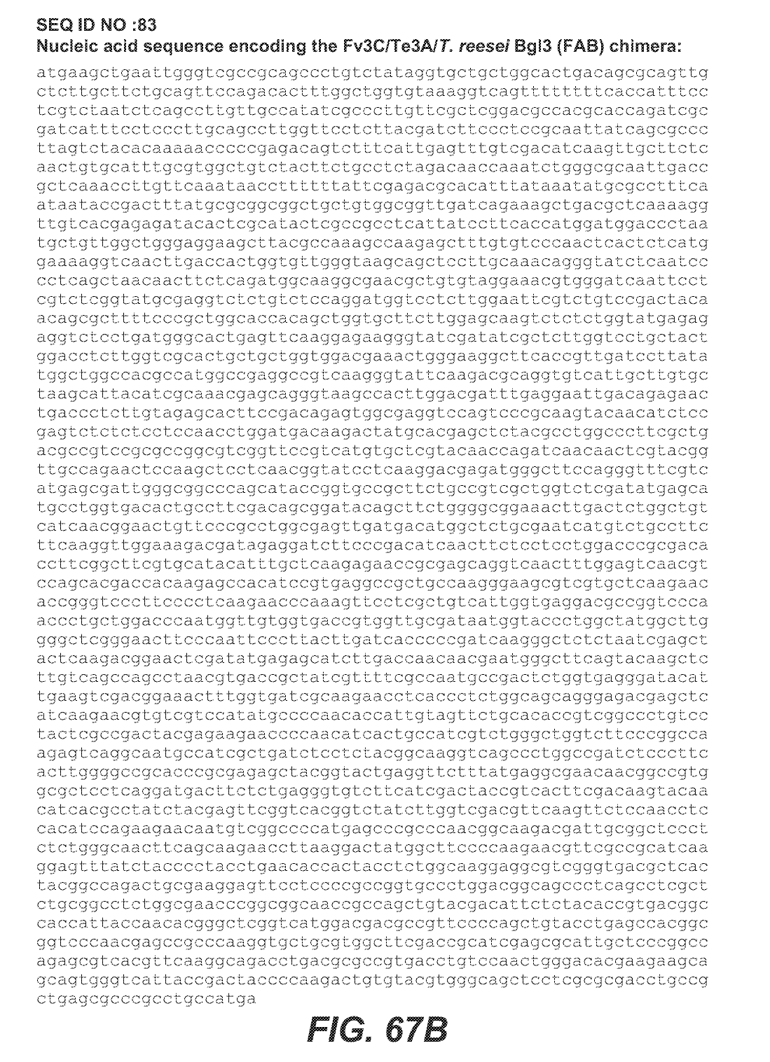

Percent glucan conversion is defined here as 100×[mg glucose+(mg cellobiose×1.056)]/[mg cellulose in substrate×1.111]. Accordingly, the % conversions were corrected for water of hydrolysis. The dose response comparison of T. reesei Bgl1 and T. reesei Bgl3 in saccharification of phosphoric acid swollen cellulose is shown in FIG. 65A. The comparison of cellobiose and glucose produced by T. reesei Bgl1 and T. reesei Bgl3 in saccharification of phosphoric acid swollen cellulose are shown in FIG. 65B.

### Example 10

### Chimeric β-Glucosidase

A. Expression in T. reesei

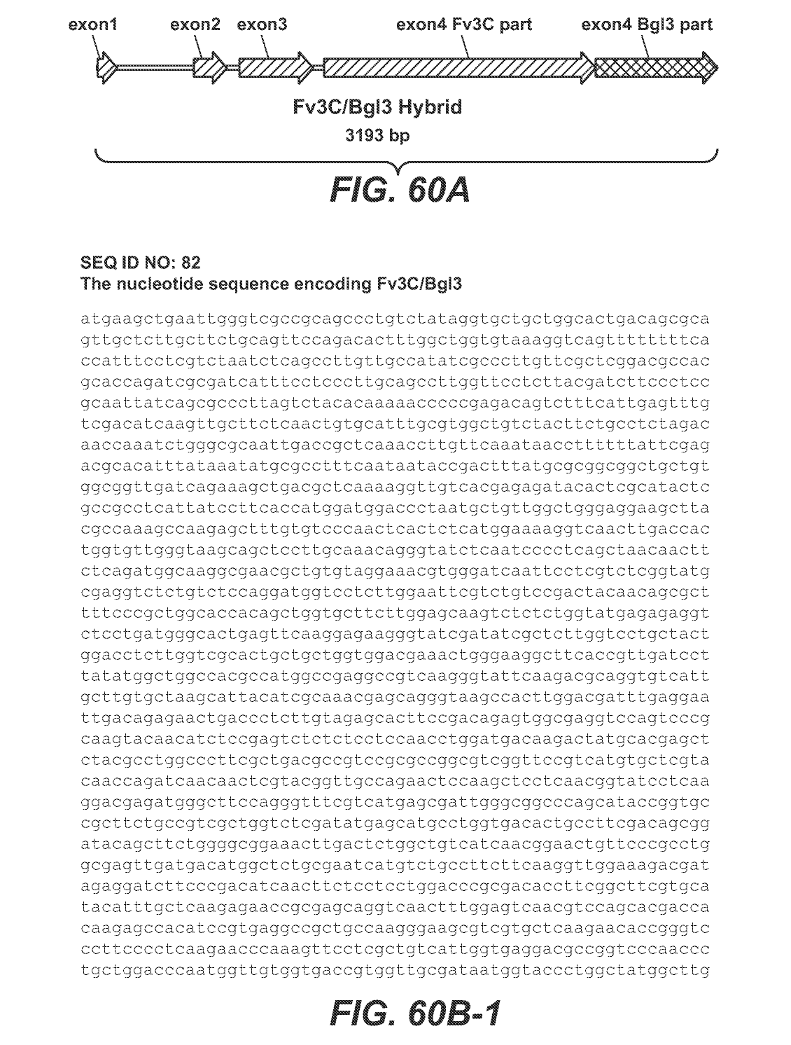

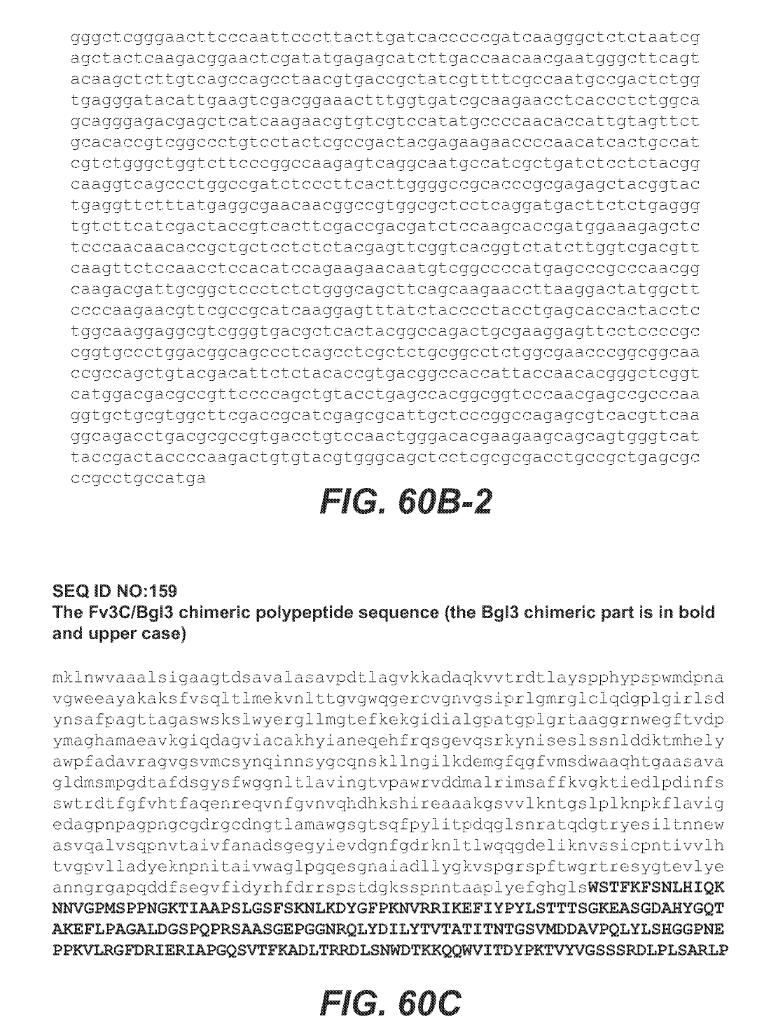

Portions of the wild type Fv3C C-terminal sequence were replaced with C-terminal sequence from T. reesei β-glucosidase, Bgl3 (Tr3B). Specifically, a contiguous stretch representing residues 1-691 of Fv3C was fused with a contiguous stretch representing residues 668-874 of Bgl3. A schematic representation of the gene encoding the Fv3C/Bgl3 chimeric/fusion polypeptide is depicted in FIG. 60A. The amino acid sequence and the polynucleotide sequence encoding the fusion/chimeric polypeptide Fv3C/Bgl3 are depicted in FIGS. 60B and 60C.

The chimeric/fusion molecule was constructed using fusion PCR. pENTR clones of the genomic Fv3C and Bgl3 coding sequences were used as PCR templates. Both entry clones were constructed in the pDonor221 vector (Invitrogen). The fusion product was assembled in two steps. First, the Fv3C chimeric part was amplified in a PCR reaction using a pENTR Fv3C clone as a template and the following oligonucleotide primers:

pDonor Forward:
(SEQ ID NO: 122)
5′-GCTAGCATGGATGTTTTCCCAGTCACGACGTTGTAAAACGA
CGGC-3′
Fv3C/Bgl3 reverse:
(SEQ ID NO: 123)
5′-GGAGGTTGGAGAACTTGAACGTCGACCAAGATAGACCGTGA
CCGAAC TCGTAG 3′

The Bgl3 chimeric part was amplified from a pENTR Bgl3 vector using the following oligonucleotide primers:

pDonor Reverse:
(SEQ ID NO: 124)
5′-TGCCAGGAAACAGCTATGACCATGTAATACGACTCACTATAGG-3′
(SEQ ID NO: 125)
Fv3C/Bgl3 forward:
5′-CTACGAGTTCGGTCACGGTCTATCTTGGTCGACGTTCAAGTTC
TCCAACCTCC-3′

In the second step, equimolar of the PCR products (about 1 μL and 0.2 μL of the initial PCR reactions, respectively) were added as templates for a subsequent fusion PCR reaction using a set nested primers as follows:

AttL1 forward:
(SEQ ID NO: 126)
5′ TAAGCTCGGGCCCCAAATAATGATTTTATTTTGACTGATAGT 3′
AttL2 rev.:
(SEQ ID NO: 127)
5′GGGATATCAGCTGGATGGCAAATAATGATTTTATTTTGACTGATA 3′

The PCR reactions were performed using a high fidelity Phusion DNA polymerase (Finnzymes OY). The resulting fused PCR product contained the intact Gateway-specific attL1, attL2 recombination sites on the ends, allowing for direct cloning into a final destination vector via a Gateway LR recombination reaction (Invitrogen).

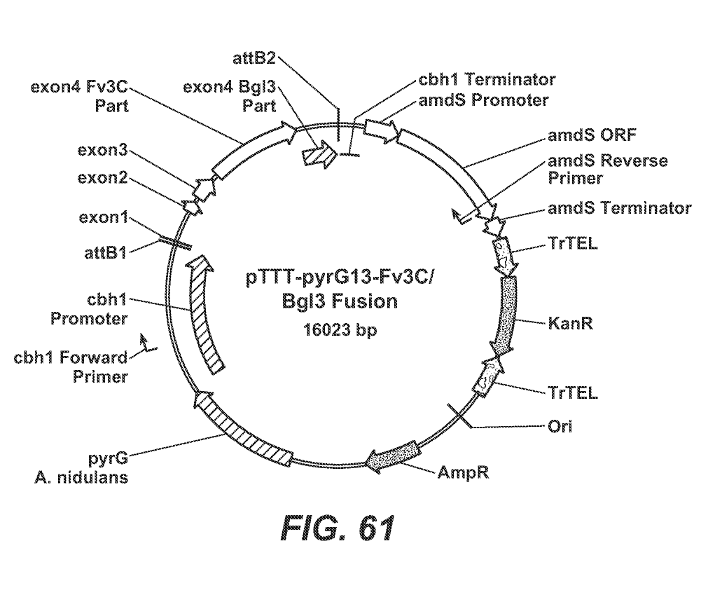

After separation of the DNA fragments on a 0.8% agarose gel, the fragments were purified using a Nucleospin® Extract PCR clean-up kit (Macherey-Nagel GmbH & Co. KG) and 100 ng of each fragment was recombined using a pTTT-pyrG13 destination vector and the LR Clonase™ II enzyme mix (Invitrogen). The resulting recombination products were transformed to E. coli Max Efficiency DH5a (Invitrogen), and clones containing the expression construct pTTT-pyrG13-Fv3C/Bgl3 fusion (FIG. 61) containing the chimeric β-glucosidase were selected on 2×YT agar plates, prepared using 16 g/L Bacto Tryptone (Difco), 10 g/L Bacto Yeast Extract (Difco), 5 g/L NaCl, 16 g/L Bacto Agar (Difco), and 100 μg/mL ampicillin. The bacteria were grown in 2×YT medium containing 100 μg/mL of ampicillin. Thereafter, the plasmids were isolated and subject to restriction digests by either BglI or EcoRV. The resulting Fv3C/Bgl3 region was sequenced using an ABI3100 sequence analyzer (Applied Biosystems) for confirmation. A plasmid having the confirmed restriction pattern and correct sequence was used as a template in a further PCR reaction to generate a DNA fragment, using a high fidelity Phusion DNA polymerase (Finnzymes OY) and the primers as follows:

(SEQ ID NO: 128
Cbhl forward: 5′ GAGTTGTGAAGTCGGTAATCCCGCTG 3′
(SEQ ID NO: 129)
AmdS reverse: 5′ CCTGCACGAGGGCATCAAGCTCACTAACCG 3′

The resulting fragment encompassed the Fv3C/Bgl3 coding region under the control of the cbh1 promoter and terminator. Specifically, 0.5-1 μg of this fragment was transformed into a T. reesei hexa-delete strain (see, supra) using the PEG-Protoplast method with slight modifications as described below. For protoplasts preparation, spores were grown for 16-24 h at 24° C. in Trichoderma Minimal Medium MM, which contained 20 g/L glucose, 15 g/L KH₂PO₄, pH 4.5, 5 g/L (NH₄)₂SO₄, 0.6 g/L MgSO₄×7H₂O, 0.6 g/L CaCl₂×2H₂O, 1 mL of 1000× T. reesei Trace elements solution (which contained 5 g/L FeSO₄×7H₂O, 1.4 g/L ZnSO₄×7H₂O, 1.6 g/L MnSO₄×H₂O, 3.7 g/L CoCl₂×6H₂O) with shaking at 150 rpm. Germinating spores were harvested by centrifugation and treated with 50 mg/mL of Glucanex G200 (Novozymes AG) solution to lyse the fungal cell walls. Further preparation of the protoplasts was performed in accordance with a method described by Penttilä et al. Gene 61 (1987) 155-164.

The transformation mixtures, which contained about 1 μg of DNA and 1−5×10⁷ protoplasts in a total volume of 200 μL, were each treated with 2 mL of 25% PEG solution, diluted with 2 volumes of 1.2 M sorbitol/10 mM Tris, pH7.5, 10 mM CaCl₂, mixed with 3% selective top agarose MM containing 5 mM uridine and 20 mM acetamide. The resulting mixtures were poured onto 2% selective agarose plate containing uridine and acetamide. Plates were incubated further for 7-10 d at 28° C. before single transformants were re-picked onto fresh MM plates containing uridine and acetamide. Spores from independent clones were used to inoculate a fermentation medium in either 96-well microtiter plates or shake flasks.

96 well filter plates (Corning) containing 250 μL of glycine production medium containing 4.7 g/L (NH₄)₂SO₄, 33 g/L 1,4-piperazinebis(propanesulfonic acid), pH 5.5, 6.0 g/L glycine, 5.0 g/L KH₂PO₄, 1.0 g/L CaCl₂×2H₂O, 1.0 g/L MgSO₄×7H₂O, 2.5 ml/L of a 400× T. reesei trace element solution, 20 g/L glucose, and 6.5 g/L sophorose were inoculated using spore suspensions of T. reesei transformants expressing the Fv3C/Bgl3 hybrid (more than 10⁴ spores per well). Plates were incubated at 28° C. and in about 80% humidity for 6-8 d. Culture supernatants were harvested by vacuum filtration and used to test performance of the hybrid as well as its expression level. Protein profile of the whole broth samples was determined by PAGE electrophoresis. Twenty (20) μL of culture supernatants were mixed with an 8 μL of a 4× sample loading buffer without a reducing agent. The samples were separated on NuPAGE® Novex 10% Bis-Tris Gel using MES SDS Running Buffer (Invitrogen).

This resulted in an Fv3C/Bgl3 (FB) chimeric β-glucosidase that is less sensitive to protease degradation when expressed in T. reesei or during storage. After 8 days of fermentation in a microtiter plate, significantly less breakdown of the expressed β-glucosidase was observed with the Fv3C/Bgl3 (FB) chimera, as compared to the Fv3C β-glucosidase under comparable conditions.

B. Expression of Fv3C and FAB in a Chrysosporium lucknowence Host Cell.

### Construction of the Expression Cassette

The Fv3C expression vectors described for T. reesei (pTrex6g/Fv3c, Example 3, FIG. 45B) and for A. niger (pRAX2-Fv3C, Example 8, FIG. 55A) are used to express Fv3C, or FAB in Chrysosporium lucknowense. The native Fv3C signal sequence is used. The vector pRAX2-Fv3C contains the fv3C gene sequence under control of the A. niger glucoamylase promoter and terminator sequences, the A. nidulans pyrG gene as a selective marker, and the A. nidulans amal sequence for autonomous replication in fungal cells. The vector pTrex6g/Fv3c contains the Fv3C open reading frame under control of the T. reesei cbh1 promoter and terminator sequences, and the T. reesei mutated acetolactate synthase selection marker (als) with its native promoter and terminator. Alternatively, selection markers such as phleomycin or hygromycin resistance, or the nutritional selection marker acetamidase (amdS) can also be used.

Transformation of C. lucknowense

C. lucknowense host cells are transformed with pTrex6g/Fv3C by protoplast fusion as described by Penttila et al. Gene 61 (1987) 155-164, with the modifications known in the art, such as those described in e.g., U.S. Pat. No. 6,573,086. Resistant transformants can then be selected on fresh chlorimuron ethyl plates. Alternatively, pyrG-(uridine auxotrophic) C. lucknowense host cells can be transformed with pRAX2-Fv3C by protoplast fusion and selected for uridine prototrophy as described in Example 8, supra.

Culturing C. lucknowense Transformants for Protein Production

Fv3C and FAB are produced by culturing C. lucknowense transformants at 27-40° C., pH 5-10, with shaking for about 5 d in the media described in, e.g., WO 98/15633, using cellulose or lactose to induce the CBHI promoter, or maltose, maltrin or starch to induce the glucoamylase promoter.

### Example 11

### Chimeric Beta-Glucosidase

SDS-PAGE and peptide mapping analysis revealed that the Fv3C/Bgl3 chimer was clipped into two fragments when it was produced in T. reesei. N-terminal sequencing indicated a clip site between residues 674 and 683 of the full length of Fv3C.

A second chimeric β-glucosidase was constructed, which comprised an N-terminal sequence derived from Fv3C, a loop region derived from the sequence of a second β-glucosidase from Talaromyces emersonii Te3A, and a C-terminal part sequence derived from T. reesei Bgl3 (or Tr3B). This was accomplished by replacing a loop region of the Fv3C/Bgl3 chimera (see, Example 10, supra). Specifically Fv3C residues 665-683 of the Fv3C/Bgl3 chimera (having a sequence of RRSPSTDGKSSPNN TAAPL (SEQ ID NO:157) were replaced with Te3A residues 634-640 (KYNITPI (SEQ ID NO:158). This hybrid molecule was constructed using a fusion PCR approach, as described in Example 10, supra.

Two N-glycosylation sites, namely S725N and S751N, were introduced into the Fv3C/Bgl3 backbone. These glycosylation mutations were introduced in the Fv3C/Bgl3 backbone using the fusion PCR amplification technique as described above, employing the pTTT-pyrG13-Fv3C/Bgl3 fusion plasmid (FIG. 61) as a template to generate the initial PCR fragments. The following pairs of primers were added in separate PCR reactions:

Pr CbhI forward:
(SEQ ID NO: 130
5′ CGGAATGAGCTAGTAGGCAAAGTCAGC 3′
and
725/751 reverse:
(SEQ ID NO: 131)
5′-CTCCTTGATGCGGCGAACGTTCTTGGGGAAGCCATAGTCCTTAA
GGTTCTTGCTGAAGTTGCCCAGAGAG 3′
725/751 forward:
(SEQ ID NO: 132)
5′-GGCTTCCCCAAGAACGTTCGCCGCATCAAGGAGTTTATCTACC
CCTACCTGAACACCACTACCTC 3′,
and
Ter CbhI reverse:
(SEQ ID NO: 133)
5′ GATACACGAAGAGCGGCGATTCTACGG 3′.

Next, the PCR fragments were fused using the Pr CbhI forward and Ter CbhI primers. The resulting fusion product included the two desired glycosylation sites, but also contained intact attB1 and attB2 sites, which allowed for recombination with the pDonor221 vector using the Gateway BP recombination reaction (Invitrogen). This resulted in a pENTR-Fv3C/Bgl3/S725N S751N clone, which was then used as a backbone for constructing the triple hybrid molecule Fv3C/Te3A/Bgl3.

To replace the loop of the Fv3C/Bgl3 hybrid at residues 665-683 with the loop sequence from Te3A, primary PCR reactions were performed using the following primer sets:

Set 1:
pDonor Forward:
(SEQ ID NO: 122)
5′-GCTAGCATGGATGTTTTCCCAGTCACGACGTTGTAAA
ACGACGGC 3′
and
Te3A reverse:
(SEQ ID NO: 160)
5′-GATAGACCGTGACCGAACTCGTAGATAGGCGTGATGTT
GTACTTGTCGAAGTGACGGTAGTCGATGAAGAC 3′;
Set 2:
Te3A2 forward:
(SEQ ID NO: 161)
5′-GTCTTCATCGACTACCGTCACTTCGACAAGTACAACATCAC
GCCTATCTACGAGTTCGGTCACGGTCTATC-3′;
and
pDonor Reverse:
(SEQ ID NO: 124)
5′ TGCCAGGAAACAGCTATGACCATGTAATACGACTCACTATAGG 3′

Fragments obtained in the primary PCR reactions were then fused using the following primers:

AttL1 forward:
(SEQ ID NO: 126)
5′ TAAGCTCGGGCCCCAAATAATGATTTTATTTTGACTGATAGT 3′
and
AttL2 reverse:
(SEQ ID NO: 127)
5′GGGATATCAGCTGGATGGCAAATAATGATTTTATTTTGACTGATA 3′.

The resulting PCR product contained the intact Gateway-specific attL1, attL2 recombination sites on the ends, allowing for direct cloning into a final destination vector using a Gateway LR recombination reaction (Invitrogen).

The DNA sequence of the Fv3C/Te3A/Bgl3 encoding gene is listed in SEQ ID No: 83] The amino acid sequence of the Fv3C/Te3A/Bgl3 (FAB) hybrid is listed in SEQ ID No:135. The gene sequence encoding the Fv3C/Te3A/Bgl3 chimera was cloned in the pTTT-pyrG13 vector and expressed in a T. reesei recipient strain as described in Example 10, supra.

### Example 12

### Improved Stability of Chimeric Beta-Glucosidases

This experiment determined the thermal denaturing temperatures of various beta-glucosidases using differential scanning calorimetry (DSC). Specifically, thermal transition temperatures were determined for purified enzymes Fv3C/Te3A/Bgl3 chimera, Fv3C, and T. reesei Bgl1. The enzymes were diluted to 500 ppm in a 50 mM sodium acetate buffer, pH 5.0. The DSC 96-well microtiter plate (MicroCal) was loaded with 500 μL of individual diluted enzyme samples. Water and buffer blanks were also included. DSC (Auto VP-DSC, MicroCal) parameters were set to a scan rate of 90° C./h; at 25° C. initial temperature, and 110° C. final temperature. The thermogram is shown in FIG. 63. Tₘ for Fv3C and the Fv3C/Te3A/Bgl3 chimera appeared similar to and perhaps somewhat lower than that of the T. reesei Bgl1.

### Example 13

### Activity of A. niger Expressed Fv3C in Saccharfication of Dilute Ammonia Pretreated Corncob

Integrated strain H3A-5 (a low β-glucosidase producer), Fv3C produced in A. niger (see Example 8), and purified T. reesei Bgl1 (also termed “T. reesei Bglu1” or “Tr3A” herein) were loaded into the saccharification assay based on mg total protein per g cellulose in the substrate. The beta-glucosidases were loaded from 0-10 mg protein/g cellulose. A constant level of 10 mg/g H3A-5 was added to each sample. Each sample was run with 5 assay replicates.

The dilute ammonia pre-treated corncob substrate was diluted to 7% cellulose in 50 mM Sodium Acetate pH 5 buffer and the pH adjusted to 5.0. The substrate was delivered into 96-well microtiter plates (65 mg per well). Thirty (30) μL of appropriately diluted enzyme mix was added per well to the 96-well plate. After addition of enzyme mix, the substrate was calculated to contain 5% cellulose. The plates were covered with 2 aluminum plate sealers. All plates were then placed in an incubator at 50° C. and 200 rpm for 48 h.

The reaction was terminated by adding 100 μL 100 mM Glycine buffer, pH 10 to each well. After thorough mixing, the contents of the plates were centrifuged and the supernatant diluted 11 fold into an HPLC plate containing 100 μL of 10 mM Glycine, pH 10. The concentrations of soluble sugars produced were then measured via HPLC. The Agilent 1100 series HPLC was equipped with a de-ashing/guard column (Biorad #125-0118) and an Aminex lead based carbohydrate column (Aminex HPX-87P) maintained at 85° C. The mobile phase was water with a 0.6 ml/min flow rate.

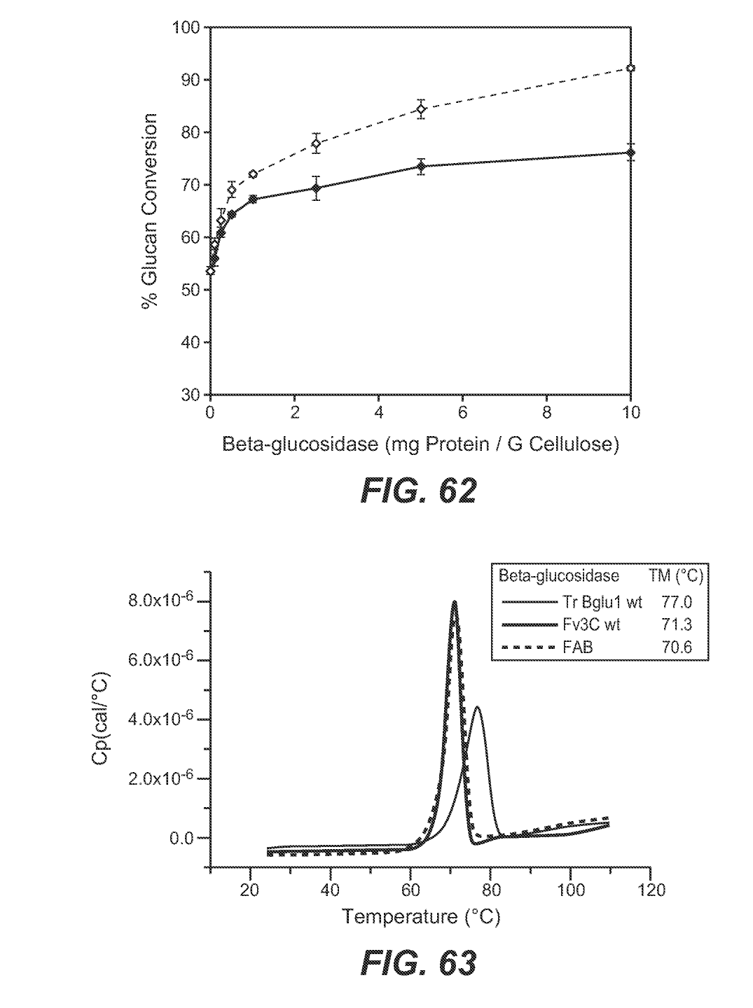

Percent glucan conversion is defined as 100×[mg glucose+(mg cellobiose×1.056)]/[mg cellulose in substrate×1.111]. In this way, the % conversions, which were corrected for water of hydrolysis, are depicted in FIG. 62.

### Example 13

### Comparison of Substrate Binding of Fv3C, Fab and T. reesei BGL1

This experiment compares the binding of each of Fv3C, the chimeric b-glucosidase molecule FAB, and T. reesei Bgl1 to certain typical biomass substrates.

Lignin, a complex biopolymer of phenylpropanoid, is the chief non-carbohydrate constituent of wood that binds to cellulose fibers to harden and strengthen cell walls of plants. Because it is cross-linked to other cell wall components, lignin minimizes the accessibility of cellulose and hemicellulose to cellulose degrading enzymes. Hence, lignin is generally associated with reduced digestibility of all plant biomass. In particular the binding of cellulases to lignin reduces the degradation of cellulose by cellulases. Lignin is hydrophobic and apparently negatively charged. Among FAB, Bgl1, and Fv3C, Fv3C has the lowest pI and is least positively charged, while Bglu1 has the highest pI and is most positively charged, and their binding to the lignocellulosic substrate was investigated.

Lignin was recovered following extensive saccharification of dilute ammonia pretreated corn cob (DACC) or corn stover (DACS) or acid pretreated corn stover (PCS or whPCS) using a saccharification mixture containing an Accellerase at 100 mg/g of cellulose and 8 mg Multifect xylanase/g cellulose. Saccharification was followed by hydrolysis of the cellulases by nonspecific serine protease addition. 0.1N HCl was added into the mixture to inactivate the protease followed by repeated washes with acetate buffer (50 mM sodium acetate pH 5) to return the sample to pH 5.

One hundred (100) μL of DACS (at about 5% glucan), DACC (at about 5% glucan), whPCS (at about 5% glucan), lignin prepared from DACC (as in 5% glucan), lignin prepared from PCS (as in 5% glucan), or 50 mM sodium acetate pH 5 buffer control were combined with 100 μL of 150 μg/mL FAB, T. reesei Bgl1, or Fv3C in a microtiter plate, which was then sealed and incubated at 50° C. for 44 h. The microtiter plate was centrifuged at high speed to separate soluble from insoluble materials. The enzyme activity in the soluble fraction was measured. Briefly, the supernatant was 5-fold diluted, then 20 uL was added into 80 uL 2 mM 2-Chloro-4-Nitrophenyl β-D-glucopyranoside (CNPG) and incubated at room temperature for 6 mins. One hundred (100) uL of 500 mM Na2CO3 pH9.5 was added to quench the reaction. OD405 was read. The percent of unbound beta-glucosidase was calculated by using OD405 of beta-glucosidase activity in the soluble fraction divided by OD405 of the control sample that was incubated in the same way in the absence of lignin and biomass substrate.

The total activity of bound and unbound β-glucosidase was measured. The microtiter plate was re-mixed, 20 uL aliquots was each added into into 80 uL sodium acetate buffer pH5, 20 uL of diluted mix was added into 80 uL 2 mM 2-Chloro-4-Nitrophenyl β-D-glucopyranoside (CNPG) and incubated at room temperature for 6 mins, and 100 uL of 500 mM Na₂CO₃ pH9.5 was added to quench the reaction. The reaction mixture was spun down and 100 uL of supernatant was transferred out into a new microtiter plate. OD405 was measured. The relative total β-glucosidase activity in the presence of biomass or lignin was calculated by using OD405 of the total mix divided by OD405 of the control sample that was incubated in the same way in the absence of lignin and biomass substrate.

In order to verify that the bound beta-glucosidase did not dissociate in the time frame of measurement, 20 uL aliquot was taken out from remixed microtiter plate into 80 uL of sodium acetate buffer pH 5 in a new microtiter plate, the plate was incubated at room temperature with shaking for half an hour for beta-glucosidase to dissociate from biomass or lignin. Then the plate was centrifuged and beta-glucosidase activity in the supernatant was measured as described above. Again, the unbound beta-glucosidase was calculated.

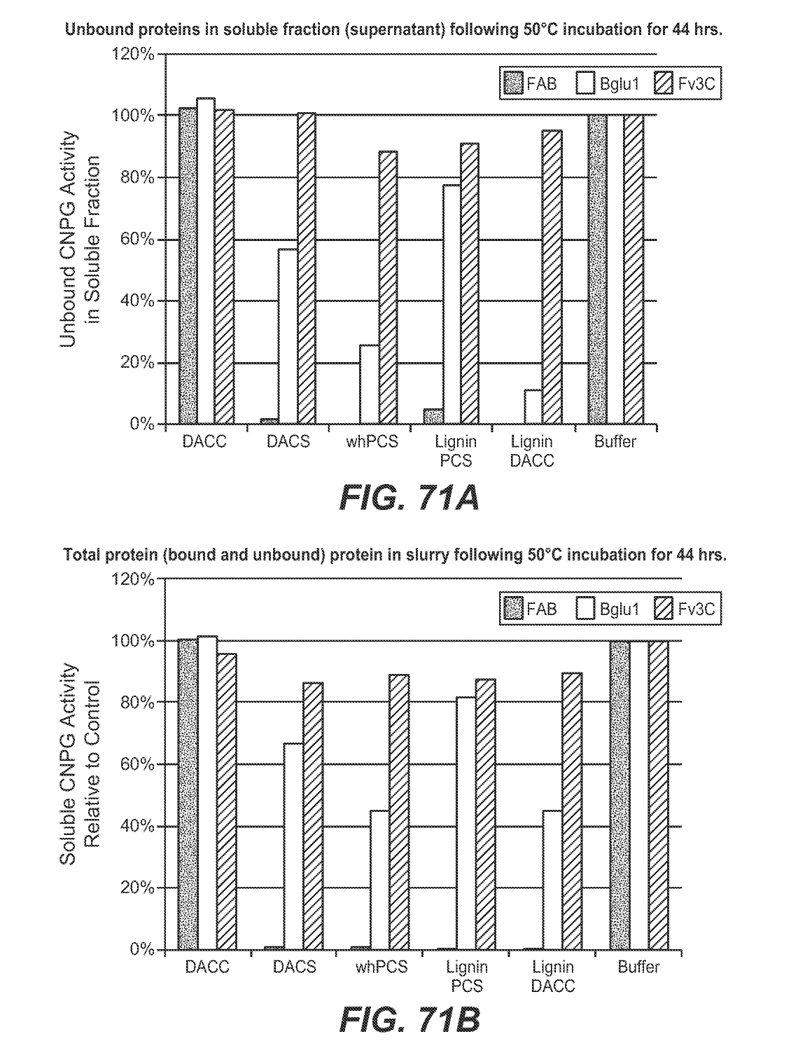

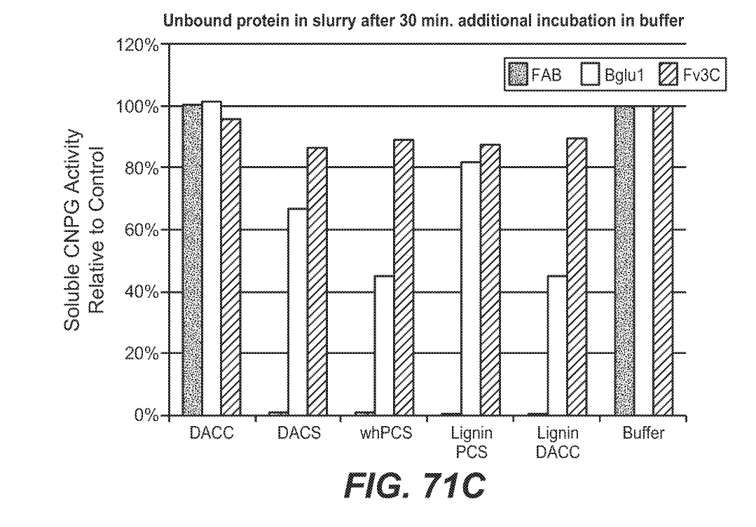

Fv3C showed least binding to biomass substrate or lignin, while both FAB and T. reesei 1 showed high levels of binding to biomass substrate and lignin (FIG. 71A). None of these three β-glucosidases bound to DACC, but both T. reesei and FAB bound to lignin prepared from complete saccharification of DACC. Surprisingly, the bound FAB or T. reesei Bgl1 remained about 50-80% active as compared to free FAB or Bgl1 (FIG. 71B). It was also observed that the bound FAB did not dissociate from the biomass or lignin, but about 20% Bgl1 did dissociate from a bound state to an unbound state during a 30-min incubation period (FIG. 71C).

1. An isolated polypeptide comprising:
a) an amino acid sequence that has at least about 70% identity to SEQ ID NO:135; or
b) an N-terminal sequence and a C-terminal sequence, wherein the N-terminal sequence comprises a first amino acid sequence derived from a first β-glucosidase, is at least 200 residues in length, and comprises one or more or all of SEQ ID NOs: 164-169, and wherein the C-terminal sequence comprises a second amino acid sequence derived from a second β-glucosidase, is at least 50 residues in length, and comprises SEQ ID NO:170,
wherein the polypeptide has β-glucosidase activity.

2. The isolated polypeptide of claim 1, comprising an amino acid sequence that has at least about 80% identity to SEQ ID NO:135 or at least about 90% identity to SEQ ID NO:135.

3. (canceled)

4. The isolated polypeptide of claim 1, comprising the N-terminal sequence derived from the first β-glucosidase and the C-terminal sequence derived from the second β-glucosidase, wherein the first β-glucosidase and the second β-glucosidase are different from each other.

5. The isolated polypeptide of claim 1, wherein the N-terminal sequence and the C-terminal sequences are not directly connected, but are functionally connected via a linker domain.

6. The isolated polypeptide of claim 5, wherein the N-terminal sequence, the C-terminal sequence, or the linker domain comprises a loop region sequence of 3, 4, 5, 6, 7, 8, 9, 10, or 11 amino acid residues in length, comprising an amino acid sequence of SEQ ID NO:171 or 172.

7. The isolated polypeptide of claim 1, which has improved stability as compared to the first β-glucosidase or to the second β-glucosidase, optionally wherein the improved stability is an increased resistance to proteolytic cleavage under storage conditions or production conditions.

8. (canceled)

9. The isolated polypeptide of claim 4, wherein:
(a) the N-terminal sequence comprises an amino acid sequence that has at least 90% sequence identity to a sequence of the same length of SEQ ID NO:54, 56, 58, 60, 62, 64, 66, 68, 70, 72, 74, 76, 78 or 79, wherein the C-terminal sequence comprises a sequence motif of SEQ ID NO:170; or
(b) the N-terminal sequence comprises one or more or all of sequence motifs SEQ ID NOs:164-169, and the C-terminal sequence comprises an amino acid sequence that has at least 90% sequence identity to a sequence of the same length of SEQ ID NO:54, 56, 58, 60, 62, 64, 66, 68, 70, 72, 74, 76, 78 or 79.

10. (canceled)

11. The isolated polypeptide of claim 9, wherein the N-terminal sequence follows 3 or more, 4 or more, 5 or more of sequence motifs SEQ ID NOs:136-148, and wherein the C-terminal sequence follows 2 or more, 3 or more, or 4 or more of sequence motifs SEQ ID NOs:149-156.

12. A composition comprising the isolated polypeptide of claim 1.

13. The composition of claim 12, further comprising:
(a) one or more cellulases, optionally wherein the one or more cellulases are selected from endoglucanases, GH61/endoglucanases, cellobiohydrolases and other beta-glucosidases; or
(b) one or more hemicellulases, optionally wherein the one or more hemicellulases are selected from xylanases, β-xylosidases, or L-α-arabinofuranosidases.

14-16. (canceled)

17. The composition of claim 12, wherein the β-glucosidase is present in an amount of 1 wt. % to 75 wt. %, relative to the total amount of proteins in the composition.

18. The composition of claim 12, wherein the composition is a culture mixture or a fermentation broth.

19. (canceled)

20. An isolated polynucleotide:
a) comprising a nucleotide sequence having at least 70% sequence identity to SEQ ID NO:83; or
b) comprising a nucleotide sequence that is capable of hybridizing to SEQ ID NO:83 or to a complement thereof under high stringency conditions; or
c) encoding an isolated polypeptide having β-glucosidase activity, comprising an amino acid sequence that has at least about 70% identity to SEQ ID NO:135; or an isolated polypeptide having β-glucosidase activity, comprising an N-terminal sequence and a C-terminal sequence, wherein the N-terminal sequence comprises a first amino acid sequence derived from a first β-glucosidase, is at least 200 residues in length, and comprises one or more or all of SEQ ID NOs: 164-169, and wherein the C-terminal sequence comprises a second amino acid sequence derived from a second β-glucosidase, is at least 50 residues in length, and comprises SEQ ID NO:170.

21. (canceled)

22. A vector comprising the polynucleotide of claim 20.

23. A recombinant host cell engineered to express the polypeptide encoded by the polynucleotide of claim 20, optionally wherein the recombinant host cell is a bacterial or fungal cell, and optionally wherein the bacterial cell is selected from a Bacillus or an E. coli, and optionally wherein the fungal cell is selected from a Trichoderma, Aspergillus, Chrysosporium, or yeast cell.

24-26. (canceled)

27. A fermentation broth or culture mixture composition prepared by fermenting the recombinant host cell of claim 23.

28. A method of hydrolyzing a cellulosic biomass material comprising contacting the biomass material with the polypeptide of claim 1.

29. The method of claim 28, wherein the biomass material is selected from seeds, grains, tubers, plant waste or byproducts of food processing or industrial processing, stalks, corn cobs, stovers, leaves, grasses, perennial canes, wood, paper, pulp, and recycled paper, potatoes, soybean barley, rye, oats, wheat, beets, and sugar cane bagasse.

30. The method of claim 28, wherein the biomass material is subjected to pretreatment, optionally wherein the pretreatment comprises an acidic pretreatment or a basic pretreatment, or a combination of an acidic pretreatment and a basic pretreatment.

31. (canceled)

32. A method of applying the polypeptide of of claim 1 in a commercial setting or an industrial setting, wherein the method follows a merchant enzyme supply model strategy or an on-site biorefinery model strategy.


SEQUENCE LISTING (178 sequences)

SEQ ID NO: 1 - DNA, 2358 bp, Fusarium verticillioides
atgctgctcaatcttcaggtcgctgccagcgctttgtcgctttctcttttaggtggattggctgaggctgctacgccatatacccttccggactgtaccaaaggacctttgagcaagaatggaatctgcgatacttcgttatctccagctaaaagagcggctgctctagttgctgctctgacgcccgaagagaaggtgggcaatctggtcaggtaaaatatacccccccccataatcactattcggagattggagctgacttaacgcagcaatgcaactggtgcaccaagaatcggacttccaaggtacaactggtggaacgaagcccttcatggcctcgctggatctccaggtggtcgctttgccgacactcctccctacgacgcggccacatcatttcccatgcctcttctcatggccgctgctttcgacgatgatctgatccacgatatcggcaacgtcgtcggcaccgaagcgcgtgcgttcactaacggcggttggcgcggagtcgacttctggacacccaacgtcaacccttttaaagatcctcgctggggtcgtggctccgaaactccaggtgaagatgcccttcatgtcagccggtatgctcgctatatcgtcaggggtctcgaaggcgataaggagcaacgacgtattgttgctacctgcaagcactatgctggaaacgactttgaggactggggaggcttcacgcgtcacgactttgatgccaagattactcctcaggacttggctgagtactacgtcaggcctttccaggagtgcacccgtgatgcaaaggttggttccatcatgtgcgcctacaatgccgtgaacggcattcccgcatgcgcaaactcgtatctgcaggagacgatcctcagagggcactggaactggacgcgcgataacaactggatcactagtgattgtggcgccatgcaggatatctggcagaatcacaagtatgtcaagaccaacgctgaaggtgcccaggtagcttttgagaacggcatggattctagctgcgagtatactactaccagcgatgtctccgattcgtacaagcaaggcctcttgactgagaagctcatggatcgttcgttgaagcgccttttcgaagggcttgttcatactggtttctttgacggtgccaaagcgcaatggaactcgctcagttttgcggatgtcaacaccaaggaagctcaggatcttgcactcagatctgctgtggagggtgctgttcttcttaagaatgacggcactttgcctctgaagctcaagaagaaggatagtgttgcaatgatcggattctgggccaacgatacttccaagctgcagggtggttacagtggacgtgctccgttcctccacagcccgctttatgcagctgagaagcttggtcttgacaccaacgtggcttggggtccgacactgcagaacagctcatctcatgataactggaccaccaatgctgttgctgcggcgaagaagtctgattacattctctactttggtggtcttgacgcctctgctgctggcgaggacagagatcgtgagaaccttgactggcctgagagccagctgacccttcttcagaagctctctagtctcggcaagccactggttgttatccagcttggtgatcaagtcgatgacaccgctcttttgaagaacaagaagattaacagtattctttgggtcaattaccctggtcaggatggcggcactgcagtcatggacctgctcactggacgaaagagtcctgctggccgactacccgtcacgcaatatcccagtaaatacactgagcagattggcatgactgacatggacctcagacctaccaagtcgttgccagggagaacttatcgctggtactcaactccagttcttccctacggctttggcctccactacaccaagttccaagccaagttcaagtccaacaagttgacgtttgacatccagaagcttctcaagggctgcagtgctcaatactccgatacttgcgcgctgccccccatccaagttagtgtcaagaacaccggccgcattacctccgactttgtctctctggtctttatcaagagtgaagttggacctaagccttaccctctcaagacccttgcggcttatggtcgcttgcatgatgtcgcgccttcatcgacgaaggatatctcactggagtggacgttggataacattgcgcgacggggagagaatggtgatttggttgtttatcctgggacttacactctgttgctggatgagcctacgcaagccaagatccaggttacgctgactggaaagaaggctattttggataagtggcctcaagaccccaagtctgcgtaa

SEQ ID NO: 2 - protein, 766 aa, Fusarium verticillioides
MLLNLQVAASALSLSLLGGLAEAATPYTLPDCTKGPLSKNGICDTSLSPAKRAAALVAALTPEEKVGNLVSNATGAPRIGLPRYNWWNEALHGLAGSPGGRFADTPPYDAATSFPMPLLMAAAFDDDLIHDIGNVVGTEARAFTNGGWRGVDFWTPNVNPFKDPRWGRGSETPGEDALHVSRYARYIVRGLEGDKEQRRIVATCKHYAGNDFEDWGGFTRHDFDAKITPQDLAEYYVRPFQECTRDAKVGSIMCAYNAVNGIPACANSYLQETILRGHWNWTRDNNWITSDCGAMQDIWQNHKYVKTNAEGAQVAFENGMDSSCEYTTTSDVSDSYKQGLLTEKLMDRSLKRLFEGLVHTGFFDGAKAQWNSLSFADVNTKEAQDLALRSAVEGAVLLKNDGTLPLKLKKKDSVAMIGFWANDTSKLQGGYSGRAPFLHSPLYAAEKLGLDTNVAWGPTLQNSSSHDNWTTNAVAAAKKSDYILYFGGLDASAAGEDRDRENLDWPESQLTLLQKLSSLGKPLVVIQLGDQVDDTALLKNKKINSILWVNYPGQDGGTAVMDLLTGRKSPAGRLPVTQYPSKYTEQIGMTDMDLRPTKSLPGRTYRWYSTPVLPYGFGLHYTKFQAKFKSNKLTFDIQKLLKGCSAQYSDTCALPPIQVSVKNTGRITSDFVSLVFIKSEVGPKPYPLKTLAAYGRLHDVAPSSTKDISLEWTLDNIARRGENGDLVVYPGTYTLLLDEPTQAKIQVTLTGKKAILDKWPQDPKSA

SEQ ID NO: 3 - DNA, 1338 bp, Penicillium funiculosum
atgcttcagcgatttgcttatattttaccactggctctattgagtgttggagtgaaagccgacaacccctttgtgcagagcatctacaccgctgatccggcaccgatggtatacaatgaccgcgtttatgtcttcatggaccatgacaacaccggagctacctactacaacatgacagactggcatctgttctcgtcagcagatatggcgaattggcaagatcatggcattccaatgagcctggccaatttcacctgggccaacgcgaatgcgtgggccccgcaagtcatccctcgcaacggccaattctacttttatgctcctgtccgacacaacgatggttctatggctatcggtgtgggagtgagcagcaccatcacaggtccataccatgatgctatcggcaaaccgctagtagagaacaacgagattgatcccaccgtgttcatcgacgatgacggtcaggcatacctgtactggggaaatccagacctgtggtacgtcaaattgaaccaagatatgatatcgtacagcgggagccctactcagattccactcaccacggctggatttggtactcgaacgggcaatgctcaacggccgaccacttttgaagaagctccatgggtatacaaacgcaacggcatctactatatcgcctatgcagccgattgttgttctgaggatattcgctactccacgggaaccagtgccactggtccgtggacttatcgaggcgtcatcatgccgacccaaggtagcagcttcaccaatcacgagggtattatcgacttccagaacaactcctactttttctatcacaacggcgctcttcccggcggaggcggctaccaacgatctgtatgtgtggagcaattcaaatacaatgcagatggaaccattccgacgatcgaaatgaccaccgccggtccagctcaaattgggactctcaacccttacgtgcgacaggaagccgaaacggcggcatggtcttcaggcatcactacggaggtttgtagcgaaggcggaattgacgtcgggtttatcaacaatggcgattacatcaaagttaaaggcgtagctttcggttcaggagcccattctttctcagcgcgggttgcttctgcaaatagcggcggcactattgcaatacacctcggaagcacaactggtacgctcgtgggcacttgtactgtccccagcactggcggttggcagacttggactaccgttacctgttctgtcagtggcgcatctgggacccaggatgtgtattttgttttcggtggtagcggaacaggatacctgttcaactttgattattggcagttcgcataa

SEQ ID NO: 4 - protein, 445 aa, Penicillium funiculosum
MLQRFAYILPLALLSVGVKADNPFVQSIYTADPAPMVYNDRVYVFMDHDNTGATYYNMTDWHLFSSADMANWQDHGIPMSLANFTWANANAWAPQVIPRNGQFYFYAPVRHNDGSMAIGVGVSSTITGPYHDAIGKPLVENNEIDPTVFIDDDGQAYLYWGNPDLWYVKLNQDMISYSGSPTQIPLTTAGFGTRTGNAQRPTTFEEAPWVYKRNGIYYIAYAADCCSEDIRYSTGTSATGPWTYRGVIMPTQGSSFTNHEGIIDFQNNSYFFYHNGALPGGGGYQRSVCVEQFKYNADGTIPTIEMTTAGPAQIGTLNPYVRQEAETAAWSSGITTEVCSEGGIDVGFINNGDYIKVKGVAFGSGAHSFSARVASANSGGTIAIHLGSTTGTLVGTCTVPSTGGWQTWTTVTCSVSGASGTQDVYFVFGGSGTGYLFNFDYWQFA

SEQ ID NO: 5 - DNA, 1593 bp, Fusarium verticillioides
atgaaggtatactggctcgtggcgtgggccacttctttgacgccggcactggctggcttgattggacaccgtcgcgccaccaccttcaacaatcctatcatctactcagactttccagataacgatgtattcctcggtccagataactactactacttctctgcttccaacttccacttcagcccaggagcacccgttttgaagtctaaagatctgctaaactgggatctcatcggccattcaattccccgcctgaactttggcgacggctatgatcttcctcctggctcacgttattaccgtggaggtacttgggcatcatccctcagatacagaaagagcaatggacagtggtactggatcggctgcatcaacttctggcagacctgggtatacactgcctcatcgccggaaggtccatggtacaacaagggaaacttcggtgataacaattgctactacgacaatggcatactgatcgatgacgatgataccatgtatgtcgtatacggttccggtgaggtcaaagtatctcaactatctcaggacggattcagccaggtcaaatctcaggtagttttcaagaacactgatattggggtccaagacttggagggtaaccgcatgtacaagatcaacgggctctactatatcctaaacgatagcccaagtggcagtcagacctggatttggaagtcgaaatcaccctggggcccttatgagtctaaggtcctcgccgacaaagtcaccccgcctatctctggtggtaactcgccgcatcagggtagtctcataaagactcccaatggtggctggtacttcatgtcattcacttgggcctatcctgccggccgtcttccggttcttgcaccgattacgtggggtagcgatggtttccccattcttgtcaagggtgctaatggcggatggggatcatcttacccaacacttcctggcacggatggtgtgacaaagaattggacaaggactgataccttccgcggaacctcacttgctccgtcctgggagtggaaccataatccggacgtcaactccttcactgtcaacaacggcctgactctccgcactgctagcattacgaaggatatttaccaggcgaggaacacgctatctcaccgaactcatggtgatcatccaacaggaatagtgaagattgatttctctccgatgaaggacggcgaccgggccgggctttcagcgtttcgagaccaaagtgcatacatcggtattcatcgagataacggaaagttcacaatcgctacgaagcatgggatgaatatggatgagtggaacggaacaacaacagacctgggacaaataaaagccacagctaatgtgccttctggaaggaccaagatctggctgagacttcaacttgataccaacccagcaggaactggcaacactatcttttcttacagttgggatggagtcaagtatgaaacactgggtcccaacttcaaactgtacaatggttgggcattctttattgcttaccgattcggcatcttcaacttcgccgagacggctttaggaggctcgatcaaggttgagtctttcacagctgcatag

SEQ ID NO: 6 - protein, 530 aa, Fusarium verticillioides
MKVYWLVAWATSLTPALAGLIGHRRATTFNNPIIYSDFPDNDVFLGPDNYYYFSASNFHFSPGAPVLKSKDLLNWDLIGHSIPRLNFGDGYDLPPGSRYYRGGTWASSLRYRKSNGQWYWIGCINFWQTWVYTASSPEGPWYNKGNFGDNNCYYDNGILIDDDDTMYVVYGSGEVKVSQLSQDGFSQVKSQVVFKNTDIGVQDLEGNRMYKINGLYYILNDSPSGSQTWIWKSKSPWGPYESKVLADKVTPPISGGNSPHQGSLIKTPNGGWYFMSFTWAYPAGRLPVLAPITWGSDGFPILVKGANGGWGSSYPTLPGTDGVTKNWTRTDTFRGTSLAPSWEWNHNPDVNSFTVNNGLTLRTASITKDIYQARNTLSHRTHGDHPTGIVKIDFSPMKDGDRAGLSAFRDQSAYIGIHRDNGKFTIATKHGMNMDEWNGTTTDLGQIKATANVPSGRTKIWLRLQLDTNPAGTGNTIFSYSWDGVKYETLGPNFKLYNGWAFFIAYRFGIFNFAETALGGSIKVESFTAA

SEQ ID NO: 7 - DNA, 1374 bp, Fusarium verticillioides
atgcactacgctaccctcaccactttggtgctggctctgaccaccaacgtcgctgcacagcaaggcacagcaactgtcgacctctccaaaaatcatggaccggcgaaggcccttggttcaggcttcatatacggctggcctgacaacggaacaagcgtcgacacctccataccagatttcttggtaactgacatcaaattcaactcaaaccgcggcggtggcgcccaaatcccatcactgggttgggccagaggtggctatgaaggatacctcggccgcttcaactcaaccttatccaactatcgcaccacgcgcaagtataacgctgactttatcttgttgcctcatgacctctggggtgcggatggcgggcagggttcaaactccccgtttcctggcgacaatggcaattggactgagatggagttattctggaatcagcttgtgtctgacttgaaggctcataatatgctggaaggtcttgtgattgatgtttggaatgagcctgatattgatatcttttgggatcgcccgtggtcgcagtttcttgagtattacaatcgcgcgaccaaactacttcggtgagtctactactgatccatacgtatttacagtgagctgactggtcgaattagaaaaacacttcccaaaactcttctcagtggcccagccatggcacattctcccattctgtccgatgataaatggcatacctggcttcaatcagtagcgggtaacaagacagtccctgatatttactcctggcatcagattggcgcttgggaacgtgagccggacagcactatccccgactttaccaccttgcgggcgcaatatggcgttcccgagaagccaattgacgtcaatgagtacgctgcacgcgatgagcaaaatccagccaactccgtctactacctctctcaactagagcgtcataaccttagaggtcttcgcgcaaactggggtagcggatctgacctccacaactggatgggcaacttgatttacagcactaccggtacctcggaggggacttactaccctaatggtgaatggcaggcttacaagtactatgcggccatggcagggcagagacttgtgaccaaagcatcgtcggacttgaagtttgatgtctttgccactaagcaaggccgtaagattaagattatagccggcacgaggaccgttcaagcaaagtataacatcaaaatcagcggtttggaagtagcaggacttcctaagatgggtacggtaaaggtccggacttatcggttcgactgggctgggccgaatggaaaggttgacgggcctgttgatttgggggagaagaagtatacttattcggccaatacggtgagcagcccctctacttga

SEQ ID NO: 8 - protein, 439 aa, Fusarium verticillioides
MHYATLTTLVLALTTNVAAQQGTATVDLSKNHGPAKALGSGFIYGWPDNGTSVDTSIPDFLVTDIKFNSNRGGGAQIPSLGWARGGYEGYLGRFNSTLSNYRTTRKYNADFILLPHDLWGADGGQGSNSPFPGDNGNWTEMELFWNQLVSDLKAHNMLEGLVIDVWNEPDIDIFWDRPWSQFLEYYNRATKLLRKTLPKTLLSGPAMAHSPILSDDKWHTWLQSVAGNKTVPDIYSWHQIGAWEREPDSTIPDFTTLRAQYGVPEKPIDVNEYAARDEQNPANSVYYLSQLERHNLRGLRANWGSGSDLHNWMGNLIYSTTGTSEGTYYPNGEWQAYKYYAAMAGQRLVTKASSDLKFDVFATKQGRKIKIIAGTRTVQAKYNIKISGLEVAGLPKMGTVKVRTYRFDWAGPNGKVDGPVDLGEKKYTYSANTVSSPST

SEQ ID NO: 9 - DNA, 1350 bp, Fusarium verticillioides
atgtggctgacctccccattgctgttcgccagcaccctcctgggcctcactggcgttgctctagcagacaaccccatcgtccaagacatctacaccgcagacccagcaccaatggtctacaatggccgcgtctacctcttcacaggccatgacaacgacggctctaccgacttcaacatgacagactggcgtctcttctcgtcagcagacatggtcaactggcagcaccatggtgtccccatgagcttaaagaccttcagctgggccaacagcagagcctgggctggtcaagtcgttgcccgaaacggaaagttttacttctatgttcctgtccgtaatgccaagacgggtggaatggctattggtgtcggtgttagtaccaacatccttgggccctacactgatgcccttggaaagccattggtcgagaacaatgagatcgacccaactgtctacatcgacactgatggccaggcctatctctactggggcaaccctggattgtactacgtcaagctcaaccaagacatgctctcctacagtggtagcatcaacaaagtatcgctcacaacagctggattcggcagccgcccgaacaacgcgcagcgtcctactactttcgaggaaggaccgtggctgtacaagcgtggaaatctctactacatgatctacgcagccaactgctgttccgaggacattcgctactcaactggacccagcgccactggaccttggacttaccgcggtgtcgtgatgaacaaggcgggtcgaagcttcaccaaccatcctggcatcatcgactttgagaacaactcgtacttcttttaccacaatggcgctcttgatggaggtagcggttatactcggtctgtggctgtcgagagcttcaagtatggttcggacggtctgatccccgagatcaagatgactacgcaaggcccagcgcagctcaagtctctgaacccatatgtcaagcaggaggccgagactatcgcctggtctgagggtatcgagactgaggtctgcagcgaaggtggtctcaacgttgctttcatcgacaatggtgactacatcaaggtcaagggagtcgactttggcagcaccggtgcaaagacgttcagcgcccgtgttgcttccaacagcagcggaggcaagattgagcttcgacttggtagcaagaccggtaagttggttggtacctgcacggtaacgactacgggaaactggcagacttataagactgtggattgccccgtcagtggtgctactggtacgagcgatctattctttgtcttcacgggctctgggtctggctctctgttcaacttcaactggtggcagtttagctaa

SEQ ID NO: 10 - protein, 449 aa, Fusarium verticillioides
MWLTSPLLFASTLLGLTGVALADNPIVQDIYTADPAPMVYNGRVYLFTGHDNDGSTDFNMTDWRLFSSADMVNWQHHGVPMSLKTFSWANSRAWAGQVVARNGKFYFYVPVRNAKTGGMAIGVGVSTNILGPYTDALGKPLVENNEIDPTVYIDTDGQAYLYWGNPGLYYVKLNQDMLSYSGSINKVSLTTAGFGSRPNNAQRPTTFEEGPWLYKRGNLYYMIYAANCCSEDIRYSTGPSATGPWTYRGVVMNKAGRSFTNHPGIIDFENNSYFFYHNGALDGGSGYTRSVAVESFKYGSDGLIPEIKMTTQGPAQLKSLNPYVKQEAETIAWSEGIETEVCSEGGLNVAFIDNGDYIKVKGVDFGSTGAKTFSARVASNSSGGKIELRLGSKTGKLVGTCTVTTTGNWQTYKTVDCPVSGATGTSDLFFVFTGSGSGSLFNFNWWQFS

SEQ ID NO: 11 - DNA, 1725 bp, Fusarium verticillioides
atgcgcttctcttggctattgtgcccccttctagcgatgggaagtgctcttcctgaaacgaagacggatgtttcgacatacaccaaccctgtccttccaggatggcactcggatccatcgtgtatccagaaagatggcctctttctctgcgtcacttcaacattcatctccttcccaggtcttcccgtctatgcctcaagggatctagtcaactggcgtctcatcagccatgtctggaaccgcgagaaacagttgcctggcattagctggaagacggcaggacagcaacagggaatgtatgcaccaaccattcgataccacaagggaacatactacgtcatctgcgaatacctgggcgttggagatattattggtgtcatcttcaagaccaccaatccgtgggacgagagtagctggagtgaccctgttaccttcaagccaaatcacatcgaccccgatctgttctgggatgatgacggaaaggtttattgtgctacccatggcatcactctgcaggagattgatttggaaactggagagcttagcccggagcttaatatctggaacggcacaggaggtgtatggcctgagggtccccatatctacaagcgcgacggttactactatctcatgattgccgagggtggaactgccgaagaccacgctatcacaatcgctcgggcccgcaagatcaccggcccctatgaagcctacaataacaacccaatcttgaccaaccgcgggacatctgagtacttccagactgtcggtcacggtgatctgttccaagataccaagggcaactggtggggtctttgtcttgctactcgcatcacagcacagggagtttcacccatgggccgtgaagctgttttgttcaatggcacatggaacaagggcgaatggcccaagttgcaaccagtacgaggtcgcatgcctggaaacctcctcccaaagccgacgcgaaacgttcccggagatgggcccttcaacgctgacccagacaactacaacttgaagaagactaagaagatccctcctcactttgtgcaccatagagtcccaagagacggtgccttctctttgtcttccaagggtctgcacatcgtgcctagtcgaaacaacgttaccggtagtgtgttgccaggagatgagattgagctatcaggacagcgaggtctagctttcatcggacgccgccaaactcacactctgttcaaatatagtgttgatatcgacttcaagcccaagtccgatgatcaggaagctggaatcaccgttttccgcacgcagttcgaccatatcgatcttggcattgttcgtcttcctacaaaccaaggcagcaacaagaaatctaagcttgccttccgattccgggccacaggagctcagaatgttcctgcaccgaaggtagtaccggtccccgatggctgggagaagggcgtaatcagtctacatatcgaggcagccaacgcgacgcactacaaccttggagcttcgagccacagaggcaagactctcgacatcgcgacagcatcagcaagtcttgtgagtggaggcacgggttcatttgttggtagtttgcttggaccttatgctacctgcaacggcaaaggatctggagtggaatgtcccaagggaggtgatgtctatgtgacccaatggacttataagcccgtggcacaagagattgatcatggtgtttttgtgaaatcagaattgtag

SEQ ID NO: 12 - protein, 574 aa, Fusarium verticillioides
MRFSWLLCPLLAMGSALPETKTDVSTYTNPVLPGWHSDPSCIQKDGLFLCVTSTFISFPGLPVYASRDLVNWRLISHVWNREKQLPGISWKTAGQQQGMYAPTIRYHKGTYYVICEYLGVGDIIGVIFKTTNPWDESSWSDPVTFKPNHIDPDLFWDDDGKVYCATHGITLQEIDLETGELSPELNIWNGTGGVWPEGPHIYKRDGYYYLMIAEGGTAEDHAITIARARKITGPYEAYNNNPILTNRGTSEYFQTVGHGDLFQDTKGNWWGLCLATRITAQGVSPMGREAVLFNGTWNKGEWPKLQPVRGRMPGNLLPKPTRNVPGDGPFNADPDNYNLKKTKKIPPHFVHHRVPRDGAFSLSSKGLHIVPSRNNVTGSVLPGDEIELSGQRGLAFIGRRQTHTLFKYSVDIDFKPKSDDQEAGITVFRTQFDHIDLGIVRLPTNQGSNKKSKLAFRFRATGAQNVPAPKVVPVPDGWEKGVISLHIEAANATHYNLGASSHRGKTLDIATASASLVSGGTGSFVGSLLGPYATCNGKGSGVECPKGGDVYVTQWTYKPVAQEIDHGVFVKSEL

SEQ ID NO: 13 - DNA, 2251 bp, Podospora anserina
atgatccacctcaagccagccctcgcggcgttgttggcgctgtcgacgcaatgtgtggctattgatttgtttgtcaagtcttcgggggggaataagacgactgatatcatgtatggtcttatgcacgaggtatgtgttttgcgagatctcccttttgtttttgcgcactgctgacatggagactgcaaacaggatatcaacaactccggcgacggcggcatctacgccgagctaatctccaaccgcgcgttccaagggagtgagaagttcccctccaacctcgacaactggagccccgtcggtggcgctacccttacccttcagaagcttgccaagcccctttcctctgcgttgccttactccgtcaatgttgccaaccccaaggagggcaagggcaagggcaaggacaccaaggggaagaaggttggcttggccaatgctgggttttggggtatggatgtcaagaggcagaagtacactggtagcttccacgttactggtgagtacaagggtgactttgaggttagcttgcgcagcgcgattaccggggagacctttggcaagaaggtggtgaagggtgggagtaagaaggggaagtggaccgagaaggagtttgagttggtgcctttcaaggatgcgcccaacagcaacaacacctttgttgtgcagtgggatgccgaggtatgtgcttctttgatattggctgagatagaagttgggttgacatgatgtggtgcagggcgcaaaggacggatctttggatctcaacttgatcagcttgttccctccgacattcaagggaaggaagaatgggctgagaattgatcttgcgcagacgatggttgagctcaagccggtaagtcctctctagtcagaaaagtagagcctttgttaacgcttgacagaccttcttgcgcttccccggtggcaacatgctcgagggtaacaccttggacacttggtggaagtggtacgagaccattggccctctgaaggatcgcccgggcatggctggtgtctgggagtaccagcaaacccttggcttgggtctggtcgagtacatggagtgggccgatgacatgaacttggagcccagtatgtgatcccattttctggagtgacttctcttgctaacgtatccacagttgtcggtgtcttcgctggtcttgccctcgatggctcgttcgttcccgaatccgagatgggatgggtcatccaacaggctctcgacgaaatcgagttcctcactggcgatgctaagaccaccaaatggggtgccgtccgcgcgaagcttggtcaccccaagccttggaaggtcaagtgggttgagatcggtaacgaggattggcttgccggacgccctgctggcttcgagtcgtacatcaactaccgcttccccatgatgatgaaggccttcaacgaaaagtaccccgacatcaagatcatcgcctcgccctccatcttcgacaacatgacaatccccgcgggtgctgccggtgatcaccacccgtacctgactcccgatgagttcgttgagcgattcgccaagttcgataacttgagcaaggataacgtgacgctcatcggcgaggctgcgtcgacgcatcctaacggtggtatcgcttgggagggagatctcatgcccttgccttggtggggcggcagtgttgctgaggctatcttcttgatcagcactgagagaaacggtgacaagatcatcggtgctacttacgcgcctggtcttcgcagcttggaccgctggcaatggagcatgacctgggtgcagcatgccgccgacccggccctcaccactcgctcgaccagttggtatgtctggagaatcctcgcccaccacatcatccgtgagacgctcccggtcgatgccccggccggcaagcccaactttgaccctctgttctacgttgccggaaagagcgagagtggcaccggtatcttcaaggctgccgtctacaactcgactgaatcgatcccggtgtcgttgaagtttgatggtctcaacgagggagcggttgccaacttgacggtgcttactgggccggaggatccgtatggatacaacgaccccttcactggtatcaatgttgtcaaggagaagaccaccttcatcaaggccggaaagggcggcaagttcaccttcaccctgccgggcttgagtgttgctgtgttggagacggccgacgcggtcaagggtggcaagggaaagggcaagggcaagggaaagggtaactga

SEQ ID NO: 14 - protein, 676 aa, Podospora anserina
MIHLKPALAALLALSTQCVAIDLFVKSSGGNKTTDIMYGLMHEDINNSGDGGIYAELISNRAFQGSEKFPSNLDNWSPVGGATLTLQKLAKPLSSALPYSVNVANPKEGKGKGKDTKGKKVGLANAGFWGMDVKRQKYTGSFHVTGEYKGDFEVSLRSAITGETFGKKVVKGGSKKGKWTEKEFELVPFKDAPNSNNTFVVQWDAEGAKDGSLDLNLISLFPPTFKGRKNGLRIDLAQTMVELKPTFLRFPGGNMLEGNTLDTWWKWYETIGPLKDRPGMAGVWEYQQTLGLGLVEYMEWADDMNLEPIVGVFAGLALDGSFVPESEMGWVIQQALDEIEFLTGDAKTTKWGAVRAKLGHPKPWKVKWVEIGNEDWLAGRPAGFESYINYRFPMMMKAFNEKYPDIKIIASPSIFDNMTIPAGAAGDHHPYLTPDEFVERFAKFDNLSKDNVTLIGEAASTHPNGGIAWEGDLMPLPWWGGSVAEAIFLISTERNGDKIIGATYAPGLRSLDRWQWSMTWVQHAADPALTTRSTSWYVWRILAHHIIRETLPVDAPAGKPNFDPLFYVAGKSESGTGIFKAAVYNSTESIPVSLKFDGLNEGAVANLTVLTGPEDPYGYNDPFTGINVVKEKTTFIKAGKGGKFTFTLPGLSVAVLETADAVKGGKGKGKGKGKGN

SEQ ID NO: 15 - DNA, 1023 bp, Gibberella zeae
atgaagtccaagttgttattcccactcctctctttcgttggtcaaagtcttgccaccaacgacgactgtcctctcatcactagtagatggactgcggatccttcggctcatgtctttaacgacaccttgtggctctacccgtctcatgacatcgatgctggatttgagaatgatcctgatggaggccagtacgccatgagagattaccatgtctactctatcgacaagatctacggttccctgccggtcgatcacggtacggccctgtcagtggaggatgtcccctgggcctctcgacagatgtgggctcctgacgctgcccacaagaacggcaaatactacctatacttccctgccaaagacaaggatgatatcttcagaatcggcgttgctgtctcaccaacccccggcggaccattcgtccccgacaagagttggatccctcacactttcagcatcgaccccgccagtttcgtcgatgatgatgacagagcctacttggcatggggtggtatcatgggtggccagcttcaacgatggcaggataagaacaagtacaacgaatctggcactgagccaggaaacggcaccgctgccttgagccctcagattgccaagctgagcaaggacatgcacactctggcagagaagcctcgcgacatgctcattcttgaccccaagactggcaagccgctcctttctgaggatgaagaccgacgcttcttcgaaggaccctggattcacaagcgcaacaagatttactacctcacctactctactggcacaacccactatcttgtctatgcgacttcaaagaccccctatggtccttacacctaccagggcagaattctggagccagttgatggctggactactcactctagtatcgtcaagtaccagggtcagtggtggctattttatcacgatgccaagacatctggcaaggactatcttcgccaggtaaaggctaagaagatttggtacgatagcaaaggaaagatcttgacaaagaagccttga

SEQ ID NO: 16 - protein, 340 aa, Gibberella zeae
MKSKLLFPLLSFVGQSLATNDDCPLITSRWTADPSAHVFNDTLWLYPSHDIDAGFENDPDGGQYAMRDYHVYSIDKIYGSLPVDHGTALSVEDVPWASRQMWAPDAAHKNGKYYLYFPAKDKDDIFRIGVAVSPTPGGPFVPDKSWIPHTFSIDPASFVDDDDRAYLAWGGIMGGQLQRWQDKNKYNESGTEPGNGTAALSPQIAKLSKDMHTLAEKPRDMLILDPKTGKPLLSEDEDRRFFEGPWIHKRNKIYYLTYSTGTTHYLVYATSKTPYGPYTYQGRILEPVDGWTTHSSIVKYQGQWWLFYHDAKTSGKDYLRQVKAKKIWYDSKGKILTKKP

SEQ ID NO: 17 - DNA, 1047 bp, Fusarium oxysporum
atgcagctcaagtttctgtcttcagcattgctgttctctctgaccagcaaatgcgctgcgcaagacactaatgacattcctcccctgatcaccgacctctggtccgcagatccctcggctcatgttttcgaaggcaagctctgggtttacccatctcacgacatcgaagccaatgttgtcaacggcacaggaggcgctcaatacgccatgagggattaccatacctactccatgaagagcatctatggtaaagatcccgttgtcgaccacggcgtcgctctctcagtcgatgacgttccctgggcgaagcagcaaatgtgggctcctgacgcagctcataagaacggcaaatattatctgtacttccccgccaaggacaaggatgagatcttcagaattggagttgctgtctccaacaagcccagcggtcctttcaaggccgacaagagctggatccctggcacgtacagtatcgatcctgctagctacgtcgacactgataacgaggcctacctcatctggggcggtatctggggcggccagctccaagcctggcaggataaaaagaactttaacgagtcgtggattggagacaaggctgctcctaacggcaccaatgccctatctcctcagatcgccaagctaagcaaggacatgcacaagatcaccgaaacaccccgcgatctcgtcattctcgcccccgagacaggcaagcctcttcaggctgaggacaacaagcgacgattcttcgagggcccttggatccacaagcgcggcaagctttactacctcatgtactccaccggtgatacccacttccttgtctacgctacttccaagaacatctacggtccttatacctaccggggcaagattcttgatcctgttgatgggtggactactcatggaagtattgttgagtataagggacagtggtggcttttctttgctgatgcgcatacgtctggtaaggattaccttcgacaggtgaaggcgaggaagatctggtatgacaagaacggcaagatcttgcttcaccgtccttag

SEQ ID NO: 18 - protein, 348 aa, Fusarium oxysporum
MQLKFLSSALLFSLTSKCAAQDTNDIPPLITDLWSADPSAHVFEGKLWVYPSHDIEANVVNGTGGAQYAMRDYHTYSMKSIYGKDPVVDHGVALSVDDVPWAKQQMWAPDAAHKNGKYYLYFPAKDKDEIFRIGVAVSNKPSGPFKADKSWIPGTYSIDPASYVDTDNEAYLIWGGIWGGQLQAWQDKKNFNESWIGDKAAPNGTNALSPQIAKLSKDMHKITETPRDLVILAPETGKPLQAEDNKRRFFEGPWIHKRGKLYYLMYSTGDTHFLVYATSKNIYGPYTYRGKILDPVDGWTTHGSIVEYKGQWWLFFADAHTSGKDYLRQVKARKIWYDKNGKILLHRP

SEQ ID NO: 19 - DNA, 1677 bp, Aspergillus fumigates
atggcagctccaagtttatcctaccccacaggtatccaatcgtataccaatcctctcttccctggttggcactccgatcccagctgtgcctacgtagcggagcaagacacctttttctgcgtgacgtccactttcattgccttccccggtcttcctctttatgcaagccgagatctgcagaactggaaactggcaagcaatattttcaatcggcccagccagatccctgatcttcgcgtcacggatggacagcagtcgggtatctatgcgcccactctgcgctatcatgagggccagttctacttgatcgtttcgtacctgggcccgcagactaagggcttgctgttcacctcgtctgatccgtacgacgatgccgcgtggagcgatccgctcgaattcgcggtacatggcatcgacccggatatcttctgggatcacgacgggacggtctatgtcacgtccgccgaggaccagatgattaagcagtacacactcgatctgaagacgggggcgattggcccggttgactacctctggaacggcaccggaggagtctggcccgagggcccgcacatttacaagagagacggatactactacctcatgatcgcagagggaggtaccgagctcggccactcggagaccatggcgcgatctagaacccggacaggtccctgggagccatacccgcacaatccgctcttgtcgaacaagggcacctcggagtacttccagactgtgggccatgcggacttgttccaggatgggaacggcaactggtgggccgtggcgttgagcacccgatcagggcctgcatggaagaactatcccatgggtcgggagacggtgctcgcccccgccgcttgggagaagggtgagtggcctgtcattcagcctgtgagaggccaaatgcaggggccgtttccaccaccaaataagcgagttcctcgcggcgagggcggatggatcaagcaacccgacaaagtggatttcaggcccggatcgaagataccggcgcacttccagtactggcgatatcccaagacagaggattttaccgtctcccctcggggccacccgaatactcttcggctcacaccctccttttacaacctcaccggaactgcggacttcaagccggatgatggcctgtcgcttgttatgcgcaaacagaccgacaccttgttcacgtacactgtggacgtgtcttttgaccccaaggttgccgatgaagaggcgggtgtgactgttttccttacccagcagcagcacatcgatcttggtattgtccttctccagacaaccgaggggctgtcgttgtccttccggttccgcgtggaaggccgcggtaactacgaaggtcctcttccagaagccaccgtgcctgttcccaaggaatggtgtggacagaccatccggcttgagattcaggccgtgagtgacaccgagtatgtctttgcggctgccccggctcggcaccctgcacagaggcaaatcatcagccgcgccaactcgttgattgtcagtggtgatacgggacggtttactggctcgcttgttggcgtgtatgccacgtcgaacgggggtgccggatccacgcccgcatatatcagcagatggagatacgaaggacggggccagatgattgattttggtcgagtggtcccgagctactga

SEQ ID NO: 20 - protein, 558 aa, Aspergillus fumigates
MAAPSLSYPTGIQSYTNPLFPGWHSDPSCAYVAEQDTFFCVTSTFIAFPGLPLYASRDLQNWKLASNIFNRPSQIPDLRVTDGQQSGIYAPTLRYHEGQFYLIVSYLGPQTKGLLFTSSDPYDDAAWSDPLEFAVHGIDPDIFWDHDGTVYVTSAEDQMIKQYTLDLKTGAIGPVDYLWNGTGGVWPEGPHIYKRDGYYYLMIAEGGTELGHSETMARSRTRTGPWEPYPHNPLLSNKGTSEYFQTVGHADLFQDGNGNWWAVALSTRSGPAWKNYPMGRETVLAPAAWEKGEWPVIQPVRGQMQGPFPPPNKRVPRGEGGWIKQPDKVDFRPGSKIPAHFQYWRYPKTEDFTVSPRGHPNTLRLTPSFYNLTGTADFKPDDGLSLVMRKQTDTLFTYTVDVSFDPKVADEEAGVTVFLTQQQHIDLGIVLLQTTEGLSLSFRFRVEGRGNYEGPLPEATVPVPKEWCGQTIRLEIQAVSDTEYVFAAAPARHPAQRQIISRANSLIVSGDTGRFTGSLVGVYATSNGGAGSTPAYISRWRYEGRGQMIDFGRVVPSY

SEQ ID NO: 21 - DNA, 2320 bp, Penicillium funiculosum
atgggaaagatgtggcattcgatcttggttgtgttgggcttattgtctgtcgggcatgccatcactatcaacgtgtcccaaagtggcggcaataagaccagtcctttgcaatatggtctgatgttcgaggtaatccttctcttataccacatataaaagttgcgtcatttctaagacaagtcaaggacataaatcacggcggtgatggcggtctgtatgcagagcttgttcgaaaccgagcattccaaggtagcaccgtctatccagcaaacctcgatggatacgactcggtcaatggagcaatcctagcgcttcagaatttgacaaaccctctatcaccctccatgcctagctctctcaacgtcgccaaggggtccaacaatggaagcatcggtttcgcaaatgaaggctggtgggggatagaagtcaagccgcaaagatacgcgggctcattctacgtccagggggactatcaaggagatttcgacatctctcttcagtcgaaattgacacaagaagtcttcgcaacggcaaaagtcaggtcctcgggcaaacacgaggactgggttcaatacaagtacgagttggtgcccaaaaaggcagcatcaaacaccaataacactctgaccattacttttgactcaaaggtatgttaaattttgggtttagttcgatgtctggcaattgtcttacgagaaacgtagggattgaaagacggatccttgaacttcaacttgatcagcctatttcccccaacttacaacaatcggcccaatggcctaagaatcgacctggttgaagctatggctgaactagagggggtaagctcttacaaatcaactttatctttacgaagactaatgtgaaaacttagaaatttctgcggtttccaggcggtagcgatgtggaaggtgtacaagctccttactggtataagtggaatgaaacggtaggagatctcaaggaccgttatagtaggcccagtgcatggacgtacgaagaaagcaatggaattggcttgattgagtacatgaattggtgtgatgacatggggcttgagccgagtgagtgtattccattcagcgtcaaatccagtgttctaatcatacacatcagttcttgccgtatgggatggacattacctttcgaacgaagtgatatcggaaaacgatttgcagccatatatcgacgacaccctcaaccaactggaattcctgatgggtgccccagatacgccatatggtagttggcgtgcgtctctgggctatccgaagccgtggacgattaactacgtcgagattggaaacgaagacaatctatacgggggactagaaacatacatcgcctaccggtttcaggcatattacgacgctataacagctaaatatccccatatgacggtcatggaatctttgacggagatgcctggtccggcggccgctgcaagcgattaccatcaatattctactcctgatgggtttgtttcccagttcaactactttgatcagatgccagtcactaatagaacactgaacggtatgaaaacccccccttttttaaatatgcttttaatggtattaaccatctttcataggagagattgcaaccgtttatccaaataatcctagtaattcggtggcctggggaagcccattccccttgtatccttggtggattgggtccgttgcagaagctgttttcctaattggtgaagagaggaattcgccaaagataatcggtgctagctacgtacggaattctacttttcgagattttaacattggataagaaggactaacctcaatacaggctccaatgttcagaaatatcaacaattggcagtggtctccaacactcatcgcttttgacgctgactcgtcgcgtacaagtcgttcaacaagctggcatgtgatcaaggtatgctaattttcctcctcattcaaacccgcagatgtgagctaactttccgaagcttctctcgacaaacaaaatcacgcaaaatttacccacgacttggagtggcggtgacataggtccattatactgggtagctggacgaaacgacaatacaggatcgaacatattcaaggccgctgtttacaacagcacctcagacgtccctgtcaccgttcaatttgcaggatgcaacgcaaagagcgcaaatttgaccatcttgtcatccgacgatccgaacgcatcgaactaccctggggggcccgaagttgtgaagactgagatccagtctgtcactgcaaatgctcatggagcatttgagttcagtctcccgaacctaagtgtggctgttctcaaaacggagtaa

SEQ ID NO: 22 - protein, 642 aa, Penicillium funiculosum
MGKMWHSILVVLGLLSVGHAITINVSQSGGNKTSPLQYGLMFEDINHGGDGGLYAELVRNRAFQGSTVYPANLDGYDSVNGAILALQNLTNPLSPSMPSSLNVAKGSNNGSIGFANEGWWGIEVKPQRYAGSFYVQGDYQGDFDISLQSKLTQEVFATAKVRSSGKHEDWVQYKYELVPKKAASNTNNTLTITFDSKGLKDGSLNFNLISLFPPTYNNRPNGLRIDLVEAMAELEGKFLRFPGGSDVEGVQAPYWYKWNETVGDLKDRYSRPSAWTYEESNGIGLIEYMNWCDDMGLEPILAVWDGHYLSNEVISENDLQPYIDDTLNQLEFLMGAPDTPYGSWRASLGYPKPWTINYVEIGNEDNLYGGLETYIAYRFQAYYDAITAKYPHMTVMESLTEMPGPAAAASDYHQYSTPDGFVSQFNYFDQMPVTNRTLNGEIATVYPNNPSNSVAWGSPFPLYPWWIGSVAEAVFLIGEERNSPKIIGASYAPMFRNINNWQWSPTLIAFDADSSRTSRSTSWHVIKLLSTNKITQNLPTTWSGGDIGPLYWVAGRNDNTGSNIFKAAVYNSTSDVPVTVQFAGCNAKSANLTILSSDDPNASNYPGGPEVVKTEIQSVTANAHGAFEFSLPNLSVAVLKTE

SEQ ID NO: 23 - DNA, 739 bp, Aspergillus fumigates
atggtttctttctcctacctgctgctggcgtgctccgccattggagctctggctgcccccgtcgaacccgagaccacctcgttcaatgagactgctcttcatgagttcgctgagcgcgccggcaccccaagctccaccggctggaacaacggctactactactccttctggactgatggcggcggcgacgtgacctacaccaatggcgccggtggctcgtactccgtcaactggaggaacgtgggcaactttgtcggtggaaagggctggaaccctggaagcgctaggtaccgagctttgtcaacgtcggatgtgcagacctgtggctgacagaagtagaaccatcaactacggaggcagcttcaaccccagcggcaatggctacctggctgtctacggctggaccaccaaccccttgattgagtactacgttgttgagtcgtatggtacatacaaccccggcagcggcggtaccttcaggggcactgtcaacaccgacggtggcacttacaacatctacacggccgttcgctacaatgctccctccatcgaaggcaccaagaccttcacccagtactggtctgtgcgcacctccaagcgtaccggcggcactgtcaccatggccaaccacttcaacgcctggagcagactgggcatgaacctgggaactcacaactaccagattgtcgccactgagggttaccagagcagcggatctgcttccatcactgtctactag

SEQ ID NO: 24 - protein, 228 aa, Aspergillus fumigates
MVSFSYLLLACSAIGALAAPVEPETTSFNETALHEFAERAGTPSSTGWNNGYYYSFWTDGGGDVTYTNGAGGSYSVNWRNVGNFVGGKGWNPGSARTINYGGSFNPSGNGYLAVYGWTTNPLIEYYVVESYGTYNPGSGGTFRGTVNTDGGTYNIYTAVRYNAPSIEGTKTFTQYWSVRTSKRTGGTVTMANHFNAWSRLGMNLGTHNYQIVATEGYQSSGSASITVY

SEQ ID NO: 25 - DNA, 1002 bp, Aspergillus fumigates
atgatctccatttcctcgctcagctttggactcgccgctatcgccggcgcatatgctcttccgagtgacaaatccgtcagcttagcggaacgtcagacgatcacgaccagccagacaggcacaaacaatggctactactattccttctggaccaacggtgccggatcagtgcaatatacaaatggtgctggtggcgaatatagtgtgacgtgggcgaaccagaacggtggtgactttacctgtgggaagggctggaatccagggagtgaccagtaggcaacgcccgagaactatagaagaggacgcaaagaaagcactaaactctctactagtgacattaccttctctggcagcttcaatccttccggaaatgcttacctgtccgtgtatggatggactaccaaccccctagtcgaatactacatcctcgagaactatggcagttacaatcctggctcgggcatgacgcacaagggcaccgtcaccagcgatggatccacctacgacatctatgagcaccaacaggtcaaccagccttcgatcgtcggcacggccaccttcaaccaatactggtccatccgccaaaacaagcgatccagcggcacagtcaccaccgcgaatcacttcaaggcctgggctagtctggggatgaacctgggtacccataactatcagattgtttccactgagggatatgagagcagcggtacctcgaccatcactgtctcgtctggtggttcttcttctggtggaagtggtggcagctcgtctactacttcctcaggcagctcccctactggtggctccggcagtgtaagtcttcttccatatggttgtggctttatgtgtattctgactgtgatagtgctctgctttgtggggccagtgcggtggaattggctggtctggtcctacttgctgctcttcgggcacttgccaggtttcgaactcgtactactcccagtgcttgtagtaccttcttgcagggttatatccaagtga

SEQ ID NO: 26 - protein, 286 aa, Aspergillus fumigates
MISISSLSFGLAAIAGAYALPSDKSVSLAERQTITTSQTGTNNGYYYSFWTNGAGSVQYTNGAGGEYSVTWANQNGGDFTCGKGWNPGSDHDITFSGSFNPSGNAYLSVYGWTTNPLVEYYILENYGSYNPGSGMTHKGTVTSDGSTYDIYEHQQVNQPSIVGTATFNQYWSIRQNKRSSGTVTTANHFKAWASLGMNLGTHNYQIVSTEGYESSGTSTITVSSGGSSSGGSGGSSSTTSSGSSPTGGSGSCSALWGQCGGIGWSGPTCCSSGTCQVSNSYYSQCL

SEQ ID NO: 27 - DNA, 1053 bp, Fusarium verticilloides
atgcagctcaagtttctgtcttcagcattgttgctgtctttgaccggcaattgcgctgcgcaagacactaatgatatccctcctctgatcaccgacctctggtctgcggatccctcggctcatgttttcgagggcaaactctgggtttacccatctcacgacatcgaagccaatgtcgtcaacggcaccggaggcgctcagtacgccatgagagattatcacacctattccatgaagaccatctatggaaaagatcccgttatcgaccatggcgtcgctctgtcagtcgatgatgtcccatgggccaagcagcaaatgtgggctcctgacgcagcttacaagaacggcaaatattatctctacttccccgccaaggataaagatgagatcttcagaattggagttgctgtctccaacaagcccagcggtcctttcaaggccgacaagagctggatccccggtacttacagtatcgatcctgctagctatgtcgacactaatggcgaggcatacctcatctggggcggtatctggggcggccagcttcaggcctggcaggatcacaagacctttaatgagtcgtggctcggcgacaaagctgctcccaacggcaccaacgccctatctcctcagatcgccaagctaagcaaggacatgcacaagatcaccgagacaccccgcgatctcgtcatcctggcccccgagacaggcaagccccttcaagcagaggacaataagcgacgatttttcgaggggccctgggttcacaagcgcggcaagctgtactacctcatgtactctaccggcgacacgcacttcctcgtctacgcgacttccaagaacatctacggtccttatacctatcagggcaagattctcgaccctgttgatgggtggactacgcatggaagtattgttgagtacaagggacagtggtggttgttctttgcggatgcgcatacttctggaaaggattatctgagacaggttaaggcgaggaagatctggtatgacaaggatggcaagattttgcttactcgtcctaagatttag

SEQ ID NO: 28 - protein, 350 aa, Fusarium verticilloides
MQLKFLSSALLLSLTGNCAAQDTNDIPPLITDLWSADPSAHVFEGKLWVYPSHDIEANVVNGTGGAQYAMRDYHTYSMKTIYGKDPVIDHGVALSVDDVPWAKQQMWAPDAAYKNGKYYLYFPAKDKDEIFRIGVAVSNKPSGPFKADKSWIPGTYSIDPASYVDTNGEAYLIWGGIWGGQLQAWQDHKTFNESWLGDKAAPNGTNALSPQIAKLSKDMHKITETPRDLVILAPETGKPLQAEDNKRRFFEGPWVHKRGKLYYLMYSTGDTHFLVYATSKNIYGPYTYQGKILDPVDGWTTHGSIVEYKGQWWLFFADAHTSGKDYLRQVKARKIWYDKDGKILLTRPKI

SEQ ID NO: 29 - DNA, 1031 bp, Penicillium funiculosum
atgagtcgcagcatccttccgtacgcctctgttttcgccctcctgggcggggctatcgccgaaccgtttttggttctcaatagcgattttcccgatcccagtctcatagagacatccagcggatactatgcattcggtaccaccggaaacggagtcaatgcgcaggttgcttcttcaccagactttaatacctggactttgctttccggcacagatgccctcccgggaccatttccgtcatgggtagcttcgtctccacaaatctgggcgccagatgttttggttaaggtatgttcttatggaataacagttttaggagtaggtcagccaggatattgacaaaattataataggccgatggtacctatgtcatgtacttttcggcatctgctgcgagtgactcgggcaaacactgcgttggtgccgcaactgcgacctcaccggaaggaccttacaccccggtcgatagcgctgttgcctgtccattagaccagggaggagctattgatgccaatggatttattgacaccgacggcactatatacgttgtatacaaaattgatggaaacagtctagacggtgatggaaccacacatcctacccccatcatgcttcaacaaatggaggcagacggaacaaccccaaccggcagcccaatccaactcattgaccgatccgacctcgacggacctttgatcgaggctcctagtttgctcctctccaatggaatctactacctcagtttctcttccaactactacaacactaattactacgacacttcatacgcctatgcctcgtcgattactggtccttggaccaaacaatctgcgccttatgcacccttgttggttactggaaccgagactagcaatgacggcgcattgagcgcccctggtggtgccgatttctccgtcgatggcaccaagatgttgttccacgcaaacctcaatggacaagatatctcgggcggacgcgccttatttgctgcgtcaattactgaggccagcgatgtggttacattgcagtag

SEQ ID NO: 30 - protein, 321 aa, Penicillium funiculosum
MSRSILPYASVFALLGGAIAEPFLVLNSDFPDPSLIETSSGYYAFGTTGNGVNAQVASSPDFNTWTLLSGTDALPGPFPSWVASSPQIWAPDVLVKADGTYVMYFSASAASDSGKHCVGAATATSPEGPYTPVDSAVACPLDQGGAIDANGFIDTDGTIYVVYKIDGNSLDGDGTTHPTPIMLQQMEADGTTPTGSPIQLIDRSDLDGPLIEAPSLLLSNGIYYLSFSSNYYNTNYYDTSYAYASSITGPWTKQSAPYAPLLVTGTETSNDGALSAPGGADFSVDGTKMLFHANLNGQDISGGRALFAASITEASDVVTLQ

SEQ ID NO: 31 - DNA, 2186 bp, Fusarium verticillioide
atggttcgcttcagttcaatcctagcggctgcggcttgcttcgtggctgttgagtcagtcaacatcaaggtcgacagcaagggcggaaacgctactagcggtcaccaatatggcttccttcacgaggttggtattgacacaccactggcgatgattgggatgctaacttggagctaggatatcaacaattccggtgatggtggcatctacgctgagctcatccgcaatcgtgctttccagtacagcaagaaataccctgtttctctatctggctggagacccatcaacgatgctaagctctccctcaaccgtctcgacactcctctctccgacgctctccccgtttccatgaacgtgaagcctggaaagggcaaggccaaggagattggtttcctcaacgagggttactggggaatggatgtcaagaagcaaaagtacactggctctttctgggttaagggcgcttacaagggccactttacagcttctttgcgatctaaccttaccgacgatgtctttggcagcgtcaaggtcaagtccaaggccaacaagaagcagtgggttgagcatgagtttgtgcttactcctaacaagaatgcccctaacagcaacaacacttttgctatcacctacgatcccaaggtgagtaacaatcaaaactgggacgtgatgtatactgacaatttgtagggcgctgatggagctcttgacttcaacctcattagcttgttccctcccacctacaagggccgcaagaacggtcttcgagttgatcttgccgaggctctcgaaggtctccaccccgtaaggtttaccgtctcacgtgtatcgtgaacagtcgctgacttgtagaaaagagcctgctgcgcttccccggtggtaacatgctcgagggcaacaccaacaagacctggtgggactggaaggataccctcggacctctccgcaaccgtcctggtttcgagggtgtctggaactaccagcagacccatggtcttggaatcttggagtacctccagtgggctgaggacatgaaccttgaaatcagtaggttctataaaattcagtgacggttatgtgcatgctaacagatttcagttgtcggtgtctacgctggcctctccctcgacggctccgtcacccccaaggaccaactccagcccctcatcgacgacgcgctcgacgagatcgaattcatccgaggtcccgtcacttcaaagtggggaaagaagcgcgctgagctcggccaccccaagcctttcagactctcctacgttgaagtcggaaacgaggactggctcgctggttatcccactggctggaactcttacaaggagtaccgcttccccatgttcctcgaggctatcaagaaagctcaccccgatctcaccgtcatctcctctggtgcttctattgaccccgttggtaagaaggatgctggtttcgatattcctgctcctggaatcggtgactaccacccttaccgcgagcctgatgttcttgttgaggagttcaacctgtttgataacaataagtatggtcacatcattggtgaggttgcttctacccaccccaacggtggaactggctggagtggtaaccttatgccttacccctggtggatctctggtgttggcgaggccgtcgctctctgcggttatgagcgcaacgccgatcgtattcccggaacattctacgctcctatcctcaagaacgagaaccgttggcagtgggctatcaccatgatccaattcgccgccgactccgccatgaccacccgctccaccagctggtatgtctggtcactcttcgcaggccaccccatgacccatactctccccaccaccgccgacttcgaccccctctactacgtcgctggtaagaacgaggacaagggaactcttatctggaagggtgctgcgtataacaccaccaagggtgctgacgttcccgtgtctctgtccttcaagggtgtcaagcccggtgctcaagctgagcttactcttctgaccaacaaggagaaggatccttttgcgttcaatgatcctcacaagggcaacaatgttgttgatactaagaagactgttctcaaggccgatggaaagggtgctttcaacttcaagcttcctaacctgagcgtcgctgttcttgagaccctcaagaagggaaagccttactctagctag

SEQ ID NO: 32 - protein, 660 aa, Fusarium verticillioide
MVRFSSILAAAACFVAVESVNIKVDSKGGNATSGHQYGFLHEDINNSGDGGIYAELIRNRAFQYSKKYPVSLSGWRPINDAKLSLNRLDTPLSDALPVSMNVKPGKGKAKEIGFLNEGYWGMDVKKQKYTGSFWVKGAYKGHFTASLRSNLTDDVFGSVKVKSKANKKQWVEHEFVLTPNKNAPNSNNTFAITYDPKGADGALDFNLISLFPPTYKGRKNGLRVDLAEALEGLHPSLLRFPGGNMLEGNTNKTWWDWKDTLGPLRNRPGFEGVWNYQQTHGLGILEYLQWAEDMNLEIIVGVYAGLSLDGSVTPKDQLQPLIDDALDEIEFIRGPVTSKWGKKRAELGHPKPFRLSYVEVGNEDWLAGYPTGWNSYKEYRFPMFLEAIKKAHPDLTVISSGASIDPVGKKDAGFDIPAPGIGDYHPYREPDVLVEEFNLFDNNKYGHIIGEVASTHPNGGTGWSGNLMPYPWWISGVGEAVALCGYERNADRIPGTFYAPILKNENRWQWAITMIQFAADSAMTTRSTSWYVWSLFAGHPMTHTLPTTADFDPLYYVAGKNEDKGTLIWKGAAYNTTKGADVPVSLSFKGVKPGAQAELTLLTNKEKDPFAFNDPHKGNNVVDTKKTVLKADGKGAFNFKLPNLSVAVLETLKKGKPYSS

SEQ ID NO: 33-40 - skipped (8 entries)

SEQ ID NO: 41 - DNA, 1352 bp, Trichoderma reesei
atgaaagcaaacgtcatcttgtgcctcctggcccccctggtcgccgctctccccaccgaaaccatccacctcgaccccgagctcgccgctctccgcgccaacctcaccgagcgaacagccgacctctgggaccgccaagcctctcaaagcatcgaccagctcatcaagagaaaaggcaagctctactttggcaccgccaccgaccgcggcctcctccaacgggaaaagaacgcggccatcatccaggcagacctcggccaggtgacgccggagaacagcatgaagtggcagtcgctcgagaacaaccaaggccagctgaactggggagacgccgactatctcgtcaactttgcccagcaaaacggcaagtcgatacgcggccacactctgatctggcactcgcagctgcctgcgtgggtgaacaatatcaacaacgcggatactctgcggcaagtcatccgcacccatgtctctactgtggttgggcggtacaagggcaagattcgtgcttgggtgagttttgaacaccacatgccccttttcttagtccgctcctcctcctcttggaacttctcacagttatagccgtatacaacattcgacaggaaatttaggatgacaactactgactgacttgtgtgtgtgatggcgataggacgtggtcaatgaaatcttcaacgaggatggaacgctgcgctcttcagtcttttccaggctcctcggcgaggagtttgtctcgattgcctttcgtgctgctcgagatgctgacccttctgcccgtctttacatcaacgactacaatctcgaccgcgccaactatggcaaggtcaacgggttgaagacttacgtctccaagtggatctctcaaggagttcccattgacggtattggtgagccacgacccctaaatgtcccccattagagtctctttctagagccaaggcttgaagccattcagggactgacacgagagccttctctacaggaagccagtcccatctcagcggcggcggaggctctggtacgctgggtgcgctccagcagctggcaacggtacccgtcaccgagctggccattaccgagctggacattcagggggcaccgacgacggattacacccaagttgttcaagcatgcctgagcgtctccaagtgcgtcggcatcaccgtgtggggcatcagtgacaaggtaagttgcttcccctgtctgtgcttatcaactgtaagcagcaacaactgatgctgtctgtctttacctaggactcgtggcgtgccagcaccaaccctcttctgtttgacgcaaacttcaaccccaagccggcatataacagcattgttggcatcttacaatag

SEQ ID NO: 42 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 43 - protein, 222 aa, Trichoderma reesei
MVSFTSLLAASPPSRASCRPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 44 - protein, 797 aa, Trichoderma reesei
MVNNAALLAALSALLPTALAQNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTIENWPLEEQQIKDATPDA

SEQ ID NO: 45 - protein, 744 aa, Trichoderma reesei
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 46 - DNA, 2031 bp, Podospora anserina
atgatccacctcaagccagccctcgcggcgttgttggcgctgtcgacgcaatgtgtggctattgatttgtttgtcaagtcttcgggggggaataagacgactgatatcatgtatggtcttatgcacgaggatatcaacaactccggcgacggcggcatctacgccgagctaatctccaaccgcgcgttccaagggagtgagaagttcccctccaacctcgacaactggagccccgtcggtggcgctacccttacccttcagaagcttgccaagcccctttcctctgcgttgccttactccgtcaatgttgccaaccccaaggagggcaagggcaagggcaaggacaccaaggggaagaaggttggcttggccaatgctgggttttggggtatggatgtcaagaggcagaagtacactggtagcttccacgttactggtgagtacaagggtgactttgaggttagcttgcgcagcgcgattaccggggagacctttggcaagaaggtggtgaagggtgggagtaagaaggggaagtggaccgagaaggagtttgagttggtgcctttcaaggatgcgcccaacagcaacaacacctttgttgtgcagtgggatgccgagggcgcaaaggacggatctttggatctcaacttgatcagcttgttccctccgacattcaagggaaggaagaatgggctgagaattgatcttgcgcagacgatggttgagctcaagccgaccttcttgcgcttccccggtggcaacatgctcgagggtaacaccttggacacttggtggaagtggtacgagaccattggccctctgaaggatcgcccgggcatggctggtgtctgggagtaccagcaaacccttggcttgggtctggtcgagtacatggagtgggccgatgacatgaacttggagcccattgtcggtgtcttcgctggtcttgccctcgatggctcgttcgttcccgaatccgagatgggatgggtcatccaacaggctctcgacgaaatcgagttcctcactggcgatgctaagaccaccaaatggggtgccgtccgcgcgaagcttggtcaccccaagccttggaaggtcaagtgggttgagatcggtaacgaggattggcttgccggacgccctgctggcttcgagtcgtacatcaactaccgcttccccatgatgatgaaggccttcaacgaaaagtaccccgacatcaagatcatcgcctcgccctccatcttcgacaacatgacaatccccgcgggtgctgccggtgatcaccacccgtacctgactcccgatgagttcgttgagcgattcgccaagttcgataacttgagcaaggataacgtgacgctcatcggcgaggctgcgtcgacgcatcctaacggtggtatcgcttgggagggagatctcatgcccttgccttggtggggcggcagtgttgctgaggctatcttcttgatcagcactgagagaaacggtgacaagatcatcggtgctacttacgcgcctggtcttcgcagcttggaccgctggcaatggagcatgacctgggtgcagcatgccgccgacccggccctcaccactcgctcgaccagttggtatgtctggagaatcctcgcccaccacatcatccgtgagacgctcccggtcgatgccccggccggcaagcccaactttgaccctctgttctacgttgccggaaagagcgagagtggcaccggtatcttcaaggctgccgtctacaactcgactgaatcgatcccggtgtcgttgaagtttgatggtctcaacgagggagcggttgccaacttgacggtgcttactgggccggaggatccgtatggatacaacgaccccttcactggtatcaatgttgtcaaggagaagaccaccttcatcaaggccggaaagggcggcaagttcaccttcaccctgccgggcttgagtgttgctgtgttggagacggccgacgcggtcaagggtggcaagggaaagggcaagggcaagggaaagggtaactga

SEQ ID NO: 47 - DNA, 2031 bp, Artificial Sequence
  synthetic codon optimized GH51 enzyme from Podospora anserina
atgatccacctcaagcccgccctcgccgccctcctcgccctcagcacccaatgcgtcgccatcgacctcttcgtcaagagcagcggcggcaacaagaccaccgacatcatgtacggcctcatgcacgaggacatcaacaacagcggcgacggcggcatctacgccgagctgatcagcaaccgcgccttccagggcagcgagaagttccccagcaacctcgacaactggtcccccgtcggcggcgccaccctcaccctccagaagctcgccaagcccctgtcctctgccctcccctactccgtcaacgtcgccaaccccaaggagggtaagggtaagggcaaggacaccaagggcaagaaggtcggcctcgccaacgccggcttttggggcatggacgtcaagcgccagaaatacaccggcagcttccacgtcaccggcgagtacaagggcgacttcgaggtcagcctccgcagcgccattaccggcgagaccttcggcaagaaggtcgtcaagggcggcagcaagaagggcaagtggaccgagaaggagttcgagctggtccccttcaaggacgcccccaacagcaacaacaccttcgtcgtccagtgggacgccgagggcgccaaggacggcagcctcgacctcaacctcatcagcctcttcccgcccaccttcaagggccgcaagaacggcctccgcatcgacctcgcccagaccatggtcgagctgaagcccaccttcctccgctttcccggcggcaacatgctcgagggcaacaccctcgacacctggtggaagtggtacgagaccatcggccccctgaaggaccgccctggcatggccggcgtctgggagtaccagcagacgctgggcctcggcctggtcgagtacatggagtgggccgacgacatgaacctcgagcccatcgtcggcgtctttgctggcctggccctggatggcagctttgtccccgagagcgagatgggctgggtcatccagcaggctctcgatgagatcgagttcctcaccggcgacgccaagaccaccaagtggggcgccgtccgcgccaagctcggccaccctaagccctggaaggtcaaatgggtcgagatcggcaacgaggactggctcgccggccgacctgccggcttcgagagctacatcaactaccgcttccccatgatgatgaaggccttcaacgagaaataccccgacatcaagatcattgccagcccctccatcttcgacaacatgaccattccagccggtgctgccggtgaccaccacccctacctcacccccgacgaatttgtcgagcgcttcgccaagttcgacaacctcagcaaggacaacgtcaccctcattggcgaggccgccagcacccaccccaacggcggcattgcctgggagggcgacctcatgcccctgccctggtggggcggcagcgtcgccgaggccatcttcctcatcagcaccgagcgcaacggcgacaagatcatcggcgccacctacgcccctggcctccgatctctcgaccgctggcagtggagcatgacctgggtccagcacgccgccgaccctgccctcaccacccgcagcaccagctggtacgtctggcgcatcctcgcccaccacatcattcgcgagaccctccccgtcgacgcccccgccggcaagcccaacttcgaccccctcttctacgtcgctggcaagtcggagagcggcaccggcatcttcaaggccgccgtctacaacagcaccgagagcatccccgtcagcctcaagttcgacggcctcaacgagggcgccgtcgccaacctcaccgtcctcaccggccccgaggacccctacggctacaacgaccccttcaccggcatcaacgtcgtcaaggaaaagaccaccttcatcaaggccggcaagggcggcaagttcacctttaccctccccggcctctctgtcgccgtcctcgagaccgccgacgccgtgaagggtggcaagggaaagggaaagggcaagggtaagggtaactaa

SEQ ID NO: 48 - DNA, 1020 bp, Gibberella zeae
atgtatcggaagttggccgtcatctcggccttcttggccacagctcgtgctaccaacgacgactgtcctctcatcactagtagatggactgcggatccttcggctcatgtctttaacgacaccttgtggctctacccgtctcatgacatcgatgctggatttgagaatgatcctgatggaggccagtacgccatgagagattaccatgtctactctatcgacaagatctacggttccctgccggtcgatcacggtacggccctgtcagtggaggatgtcccctgggcctctcgacagatgtgggctcctgacgctgcccacaagaacggcaaatactacctatacttccctgccaaagacaaggatgatatcttcagaatcggcgttgctgtctcaccaacccccggcggaccattcgtccccgacaagagttggatccctcacactttcagcatcgaccccgccagtttcgtcgatgatgatgacagagcctacttggcatggggtggtatcatgggtggccagcttcaacgatggcaggataagaacaagtacaacgaatctggcactgagccaggaaacggcaccgctgccttgagccctcagattgccaagctgagcaaggacatgcacactctggcagagaagcctcgcgacatgctcattcttgaccccaagactggcaagccgctcctttctgaggatgaagaccgacgcttcttcgaaggaccctggattcacaagcgcaacaagatttactacctcacctactctactggcacaacccactatcttgtctatgcgacttcaaagaccccctatggtccttacacctaccagggcagaattctggagccagttgatggctggactactcactctagtatcgtcaagtaccagggtcagtggtggctattttatcacgatgccaagacatctggcaaggactatcttcgccaggtaaaggctaagaagatttggtacgatagcaaaggaaagatcttgacaaagaagccttga

SEQ ID NO: 49 - DNA, 1038 bp, Fusarium oxysporum
atgtatcggaagttggccgtcatctcggccttcttggccacagctcgtgctcaagacactaatgacattcctcccctgatcaccgacctctggtccgcagatccctcggctcatgttttcgaaggcaagctctgggtttacccatctcacgacatcgaagccaatgttgtcaacggcacaggaggcgctcaatacgccatgagggattaccatacctactccatgaagagcatctatggtaaagatcccgttgtcgaccacggcgtcgctctctcagtcgatgacgttccctgggcgaagcagcaaatgtgggctcctgacgcagctcataagaacggcaaatattatctgtacttccccgccaaggacaaggatgagatcttcagaattggagttgctgtctccaacaagcccagcggtcctttcaaggccgacaagagctggatccctggcacgtacagtatcgatcctgctagctacgtcgacactgataacgaggcctacctcatctggggcggtatctggggcggccagctccaagcctggcaggataaaaagaactttaacgagtcgtggattggagacaaggctgctcctaacggcaccaatgccctatctcctcagatcgccaagctaagcaaggacatgcacaagatcaccgaaacaccccgcgatctcgtcattctcgcccccgagacaggcaagcctcttcaggctgaggacaacaagcgacgattcttcgagggcccttggatccacaagcgcggcaagctttactacctcatgtactccaccggtgatacccacttccttgtctacgctacttccaagaacatctacggtccttatacctaccggggcaagattcttgatcctgttgatgggtggactactcatggaagtattgttgagtataagggacagtggtggcttttctttgctgatgcgcatacgtctggtaaggattaccttcgacaggtgaaggcgaggaagatctggtatgacaagaacggcaagatcttgcttcaccgtccttag

SEQ ID NO: 50 - DNA, 1920 bp, Penicillium funiculosum
atgtaccggaagctcgccgtgatcagcgccttcctggcgactgctcgcgccatcaccatcaacgtcagccagagcggcggcaacaagaccagcccgctccagtacggcctcatgttcgaggacatcaaccacggcggcgacggcggcctctacgccgagctggtccggaaccgggccttccagggcagcaccgtctacccggccaacctcgacggctacgactcggtgaacggcgcgattctcgcgctccagaacctcaccaacccgctcagcccgagcatgccctcgtcgctgaacgtcgccaagggctcgaacaacggcagcatcggcttcgccaacgaggggtggtggggcatcgaggtcaagccgcagcggtacgccggcagcttctacgtccagggcgactaccagggcgacttcgacatcagcctccagagcaagctcacccaggaggtcttcgcgacggcgaaggtccggtcgagcggcaagcacgaggactgggtccagtacaagtacgagctggtcccgaagaaggccgccagcaacaccaacaacaccctcaccatcaccttcgacagcaagggcctcaaggacggcagcctcaacttcaacctcatcagcctcttcccgccgacctacaacaaccggccgaacggcctccggatcgacctcgtcgaggccatggcggagctggagggcaagttcctccgcttccccggcggctcggacgtggagggcgtccaggccccgtactggtacaagtggaacgagaccgtcggcgacctcaaggaccgctactcgcgcccgagcgcctggacctacgaggagagcaacggcatcggcctcatcgagtacatgaactggtgcgacgacatgggcctcgagccgatcctcgccgtctgggacggccactacctcagcaacgaggtcatcagcgagaacgacctccagccgtacatcgacgacaccctcaaccagctcgagttcctcatgggcgccccggacactccctacgggtcttggagggctagcctcggctacccgaagccgtggaccatcaactacgtcgagatcggcaacgaggacaacctctacggcggcctcgagacctacatcgcctaccggttccaggcctactacgacgccatcaccgccaagtacccgcacatgaccgtcatggagagcctcaccgagatgcccggccccgctgccgcggcgtcggactaccaccagtactcgacgcccgacggcttcgtcagccagttcaactacttcgaccagatgccggtcaccaaccgcacgctgaacggcgagatcgccaccgtctaccccaacaacccgagcaactcggtggcgtggggcagcccgttcccgctctacccgtggtggatcgggtccgtggctgaggccgtcttcctcatcggcgaggagcggaacagcccgaagatcatcggcgccagctacgcccccatgttccgcaacattaacaactggcagtggagcccgaccctgatcgccttcgacgccgacagcagccggacgtcgcgctctacttcctggcacgtcatcaagctcctcagcaccaacaagatcacccagaacctgcccacgacgtggtctgggggggacatcggcccgctctactgggtcgccggccggaacgacaacaccggcagcaacatcttcaaggccgccgtctacaacagcaccagcgacgtcccggtcaccgtccagttcgccggctgcaacgccaagagcgccaacctcaccatcctctcgtcggacgaccccaacgccagcaactacccgggcggccccgaggtcgtcaagaccgagatccagagcgtcaccgccaacgcccacggcgccttcgagttcagcctcccgaacctgtcggtggctgtgctgaagacggagtag

SEQ ID NO: 51 - DNA, 1044 bp, Trichoderma reesei
atgatccagaagctttccaaccttcttctcaccgcactagcggtggcaaccggtgttgttggacacggacacatcaacaacattgtcgtcaacggagtgtactaccagggatatgatcctacatcgttcccatatgaatctgacccgcccatagtggtgggctggacggctgccgatcttgacaacggcttcgtctcacccgacgcatatcagagcccggacatcatctgccacaagaatgccaccaacgccaaaggacacgcgtccgtcaaggccggagacactattcccctccagtgggtgccagttccttggccgcacccaggccccatcgtcgactacctggccaactgcaacggcgactgcgagaccgtggacaagacgtcccttgagttcttcaagattgacggcgtcggtctcatcagcggcggagatccgggcaactgggcctcggacgtgttgattgccaacaacaacacctgggttgtcaagatccccgaggatctcgccccgggcaactacgtgcttcgccacgagatcatcgccttgcacagcgccgggcaggcggacggcgctcagaactaccctcagtgcttcaacctcgccgtcccaggctccggatctctgcagccgagcggcgtcaagggaaccgcgctctaccactccgatgaccccggtgtcctcatcaacatctacaccagccctcttgcgtacaccattcctggaccttccgtggtatcaggcctccccacgagtgtcgcccagggcagctccgccgcgacggccactgccagcgccactgttcctggcggtagcggaccgggaaacccgaccagtaagactacgacgacggcgaggacgacacaggcctcctctagcagggccagctctactcctcctgctactacgtcggcacctggtggaggcccaacccagactttgtacggccagtgtggtggcagcggctacagtggtcctactcgatgcgcgccgccggccacttgctctaccttgaacccatactacgcccagtgccttaactag

SEQ ID NO: 52 - protein, 344 aa, Trichoderma reesei
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 53 - DNA, 2260 bp, Podospora anserina
atggctcttcaaaccttcttcctgctggcggcagccatgctggccaacgcagagacaacaggcgaaaaggtctctcggcaagcaccgtctggcgctcaagcatgggccgccgcccactcccaggctgccgccactctggccagaatgtcacagcaagacaagatcaacatggtcacgggcattggctgggacagagggccttgcgtgggaaacacagctgccatcagctccatcaactatcctcaaatctgtcttcaggatggaccattgggcattcgcttcggcactggtaccaccgccttcacacctggcgtccaagctgcttcgacatgggacgttgatctgatccggcagcgcggtgcttacctgggcgccgaagccaagggctgcggcattcacatccttttggggcccgttgccggtgccctgggcaagattccccacggcggtcgcaactgggagggatttggcgccgacccctaccttgccggtattgccatgaaggagaccatcgagggtattcagtcagcaggcgtccaggccaacgccaagcactacattgcaaacgaacaagagctcaaccgcgagaccatgagcagcaatgtggatgaccgcactcagcacgagctctacctctggccctttgccgacgccgtgcacgccaacgtcgccagcgtcatgtgcagttacaacaagctcaatggcacgtgggcttgcgagaatgacaaggctctgaatcagatcttgaagaaggagctcggattccagggctacgttctcagcgactggaatgctcagcacagcactgctctgtctgctaacagtggtctggacatgactatgcccggtaccgatttcaacggccgcaatgtctactggggccctcaactgaacaacgctgtcaacgccggccaggttcagagatccagactagacgacatgtgcaagagaatcttggctggctggtacttgctcggtcagaaccagggctatcccgccatcaacatcagggccaacgttcagggcaaccataaggagaacgtacgtgctgttgccagagacggcatcgtcttgctgaagaacgatggaattctgccgctttccaagccgagaaagattgctgtcgtgggctcccactccgtcaacaatccccagggaatcaacgcctgtgttgacaagggctgcaatgttggcacccttggcatgggctggggttcaggcagcgtcaactacccctatctcgtgtccccgtacgatgctctccggactcgtgctcaggccgatggcacacaaatcagcctccacaacactgacagcaccaacggtgtgtcaaacgttgtgtctgacgctgatgctgttgttgttgtcatcactgccgattctggtgaagggtacatcactgtcgagggccacgctggcgaccgcagccaccttgacccgtggcacaatggcaaccaacttgttcaggctgccgcggctgccaacaagaacgtcatcgttgttgtgcacagtgttggccagatcaccctggagactatcctcaacaccaatggagtccgcgcgattgtgtgggctggtcttccgggccaagagaatggcaacgctcttgttgatgttctctacggcttggtttcgccatctggaaagcttccctacaccattggcaagagggagtcggactatggcacagccgttgttcgtggggatgataacttcagggagggcctttttgttgactaccgtcactttgacaatgccaggatcgagccgcgctatgagtttggctttggtctttgtaagttccagcggcggagttgggtttgatttcaagctttcctaacctgataaaacagcttacaccaatttcaccttctccgacatcaagattacttccaatgtcaagccggggcccgctactggccagaccattcccggcggacctgccgacctgtgggaggacgttgcgacagtcactgcaaccatcaccaactcgggtgctgtcgagggcgctgaggttgcccagctttacatcggcctgccgtcctcggctcctgcctctcccccgaagcagctgcgtggattttccaagctgaagctggccccgggtgccagcggcactgccacattcaacctcagacgcagagatctcagctattgggatacccgcctccagaactgggtcgtgcccagcggcaactttgtcgtcagcgtcggcgccagctcgagagatatccgcttgacgggcaccatcacggcgtag

SEQ ID NO: 54 - protein, 733 aa, Podospora anserina
MALQTFFLLAAAMLANAETTGEKVSRQAPSGAQAWAAAHSQAAATLARMSQQDKINMVTGIGWDRGPCVGNTAAISSINYPQICLQDGPLGIRFGTGTTAFTPGVQAASTWDVDLIRQRGAYLGAEAKGCGIHILLGPVAGALGKIPHGGRNWEGFGADPYLAGIAMKETIEGIQSAGVQANAKHYIANEQELNRETMSSNVDDRTQHELYLWPFADAVHANVASVMCSYNKLNGTWACENDKALNQILKKELGFQGYVLSDWNAQHSTALSANSGLDMTMPGTDFNGRNVYWGPQLNNAVNAGQVQRSRLDDMCKRILAGWYLLGQNQGYPAINIRANVQGNHKENVRAVARDGIVLLKNDGILPLSKPRKIAVVGSHSVNNPQGINACVDKGCNVGTLGMGWGSGSVNYPYLVSPYDALRTRAQADGTQISLHNTDSTNGVSNVVSDADAVVVVITADSGEGYITVEGHAGDRSHLDPWHNGNQLVQAAAAANKNVIVVVHSVGQITLETILNTNGVRAIVWAGLPGQENGNALVDVLYGLVSPSGKLPYTIGKRESDYGTAVVRGDDNFREGLFVDYRHFDNARIEPRYEFGFGLSYTNFTFSDIKITSNVKPGPATGQTIPGGPADLWEDVATVTATITNSGAVEGAEVAQLYIGLPSSAPASPPKQLRGFSKLKLAPGASGTATFNLRRRDLSYWDTRLQNWVVPSGNFVVSVGASSRDIRLTGTITA

SEQ ID NO: 55 - DNA, 2551 bp, Fusarium verticillioides
atgtttccttcttccatatcttgtttggcggccctgagtctgatgagccagggtctactagctcagagccaaccggaaaatgtcatcaccgatgatacctacttctacggtcaatcgccaccagtgtatcctacacgtaagcactctctctgatttcccaacgaaagcaatactgatctcttgaccagcggaacaggtagacaccggctcatgggctgccgctgtagccaaagccaagaacttggtgtcccagttgactcttgaagagaaagtcaacttgactacaggaggccagacgaccaccggctgctctggcttcatccctggcattccccgtgtaggctttccaggactgtgtttagcagacgctggcaacggtgtccgcaacacagattatgtgagctcgtttccctccgggattcatgtcggtgcaagctggaatccggagttgacctacagccggagctactacatgggtgctgaggccaaagccaagggcgttaacatccttctcggtccagtatttggacctttgggccgagtagttgaaggtggacgcaactgggaggggttttccaatgatccctacctggcgggtaaattagggcatgaagctgtcgccggtatccaagacgccggagttgttgcatgcggaaaacatttccttgctcaagagcaggagacccatagacttgcggcgtctgtcactggggctgatgcaatctcatcaaatctcgatgacaagacactccatgaattatatctctggtaagcacatcatatcttggctgagtagatgaaccttactaacacccgaactgggcttttcgctgatgcagtccacgccggacttgccagtgtgatgtgcagctacaacagagcaaacaattcacacgcctgccaaaactcgaagcttctcaatggccttctcaagggcgagttaggattccagggttttgtcgtctcggactggggcgcacagcaatctggtatggcttcagcattggctggcctggatgttgtcatgcccagctcgatcttgtggggtgccaaccttacccttggtgtgaacaacggaactattcccgagtcacaggttgacaatatggttacacggtacgcgaagtctcagccttacttctcaattcttttgaactgacaatcgtgtaggctccttgcaacttggtatcagttgaaccaggaccaagacaccgaagccccaggtcacggactcgctgccaagctttgggagcctcacccagtagtcgacgctcgcaacgcaagctccaagcctactatctgggacggtgcagtcgagggccatgttcttgttaagaacaccaacaacgcactgccattcaagcccaacatgaaactcgtttctttgttcggatactctcacaaagctcctgataagaacatcccagaccccgcccaaggcatgttctccgcttggtctatcggtgcccaatccgccaacatcactgagctgaacctcggctttctcggaaatttgagtctcacatactccgccatcgcgcccaacggaaccatcatctcgggtggaggctcgggtgccagcgcttggactctgttcagctcacccttcgatgcattcgtttctcgggcgaagaaagagggtactgcgcttttctgggattttgagagctgggatccttatgtgaaccctacatctgaagcttgcatcgttgctggtaatgcatgggctagcgaaggctgggatagacctgcaacctatgatgcctatactgatgagctcatcaataacgtcgctgacaagtgcgctaacactattgttgttcttcacaatgctggaacacgacttgtggatggcttctttggtcaccccaacgtcaccgctattatctacgctcatctcccaggtcaggatagtggagatgctctggtatctttgctctatggcgatgagaacccatctggtcgcctcccttacaccgttgcccgcaacgagacggattatggtcacctgctgaagccagacttgactctcgcccccaaccagtaccaacactttccccagtccgacttctccgagggtattttcattgactaccgacatttcgatgctaagaacatcacgcctcgcttcgagtttggtttcggcttgagctacacaacctttgagtacgctagtctccagatctcaaagtcccaggcccagacaccggaatacccagctggtgctcttaccgagggaggccgttcagatttgtgggacgtcgttgctactgtcacagcaagcgtcaggaacactgggtctgtcgacggcaaggaggttgcacagctatacgttggtgttccaggtggtcctatgagacagctacgtggctttacgaaaccagctattaaggctggagagacggctacagtgacctttgagcttactcgccgcgacttgagtgtctgggatgttaatgcgcaggagtggcaacttcagcaaggcaactatgctatctacgttggccgaagtagtcgagatttgcctctgcaaagtaccttgagcatctag

SEQ ID NO: 56 - protein, 780 aa, Fusarium verticillioides
MFPSSISCLAALSLMSQGLLAQSQPENVITDDTYFYGQSPPVYPTHTGSWAAAVAKAKNLVSQLTLEEKVNLTTGGQTTTGCSGFIPGIPRVGFPGLCLADAGNGVRNTDYVSSFPSGIHVGASWNPELTYSRSYYMGAEAKAKGVNILLGPVFGPLGRVVEGGRNWEGFSNDPYLAGKLGHEAVAGIQDAGVVACGKHFLAQEQETHRLAASVTGADAISSNLDDKTLHELYLCVMCSYNRANNSHACQNSKLLNGLLKGELGFQGFVVSDWGAQQSGMASALAGLDVVMPSSILWGANLTLGVNNGTIPESQVDNMVTRLLATWYQLNQDQDTEAPGHGLAAKLWEPHPVVDARNASSKPTIWDGAVEGHVLVKNTNNALPFKPNMKLVSLFGYSHKAPDKNIPDPAQGMFSAWSIGAQSANITELNLGFLGNLSLTYSAIAPNGTIISGGGSGASAWTLFSSPFDAFVSRAKKEGTALFWDFESWDPYVNPTSEACIVAGNAWASEGWDRPATYDAYTDELINNVADKCANTIVVLHNAGTRLVDGFFGHPNVTAIIYAHLPGQDSGDALVSLLYGDENPSGRLPYTVARNETDYGHLLKPDLTLAPNQYQHFPQSDFSEGIFIDYRHFDAKNITPRFEFGFGLSYTTFEYASLQISKSQAQTPEYPAGALTEGGRSDLWDVVATVTASVRNTGSVDGKEVAQLYVGVPGGPMRQLRGFTKPAIKAGETATVTFELTRRDLSVWDVNAQEWQLQQGNYAIYVGRSSRDLPLQSTLSI

SEQ ID NO: 57 - DNA, 2487 bp, Fusarium verticillioides
atggctagcattcgatctgtgttggtctcgggtcttttggccgcgggtgtcaatgcccaagcctacgatgcgagtgatcgcgctgaagatgctttcagctgggtccagcccaagaacaccactattcttggacagtacggccattcgcctcattaccctgccagtatgttcaccaactacaccaagtgacactgaggctgtactgacattctagacaatgctactggcaagggctgggaagatgccttcgccaaggctcaaaactttgtctcccaactaaccctcgaggaaaaggccgacatggtcacaggaactccaggtccttgcgtcggcaacatcgtcgccattccccgtctcaacttcaacggtctctgtcttcacgacggccccctcgccatccgagtagcagactacgccagtgttttccccgctggtgtatcagccgcttcatcgtgggacaaggacctcctctaccagcgcggtctcgccatgggtcaagagttcaaggccaagggtgctcacatcctcctcggccccgtcgccggtcctcttggccgctcggcatactctggtcgtaactgggagggtttctcgccggacccttacctcactggtattgcgatggaggagactatcatgggacatcaagatgctggtgttcaggctactgcgaagcactttatcggtaatgagcaggaggtcatgcgaaaccctacttttgtcaaggatgggtatattggtgaggttgacaaggaggctctttcgtctaacatggatgatcgaaccatgcacgagctttacctctggccctttgccaatgctgttcatgccaaggcttccagcatgatgtgctcgtaccagcgtctcaacggctcctacgcctgccagaactcaaaggtcctcaacggaattctgcgtgatgagcttggtttccagggctacgtcatgtcagattggggtgccacccacgccggtgttgctgccatcaacagcggtctcgacatggacatgcccggtggtatcggtgcctacggaacatactttaccaagtccttcttcggcggcaacctcacccgcgccgtcaccaacggcaccctcgacgagacccgcgtcaacgacatgatcacccgcatcatgactccctacttctggctcggccaggacaaggactatccctccgtcgacccctccagcggtgatctcaacaccttcagccccaagagctcctggttccgcgagttcaacctcaccggcgagcgcagccgtgacgtccgcggtaaccacggcgacttgatccgcaagcacggcgccgagtctaccgtccttctcaagaacgagaagaacgcccttcccctcaagaagcccaagtccatcgctgtctttggcaacgatgctggtgatatcactgagggtttctacaaccagaatgactacgaatttggcactcttgttgctggtggtggctctggaactggtcgtttgacataccttgtttcgcctctagccgccatcaatgctcgtgctaagcaggacggtactcttgttcagcagtggatgaacaacactcttattgctaccaccaacgtcactgatctctggatccctgctactcccgatgtctgcctcgttttcttgaagacttgggctgaggaggctgctgatcgtgagcacctctccgttgactgggacggtaatgatgttgttgagtctgttgccaagtactgcaataacactgtcgtcgtcactcactcttctggtatcaacactcttccttgggctgaccaccccaacgtcaccgctattctcgctgcccacttccccggtcaggagtctggcaactccctcgttgacctcctctacggcgatgtcaacccctctggtcgtcttccctacaccatcgccttcaacggcaccgactacaacgctccccccaccactgccgtcaacaccaccggcaaggaggactggcagtcttggttcgacgagaagctcgagattgactaccgctacttcgacgcgcacaacatctccgtccgctacgaattcggcttcggtctctcctactccaccttcgaaatctccgacatctccgctgagccactcgcatccgacattacctcccagcccgaggatctccccgtgcagcccggcggcaaccccgccctctgggagaccgtctacaacgtgaccgtctccgtctccaacacgggcaaggtcgacggcgccactgtcccccagctatacgtgacattccccgacagcgcgcctgccggtacaccacccaagcagctccgtgggttcgacaaggtcttccttgaggctggcgagagcaagagtgtcagctttgagctgatgcgccgtgatctgagctactgggatatcatttctcagaagtggctcatccctgagggagagtttactattcgtgttggattcagcagtcgggacttgaaggaggagacaaaggttactgttgttgaggcgtaa

SEQ ID NO: 58 - protein, 811 aa, Fusarium verticillioides
MASIRSVLVSGLLAAGVNAQAYDASDRAEDAFSWVQPKNTTILGQYGHSPHYPANNATGKGWEDAFAKAQNFVSQLTLEEKADMVTGTPGPCVGNIVAIPRLNFNGLCLHDGPLAIRVADYASVFPAGVSAASSWDKDLLYQRGLAMGQEFKAKGAHILLGPVAGPLGRSAYSGRNWEGFSPDPYLTGIAMEETIMGHQDAGVQATAKHFIGNEQEVMRNPTFVKDGYIGEVDKEALSSNMDDRTMHELYLWPFANAVHAKASSMMCSYQRLNGSYACQNSKVLNGILRDELGFQGYVMSDWGATHAGVAAINSGLDMDMPGGIGAYGTYFTKSFFGGNLTRAVTNGTLDETRVNDMITRIMTPYFWLGQDKDYPSVDPSSGDLNTFSPKSSWFREFNLTGERSRDVRGNHGDLIRKHGAESTVLLKNEKNALPLKKPKSIAVFGNDAGDITEGFYNQNDYEFGTLVAGGGSGTGRLTYLVSPLAAINARAKQDGTLVQQWMNNTLIATTNVTDLWIPATPDVCLVFLKTWAEEAADREHLSVDWDGNDVVESVAKYCNNTVVVTHSSGINTLPWADHPNVTAILAAHFPGQESGNSLVDLLYGDVNPSGRLPYTIAFNGTDYNAPPTTAVNTTGKEDWQSWFDEKLEIDYRYFDAHNISVRYEFGFGLSYSTFEISDISAEPLASDITSQPEDLPVQPGGNPALWETVYNVTVSVSNTGKVDGATVPQLYVTFPDSAPAGTPPKQLRGFDKVFLEAGESKSVSFELMRRDLSYWDIISQKWLIPEGEFTIRVGFSSRDLKEETKVTVVEA

SEQ ID NO: 59 - DNA, 3269 bp, Fusarium verticillioides
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgaccgacgatctccaagcaccgatggaaagagctctcccaacaacaccgctgctcctctctacgagttcggtcacggtctatcttggtccacctttgagtactctgacctcaacatccagaagaacgtcgagaacccctactctcctcccgctggccagaccatccccgccccaacctttggcaacttcagcaagaacctcaacgactacgtgttccccaagggcgtccgatacatctacaagttcatctaccccttcctcaacacctcctcatccgccagcgaggcatccaacgatggtggccagtttggtaagactgccgaagagttcctccctcccaacgccctcaacggctcagcccagcctcgtcttcccgcctctggtgccccaggtggtaaccctcaattgtgggacatcttgtacaccgtcacagccacaatcaccaacacaggcaacgccacctccgacgagattccccagctgtatgtcagcctcggtggcgagaacgagcccatccgtgttctccgcggtttcgaccgtatcgagaacattgctcccggccagagcgccatcttcaacgctcaattgacccgtcgcgatctgagtaactgggatacaaatgcccagaactgggtcatcactgaccatcccaagactgtctgggttggaagcagctctcgcaagctgcctctcagcgccaagttggagtaagaaagccaaacaagggttgttttttggactgcaattttttgggaggacatagtagccgcgcgccagttacgtc

SEQ ID NO: 60 - protein, 899 aa, Fusarium verticillioides
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSSPNNTAAPLYEFGHGLSWSTFEYSDLNIQKNVENPYSPPAGQTIPAPTFGNFSKNLNDYVFPKGVRYIYKFIYPFLNTSSSASEASNDGGQFGKTAEEFLPPNALNGSAQPRLPASGAPGGNPQLWDILYTVTATITNTGNATSDEIPQLYVSLGGENEPIRVLRGFDRIENIAPGQSAIFNAQLTRRDLSNWDTNAQNWVITDHPKTVWVGSSSRKLPLSAKLE

SEQ ID NO: 61 - DNA, 2370 bp, Trichoderma reesei
atgcgttaccgaacagcagctgcgctggcacttgccactgggccctttgctagggcagacagtcagtatagctggtcccatactgggatgtgatatgtatcctggagacaccatgctgactcttgaatcaaggtagctcaacatcgggggcctcggctgaggcagttgtacctcctgcagggactccatggggaaccgcgtacgacaaggcgaaggccgcattggcaaagctcaatctccaagataaggtcggcatcgtgagcggtgtcggctggaacggcggtccttgcgttggaaacacatctccggcctccaagatcagctatccatcgctatgccttcaagacggacccctcggtgttcgatactcgacaggcagcacagcctttacgccgggcgttcaagcggcctcgacgtgggatgtcaatttgatccgcgaacgtggacagttcatcggtgaggaggtgaaggcctcggggattcatgtcatacttggtcctgtggctgggccgctgggaaagactccgcagggcggtcgcaactgggagggcttcggtgtcgatccatatctcacgggcattgccatgggtcaaaccatcaacggcatccagtcggtaggcgtgcaggcgacagcgaagcactatatcctcaacgagcaggagctcaatcgagaaaccatttcgagcaacccagatgaccgaactctccatgagctgtatacttggccatttgccgacgcggttcaggccaatgtcgcttctgtcatgtgctcgtacaacaaggtcaataccacctgggcctgcgaggatcagtacacgctgcagactgtgctgaaagaccagctggggttcccaggctatgtcatgacggactggaacgcacagcacacgactgtccaaagcgcgaattctgggcttgacatgtcaatgcctggcacagacttcaacggtaacaatcggctctggggtccagctctcaccaatgcggtaaatagcaatcaggtccccacgagcagagtcgacgatatggtgactcgtatcctcgccgcatggtacttgacaggccaggaccaggcaggctatccgtcgttcaacatcagcagaaatgttcaaggaaaccacaagaccaatgtcagggcaattgccagggacggcatcgttctgctcaagaatgacgccaacatcctgccgctcaagaagcccgctagcattgccgtcgttggatctgccgcaatcattggtaaccacgccagaaactcgccctcgtgcaacgacaaaggctgcgacgacggggccttgggcatgggttggggttccggcgccgtcaactatccgtacttcgtcgcgccctacgatgccatcaataccagagcgtcttcgcagggcacccaggttaccttgagcaacaccgacaacacgtcctcaggcgcatctgcagcaagaggaaaggacgtcgccatcgtcttcatcaccgccgactcgggtgaaggctacatcaccgtggagggcaacgcgggcgatcgcaacaacctggatccgtggcacaacggcaatgccctggtccaggcggtggccggtgccaacagcaacgtcattgttgttgtccactccgttggcgccatcattctggagcagattcttgctcttccgcaggtcaaggccgttgtctgggcgggtcttccttctcaggagagcggcaatgcgctcgtcgacgtgctgtggggagatgtcagcccttctggcaagctggtgtacaccattgcgaagagccccaatgactataacactcgcatcgtttccggcggcagtgacagcttcagcgagggactgttcatcgactataagcacttcgacgacgccaatatcacgccgcggtacgagttcggctatggactgtgtaagtttgctaacctgaacaatctattagacaggttgactgacggatgactgtggaatgatagcttacaccaagttcaactactcacgcctctccgtcttgtcgaccgccaagtctggtcctgcgactggggccgttgtgccgggaggcccgagtgatctgttccagaatgtcgcgacagtcaccgttgacatcgcaaactctggccaagtgactggtgccgaggtagcccagctgtacatcacctacccatcttcagcacccaggacccctccgaagcagctgcgaggctttgccaagctgaacctcacgcctggtcagagcggaacagcaacgttcaacatccgacgacgagatctcagctactgggacacggcttcgcagaaatgggtggtgccgtcggggtcgtttggcatcagcgtgggagcgagcagccgggatatcaggctgacgagcactctgtcggtagcgtag

SEQ ID NO: 62 - protein, 744 aa, Trichoderma reesei; identical to SEQ ID NO: 45

SEQ ID NO: 63 - DNA, 2625 bp, Trichoderma reesei
atgaagacgttgtcagtgtttgctgccgcccttttggcggccgtagctgaggccaatccctacccgcctcctcactccaaccaggcgtactcgcctcctttctacccttcgccatggatggaccccagtgctccaggctgggagcaagcctatgcccaagctaaggagttcgtctcgggcttgactctcttggagaaggtcaacctcaccaccggtgttggctggatgggtgagaagtgcgttggaaacgttggtaccgtgcctcgcttgggcatgcgaagtctttgcatgcaggacggccccctgggtctccgattcaacacgtacaacagcgctttcagcgttggcttgacggccgccgccagctggagccgacacctttgggttgaccgcggtaccgctctgggctccgaggcaaagggcaagggtgtcgatgttcttctcggacccgtggctggccctctcggtcgcaaccccaacggaggccgtaacgtcgagggtttcggctcggatccctatctggcgggtttggctctggccgataccgtgaccggaatccagaacgcgggcaccatcgcctgtgccaagcacttcctcctcaacgagcaggagcatttccgccaggtcggcgaagctaacggttacggataccccatcaccgaggctctgtcttccaacgttgatgacaagacgattcacgaggtgtacggctggcccttccaggatgctgtcaaggctggtgtcgggtccttcatgtgctcgtacaaccaggtcaacaactcgtacgcttgccaaaactccaagctcatcaacggcttgctcaaggaggagtacggtttccaaggctttgtcatgagcgactggcaggcccagcacacgggtgtcgcgtctgctgttgccggtctcgatatgaccatgcctggtgacaccgccttcaacaccggcgcatcctactttggaagcaacctgacgcttgctgttctcaacggcaccgtccccgagtggcgcattgacgacatggtgatgcgtatcatggctcccttcttcaaggtgggcaagacggttgacagcctcattgacaccaactttgattcttggaccaatggcgagtacggctacgttcaggccgccgtcaatgagaactgggagaaggtcaactacggcgtcgatgtccgcgccaaccatgcgaaccacatccgcgaggttggcgccaagggaactgtcatcttcaagaacaacggcatcctgccccttaagaagcccaagttcctgaccgtcattggtgaggatgctggcggcaaccctgccggccccaacggctgcggtgaccgcggctgtgacgacggcactcttgccatggagtggggatctggtactaccaacttcccctacctcgtcacccccgacgcggccctgcagagccaggctctccaggacggcacccgctacgagagcatcctgtccaactacgccatctcgcagacccaggcgctcgtcagccagcccgatgccattgccattgtctttgccaactcggatagcggcgagggctacatcaacgtcgatggcaacgagggcgaccgcaagaacctgacgctgtggaagaacggcgacgatctgatcaagactgttgctgctgtcaaccccaagacgattgtcgtcatccactcgaccggccccgtgattctcaaggactacgccaaccaccccaacatctctgccattctgtgggccggtgctcctggccaggagtctggcaactcgctggtcgacattctgtacggcaagcagagcccgggccgcactcccttcacctggggcccgtcgctggagagctacggagttagtgttatgaccacgcccaacaacggcaacggcgctccccaggataacttcaacgagggcgccttcatcgactaccgctactttgacaaggtggctcccggcaagcctcgcagctcggacaaggctcccacgtacgagtttggcttcggactgtcgtggtcgacgttcaagttctccaacctccacatccagaagaacaatgtcggccccatgagcccgcccaacggcaagacgattgcggctccctctctgggcagcttcagcaagaaccttaaggactatggcttccccaagaacgttcgccgcatcaaggagtttatctacccctacctgagcaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 64 - protein, 874 aa, Trichoderma reesei
MKTLSVFAAALLAAVAEANPYPPPHSNQAYSPPFYPSPWMDPSAPGWEQAYAQAKEFVSGLTLLEKVNLTTGVGWMGEKCVGNVGTVPRLGMRSLCMQDGPLGLRFNTYNSAFSVGLTAAASWSRHLWVDRGTALGSEAKGKGVDVLLGPVAGPLGRNPNGGRNVEGFGSDPYLAGLALADTVTGIQNAGTIACAKHFLLNEQEHFRQVGEANGYGYPITEALSSNVDDKTIHEVYGWPFQDAVKAGVGSFMCSYNQVNNSYACQNSKLINGLLKEEYGFQGFVMSDWQAQHTGVASAVAGLDMTMPGDTAFNTGASYFGSNLTLAVLNGTVPEWRIDDMVMRIMAPFFKVGKTVDSLIDTNFDSWTNGEYGYVQAAVNENWEKVNYGVDVRANHANHIREVGAKGTVIFKNNGILPLKKPKFLTVIGEDAGGNPAGPNGCGDRGCDDGTLAMEWGSGTTNFPYLVTPDAALQSQALQDGTRYESILSNYAISQTQALVSQPDAIAIVFANSDSGEGYINVDGNEGDRKNLTLWKNGDDLIKTVAAVNPKTIVVIHSTGPVILKDYANHPNISAILWAGAPGQESGNSLVDILYGKQSPGRTPFTWGPSLESYGVSVMTTPNNGNGAPQDNFNEGAFIDYRYFDKVAPGKPRSSDKAPTYEFGFGLSWSTFKFSNLHIQKNNVGPMSPPNGKTIAAPSLGSFSKNLKDYGFPKNVRRIKEFIYPYLSTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 65 - DNA, 2577 bp, Artificial Sequence
  synthetic codon optimized GH3 family beta- glucosidase from Talaromyces emersonii
atgcgcaacggcctcctcaaggtcgccgccttagccgctgccagcgccgtcaacggcgagaacctcgcctacagcccccccttctaccccagcccctgggccaacggccagggcgactgggccgaggcctaccagaaggccgtccagttcgtcagccagctcaccctcgccgagaaggtcaacctcaccaccggcaccggctgggagcaggaccgctgcgtcggccaggtcggcagcatcccccgcttaggcttccccggcctctgcatgcaggacagccccctcggcgtccgcgacaccgactacaacagcgccttccctgccggcgttaacgtcgccgccacctgggaccgcaacttagcctaccgcagaggcgtcgccatgggcgaggaacaccgcggcaagggcgtcgacgtccagttaggccccgtcgccggccccttaggccgctctcctgatgccggccgcaactgggagggcttcgcccccgaccccgtcctcaccggcaacatgatggccagcaccatccagggcatccaggatgctggcgtcattgcctgcgccaagcacttcatcctctacgagcaggaacacttccgccagggcgcccaggacggctacgacatcagcgacagcatcagcgccaacgccgacgacaagaccatgcacgagttatacctctggcccttcgccgatgccgtccgcgccggtgtcggcagcgtcatgtgcagctacaaccaggtcaacaacagctacgcctgcagcaacagctacaccatgaacaagctcctcaagagcgagttaggcttccagggcttcgtcatgaccgactggggcggccaccacagcggcgtcggctctgccctcgccggcctcgacatgagcatgcccggcgacattgccttcgacagcggcacgtctttctggggcaccaacctcaccgttgccgtcctcaacggctccatccccgagtggcgcgtcgacgacatggccgtccgcatcatgagcgcctactacaaggtcggccgcgaccgctacagcgtccccatcaacttcgacagctggaccctcgacacctacggccccgagcactacgccgtcggccagggccagaccaagatcaacgagcacgtcgacgtccgcggcaaccacgccgagatcatccacgagatcggcgccgcctccgccgtcctcctcaagaacaagggcggcctccccctcactggcaccgagcgcttcgtcggtgtctttggcaaggatgctggcagcaacccctggggcgtcaacggctgcagcgaccgcggctgcgacaacggcaccctcgccatgggctggggcagcggcaccgccaactttccctacctcgtcacccccgagcaggccatccagcgcgaggtcctcagccgcaacggcaccttcaccggcatcaccgacaacggcgccttagccgagatggccgctgccgcctctcaggccgacacctgcctcgtctttgccaacgccgactccggcgagggctacatcaccgtcgatggcaacgagggcgaccgcaagaacctcaccctctggcagggcgccgaccaggtcatccacaacgtcagcgccaactgcaacaacaccgtcgtcgtcttacacaccgtcggccccgtcctcatcgacgactggtacgaccaccccaacgtcaccgccatcctctgggccggtttacccggtcaggaaagcggcaacagcctcgtcgacgtcctctacggccgcgtcaaccccggcaagacccccttcacctggggcagagcccgcgacgactatggcgcccctctcatcgtcaagcctaacaacggcaagggcgccccccagcaggacttcaccgagggcatcttcatcgactaccgccgcttcgacaagtacaacatcacccccatctacgagttcggcttcggcctcagctacaccaccttcgagttcagccagttaaacgtccagcccatcaacgcccctccctacacccccgccagcggctttacgaaggccgcccagagcttcggccagccctccaatgccagcgacaacctctaccctagcgacatcgagcgcgtccccctctacatctacccctggctcaacagcaccgacctcaaggccagcgccaacgaccccgactacggcctccccaccgagaagtacgtcccccccaacgccaccaacggcgacccccagcccattgaccctgccggcggtgcccctggcggcaaccccagcctctacgagcccgtcgcccgcgtcaccaccatcatcaccaacaccggcaaggtcaccggcgacgaggtcccccagctctatgtcagcttaggcggccctgacgacgcccccaaggtcctccgcggcttcgaccgcatcaccctcgcccctggccagcagtacctctggaccaccaccctcactcgccgcgacatcagcaactgggaccccgtcacccagaactgggtcgtcaccaactacaccaagaccatctacgtcggcaacagcagccgcaacctccccctccaggcccccctcaagccctaccccggcatctgatga

SEQ ID NO: 66 - protein, 857 aa, Talaromyces emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 67 - DNA, 2586 bp, Aspergillus niger
atgcgcttcaccagcatcgaggccgtcgccctcaccgccgtcagcctcgccagcgccgacgagttagcctacagccccccctactaccccagcccctgggccaacggccagggcgactgggccgaggcctaccagcgcgccgtcgacatcgtcagccagatgaccctcgccgagaaggtcaacctcaccaccggcaccggctgggagttagagttatgcgtcggccagactggtggcgtcccccgcctcggcatccccggcatgtgcgcccaggacagccccctcggcgtccgcgacagcgactacaacagcgccttccctgccggcgtcaacgtcgccgccacctgggacaagaacctcgcctacctccgcggccaggccatgggccaggaattcagcgacaagggcgccgacatccagttaggccccgctgccggccctttaggccgctctcccgacggcggcagaaactgggagggcttcagccccgaccccgctctcagcggcgtcctcttcgccgagactatcaagggcatccaggatgctggcgtcgtcgccaccgccaagcactacattgcctacgagcaggaacacttccgccaggcccccgaggcccagggctacggcttcaacatcaccgagagcggcagcgccaacctcgacgacaagaccatgcacgagttatacctctggcccttcgccgacgccattagagctggcgctggtgctgtcatgtgcagctacaaccagatcaacaacagctacggctgccagaacagctacaccctcaacaagctcctcaaggccgagttaggcttccagggcttcgtcatgtccgactgggccgcccaccacgccggcgtcagcggcgccttagccggcctcgacatgagcatgcccggcgacgtcgactacgacagcggcaccagctactggggcaccaacctcaccatcagcgtcctcaacggcaccgtcccccagtggcgcgtcgacgacatggccgtccgcatcatggccgcctactacaaggtcggccgcgaccgcctctggaccccccccaacttcagcagctggacccgcgacgagtacggcttcaagtactactacgtcagcgagggcccctatgagaaggtcaaccagttcgtcaacgtccagcgcaaccacagcgagttaatccgccgcatcggcgccgacagcaccgtcctcctcaagaacgacggcgccctccccctcaccggcaaggaacgcctcgtcgccctcatcggcgaggacgccggcagcaacccctacggcgccaacggctgcagcgaccgcggctgcgacaacggcaccctcgccatgggctggggcagcggcaccgccaacttcccttacctcgtcacccccgagcaggccatcagcaacgaggtcctcaagaacaagaacggcgtctttaccgccaccgacaactgggccatcgaccagatcgaggccttagccaagaccgcctctgtcagcctcgtctttgtcaacgccgacagcggcgagggctacatcaacgtcgacggcaacctcggcgaccgccgcaacctcaccctctggcgcaacggcgacaacgtcatcaaggccgccgccagcaactgcaacaacaccatcgtcatcatccacagcgtcggccccgtcctcgtcaacgagtggtacgacaaccccaacgtcaccgccatcctctggggcggcttacccggccaggaaagcggcaacagcctcgccgacgtcctctacggccgcgtcaaccctggcgccaagagccccttcacctggggcaagacccgcgaggcctatcaggactacctctacaccgagcccaacaacggcaacggcgccccccaggaagatttcgtcgagggcgtctttatcgactaccgcggctttgacaagcgcaacgagactcccatctacgagttcggctacggcctcagctacaccaccttcaactacagcaacctccaggtcgaggtcctcagcgcccctgcctacgagcccgccagcggcgagactgaggccgcccccaccttcggcgaggtcggcaacgccagcgactacttataccccgacggcctccagcgcatcaccaagttcatctacccctggctcaacagcaccgacctcgaggccagcagcggcgacgcctcttacggccaggacgcctccgactacctccccgagggtgccaccgacggcagcgctcagcccatcttacctgccggtggcggtgctggcggcaaccccagactctacgacgagctgatccgcgtcagcgtcaccatcaagaacaccggcaaggtcgctggtgacgaggtcccccagctctacgtcagcttaggcggccctaacgagcccaagatcgtcctccgccagttcgagcgcatcaccctccagcccagcaaggaaactcagtggagcaccaccctcactcgccgcgacctcgccaactggaacgtcgagactcaggactgggagatcaccagctaccccaagatggtctttgccggcagcagcagccgcaagctccccctccgcgccagcctccccaccgtccactgatga

SEQ ID NO: 68 - protein, 860 aa, Aspergillus niger
MRFTSIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSKETQWSTTLTRRDLANWNVETQDWEITSYPKMVFAGSSSRKLPLRASLPTVH

SEQ ID NO: 69 - DNA, 3203 bp, Fusarium oxysporum
atgaagctgaactgggtcgccgcagccctctctataggtgctgctggcactgatggtgcagttgctcttgcttctgaagttccaggcactttggctggtgtaaaggtcggtttttttaccatttcctcacctaatctcagccttgttgccatatcgcccttattcgctcggacgctacgcaccaaatcgcgatcatttcctcccttgcagccttgttttcttttttcgatcttccctccgcaatcgccagcacccttagcctacacaaaaacccccgagacagtctcattgagtttgtcgacatcaagttgcttctcaagtgtgcatttgcgtggctgtctacttctgcctctagaccaccaaatctgggcgcaattgatcgctcaaaccttgttcgaataagccttttattcgagacgtccaatttttacagagaatgtacctttcaataataccgacgttatgcgcggcggtggctgctgtgatggttgttgatcagaatactgacgctcaaaaggttgtcacgagagatacactcgcacactcacctcctcactatccttcaccatggatggatcctaatgccattggctgggaggaagcttacgccaaagcaaagaactttgtgtcccagctcactctcctcgaaaaggtcaacttgaccactggtgttgggtaagtagctccttgcgaacagtgcatctcggtctccttgactaacgactctctcaggtggcaaggcgaacgctgtgtaggaaacgtgggatcaattcctcgtcttggtatgcgaggtctttgtcttcaggatggtcctcttggaattcgtctgtccgattacaacagtgcttttcccgctggcaccacagctggtgcttcttggagcaagtctctctggtatgagaggggtcttctgatgggaactgagttcaaggggaagggtatcgatatcgctcttggccctgctactggtcctcttggccgcactgctgctggtggacgaaactgggagggctttaccgttgatccttatatggctggccatgccatggccgaggccgtcaagggcatccaagacgcaggtgtcattgcttgtgctaagcattacatcgcaaacgagcaaggtaagccaattggacggtttgggaaatcgacagagaactgacccccttgtagagcacttccgacagagtggcgaggtccagtcccgcaagtacaacatctccgagtctctctcctccaacctggacgacaagactttgcacgagctctacgcctggccctttgctgatgccgtccgcgctggcgtcggttcagtcatgtgctcttacaatcagatcaacaactcgtacggttgccagaactccaagctcctcaacggtatcctcaaggacgagatgggtttccagggcttcgtcatgagcgattgggcggcccagcacaccggtgctgcttctgccgtcgctggtcttgatatgagcatgcctggtgacaccgcgttcgacagtggatatagcttctggggtggaaacctgactcttgctgtcatcaacggaactgttcccgcctggcgagttgatgacatggctctgcgaatcatgtcggccttcttcaaggttggaaagacggtagaggacctccccgacatcaacttctcctcctggacccgcgacaccttcggcttcgtccaaacatttgctcaagagaaccgcgaacaagtcaactttggagttaacgtccagcacgaccacaagaaccacatccgtgagtctgccgccaagggaagcgtcatcctcaagaacaccggctcccttcccctcaacaatcccaagttcctcgctgtcattggtgaggacgccggtcccaaccctgctggacccaatggttgcggcgaccgtggttgcgacaatggtaccctggctatggcttggggctcgggaacttctcaattcccttacttgatcacacccgaccaaggtctccagaaccgagctgcccaagacggaactcgatatgagagcatcttgaccaacaacgaatgggcccagacacaggctcttgtcagccaacccaacgtgaccgctatcgtttttgccaacgccgactctggtgagggttacattgaagtcgacggaaacttcggtgatcgcaagaacctcaccctctggcaacagggagacgagctcatcaagaacgtctcgtccatctgccccaacaccattgtcgttctgcataccgtcggccctgtcctgctcgccgactacgagaagaaccccaacatcaccgccatcgtctgggctggtcttcccggccaagagtctggcaatgccatcgctgatctcctctacggcaaggtaagccctggccgatctcccttcacttggggccgcacccgtgagagctacggtaccgaggttctttatgaggcgaacaacggccgtggcgctcctcaggatgacttctcggagggtgtcttcattgactaccgtcactttgatcgacgatctcccagcaccgatggcaagagcgctcccaacaacaccgctgctcctctctacgagttcggtcatggtctgtcttggactacctttgagtattcagacctcaacatccagaagaacgttaactccacctactctcctcctgctggtcagaccattcctgccccaacctttggcaacttcagcaagaacctcaacgactacgtgttccctaagggtgtccgatacatctacaagttcatctaccccttcctgaacacttcctcatccgccagcgaggcatctaacgacggcggccagtttggtaagactgccgaagagttcctacctccaaacgccctcaacggctcagcccagcctcgtcttccctcttctggtgccccaggcggtaaccctcaattgtgggatatcctgtacaccgtcacagccacaatcaccaacacaggcaacgccacctccgacgagattccccagctgtatgtcagcctcggtggcgagaacgaacccgttcgtgtcctccgcggtttcgaccgtatcgagaacattgctcccggccagagcgccatcttcaacgctcaattgacccgtcgcgatctgagcaactgggatgtggatgcccagaactgggttatcaccgaccatccaaagacggtgtgggttggaagtagttctcgcaagctgcctctcagcgccaagttggaataa

SEQ ID NO: 70 - protein, 899 aa, Fusarium oxysporum
MKLNWVAAALSIGAAGTDGAVALASEVPGTLAGVKNTDAQKVVTRDTLAHSPPHYPSPWMDPNAIGWEEAYAKAKNFVSQLTLLEKVNLTTGVGWQGERCVGNVGSIPRLGMRGLCLQDGPLGIRLSDYNSAFPAGTTAGASWSKSLWYERGLLMGTEFKGKGIDIALGPATGPLGRTAAGGRNWEGFTVDPYMAGHAMAEAVKGIQDAGVIACAKHYIANEQEHFRQSGEVQSRKYNISESLSSNLDDKTLHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTVEDLPDINFSSWTRDTFGFVQTFAQENREQVNFGVNVQHDHKNHIRESAAKGSVILKNTGSLPLNNPKFLAVIGEDAGPNPAGPNGCGDRGCDNGTLAMAWGSGTSQFPYLITPDQGLQNRAAQDGTRYESILTNNEWAQTQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSAPNNTAAPLYEFGHGLSWTTFEYSDLNIQKNVNSTYSPPAGQTIPAPTFGNFSKNLNDYVFPKGVRYIYKFIYPFLNTSSSASEASNDGGQFGKTAEEFLPPNALNGSAQPRLPSSGAPGGNPQLWDILYTVTATITNTGNATSDEIPQLYVSLGGENEPVRVLRGFDRIENIAPGQSAIFNAQLTRRDLSNWDVDAQNWVITDHPKTVWVGSSSRKLPLSAKLE

SEQ ID NO: 71 - DNA, 3134 bp, Gibberella zeae
atgaaggccaattggcttgccgcggccgtttatttggctgctggcaccgatgctgcagtccctgacactttggcaggagtcaatgtaagctactcttcaatttcatctcatctcaactttgccaggccacaacaacttttcttcactcacgatcttttcaccataaacgcaacagtttcacaaaaaataaagcccaaatcatgtctctgatcgttgaactcgccatcttcgtttacatcgcggttgtctttttcttcttgtacttctcattcgttgttgttctctacattttcgactggctgtttagccttgagattcttctcactccccgtgatgcctagatcactctctgaggcgtttaatctacttgtagagatgcgcctctcatttgttgtgtcgctagtcgcgatagttgctggaattgcagtccttgatcttcctactgacactcaaaagctcgttgcgcgggacacactcgctcactctcctcctcactatccctcgccatggatggaccctaacgctgtcggctgggaggacgcctacgccaaggccaaggactttgtctcccagatgactctcctagaaaaggtcaacttgaccactggtgttgggtaagtaacgagcgacaagacgtctacaatccactaacacgatctctagatggcagggcgaacgttgtgttggaaacgtgggatctatccctcgtctcggtatgcgaggcctctgtctccaggatggtcctctcggaattcgcttctccgactacaacagcgctttccctactggtgtcaccgctggtgcttcttggagtaaggccctttggtacgagcgaggacgattgatgggtaccgagtttaaggagaagggtatcgatattgctctcggccctgcaactggtcctctcggtcgccacgctgctggtggacgaaactgggaaggcttcactgtcgacccctacgccgctggccatgctatggctgagactgtcaagggtatccaagattctggagtcattgcttgtgctaagcattacatcgcaaacgagcaaggtatgtacaggcccattcaatggcttcaggaacgaaaactaactcttaatagaacacttccgtcaacgaggcgatgtcatgtctcaaaagttcaacatttccgagtctctgtcttccaaccttgacgataagactatgcacgagctctacaactggcctttcgccgacgccgtccgcgccggtgttggctccattatgtgctcttacaaccaggtcaacaactcatatgcttgccagaactccaagctcctcaacggcatcctcaaggacgagatgggtttccagggtttcgtcatgagcgattggcaggctcagcacaccggtgccgcctccgctgttgccggtcttgacatgaccatgcctggtgacaccgagttcaacactggcttcagcttctggggtggaaacctgaccctcgctgttatcaacggtactgttcccgcctggagaatcgacgacatggctacccgaattatggctgctttcttcaaggttggccgatctgttgaggaggaacccgacatcaacttctcagcttggactcgtgatgagtatggcttcgtccagacctacgcccaagagaaccgagaaaaggtcaactttgctgttaatgtccagcacgaccacaagcgccacattcgcgaggctggcgcaaagggatccgtcgtcctcaagaacactggctcacttcctcttaagaagccccagttcctcgctgtcattggagaggacgctggttccaaccctgccggacccaacggttgcgctgaccgtggatgcgacaacggtactcttgccatggcatggggttccggaacctctcaattcccctaccttgtcacccccgaccaaggcatctcgctccaggctattcaggacggtactcgttatgagagcatcctcaacaacaaccagtggccccagacacaagctcttgtcagccagcccaacgtcaccgccattgtctttgccaatgccgattctggtgagggctacatcgaggttgacggcaactacggcgaccgcaagaacctcactctgtggaagcaaggcgatgagctcatcaagaacgtctctgctatctgccccaacaccattgtggtccttcacaccgttggccccgtccttctaaccgagtggcacaacaaccccaacatcaccgccattgtttgggctggtgtgcctggacaggagtccggtaacgccatcgccgacatcctctacggcaagaccagccctggacgttctcccttcacctggggtcgcacttatgacagctatggcaccaaggttctctacaaggccaacaatggagagggtgcccctcaagaggactttgtcgagggcaacttcatcgactaccgccactttgaccgacaatcccccagcaccaacggaaagagtgccaccaacgactcttctgctcctctctacgagttcggtttcggtctgtcctggactacctttgagtactctgatctcaaagtcgagtctgtcagcaacgcctcttacagcccctctgtcggaaacaccattcctgcccctacctacggcaacttcagcaagaacctggacgattacacattcccctcaggtgtccgatacctctacaagttcatctacccctacctcaacacctcttcctccgctgagaaggcttccggcgatgtcaagggcagatttggtgagaccggcgacgagttcctccctcccaacgctctcaacggttcatcgcagcctcgtcttccttccagtggtgctcccggcggtaaccctcagctctgggacattatgtacaccgtcactgccaccatcaccaacactggtgacgctacctcggatgaggttccccagctgtacgtcagcctcggtggtgagggcgagcctgtccgtgtcctccgtggcttcgagcgtcttgaaaacattgctcctggtgagagtgccacattcaccgctcagcttactcgccgtgacctgagcaactgggacgtcaacgtccagaactgggtcatcaccgatcacgccaagaagatctgggtcggcagcagctctcgcaatctgcccctcagcgccgacctgtag

SEQ ID NO: 72 - protein, 886 aa, Gibberella zeae
MKANWLAAAVYLAAGTDAAVPDTLAGVNLVARDTLAHSPPHYPSPWMDPNAVGWEDAYAKAKDFVSQMTLLEKVNLTTGVGWQGERCVGNVGSIPRLGMRGLCLQDGPLGIRFSDYNSAFPTGVTAGASWSKALWYERGRLMGTEFKEKGIDIALGPATGPLGRHAAGGRNWEGFTVDPYAAGHAMAETVKGIQDSGVIACAKHYIANEQEHFRQRGDVMSQKFNISESLSSNLDDKTMHELYNWPFADAVRAGVGSIMCSYNQVNNSYACQNSKLLNGILKDEMGFQGFVMSDWQAQHTGAASAVAGLDMTMPGDTEFNTGFSFWGGNLTLAVINGTVPAWRIDDMATRIMAAFFKVGRSVEEEPDINFSAWTRDEYGFVQTYAQENREKVNFAVNVQHDHKRHIREAGAKGSVVLKNTGSLPLKKPQFLAVIGEDAGSNPAGPNGCADRGCDNGTLAMAWGSGTSQFPYLVTPDQGISLQAIQDGTRYESILNNNQWPQTQALVSQPNVTAIVFANADSGEGYIEVDGNYGDRKNLTLWKQGDELIKNVSAICPNTIVVLHTVGPVLLTEWHNNPNITAIVWAGVPGQESGNAIADILYGKTSPGRSPFTWGRTYDSYGTKVLYKANNGEGAPQEDFVEGNFIDYRHFDRQSPSTNGKSATNDSSAPLYEFGFGLSWTTFEYSDLKVESVSNASYSPSVGNTIPAPTYGNFSKNLDDYTFPSGVRYLYKFIYPYLNTSSSAEKASGDVKGRFGETGDEFLPPNALNGSSQPRLPSSGAPGGNPQLWDIMYTVTATITNTGDATSDEVPQLYVSLGGEGEPVRVLRGFERLENIAPGESATFTAQLTRRDLSNWDVNVQNWVITDHAKKIWVGSSSRNLPLSADL

SEQ ID NO: 73 - DNA, 2796 bp, Nectria haematococca
atgcggttcaccgtccttctcgcggcattttcggggcttgtccccatggttggttcgcaagctgaccagaaaccactacagctcggtgtgaacaataacactctggcgcattcacctcctcactatccttcgccatggatggatcctgctgctcctggctgggaggaagcctatctcaaggcgaaagattttgtttcacagcttacccttcttgaaaaggtcaacttgaccactggtgttgggtgagtcacttgttttcctctctcctgacgtgacactttgctttggcctgcttcctatatcgtctactagcattgctaacactcgaggcagatggatgggcgaacgttgcgtcggcaacgtgggttcactccctcgttttggaatgcgtggtctctgcatgcaggatggccccctcggcatccgcttgtctgactataactctgcctttcctactggtattacagctggtgcctcttggagccgtgccctttggtaccaacgtggcctcctgatgggcaccgagcatcgtgaaaaaggcatcgacgttgcacttgggcctgctactggtcctcttggtcgtactcctactggcggccgcaactgggagggtttctcggttgatccctacgttgctggcgttgccatggccgagactgttagcggcattcaagatggtggtactatcgcctgtgctaagcactacatcggcaacgaacaaggtatgcctcttcacttctcctcgctgataaatctgctcacaacaacctagagcaccatcgccaagcccccgaatccattggccgcggctacaacatcaccgagtccctgtcgtcgaacgttgatgacaagaccctccacgagctctatctctggccgttcgcagatgccgtcaaggctggtgttggtgctatcatgtgttcctaccagcagctgaacaactcttacggttgccaaaactctaagcttctcaacggaattctcaaggacgagctaggattccagggcttcgtcatgagtgactggcaagcccaacatgctggagctgctaccgctgttgcaggccttgacatgaccatgcccggtgacactttgttcaacaccggatacagcttctggggtggtaacctgaccctcgctgtagtcaatggcactgttcccgactggcgtattgacgacatggctatgagaatcatggcagctttcttcaaggttggcaagactgttgaggaccttcctgacatcaacttttcttcttggtctcgagacacttttggctacgttcaagccgctgcccaagagaactgggaacagatcaacttcggagttgatgttcgtcacgaccacagcgaacacattcgactctcggccgccaagggcaccgtcctccttaagaactctggctcattgcctctgaagaagcccaagttccttgccgtcgttggcgaggacgccggcccgaaccctgctggccccaacggctgtaacgaccgcggatgtaacaacggcactctggccatgtcctggggctcaggaacagcccagttcccttacctcgttactcccgactcagcgctacagaaccaggctgtcctcgacggcactcgctacgagagtgtcttgcggaacaaccagtgggaacagacacgcagtctcattagccaacctaacgtgacggctattgtgtttgccaatgccaattccggagagggatatatcgatgttgacggcaacgaaggcgatcggaagaatttgaccttgtggaacgagggtgatgacctaattaagaacgtctcctcaatctgccccaacaccattgttgttctgcacactgttggccctgtcatcctgacggaatggtatgacaacccgaacattaccgccatagtgtgggctggtgtacctggacaggagtccggcaatgctcttgtggacatcctttatggcaaaacaagccctggtcgctctcccttcacatggggtcgcacccgaaagagttacggcactgatgtcctatacgagcccaacaatggtcagggtgctcctcaagatgatttcacggagggagtctttatcgactatcgtcattttgaccaggtttctcctagcaccgacggcagcaagtctaatgatgagtccagtcccatctacgagtttggccatggtctgtcctggaccacgtttgagtactctgaactcaacattcaagctcacaacaagattcccttcgatcctcctattggcgagacgattgccgctccggtccttggcaactacagtaccgaccttgccgattacacgttccccgatggaattcgctacatctaccagttcatctatccctggttgaatacttcttcttccggaagagaggcttctggcgatcccgactacggaaagacggccgaagagttcctgccccccggagctctcgacgggtcagctcagccgcgacctccatcctctggtgctccaggtggaaaccctcatctttgggatgtgttgtacactgttagtgctatcatcaccaacactggcaacgccacctcggacgagatcccgcagctctacgttagtctcggtggcgagaacgagcccgtccgcgtccttcgcgggttcgaccgaattgagaacattgcgcctggccagagtgtcagattcacaactgacatcactcgccgcgacctgagcaactgggacgtcgtctctcagaactgggtcattacagactacgagaagaccgtatatgtcgggagcagctcccgcaacctgcctctcaaggcaaccctgaagtaa

SEQ ID NO: 74 - protein, 880 aa, Nectria haematococca
MRFTVLLAAFSGLVPMVGSQADQKPLQLGVNNNTLAHSPPHYPSPWMDPAAPGWEEAYLKAKDFVSQLTLLEKVNLTTGVGWMGERCVGNVGSLPRFGMRGLCMQDGPLGIRLSDYNSAFPTGITAGASWSRALWYQRGLLMGTEHREKGIDVALGPATGPLGRTPTGGRNWEGFSVDPYVAGVAMAETVSGIQDGGTIACAKHYIGNEQEHHRQAPESIGRGYNITESLSSNVDDKTLHELYLWPFADAVKAGVGAIMCSYQQLNNSYGCQNSKLLNGILKDELGFQGFVMSDWQAQHAGAATAVAGLDMTMPGDTLFNTGYSFWGGNLTLAVVNGTVPDWRIDDMAMRIMAAFFKVGKTVEDLPDINFSSWSRDTFGYVQAAAQENWEQINFGVDVRHDHSEHIRLSAAKGTVLLKNSGSLPLKKPKFLAVVGEDAGPNPAGPNGCNDRGCNNGTLAMSWGSGTAQFPYLVTPDSALQNQAVLDGTRYESVLRNNQWEQTRSLISQPNVTAIVFANANSGEGYIDVDGNEGDRKNLTLWNEGDDLIKNVSSICPNTIVVLHTVGPVILTEWYDNPNITAIVWAGVPGQESGNALVDILYGKTSPGRSPFTWGRTRKSYGTDVLYEPNNGQGAPQDDFTEGVFIDYRHFDQVSPSTDGSKSNDESSPIYEFGHGLSWTTFEYSELNIQAHNKIPFDPPIGETIAAPVLGNYSTDLADYTFPDGIRYIYQFIYPWLNTSSSGREASGDPDYGKTAEEFLPPGALDGSAQPRPPSSGAPGGNPHLWDVLYTVSAIITNTGNATSDEIPQLYVSLGGENEPVRVLRGFDRIENIAPGQSVRFTTDITRRDLSNWDVVSQNWVITDYEKTVYVGSSSRNLPLK

SEQ ID NO: 75 - DNA, 3169 bp, Verticillium dahliae
atgaagctgaccctcgctactgccttactggcagccagcgggtgtgtctctgcgggacaacccaagctcaaggtacgtacttgcctctttttcacaaggaaaccaaacccgcaccataatggtgattgagcagtcgtgctttcctcaacccgaatcaaacccatgccgtgttcgcgcatgccctttcgatcgtctgttgtgtgtgaacccacgctcttcaagcatcgcacatagcaccactccatcttcattttcgagcaatttcgggccgcagagagcggtctttcacttcaccacaatcgttcatgcctcgtgccccactgccatgtttcttcccagtattctacttctgagagccttgaccaccgttgtcgacatctcgtcgccaaggctcgttgacacggactctgtttcccttggaattaatattcgaaacaatgctgaccagcatcctcagcgccagactaacagctctagcgagctcgccttttcccctccgcactacccttctccatggatgaacccccaagcgactgggtgggaggacgcctacgcccgtgccagagaggtggtagagcagatgactctgctcgaaaaggtcaacctgacgacaggtgtcgggtaagcttcacagaccccgtcttgccatccaaagtcatctgacagaatcctagctggagcggtgatctctgcgtcggaaacgtcggctcgatcccccgaatcggctggagggggctttgtttgcaggatggcccacagggtatccgtttcgcggactacgtctcgtacttcacttcgagccagacagccggcgctacctgggaccgagggcttctgtaccagcgcgctcacgccattggcgccgaaggagtagccaagggcgtcgacgtcgtcctcgggcccgccattggccctctaggtcgccttcccgccggaggtcgtaactgggagggtttcgccgtggacccttacctcagtggcgttgctgtcgccgaatccgtcaggggcatccaggatgctggtgctattgccaacgtcaagcactacatcgtcaatgagcaggaacatttccgccaggctggcgaggctcaaggttacggctacgatgtcgacgaggcattatcgtcgaacgttgacgacaagaccatgcatgagctttacctttggccatttgcagacgctgtccgtgctggagccggcagtgtcatgtgttcttatcaacaggtgggggcaataccattctctcctctttccttgcagacagtgcactgaccgaccttttttgcccaagatcaacaacagttacggctgtcaaaactcacatcttctgaatgggctcctcaaggacgaactcggctttcaggggttcgtcctcagcgattggcaagcgcagcatgctggtgctgccactgccgttgctggacttgacatggccatgcccggtgacactcgcttcaacaccggagtcgccttctggggcgctaaccttaccaatgccattttgaacggcaccgttcccgaatatcggctcgatgacatggccatgcgtattatggcggcctttttcaaagttggaaagaccctggacgatgttcctgacatcaacttctcgtcttggacaaaagacaccatcggcccgctgcactgggcggcccaggacaatgtgcaggtcatcaaccaacacgttgatgtccgtcaagaccacggcgccctcattcgcaccatcgctgcccgcggtactgtcttactaaaaaatgagggatcactgcctctgaacaagccgaaatttgttgctgtcattggtgaagatgctggccctcgtcctgttggtcccaatggctgccctgatcagggttgcaataacggcactctggctgctggatggggatctggcaccgccagtttcccttatctcatcactcctgatagtgctcttcagtttcaagccgtttcggatggctcgcgatacgaaagcatcctcagcaactgggattatgagcgcacagaggccttggtttcccaggcggatgctactgctctggttttcgtcaatgcaaactctggcgaaggatatatcagcgttgatggaaacgaaggtgatcgcaagaacctcactctctggaatggaggagacgagcttattcaacgagtcgctgcggccaacaacaacaccatcgtcatcatccattcggttggtcccgttctagtcactgactggtacgagaatcccaatatcacggctatcatctgggccggcttacccggacaggagtctggcaactctatcgccgatattctttacggccgcgtgaaccctggtggcaagacacctttcacctggggtccaactgttgagagctacggcgttgacgtcctgagagagcccaacaatggcaatggtgctccccagagcgatttcgacgagggagtcttcatcgattaccgttggtttgaccggcagtcgggtgttgataacaatgcatcagcgccgaggaacagcagcagcagccacgccccaatcttcgagtttggctatggcctttcgtacacaacctttgaattctccaatcttcagattgagaggcatgacgttcacgattacgtccctaccactgggcagacgagccctgcgccgagatttggtgctaactacagtacgaactacgacgactacgtctttcccgagggcgaaatccgttacatctatcaacacatctacccatacctcaattcctcagacccaaaggaggcattggctgatcctaaatacggccaaactgcagaagagttcctcccagagggcgctcttgatgcctcaccgcagcctaggctcccagcttctggagggcccggaggcaacccaatgctttgggacgtcatattcacggtcaccgcgaccgtgaccaacacgggtaaggttgctggggacgaagtggcacagctttacgtttctcttggtggacctgacgatccgattcgagtcctccgtgggttcgaccgcattcacatcgcgcctggagcctcgcaaaccttccgtgcggaactcacgcgccgggacctcagcaactgggatgttgtcacgcaaaattggttcatcagccagtacgaaaagacggtctttgtcgggagctcatcccgaaacctccctctcagcactcgcctcgaatag

SEQ ID NO: 76 - protein, 890 aa, Verticillium dahliae
MKLTLATALLAASGCVSAGQPKLKHPQRQTNSSSELAFSPPHYPSPWMNPQATGWEDAYARAREVVEQMTLLEKVNLTTGVGWSGDLCVGNVGSIPRIGWRGLCLQDGPQGIRFADYVSYFTSSQTAGATWDRGLLYQRAHAIGAEGVAKGVDVVLGPAIGPLGRLPAGGRNWEGFAVDPYLSGVAVAESVRGIQDAGAIANVKHYIVNEQEHFRQAGEAQGYGYDVDEALSSNVDDKTMHELYLWPFADAVRAGAGSVMCSYQQINNSYGCQNSHLLNGLLKDELGFQGFVLSDWQAQHAGAATAVAGLDMAMPGDTRFNTGVAFWGANLTNAILNGTVPEYRLDDMAMRIMAAFFKVGKTLDDVPDINFSSWTKDTIGPLHWAAQDNVQVINQHVDVRQDHGALIRTIAARGTVLLKNEGSLPLNKPKFVAVIGEDAGPRPVGPNGCPDQGCNNGTLAAGWGSGTASFPYLITPDSALQFQAVSDGSRYESILSNWDYERTEALVSQADATALVFVNANSGEGYISVDGNEGDRKNLTLWNGGDELIQRVAAANNNTIVIIHSVGPVLVTDWYENPNITAIIWAGLPGQESGNSIADILYGRVNPGGKTPFTWGPTVESYGVDVLREPNNGNGAPQSDFDEGVFIDYRWFDRQSGVDNNASAPRNSSSSHAPIFEFGYGLSYTTFEFSNLQIERHDVHDYVPTTGQTSPAPRFGANYSTNYDDYVFPEGEIRYIYQHIYPYLNSSDPKEALADPKYGQTAEEFLPEGALDASPQPRLPASGGPGGNPMLWDVIFTVTATVTNTGKVAGDEVAQLYVSLGGPDDPIRVLRGFDRIHIAPGASQTFRAELTRRDLSNWDVVTQNWFISQYEKTVFVGSSSRNLPLSTRLE

SEQ ID NO: 77 - DNA, 2418 bp, Podospora anserina
atgaaactcaataagccattcctggccatttatttggctttcaacttggccgaggcttcgaaaactccggattgcatcagtggtccgctggcaaagaccttggcatgtgatacaacggcgtcacctcctgcgcgagcagctgctcttgtgcaggctttaaatatcacggaaaagcttgtgaatctagtggagtatgtcaagtcaagagaagctcctttagggatttcaattcagctaatcactcctcatagcatgagcctcggtgcagaaaggatcggccttccagcttatgcttggtggaacgaagctcttcatggtgttgccgcgtcgcctggggtctccttcaatcaggccggacaagaattctcacacgctacttcatttgcgaatactattacgctagcagccgcctttgacaatgacctggtttacgaggtggcggataccatcagcactgaagcgcgagcgttcagcaatgccgagctcgctggactggattactggacgcctaacatcaacccgtacaaagatccgagatgggggaggggccatgaggtttgttaccttagccttcttttccgtgccgtgcagttgctgagaactcaaaagacacccggagaagatccggtacacatcaaaggctacgtccaagcacttctcgagggtctagaagggagagacaagatcagaaaggtgattgccacttgtaaacactttgcagcctatgatttggagagatggcaaggggctcttagatacaggttcaatgctgttgtgacctcgcaggatctttcggagtactacctccaaccgtttcaacaatgcgctcgagacagcaaggtcgggtctttcatgtgctcatataatgcgctcaacggaacaccggcatgtgcaagcacgtatttgatggacgacatccttcgaaaacactggaattggaccgagcacaacaactatataacgagcgactgtaatgctattcaggacttcctccccaactttcacaacttcagccaaactccagctcaagccgccgctgatgcttataacgccggtacagacaccgtctgtgaggtgcctggataccccccactcacagatgtaatcggagcatacaatcagtctctgctgtcagaggaaattatcgaccgagcacttcgcagattatacgaaggcctcatccgagctggctatctcgactcagcctccccacatccatacaccaaaatctcatggtcccaagtaaacacccccaaagcccaagccctggctctccagtccgccaccgacgggatagtccttctcaaaaacaacggcctccttcccctagacctcaccaacaaaaccatagccctcataggccactgggccaatgcaacccgccaaatgctaggcggctacagcggtatccccccttactacgccaacccaatctatgcagccacccagctcaacgtcacttttcatcacgccccaggaccggtgaaccagtcatctccctccacaaatgacacctggacctcccccgccctctccgcggcttccaaatcggatatcatcctctacctcggcggcaccgacctctccatcgcagccgaagaccgagacagagactccatcgcctggccatccgctcaactttccttgttaacctccctcgcccagatgggaaaacccacaatcgtagcaagactaggcgaccaagtagacgacacccccctgctctccaacccaaacatctcctccatcctatgggtaggctacccaggccaatcaggcggaacagccctcttgaacatcatcaccggagtcagctcccccgccgctcgactgcccgtcacagtctacccagaaacttacacctccctcatccccctgacagccatgtccctccgcccaacctccgcccgcccaggccggacttacaggtggtacccctcccccgtgctccccttcggccacggcctccactacacaacctttaccgccaaattcggcgtctttgagtccctcaccatcaacattgccgaactcgtttccaactgtaacgaacgatacctcgacctctgccggttcccgcaggtgtccgtctgggtgtcgaatacgggagaactcaaatctgactatgtcgcccttgtttttgtcaggggtgagtacggaccggagccgtacccgatcaagacgctggtggggtacaagcggataagggatatcgagccggggactacgggggcggcgccggtgggggtggtggtgggggatttggctagggtggatttgggggggaatagggttttgtttccggggaagtatgagtttctgctggatgtggagggggggagggatagggttgtgatcgagttggttggggaggaggtggtgttggagaagttccctcagccgcctgcggcgggttga

SEQ ID NO: 78 - protein, 805 aa, Podospora anserina
MKLNKPFLAIYLAFNLAEASKTPDCISGPLAKTLACDTTASPPARAAALVQALNITEKLVNLVEYVKSREAPLGISIQLITPHSMSLGAERIGLPAYAWWNEALHGVAASPGVSFNQAGQEFSHATSFANTITLAAAFDNDLVYEVADTISTEARAFSNAELAGLDYWTPNINPYKDPRWGRGHEVCYLSLLFRAVQLLRTQKTPGEDPVHIKGYVQALLEGLEGRDKIRKVIATCKHFAAYDLERWQGALRYRFNAVVTSQDLSEYYLQPFQQCARDSKVGSFMCSYNALNGTPACASTYLMDDILRKHWNWTEHNNYITSDCNAIQDFLPNFHNFSQTPAQAAADAYNAGTDTVCEVPGYPPLTDVIGAYNQSLLSEEIIDRALRRLYEGLIRAGYLDSASPHPYTKISWSQVNTPKAQALALQSATDGIVLLKNNGLLPLDLTNKTIALIGHWANATRQMLGGYSGIPPYYANPIYAATQLNVTFHHAPGPVNQSSPSTNDTWTSPALSAASKSDIILYLGGTDLSIAAEDRDRDSIAWPSAQLSLLTSLAQMGKPTIVARLGDQVDDTPLLSNPNISSILWVGYPGQSGGTALLNIITGVSSPAARLPVTVYPETYTSLIPLTAMSLRPTSARPGRTYRWYPSPVLPFGHGLHYTTFTAKFGVFESLTINIAELVSNCNERYLDLCRFPQVSVWVSNTGELKSDYVALVFVRGEYGPEPYPIKTLVGYKRIRDIEPGTTGAAPVGVVVGDLARVDLGGNRVLFPGKYEFLLDVEGGRDRVVIELVGEEVVLEKFPQPPAAG

SEQ ID NO: 79 - protein, 721 aa, Thermotoga neapolitana
MEKVNEILSQLTLEEKVKLVVGVGLPGLFGNPHSRVAGAAGETHPVPRVGLPAFVLADGPAGLRINPTRENDENTYYTTAFPVEIMLASTWNRELLEEVGKAMGEEVREYGVDVLLAPAMNIHRNPLCGRNFEYYSEDPVLSGEMASSFVKGVQSQGVGACIKHFVANNQETNRMVVDTIVSERALREIYLRGFEIAVKKSKPWSVMSAYNKLNGKYCSQNEWLLKKVLREEWGFEGFVMSDWYAGDNPVEQLKAGNDLIMPGKAYQVNTERRDEIEEIMEALKEGKLSEEVLDECVRNILKVLVNAPSFKNYRYSNKPDLEKHAKVAYEAGAEGVVLLRNEEALPLSENSKIALFGTGQIETIKGGTGSGDTHPRYAISILEGIKERGLNFDEELAKTYEDYIKKMRETEEYKPRRDSWGTIIKPKLPENFLSEKEIHKLAKKNDVAVIVISRISGEGYDRKPVKGDFYLSDDETDLIKTVSREFHEQGKKVIVLLNIGSPVEVVSWRDLVDGILLVWQAGQETGRIVADVLTGRINPSGKLPTTFPRDYSDVPSWTFPGEPKDNPQKVVYEEDIYVGYRYYDTFGVEPAYEFGYGLSYTTFEYSDLNVSFDGETLRVQYRIENTGGRAGKEVSQVYIKAPKGKIDKPFQELKAFHKTRLLNPGESEEVVLEIPVRDLASFNGEEWVVEAGEYEVRVGASSRNIKLKGTFSVGEERRFKP

SEQ ID NO: 80 - protein, 871 aa, Podospora anserina
MAYRSLVLGAFASTSLAASVVTPRDPVPPGFVAAPYYPAPHGGWVASWEEAYSKAEALVSQMTLAEKTNITSGIGIFMGNTGSAERLGFPRMCLQDSALGVSSADNVTAFPAGITTGATFDKKLIYARGVAIGEEHRGKGTNVYLGPSVGPLGRKPLGGRNWEGFGSDPVLQAKAAALTIKGVQEQGIIATIKHLIGNEQEMYRMYNPFQPGYSANIDDRTLHELYLWPFAESVHAGVGSAMTAYNAVNGSACSQHSYLINGILKDELGFQGFVMSDWLSHISGVDSALAGLDMNMPGDTNIPLFGFSNWHYELSRSVLNGSVPLDRLNDMVTRIVATWYKFGQDRDHPRPNFSSNTRDRDGLLYPAALFSPKGQVNWFVNVQADHYLIAREVAQDAITLLKNNGSFLPLTTSQSLHVFGTAAQVNPDGPNACMNRACNKGTLGMGWGSGVADYPYLDDPISAIRKRVPDVKFFNTDGFPWFHPTPSPDDVAIVFITSDAGENSFTVEGNNGDRNSAKLAAWHNGDELVRKTAEKYNNVIVVAQTVGPLDLESWIDNPRVKGVLFQHLPGQEAGESLANILFGDVSPSGHLPYSITKRANDFPDSIANLRGFAFGQVQDTYSEGLYIDYRWLNKEKIRPRFAFGHGLSYTNFSFDATIESVTPLSLVPPARAPKGSTPVYSTEIPPASEAYWPEGFNRIWRYLYSWLNKNDADNAYAVGIAGVKKYNYPAGYSTAQKPGPAAGGGEGGNPALWDIAFRVPVTVKNTGDTFSGRASVQAYVQYPEGIPYDTPVVQLRDFEKTRVLAPGEEETVTVELTRKDLSVWDTELQNWVVPGVGGKRYTVWIGEASDRLFTACYTDTGVCEGGRVPPV

SEQ ID NO: 81 - DNA, 2799 bp, Podospora anserina
atggcataccgctcattagtcttgggcgccttcgcctccacctctcttgccgccagcgtcgtgacgcctcgagatcctgttccgcctggattcgtcgctgccccatactatccagcgcctcatggaggatgggtcgcttcgtgggaagaggcttacagcaaggccgaagccttggtctcgcagatgaccttggctgaaaagaccaacatcacctcaggcattggcatctttatgggtgagttattaaccagacatggcttatataaaagcacaagagactgactgacatgtgaatagggtcagtgccaccaccctaatgagacgtttttctgattttgactaacacatgatacgctagtccatgcgtaggaaatactggaagcgcagaaagattggggttcccgcgcatgtgtcttcaggactctgcgttgggtgtgtcgtcggctgacaacgtcactgcgtttcctgctggcatcaccactggtgcaacgtttgacaagaagctgatctatgctcgtggtgttgctattggtgaagagcatcgcggcaagggcacaaatgtctatctgggtccttccgtaggccctcttgggcggaagcctttgggtggccgcaactgggagggctttggatctgacccagttcttcaagccaaggctgctgccctgacgatcaagggcgttcaggaacaaggcatcattgctactatcaagcatctgatcggcaacgagcaggagatgtatagaatgtacaaccccttccagcctggatatagcgccaatattggtgagtggactcttgctctttgacggactaaaaggctgactccccacagatgatcggactctgcacgagctctacctgtggccctttgccgaatccgtccatgccggtgttgggtcggcaatgacagcttacaatgctgtaaacgggtctgcttgctctcagcacagctatctcatcaacggtattttgaaggatgagcttggattccagggcttcgtcatgtctgactggctgtcccacatctccggagtcgactccgcgttggcaggtctcgacatgaacatgccaggtgacaccaacattcccctatttggtttcagcaactggcactatgagctcagcagatcggttctcaacgggtctgtgcctcttgacagactgaacgacatggtcaccagaatcgtcgcgacatggtacaagttcggtcaggatagggaccacccaaggcctaacttctcgtcaaacacccgtgaccgtgacggtctgctttatcctgcagctctcttctcccccaagggtcaggtgaactggtttgtcaatgttcaggctgatcattatttgatcgccagagaggtcgcccaggatgccatcacccttctcaagaacaatgggagcttccttcccctgacgacttcgcagtctctccatgtcttcggtactgctgcccaggtcaaccccgatgggcccaacgcttgcatgaaccgcgcctgcaacaaaggaacacttggcatgggctggggttctggtgttgccgattatccttacttggatgacccgatctcggctatcaggaagcgggttcccgacgtcaagttcttcaacaccgacggcttcccttggttccaccctacaccgtcgcccgatgacgttgccatcgtgttcatcacctccgatgctggagagaactcgttcactgttgagggcaacaacggtgatcgcaacagtgccaagctggctgcgtggcataacggtgacgagctggtcaggaagactgccgagaagtacaacaacgttattgtggtagctcaaaccgtcggccctctcgatctcgaatcctggatcgacaaccctcgcgtcaagggcgtcctgtttcagcaccttcccggtcaagaagcgggcgagtcgttggccaacattctctttggcgatgtctcccctagcggtcaccttccctactccatcaccaagcgcgccaacgacttccccgacagcatcgccaacctccgtggctttgcctttggtcaggtccaggacacgtacagcgagggcctgtacattgactaccgctggctcaacaaggagaagatcaggccccgctttgcttttggccacggtctcagctacaccaacttctcgtttgatgccaccatcgagtctgtcactccactgtctctggttcctcctgcccgtgcccccaagggctcaacgccggtgtactcgaccgaaatcccccccgcctcagaggcgtactggccggaagggttcaacaggatctggcggtacctctactcctggctcaacaagaacgacgcggataacgcctacgctgttggtatcgccggggtgaagaagtataactatcccgctgggtacagcaccgcccagaagcccggtcccgcagccggtggcggggaggggggtaatcctgcgctttgggatattgctttccgtgtcccagttacggtcaagaacactggggatacgttctcgggacgggcttcggtgcaggcttatgttcagtatcctgaggggatcccgtatgatacgcctgttgtgcagctgagggactttgagaagacgagggttttggctccgggggaggaggagacggtgacggttgagctgaccaggaaggacttgagcgtgtgggacacggagctgcagaactgggttgtgccgggggttggggggaagaggtatacggtttggattggggaggcgagcgataggttgtttacggcttgttatacggatacgggggtttgtgagggggggagggtgccgcctgtttaa

SEQ ID NO: 82 - DNA, 3193 bp, Artificial Sequence
  synthetic chimeric Fv3c/Bgl3 sequence
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgaccgacgatctccaagcaccgatggaaagagctctcccaacaacaccgctgctcctctctacgagttcggtcacggtctatcttggtcgacgttcaagttctccaacctccacatccagaagaacaatgtcggccccatgagcccgcccaacggcaagacgattgcggctccctctctgggcagcttcagcaagaaccttaaggactatggcttccccaagaacgttcgccgcatcaaggagtttatctacccctacctgagcaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 83 - DNA, 3157 bp, Artificial Sequence
  synthetic Fv3C/Te3A/T. reesei Bgl3 (FAB) chimera sequence
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgacaagtacaacatcacgcctatctacgagttcggtcacggtctatcttggtcgacgttcaagttctccaacctccacatccagaagaacaatgtcggccccatgagcccgcccaacggcaagacgattgcggctccctctctgggcaacttcagcaagaaccttaaggactatggcttccccaagaacgttcgccgcatcaaggagtttatctacccctacctgaacaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 84 - protein, 19 aa, Artificial Sequence
  synthetic GH61 endoglucanase family motif
  MISC_FEATURE (1)..(1): Xaa can be Ile, Leu, Met or Val
  misc_feature (3)..(6): Xaa can be any naturally occurring amino acid
  misc_feature (8)..(8): Xaa can be any naturally occurring amino acid
  MISC_FEATURE (10)..(10): Xaa can be Ile, Leu, Met or Val
  misc_feature (11)..(11): Xaa can be any naturally occurring amino acid
  misc_feature (13)..(13): Xaa can be any naturally occurring amino acid
  MISC_FEATURE (14)..(14): Xaa can be Glu or Gln
  misc_feature (15)..(18): Xaa can be any naturally occurring amino acid
  MISC_FEATURE (19)..(19): Xaa can be His, Asn or Gln
XPXXXXGXYXXRXXXXXXX

SEQ ID NO: 85 - protein, 20 aa, Artificial Sequence
  synthetic GH61 endoglucanase family motif
  MISC_FEATURE (1)..(1): Xaa can be Ile, Leu, Met or Val
  misc_feature (3)..(7): Xaa can be any naturally occurring amino acid
  misc_feature (9)..(9): Xaa can be any naturally occurring amino acid
  MISC_FEATURE (11)..(11): Xaa can be Ile, Leu, Met or Val
  misc_feature (12)..(12): Xaa can be any naturally occurring amino acid
  misc_feature (14)..(14): Xaa can be any naturally occurring amino acid
  MISC_FEATURE (15)..(15): Xaa can be Glu or Gln
  misc_feature (16)..(19): Xaa can be any naturally occurring amino acid
  MISC_FEATURE (20)..(20): Xaa can be His, Asn or Gln
XPXXXXXGXYXXRXXXXXXX

SEQ ID NO: 86 - protein, 19 aa, Artificial Sequence
  synthetic GH61 endoglucanase family motif
  MISC_FEATURE (1)..(1): Xaa can be Ile, Leu, Met or Val
  misc_feature (3)..(6): Xaa can be any naturally occurring amino acid
  misc_feature (8)..(8): Xaa can be any naturally occurring amino acid
  MISC_FEATURE (10)..(10): Xaa can be Ile, Leu, Met or Val
  misc_feature (11)..(11): Xaa can be any naturally occurring amino acid
  misc_feature (13)..(13): Xaa can be any naturally occurring amino acid
  MISC_FEATURE (14)..(14): Xaa can be Glu or Gln
  misc_feature (15)..(17): Xaa can be any naturally occurring amino acid
  MISC_FEATURE (19)..(19): Xaa can be His, Asn or Gln
XPXXXXGXYXXRXXXXXAX

SEQ ID NO: 87 - protein, 20 aa, Artificial Sequence
  synthetic GH61 endoglucanase family motif
  MISC_FEATURE (1)..(1): Xaa can be Ile, Leu, Met or Val
  misc_feature (3)..(7): Xaa can be any naturally occurring amino acid
  misc_feature (9)..(9): Xaa can be any naturally occurring amino acid
  MISC_FEATURE (11)..(11): Xaa can be Ile, Leu, Met or Val
  misc_feature (12)..(12): Xaa can be any naturally occurring amino acid
  misc_feature (14)..(14): Xaa can be any naturally occurring amino acid
  MISC_FEATURE (15)..(15): Xaa can be Glu or Gln
  misc_feature (16)..(18): Xaa can be any naturally occurring amino acid
  MISC_FEATURE (20)..(20): Xaa can be His, Asn or Gln
XPXXXXXGXYXXRXXXXXAX

SEQ ID NO: 88 - protein, 4 aa, Artificial Sequence
  synthetic GH61 endoglucanase family motif
  MISC_FEATURE (1)..(1): Xaa can be Phe or Trp
  MISC_FEATURE (2)..(2): Xaa can be Phe or Thr
  MISC_FEATURE (4)..(4): Xaa can be Ala, Ile or Val
XXKX

SEQ ID NO: 89 - protein, 10 aa, Artificial Sequence
  synthetic GH61 endoglucanase family motif
  misc_feature (2)..(3): Xaa can be any naturally occurring amino acid
  misc_feature (6)..(8): Xaa can be any naturally occurring amino acid
  MISC_FEATURE (9)..(9): Xaa can be Tyr or Trp
  MISC_FEATURE (10)..(10): Xaa can be Ala, Ile, Leu, Met or Val
HXXGPXXXXX

SEQ ID NO: 90 - protein, 9 aa, Artificial Sequence
  synthetic GH61 endoglucanase family motif
  misc_feature (2)..(2): Xaa can be any naturally occurring amino acid
  misc_feature (5)..(7): Xaa can be any naturally occurring amino acid
  MISC_FEATURE (8)..(8): Xaa can be Tyr or Trp
  MISC_FEATURE (9)..(9): Xaa can be Ala, Ile, Leu, Met or Val
HXGPXXXXX

SEQ ID NO: 91 - protein, 11 aa, Artificial Sequence
  synthetic GH61 endoglucanase family motif
  MISC_FEATURE (1)..(1): Xaa can be Glu or Gln
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  MISC_FEATURE (8)..(8): Xaa can be Glu, His, Gln or Asn
  MISC_FEATURE (9)..(9): Xaa can be Phe, Ile, Leu or Val
  misc_feature (10)..(10): Xaa can be any naturally occurring amino acid
  MISC_FEATURE (11)..(11): Xaa can be Ile, Leu or Val
XXYXXCXXXXX

SEQ ID NO: 92 - DNA, 28 bp, Artificial Sequence
  synthetic primer
caccatgagatatagaacagctgccgct

SEQ ID NO: 93 - DNA, 40 bp, Artificial Sequence
  synthetic primer
cgaccgccctgcggagtcttgcccagtggtcccgcgacag

SEQ ID NO: 94 - DNA, 40 bp, Artificial Sequence
  synthetic primer
ctgtcgcgggaccactgggcaagactccgcagggcggtcg

SEQ ID NO: 95 - DNA, 20 bp, Artificial Sequence
  synthetic primer
cctacgctaccgacagagtg

SEQ ID NO: 96 - DNA, 20 bp, Artificial Sequence
  synthetic primer
gtctagactggaaacgcaac

SEQ ID NO: 97 - DNA, 21 bp, Artificial Sequence
  synthetic primer
gagttgtgaagtcggtaatcc

SEQ ID NO: 98 - DNA, 35 bp, Artificial Sequence
  synthetic primer
caccatgaaagcaaacgtcatcttgtgcctcctgg

SEQ ID NO: 99 - DNA, 43 bp, Artificial Sequence
  synthetic primer
ctattgtaagatgccaacaatgctgttatatgccggcttgggg

SEQ ID NO: 100 - DNA, 21 bp, Artificial Sequence
  synthetic primer
gagttgtgaagtcggtaatcc

SEQ ID NO: 101 - DNA, 18 bp, Artificial Sequence
  synthetic primer
cacgaagagcggcgattc

SEQ ID NO: 102 - DNA, 23 bp, Artificial Sequence
  synthetic primer
cacccatgctgctcaatcttcag

SEQ ID NO: 103 - DNA, 23 bp, Artificial Sequence
  synthetic primer
ttacgcagacttggggtcttgag

SEQ ID NO: 104 - DNA, 20 bp, Artificial Sequence
  synthetic primer
gcttgagtgtatcgtgtaag

SEQ ID NO: 105 - DNA, 21 bp, Artificial Sequence
  synthetic primer
gcaacggcaaagccccacttc

SEQ ID NO: 106 - DNA, 32 bp, Artificial Sequence
  synthetic primer
gtagcggccgcctcatctcatctcatccatcc

SEQ ID NO: 107 - DNA, 24 bp, Artificial Sequence
  synthetic primer
caccatgcagctcaagtttctgtc

SEQ ID NO: 108 - DNA, 32 bp, Artificial Sequence
  synthetic primer
ggttactagtcaactgcccgttctgtagcgag

SEQ ID NO: 109 - DNA, 29 bp, Artificial Sequence
  synthetic primer
catgcgatcgcgacgttttggtcaggtcg

SEQ ID NO: 110 - DNA, 40 bp, Artificial Sequence
  synthetic primer
gacagaaacttgagctgcatggtgtgggacaacaagaagg

SEQ ID NO: 111 - DNA, 29 bp, Artificial Sequence
  synthetic primer
caccatggttcgcttcagttcaatcctag

SEQ ID NO: 112 - DNA, 22 bp, Artificial Sequence
  synthetic primer
gtggctagaagatatccaacac

SEQ ID NO: 113 - DNA, 29 bp, Artificial Sequence
  synthetic primer
catgcgatcgcgacgttttggtcaggtcg

SEQ ID NO: 114 - DNA, 39 bp, Artificial Sequence
  synthetic primer
gaactgaagcgaaccatggtgtgggacaacaagaaggac

SEQ ID NO: 115 - DNA, 21 bp, Artificial Sequence
  synthetic primer
gtagttatgcgcatgctagac

SEQ ID NO: 116 - DNA, 24 bp, Artificial Sequence
  synthetic primer
caccatgaagctgaattgggtcgc

SEQ ID NO: 117 - DNA, 19 bp, Artificial Sequence
  synthetic primer
ttactccaacttggcgctg

SEQ ID NO: 118 - DNA, 20 bp, Artificial Sequence
  synthetic primer
aagccaagagctttgtgtcc

SEQ ID NO: 119 - DNA, 20 bp, Artificial Sequence
  synthetic primer
tatgcacgagctctacgcct

SEQ ID NO: 120 - DNA, 20 bp, Artificial Sequence
  synthetic primer
atggtaccctggctatggct

SEQ ID NO: 121 - DNA, 20 bp, Artificial Sequence
  synthetic primer
cggtcacggtctatcttggt

SEQ ID NO: 122 - DNA, 45 bp, Artificial Sequence
  synthetic primer
gctagcatggatgttttcccagtcacgacgttgtaaaacgacggc

SEQ ID NO: 123 - DNA, 53 bp, Artificial Sequence
  synthetic primer
ggaggttggagaacttgaacgtcgaccaagatagaccgtgaccgaactcgtag

SEQ ID NO: 124 - DNA, 43 bp, Artificial Sequence
  synthetic primer
tgccaggaaacagctatgaccatgtaatacgactcactatagg

SEQ ID NO: 125 - DNA, 53 bp, Artificial Sequence
  synthetic primer
ctacgagttcggtcacggtctatcttggtcgacgttcaagttctccaacctcc

SEQ ID NO: 126 - DNA, 42 bp, Artificial Sequence
  synthetic primer
taagctcgggccccaaataatgattttattttgactgatagt

SEQ ID NO: 127 - DNA, 45 bp, Artificial Sequence
  synthetic primer
gggatatcagctggatggcaaataatgattttattttgactgata

SEQ ID NO: 128 - DNA, 26 bp, Artificial Sequence
  synthetic primer
gagttgtgaagtcggtaatcccgctg

SEQ ID NO: 129 - DNA, 30 bp, Artificial Sequence
  synthetic primer
cctgcacgagggcatcaagctcactaaccg

SEQ ID NO: 130 - DNA, 27 bp, Artificial Sequence
  synthetic primer
cggaatgagctagtaggcaaagtcagc

SEQ ID NO: 131 - DNA, 70 bp, Artificial Sequence
  synthetic primer
ctccttgatgcggcgaacgttcttggggaagccatagtccttaaggttcttgctgaagttgcccagagag

SEQ ID NO: 132 - DNA, 65 bp, Artificial Sequence
  synthetic primer
ggcttccccaagaacgttcgccgcatcaaggagtttatctacccctacctgaacaccactacctc

SEQ ID NO: 133 - DNA, 27 bp, Artificial Sequence
  synthetic primer
gatacacgaagagcggcgattctacgg

SEQ ID NO: 134 - DNA, 24 bp, Artificial Sequence
  synthetic primer
caccatgaagctgaattgggtcgc

SEQ ID NO: 135 - protein, 886 aa, Artificial Sequence
  synthetic chimeric Fv3c/Te3A/T. reesei Bgl3 (FAB) sequence
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDKYNITPIYEFGHGLSWSTFKFSNLHIQKNNVGPMSPPNGKTIAAPSLGNFSKNLKDYGFPKNVRRIKEFIYPYLNTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 136 - protein, 23 aa, Artificial Sequence
  synthetic chimeric beta-glucosidase motif
  misc_feature (2)..(2): Xaa can be any naturally occurring amino acid
  misc_feature (6)..(6): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(21): Xaa can be any naturally occurring amino acid
AXSPPXYPSPWMDPXAXGWEXAY

SEQ ID NO: 137 - protein, 32 aa, Artificial Sequence
  synthetic chimeric beta-glucosidase motif
  misc_feature (3)..(3): Xaa can be any naturally occurring amino acid
  misc_feature (7)..(8): Xaa can be any naturally occurring amino acid
  misc_feature (11)..(11): Xaa can be any naturally occurring amino acid
  misc_feature (23)..(23): Xaa can be any naturally occurring amino acid
  misc_feature (26)..(26): Xaa can be any naturally occurring amino acid
AKXFVSXXTLXEKVNLTTGVGWXGEXCVGNVG

SEQ ID NO: 138 - protein, 18 aa, Artificial Sequence
  synthetic chimeric beta-glucosidase motif
  misc_feature (3)..(3): Xaa can be any naturally occurring amino acid
  misc_feature (7)..(7): Xaa can be any naturally occurring amino acid
  misc_feature (10)..(10): Xaa can be any naturally occurring amino acid
  misc_feature (17)..(17): Xaa can be any naturally occurring amino acid
PRXGMRXLCXQDGPLGXR

SEQ ID NO: 139 - protein, 16 aa, Artificial Sequence
  synthetic chimeric beta-glucosidase motif
  misc_feature (6)..(7): Xaa can be any naturally occurring amino acid
  misc_feature (9)..(9): Xaa can be any naturally occurring amino acid
  misc_feature (12)..(12): Xaa can be any naturally occurring amino acid
YNSAFXXGXTAXASWS

SEQ ID NO: 140 - protein, 19 aa, Artificial Sequence
  synthetic chimeric beta-glucosidase motif
  misc_feature (2)..(2): Xaa can be any naturally occurring amino acid
  misc_feature (9)..(11): Xaa can be any naturally occurring amino acid
  misc_feature (17)..(17): Xaa can be any naturally occurring amino acid
GXIACAKHXXXNEQEHXRQ

SEQ ID NO: 141 - protein, 27 aa, Artificial Sequence
  synthetic chimeric beta-glucosidase motif
  misc_feature (5)..(5): Xaa can be any naturally occurring amino acid
  misc_feature (10)..(10): Xaa can be any naturally occurring amino acid
  misc_feature (13)..(13): Xaa can be any naturally occurring amino acid
  misc_feature (15)..(15): Xaa can be any naturally occurring amino acid
  misc_feature (19)..(19): Xaa can be any naturally occurring amino acid
  misc_feature (23)..(23): Xaa can be any naturally occurring amino acid
LSSNXDDKTXHEXYXWPFXDAVXAGVG

SEQ ID NO: 142 - protein, 21 aa, Artificial Sequence
  synthetic chimeric beta-glucosidase motif
  misc_feature (5)..(5): Xaa can be any naturally occurring amino acid
  misc_feature (7)..(7): Xaa can be any naturally occurring amino acid
  misc_feature (12)..(12): Xaa can be any naturally occurring amino acid
  misc_feature (19)..(19): Xaa can be any naturally occurring amino acid
MCSYXQXNNSYXCQNSKLXNG

SEQ ID NO: 143 - protein, 32 aa, Artificial Sequence
  synthetic chimeric beta-glucosidase motif
  misc_feature (11)..(11): Xaa can be any naturally occurring amino acid
  misc_feature (15)..(15): Xaa can be any naturally occurring amino acid
  misc_feature (17)..(17): Xaa can be any naturally occurring amino acid
  misc_feature (19)..(19): Xaa can be any naturally occurring amino acid
  misc_feature (27)..(27): Xaa can be any naturally occurring amino acid
GFQGFVMSDWXAQHXGXAXAVAGLDMXMPGDT

SEQ ID NO: 144 - protein, 19 aa, Artificial Sequence
  synthetic chimeric beta-glucosidase motif
  misc_feature (7)..(7): Xaa can be any naturally occurring amino acid
  misc_feature (13)..(13): Xaa can be any naturally occurring amino acid
  misc_feature (16)..(16): Xaa can be any naturally occurring amino acid
NLTLAVXNGTVPXWRXDDM

SEQ ID NO: 145 - protein, 26 aa, Artificial Sequence
  synthetic chimeric beta-glucosidase motif
  misc_feature (2)..(2): Xaa can be any naturally occurring amino acid
  misc_feature (5)..(5): Xaa can be any naturally occurring amino acid
  misc_feature (7)..(7): Xaa can be any naturally occurring amino acid
  misc_feature (13)..(13): Xaa can be any naturally occurring amino acid
  misc_feature (22)..(22): Xaa can be any naturally occurring amino acid
PXFLXVXGEDAGXNPAGPNGCXDRGC

SEQ ID NO: 146 - protein, 16 aa, Artificial Sequence
  synthetic chimeric beta-glucosidase motif
  misc_feature (6)..(6): Xaa can be any naturally occurring amino acid
  misc_feature (12)..(12): Xaa can be any naturally occurring amino acid
GTLAMXWGSGTXFPYL

SEQ ID NO: 147 - protein, 29 aa, Artificial Sequence
  synthetic chimeric beta-glucosidase motif
  misc_feature (7)..(8): Xaa can be any naturally occurring amino acid
  misc_feature (15)..(15): Xaa can be any naturally occurring amino acid
  misc_feature (20)..(20): Xaa can be any naturally occurring amino acid
AIVFANXXSGEGYIXVDGNXGDRKNLTLW

SEQ ID NO: 148 - protein, 17 aa, Artificial Sequence
  synthetic chimeric beta-glucosidase motif
  misc_feature (2)..(2): Xaa can be any naturally occurring amino acid
  misc_feature (7)..(7): Xaa can be any naturally occurring amino acid
  misc_feature (12)..(12): Xaa can be any naturally occurring amino acid
DXLYGKXSPGRXPFTWG

SEQ ID NO: 149 - protein, 19 aa, Artificial Sequence
  synthetic chimeric beta-glucosidase motif
  misc_feature (2)..(2): Xaa can be any naturally occurring amino acid
  misc_feature (7)..(7): Xaa can be any naturally occurring amino acid
  misc_feature (12)..(12): Xaa can be any naturally occurring amino acid
  misc_feature (15)..(16): Xaa can be any naturally occurring amino acid
  misc_feature (18)..(18): Xaa can be any naturally occurring amino acid
PXYEFGXGLSWXTFXXSXL

SEQ ID NO: 150 - protein, 7 aa, Artificial Sequence
  synthetic chimeric beta-glucosidase motif
  misc_feature (2)..(2): Xaa can be any naturally occurring amino acid
  misc_feature (5)..(5): Xaa can be any naturally occurring amino acid
LXDYXFP

SEQ ID NO: 151 - protein, 15 aa, Artificial Sequence
  synthetic chimeric beta-glucosidase motif
  misc_feature (5)..(6): Xaa can be any naturally occurring amino acid
  misc_feature (9)..(9): Xaa can be any naturally occurring amino acid
  misc_feature (12)..(12): Xaa can be any naturally occurring amino acid
EFLPXXALXGSXQPR

SEQ ID NO: 152 - protein, 12 aa, Artificial Sequence
  synthetic chimeric beta-glucosidase motif
  misc_feature (3)..(3): Xaa can be any naturally occurring amino acid
  misc_feature (8)..(9): Xaa can be any naturally occurring amino acid
  misc_feature (11)..(11): Xaa can be any naturally occurring amino acid
SGXPGGNXXLXD

SEQ ID NO: 153 - protein, 11 aa, Artificial Sequence
  synthetic chimeric beta-glucosidase motif
  misc_feature (4)..(4): Xaa can be any naturally occurring amino acid
  misc_feature (6)..(6): Xaa can be any naturally occurring amino acid
YTVXAXITNTG

SEQ ID NO: 154 - protein, 16 aa, Artificial Sequence
  synthetic chimeric beta-glucosidase motif
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
  misc_feature (15)..(15): Xaa can be any naturally occurring amino acid
VLRGFXRXEXIAPGXS

SEQ ID NO: 155 - protein, 19 aa, Artificial Sequence
  synthetic chimeric beta-glucosidase motif
  misc_feature (10)..(12): Xaa can be any naturally occurring amino acid
  misc_feature (14)..(14): Xaa can be any naturally occurring amino acid
TRRDLSNWDXXXQXWVITD

SEQ ID NO: 156 - protein, 14 aa, Artificial Sequence
  synthetic chimeric beta-glucosidase motif
  misc_feature (7)..(7): Xaa can be any naturally occurring amino acid
  misc_feature (11)..(11): Xaa can be any naturally occurring amino acid
  misc_feature (13)..(13): Xaa can be any naturally occurring amino acid
VGSSSRXLPLXAXL

SEQ ID NO: 157 - protein, 19 aa, Fusarium verticillioides
RRSPSTDGKSSPNNTAAPL

SEQ ID NO: 158 - protein, 7 aa, Talaromyces emersonii
KYNITPI

SEQ ID NO: 159 - protein, 898 aa, Artificial Sequence
  synthetic chimeric Fv3c/Bgl3 sequence
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSSPNNTAAPLYEFGHGLSWSTFKFSNLHIQKNNVGPMSPPNGKTIAAPSLGSFSKNLKDYGFPKNVRRIKEFIYPYLSTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 160 - DNA, 71 bp, Artificial Sequence
  synthetic primer
gatagaccgtgaccgaactcgtagataggcgtgatgttgtacttgtcgaagtgacggtagtcgatgaagac

SEQ ID NO: 161 - DNA, 71 bp, Artificial Sequence
  synthetic primer
gtcttcatcgactaccgtcacttcgacaagtacaacatcacgcctatctacgagttcggtcacggtctatc

SEQ ID NO: 162 - DNA, 780 bp, Trichoderma reesei
atggtctccttcacctccctcctcgccggcgtcgccgccatctcgggcgtcttggccgctcccgccgccgaggtcgaatccgtggctgtggagaagcgccagacgattcagcccggcacgggctacaacaacggctacttctactcgtactggaacgatggccacggcggcgtgacgtacaccaatggtcccggcgggcagttctccgtcaactggtccaactcgggcaactttgtcggcggcaagggatggcagcccgggaccaagaacaagtaagactacctactcttaccccctttgaccaacacagcacaacacaatacaacacatgtgactaccaatcatggaatcggatctaacagctgtgttttaaaaaaaagggtcatcaacttctcgggaagctacaaccccaacggcaacagctacctctccgtgtacggctggtcccgcaaccccctgatcgagtactacatcgtcgagaactttggcacctacaacccgtccacgggcgccaccaagctgggcgaggtcacctccgacggcagcgtctacgacatttaccgcacgcagcgcgtcaaccagccgtccatcatcggcaccgccaccttttaccagtactggtccgtccgccgcaaccaccgctcgagcggctccgtcaacacggcgaaccacttcaacgcgtgggctcagcaaggcctgacgctcgggacgatggattaccagattgttgccgtggagggttactttagctctggctctgcttccatcaccgtcagctaa

SEQ ID NO: 163 - DNA, 2394 bp, Trichoderma reesei
atggtgaataacgcagctcttctcgccgccctgtcggctctcctgcccacggccctggcgcagaacaatcaaacatacgccaactactctgctcagggccagcctgatctctaccccgagacacttgccacgctcacactctcgttccccgactgcgaacatggccccctcaagaacaatctcgtctgtgactcatcggccggctatgtagagcgagcccaggccctcatctcgctcttcaccctcgaggagctcattctcaacacgcaaaactcgggccccggcgtgcctcgcctgggtcttccgaactaccaagtctggaatgaggctctgcacggcttggaccgcgccaacttcgccaccaagggcggccagttcgaatgggcgacctcgttccccatgcccatcctcactacggcggccctcaaccgcacattgatccaccagattgccgacatcatctcgacccaagctcgagcattcagcaacagcggccgttacggtctcgacgtctatgcgccaaacgtcaatggcttccgaagccccctctggggccgtggccaggagacgcccggcgaagacgcctttttcctcagctccgcctatacttacgagtacatcacgggcatccagggtggcgtcgaccctgagcacctcaaggttgccgccacggtgaagcactttgccggatacgacctcgagaactggaacaaccagtcccgtctcggtttcgacgccatcataactcagcaggacctctccgaatactacactccccagttcctcgctgcggcccgttatgcaaagtcacgcagcttgatgtgcgcatacaactccgtcaacggcgtgcccagctgtgccaacagcttcttcctgcagacgcttttgcgcgagagctggggcttccccgaatggggatacgtctcgtccgattgcgatgccgtctacaacgttttcaaccctcatgactacgccagcaaccagtcgtcagccgccgccagctcactgcgagccggcaccgatatcgactgcggtcagacttacccgtggcacctcaacgagtcctttgtggccggcgaagtctcccgcggcgagatcgagcggtccgtcacccgtctgtacgccaacctcgtccgtctcggatacttcgacaagaagaaccagtaccgctcgctcggttggaaggatgtcgtcaagactgatgcctggaacatctcgtacgaggctgctgttgagggcatcgtcctgctcaagaacgatggcactctccctctgtccaagaaggtgcgcagcattgctctgatcggaccatgggccaatgccacaacccaaatgcaaggcaactactatggccctgccccatacctcatcagccctctggaagctgctaagaaggccggctatcacgtcaactttgaactcggcacagagatcgccggcaacagcaccactggctttgccaaggccattgctgccgccaagaagtcggatgccatcatctacctcggtggaattgacaacaccattgaacaggagggcgctgaccgcacggacattgcttggcccggtaatcagctggatctcatcaagcagctcagcgaggtcggcaaaccccttgtcgtcctgcaaatgggcggtggtcaggtagactcatcctcgctcaagagcaacaagaaggtcaactccctcgtctggggcggatatcccggccagtcgggaggcgttgccctcttcgacattctctctggcaagcgtgctcctgccggccgactggtcaccactcagtacccggctgagtatgttcaccaattcccccagaatgacatgaacctccgacccgatggaaagtcaaaccctggacagacttacatctggtacaccggcaaacccgtctacgagtttggcagtggtctcttctacaccaccttcaaggagactctcgccagccaccccaagagcctcaagttcaacacctcatcgatcctctctgctcctcaccccggatacacttacagcgagcagattcccgtcttcaccttcgaggccaacatcaagaactcgggcaagacggagtccccatatacggccatgctgtttgttcgcacaagcaacgctggcccagccccgtacccgaacaagtggctcgtcggattcgaccgacttgccgacatcaagcctggtcactcttccaagctcagcatccccatccctgtcagtgctctcgcccgtgttgattctcacggaaaccggattgtataccccggcaagtatgagctagccttgaacaccgacgagtctgtgaagcttgagtttgagttggtgggagaagaggtaacgattgagaactggccgttggaggagcaacagatcaaggatgctacacctgacgcataa

SEQ ID NO: 164 - protein, 8 aa, Artificial Sequence
  synthetic amino acid sequence motif
YPSPWMDP

SEQ ID NO: 165 - protein, 11 aa, Artificial Sequence
  synthetic amino acid sequence motif
EKVNLTTGVGW

SEQ ID NO: 166 - protein, 5 aa, Artificial Sequence
  synthetic amino acid sequence motif
  MISC_FEATURE (3)..(3): Xaa can be Ile or Val
  MISC_FEATURE (5)..(5): Xaa can be Ile or Val
KGXDX

SEQ ID NO: 167 - protein, 9 aa, Artificial Sequence
  synthetic amino acid sequence motif
  misc_feature (7)..(7): Xaa can be any naturally occurring amino acid
CQNSKLXNG

SEQ ID NO: 168 - protein, 14 aa, Artificial Sequence
  synthetic amino acid sequence motif
  MISC_FEATURE (7)..(7): Xaa can be Leu, Ile or Val
  MISC_FEATURE (10)..(10): Xaa can be Ser or Thr
  MISC_FEATURE (11)..(11): Xaa can be Ile or Val
  misc_feature (13)..(13): Xaa can be any naturally occurring amino acid
NLTLAVXNGXXPXW

SEQ ID NO: 169 - protein, 8 aa, Artificial Sequence
  synthetic amino acid sequence motif
  MISC_FEATURE (3)..(3): Xaa can be Ser or Thr
  misc_feature (4)..(4): Xaa can be any naturally occurring amino acid
  MISC_FEATURE (7)..(7): Xaa can be Phe or Tyr
SWXXDTXG

SEQ ID NO: 170 - protein, 15 aa, Artificial Sequence
  synthetic amino acid sequence motif
  misc_feature (5)..(6): Xaa can be any naturally occurring amino acid
  misc_feature (9)..(9): Xaa can be any naturally occurring amino acid
  misc_feature (12)..(12): Xaa can be any naturally occurring amino acid
EFLPXXALXGSXQPR

SEQ ID NO: 171 - protein, 7 aa, Artificial Sequence
  synthetic loop sequence
FDRRSPG

SEQ ID NO: 172 - protein, 7 aa, Artificial Sequence
  synthetic loop sequence
  misc_feature (3)..(3): Xaa can be Arg or Lys
FDXYNIT

SEQ ID NO: 173 - protein, 17 aa, Trichoderma reesei
MYRKLAVISAFLATARA

SEQ ID NO: 174 - protein, 884 aa, Nectria haematococca
MRFTVLLAAFSGLVPMVGSQADQKPLQLGVNNNTLAHSPPHYPSPWMDPAAPGWEEAYLKAKDFVSQLTLLEKVNLTTGVGWMGERCVGNVGSLPRFGMRGLCMQDGPLGIRLSDYNSAFPTGITAGASWSRALWYQRGLLMGTEHREKGIDVALGPATGPLGRTPTGGRNWEGFSVDPYVAGVAMAETVSGIQDGGTIACAKHYIGNEQEHHRQAPESIGRGYNITESLSSNVDDKTLHELYLWPFADAVKAGVGAIMCSYQQLNNSYGCQNSKLLNGILKDELGFQGFVMSDWQAQHAGAATAVAGLDMTMPGDTLFNTGYSFWGGNLTLAVVNGTVPDWRIDDMAMRIMAAFFKVGKTVEDLPDINFSSWSRDTFGYVQAAAQENWEQINFGVDVRHDHSEHIRLSAAKGTVLLKNSGSLPLKKPKFLAVVGEDAGPNPAGPNGCNDRGCNNGTLAMSWGSGTAQFPYLVTPDSALQNQAVLDGTRYESVLRNNQWEQTRSLISQPNVTAIVFANANSGEGYIDVDGNEGDRKNLTLWNEGDDLIKNVSSICPNTIVVLHTVGPVILTEWYDNPNITAIVWAGVPGQESGNALVDILYGKTSPGRSPFTWGRTRKSYGTDVLYEPNNGQGAPQDDFTEGVFIDYRHFDQVSPSTDGSKSNDESSPIYEFGHGLSWTTFEYSELNIQAHNKIPFDPPIGETIAAPVLGNYSTDLADYTFPDGIRYIYQFIYPWLNTSSSGREASGDPDYGKTAEEFLPPGALDGSAQPRPPSSGAPGGNPHLWDVLYTVSAIITNTGNATSDEIPQLYVSLGGENEPVRVLRGFDRIENIAPGQSVRFTTDITRRDLSNWDVVSQNWVITDYEKTVYVGSSSRNLPLKATLK

SEQ ID NO: 175 - protein, 869 aa, Podospora anserina
MKFSVVVAAALASGALATPQYPPKLIKRDLAYSPPVYPSPWMNPEADGWAEAYVKAREFVSQMTLLEKVNLTTGTGWASEQCVGQVGAIPRLGLRSLCMHDAPLGIRGTDYNSAFPSGQTAAATWDRQLMYRRGYAIGKEAKGKGINVILGPVAGPLGRMPAAGRNWEGFSPDPVLTGVGMAETVKGHQDAGVIACAKHFIGNEQEHFRQVGEARGYGFNISETLSSNIDDKTMHELYLWPFADAVRAGAGSFMCSYQQVNNSYGCQNSKLMNGLLKDELGFQGFVLSDWQAQHTGAAAAAAGLDMSMPGDTEFNTGVSFWGTNLTVAVLNGTVPAYRIDDMAMRIMAAFFKVEKSIELDPINFSFWSLDTYGPIHWAAGEGHQQINYHVDVRADHANLIREIAAKGTVLLKNTGSLPLNKPKFVAVIGEDAGPNPNGPNSCADRGCNNGTLAMGWGSGTANFPYLITPDAALQAQAIKDGSRYESILTNYAASQTRALVSQDNVTAIVFVNADSGEGYINFEGNMGDRNNLTLWRGGDDLVKNVSSWCSNTIVVIHSTGPVLISEWYDSPNITAILWAGLPGQESGNSITDVLYGKVNPSGKSPFTWGATREGYGADVLYTPNNGEGAPQQDFSEGVFIDYRYFDKANTSVIYEFGHGLSYTTFEYSNIQVTKKNAGPYKPTTGQTAPAPTFGNFSTDLSDYLFPDEEFPYVYQYIYPYLNTTDPRNASGDPHFGQTAEEFMPPHAIDDSPQPLLPSSGKNSPGGNRALYDILYEVTADITNTGEIVGDEVVQLYVSLGGPDDPKVVLRDFGKLRIEPGQTAKFRGLLTRRDLSNWDVVSQDWVISEHTKTVFVGKSSRDLGLSAVLE

SEQ ID NO: 176 - protein, 302 aa, Penicillium simplicissimum
QASVSIDAKFKAHGKKYLGTIGDQYTLTKNTKNPAIIKADFGQLTPENSMKWDATEPNRGQFTFSGSDYLVNFAQSNGKLIRGHTLVWHSQLPGWVSSITDKNTLISVLKNHITTVMTRYKGKIYAWDVLNEIFNEDGSLRNSVFYNVIGEDYVRIAFETARSVDPNAKLYINDYNLDSAGYSKVNGMVSHVKKWLAAGIPIDGIGSQTHLGAGAGSAVAGALNALASAGTKEIAITELDIAGASSTDYVNVVNACLNQAKCVGITVWGVADPDSWRSSSSPLLFDGNYNPKAAYNAIANAL

SEQ ID NO: 177 - protein, 329 aa, Thermoascus aurantiacus
MVRPTILLTSLLLAPFAAASPILEERQAAQSVDQLIKARGKVYFGVATDQNRLTTGKNAAIIQADFGQVTPENSMKWDATEPSQGNFNFAGADYLVNWAQQNGKLIRGHTLVWHSQLPSWVSSITDKNTLTNVMKNHITTLMTRYKGKIRAWDVVNEAFNEDGSLRQTVFLNVIGEDYIPIAFQTARAADPNAKLYINDYNLDSASYPKTQAIVNRVKQWRAAGVPIDGIGSQTHLSAGQGAGVLQALPLLASAGTPEVAITELDVAGASPTDYVNVVNACLNVQSCVGITVWGVADPDSWRASTTPLLFDGNFNPKPAYNAIVQDLQQ

SEQ ID NO: 178 - protein, 713 aa, Trichoderma reesei
VVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA